(19) United States
(12) Patent Application Publication
(10) Pub. No.: US 2024/0059799 A1
(43) Pub. Date: Feb. 22, 2024

(54) ANTI-TL1A ANTIBODIES AND METHODS OF USE THEREOF

(71) Applicants: Pfizer Inc., New York, NY (US); Bristol-Myers Squibb Company, Princeton, NJ (US)

(72) Inventors: James Reasoner APGAR, Newton, MA (US); Laird BLOOM, Needham, MA (US); Fang JIN, Waban, MA (US); Fridrik KARLSSON, Wayland, MA (US); Kimberly Ann MARQUETTE, Somerville, MA (US); Jessica Haewon MIN DEBARTOLO, Waltham, MA (US); Nicole Melissa PICHE-NICHOLAS, Waltham, MA (US); Lioudmila TCHISTIAKOVA, Stoneham, MA (US)

(73) Assignees: Pfizer Inc., New York, NY (US); Bristol-Myers Squibb Company, Princeton, NJ (US)

(21) Appl. No.: 18/314,339

(22) Filed: May 9, 2023

Related U.S. Application Data
(60) Provisional application No. 63/364,559, filed on May 11, 2022.
(60) Provisional application No. 63/371,697, filed on Aug. 17, 2022.
(60) Provisional application No. 63/495,762, filed on Apr. 12, 2023.

(51) Int. Cl. C07K 16/46; A61P 37/02; C12N 15/63

(52) U.S. Cl. CPC C07K 16/468; A61P 37/02; C12N 15/63; C07K 2317/14; C07K 2317/55; C07K 2317/565; C07K 2317/567; C07K 2317/92

(57) ABSTRACT

The present invention discloses an isolated antibody that specifically binds to TL1A, comprising a heavy chain variable region (TL1A-VH) and a light chain variable region (TL1A-VL), comprising the CDR-H1, CDR-H2, and CDR-H3 sequences of a TL1A-VH sequence selected from the group consisting of SEQ ID NO: 42, SEQ ID NO: 44, SEQ ID NO: 47, SEQ ID NO: 49, SEQ ID NO: 52, SEQ ID NO: 54, SEQ ID NO: 57, SEQ ID NO: 70, SEQ ID NO: 72, SEQ ID NO: 78, SEQ ID NO: 80, SEQ ID NO: 89, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 110, SEQ ID NO: 120; and the CDR-L1, CDR-L2, and CDR-L3 sequences of SEQ ID NO: 18.

### CROSS-REFERENCE TO RELATED APPLICATIONS

This application claims the benefit of U.S. Provisional Application No. 63/364,559 filed on May 11, 2022, U.S. Provisional Application No. 63/371,697 filed on Aug. 17, 2022 and U.S. Provisional Application No. 63/495,762 filed on Apr. 12, 2023, the entire contents of each of which are incorporated herein by reference.

### FIELD

The present invention relates to antibodies that specifically bind to TL1A. The present invention further relates to antibodies that bind to TL1A and p40. The invention further relates to monovalent antibodies that specifically bind to TL1A. The present invention also pertains to related molecules, e.g. nucleic acids which encode such antibodies, compositions, and related methods, e.g., methods for producing and purifying such antibodies, and their use in diagnostics and therapeutics.

### INCORPORATION BY REFERENCE OF SEQUENCE LISTING

The sequence listing in an XML, named as 42065_Sequence_Listing.xml of 344 KB, created on May 4, 2023, and submitted to the United States Patent and Trademark Office via Patent Center, is incorporated herein by reference.

### BACKGROUND

Inflammatory bowel disease (IBD), which encompasses Crohn's disease and ulcerative colitis (UC), is a chronic inflammatory disorder affecting ˜1.6 million people in the USA, and ˜2.5-3 million people in Europe. To induce remission in patients with moderately to severely active UC, treatment recommendations include appropriate doses of oral corticosteroids, biologic therapies such as the tumor necrosis factor inhibitors (TNFi) infliximab (monotherapy or in combination with azathioprine), adalimumab, ustekinumab, golimumab, the integrin receptor antagonist vedolizumab, and the oral, small molecule Janus kinase inhibitor tofacitinib¹⁷. However, non-response and loss of response to treatments have been observed in UC and, therefore, the development of novel treatments for UC and IBD is still an unmet clinical need¹⁸.

A range of mucosal immune system components—including epithelial cells, innate and adaptive immune cells, cytokines and chemokines—contribute to the pathogenesis of IBD¹⁹. One of the immune components involved in the pathogenesis of IBD is TNF-like ligand 1A (TL1A, or tissue necrosis factor superfamily member 15 (TNFSF15)).

Tumor necrosis factor (TNF)-like ligand 1A (TL1A) is a member of the TNF family of cytokines also known as TNFSF15. TL1A is the only known ligand for its receptor Death Receptor 3 (DR3) also known as TNFRSF25. TL1A expression on antigen presenting cells (monocytes, macrophages, dendritic cells) and DR3 expression on effector cells (T cells, NK and NKT cells) is highly dependent on pro-inflammatory conditions²⁹⁻²². In vivo and in vitro evidence support a co-stimulatory role for the TL1A/DR3 pathway on T cells and in enhancing effector cell functions, inflammatory cell expansion and cytokine secretion. Further, this pathway has been implicated in the regulation of pathogenic Th1, Th2, and Th17 T-helper responses, and of NK and NK-T cell responses, in immune-mediated diseases^{21,23-26}.

Studies of DR3 or TL1A gene-deficient mice or mice treated with anti-TL1A antibodies demonstrate a role for this pathway in a number of autoimmune disease models, such as IBD, asthma, multiple sclerosis, and arthritis^{25, 27-28}.

Moreover, significant literature from studies involving nonclinical species and humans implicates TL1A most prominently in the pathophysiology of inflammatory bowel disease (IBD), such as, ulcerative colitis (UC) and Crohn's Disease (CD). That is, numerous genome-wide association studies have linked several polymorphisms of the TL1A gene to UC and CD in patient populations of Japanese, European, and Asian origin²⁹⁻³³.

Additionally, human inflamed IBD tissues show high levels of TL1A and DR3 expression and several independent laboratories have demonstrated that antibody blockade of TL1A prevents or attenuates established gut inflammation in a number of murine IBD models^{22, 24, 26, 34-39}.

Although the exact cause of IBD, e.g., CD and UC, remains unclear, inhibition of pro-inflammatory cytokines and adhesion molecules have been shown to provide some therapeutic benefit. However, despite current medical therapy, most CD patients may ultimately require surgery, and, over time, repeated resections can result in short gut syndrome, ultimately committing the patient to life-long parenteral nutrition and its associated complications. Thus, there is a long-felt unmet need for more robust therapies for CD patients. Further, there is a long-felt unmet need for novel therapeutics to treat or ameliorate IBD, including UC and CD, as well as to treat other TL1A-mediated diseases and conditions. The present invention meets these needs.

IL-12 and IL-23 are implicated in type 1 and type 3 (Th17) responses, respectively (77). IL-12 drives T helper 1 (Th1) cell differentiation and interferon-γ (IFN-γ) production, whereas IL-23 promotes the maintenance of Th17 cells that produce IL-17 and other type 3 cytokines. Type 1 and type 3 responses have been implicated in a range of human inflammatory and autoimmune diseases. A causal role for IL-12p40-containing cytokines has been established through numerous drug approvals (75) (IL-12p40 is hereinafter referred to simply as p40). The p40 neutralizing agent Stelara® (ustekinumab; Janssen) neutralizes both IL-12 and IL-23, and is approved for the treatment of plaque psoriasis, psoriatic arthritis, Crohn's disease, and ulcerative colitis. The IL-23-selective anti-IL-23p19 blocking agents Tremfya® (guselkumab; Janssen), Skyrizi® (risankizumab; Boehringer Ingelheim/AbbVie), and Ilumya® (tildrakizumab; Sun Pharmaceutical) are approved for a range of psoriatic disorders.

Despite the effectiveness of current treatments, an unmet need remains for safe and effective therapeutics for numerous diseases characterized by inflammatory responses, that address a broad range of pathogenic mechanisms.

### SUMMARY

Provided herein are antibodies (including antigen-binding fragments thereof) that specifically bind to TL1A, including for example without limitation, bispecific TL1A-p40 antibodies, monovalent TL1A antibodies thereof, other related antibodies, related nucleic acids, uses, and associated methods thereof. The disclosure also provides processes for making, preparing, and producing antibodies disclosed herein, including antibodies that bind to one or both of TL1A and p40. Antibodies of the disclosure are useful in one or more of diagnosis, prophylaxis, or treatment of disorders or conditions mediated by, or associated with one or more of TL1A and TL1A/p40 activity, including, but not limited to in the manufacture of a medicament for treating a disease, disorder or condition mediated by TL1A. In some embodiments, the disease, disorder or condition is at least one selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis.

The disclosure further encompasses expression of antibodies, and preparation and manufacture of compositions comprising antibodies of the disclosure, such as medicaments for the use of the antibodies.

Polynucleotides encoding antibodies that specifically bind to TL1A, as well as bispecific TL1A-p40 antibodies, monovalent TL1A antibodies thereof, other related antibodies, are also provided. Polynucleotides encoding antibody heavy chains or light chains, or both are also provided. Host cells that express the antibodies are provided. Methods of treatment using the antibodies are provided. Such methods include, but are not limited to, one or more of methods of treating or methods of preventing diseases associated with or mediated by either or both of TL1A, and TL1A and p40 expression, or one or more diseases selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis.

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting. Those skilled in the art will recognize or be able to ascertain using no more than routine experimentation, many equivalents to the specific embodiments of the invention described herein. Such equivalents are intended to be encompassed by the following embodiments (E).

E1. An isolated antibody that specifically binds to TL1A, comprising a heavy chain variable region (TL1A-VH) and a light chain variable region (TL1A-VL), comprising the CDR-H1, CDR-H2, and CDR-H3 sequences of a TL1A-VH sequence selected from the group consisting of SEQ ID NO: 42, SEQ ID NO: 44, SEQ ID NO: 47, SEQ ID NO:49, SEQ ID NO:52, SEQ ID NO: 54, SEQ ID NO: 57, SEQ ID NO: 70, SEQ ID NO: 72, SEQ ID NO: 78, SEQ ID NO: 80, SEQ ID NO: 89, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 110, SEQ ID NO: 120; and the CDR-L1, CDR-L2, and CDR-L3 sequences of SEQ ID NO: 18.
E2. An isolated antibody that specifically binds to TL1A, comprising a heavy chain variable region (TL1A-VH) and a light chain variable region (TL1A-VL), comprising the CDR-H1, CDR-H2, and CDR-H3 sequences according to SEQ ID NO: 42, and the CDR-L1, CDR-L2, and CDR-L3 sequences according to SEQ ID NO: 18.
E3. An isolated antibody that specifically binds to TL1A, comprising a heavy chain variable region (TL1A-VH) and a light chain variable region (TL1A-VL), comprising a CDR-L1 sequence according to SEQ ID NO: 11; a CDR-L2 sequence according to SEQ ID NO: 12, and a CDR-L3 sequence according to SEQ ID NO: 13, and comprising a set of heavy chain CDRs selected from the list:
(i) a CDR-H1 sequence according to SEQ ID NO: 30; a CDR-H2 sequence according to SEQ ID NO: 41; a CDR-H3 sequence according to SEQ ID NO: 32;
(ii) a CDR-H1 sequence according to SEQ ID NO: 30; a CDR-H2 sequence according to SEQ ID NO: 41; a CDR-H3 sequence according to SEQ ID NO: 35;
(iii) a CDR-H1 sequence according to SEQ ID NO: 30; a CDR-H2 sequence according to SEQ ID NO: 46; a CDR-H3 sequence according to SEQ ID NO: 32;
(iv) a CDR-H1 sequence according to SEQ ID NO: 30; a CDR-H2 sequence according to SEQ ID NO: 46; a CDR-H3 sequence according to SEQ ID NO: 35;
(v) a CDR-H1 sequence according to SEQ ID NO: 30; a CDR-H2 sequence according to SEQ ID NO: 51; a CDR-H3 sequence according to SEQ ID NO: 32;
(vi) a CDR-H1 sequence according to SEQ ID NO: 30; a CDR-H2 sequence according to SEQ ID NO: 51; a CDR-H3 sequence according to SEQ ID NO: 35;
(vii) a CDR-H1 sequence according to SEQ ID NO: 30; a CDR-H2 sequence according to SEQ ID NO: 56; a CDR-H3 sequence according to SEQ ID NO: 32;
(viii) a CDR-H1 sequence according to SEQ ID NO: 30; a CDR-H2 sequence according to SEQ ID NO: 56; a CDR-H3 sequence according to SEQ ID NO: 35;
(ix) a CDR-H1 sequence according to SEQ ID NO: 61; a CDR-H2 sequence according to SEQ ID NO: 41; a CDR-H3 sequence according to SEQ ID NO: 32;
(x) a CDR-H1 sequence according to SEQ ID NO: 61; a CDR-H2 sequence according to SEQ ID NO: 41; a CDR-H3 sequence according to SEQ ID NO: 35;
(xi) a CDR-H1 sequence according to SEQ ID NO: 61; a CDR-H2 sequence according to SEQ ID NO: 51; a CDR-H3 sequence according to SEQ ID NO: 32;
(xii) a CDR-H1 sequence according to SEQ ID NO: 61; a CDR-H2 sequence according to SEQ ID NO: 56; a CDR-H3 sequence according to SEQ ID NO: 32;
(xiii) a CDR-H1 sequence according to SEQ ID NO: 86; a CDR-H2 sequence according to SEQ ID NO: 31; a CDR-H3 sequence according to SEQ ID NO: 35;
(xiv) a CDR-H1 sequence according to SEQ ID NO: 86; a CDR-H2 sequence according to SEQ ID NO: 38; a CDR-H3 sequence according to SEQ ID NO: 35;
(xv) a CDR-H1 sequence according to SEQ ID NO: 86; a CDR-H2 sequence according to SEQ ID NO: 41; a CDR-H3 sequence according to SEQ ID NO: 32;
(xvi) a CDR-H1 sequence according to SEQ ID NO: 86; a CDR-H2 sequence according to SEQ ID NO: 41; a CDR-H3 sequence according to SEQ ID NO: 35;
(xvii) a CDR-H1 sequence according to SEQ ID NO: 86; a CDR-H2 sequence according to SEQ ID NO: 46; a CDR-H3 sequence according to SEQ ID NO: 35;
(xviii) a CDR-H1 sequence according to SEQ ID NO: 86; a CDR-H2 sequence according to SEQ ID NO: 51; a CDR-H3 sequence according to SEQ ID NO: 32;
(xix) a CDR-H1 sequence according to SEQ ID NO: 86; a CDR-H2 sequence according to SEQ ID NO: 51; a CDR-H3 sequence according to SEQ ID NO: 35;
(xx) a CDR-H1 sequence according to SEQ ID NO: 86; a CDR-H2 sequence according to SEQ ID NO: 56; a CDR-H3 sequence according to SEQ ID NO: 32;
(xxi) a CDR-H1 sequence according to SEQ ID NO: 109; a CDR-H2 sequence according to SEQ ID NO: 41; a CDR-H3 sequence according to SEQ ID NO: 35; or
(xxii) a CDR-H1 sequence according to SEQ ID NO: 109; a CDR-H2 sequence according to SEQ ID NO: 56; a CDR-H3 sequence according to SEQ ID NO: 32.
   E4. An isolated antibody that specifically binds to TL1A, comprising a heavy chain variable region (TL1A-VH) and a light chain variable region (TL1A-VL), wherein a CDR-L1 sequence according to SEQ ID NO: 11; a CDR-L2 sequence according to SEQ ID NO: 12, and a CDR-L3 sequence according to SEQ ID NO: 13, and wherein a CDR-H1 sequence according to SEQ ID NO: 30; a CDR-H2 sequence according to SEQ ID NO: 41; a CDR-H3 sequence according to SEQ ID NO: 32.
   E5. The antibody of any one of E1-E4, comprising a TL1A-VH framework sequence derived from a human germline VH sequence selected from the group consisting of IGHV1-18*01, IGHV1-46*01, IGHV3-7*01, IGHV1-2*02.
   E6. The antibody of any one of E1-E5, comprising a TL1A-VH framework sequence derived from a human IGHV1-18*01 germline sequence.
   E7. The antibody of any one of E1-E6, comprising a TL1A-VL framework sequence derived from a human germline VL sequence selected from the group consisting of IGKV1-39*01, IGKV3-11*01, IGKV3D-7*01, IGKV3-11*01.
   E8. The antibody of any one of E1-E7, comprising a TL1A-VL framework sequence derived from a human germline IGKV1-39*01, sequence.
   E9. The antibody of any one of E1-E8, comprising a TL1A-VL framework sequence and a TL1A-VH framework sequence wherein one or both of the TL1A-VL framework sequence and the TL1A-VH framework sequence is at least 90%, 91%, 92%, 93%, 94%, 95%, 96%, 97%, 98%, 99%, or 100% identical to the human germline sequence from which it was derived.
   E10. The antibody of any one of E1-E9, comprising a TL1A-VL framework sequence and a TL1A-VH framework sequence, and wherein one or both of the TL1A-VL framework sequence or the TL1A-VH framework sequence is identical to the human germline sequence from which it was derived.
   E11. The antibody of any one of E1-E10, wherein the TL1A-VL comprises an amino acid sequence according to SEQ ID NO: 18, and wherein the TL1A-VH comprises an amino acid sequence according to a sequence selected from the group consisting of SEQ ID NO: 42, SEQ ID NO: 44, SEQ ID NO: 47, SEQ ID NO:49, SEQ ID NO:52, SEQ ID NO: 54, SEQ ID NO: 57, SEQ ID NO: 70, SEQ ID NO: 72, SEQ ID NO: 78, SEQ ID NO: 80, SEQ ID NO: 89, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 110, and SEQ ID NO:120.
   E12. The antibody of any one of E1-E1, comprising a TL1A-VH sequence at least 90%, 91%, 92%, 93%, 94%, 95%, 96%, 97%, 98%, 99%, or 100% identical to SEQ ID NO: 42, and comprising a TL1A-VL sequence at least 90%, 91%, 92%, 93%, 94%, 95%, 96%, 97%, 98%, 99%, or 100% identical to SEQ ID NO: 18.
   E13. The antibody of any one of E1-E12, comprising the TL1A-VH sequence of SEQ ID NO: 42, and the TL1A-VL sequence of SEQ ID NO: 18.
   E14. The antibody of any one of E1-E13, comprising a TL1A-VH sequence encoded by a nucleic acid sequence of SEQ ID NO: 228.
   E15. The antibody of any one of E1-E14, comprising a TL1A-VL sequence encoded by a nucleic acid sequence of SEQ ID NO: 229.
   E16. The antibody of any one of E1-E15, comprising a TL1A-VH sequence encoded by the plasmid deposited at the ATCC and having ATCC Accession No. PTA-127347
   E17. The antibody of any one of E1-E16, comprising a TL1A-VL sequence encoded by the plasmid deposited at the ATCC and having ATCC Accession No. PTA-127348
   E18. An antibody comprising a TL1A-VH sequence encoded by the plasmid deposited at the ATCC and having ATCC Accession No. PTA-127347 and a TL1A-VL sequence encoded by the plasmid deposited at the ATCC and having ATCC Accession No. PTA-127438.
   E19. The antibody of E1-E18, wherein the TL1A IC₅₀ is less than 20 nM in a competition ELISA.
   E20. The antibody of E1-E19, wherein the TL1A IC₅₀ is less than 10 nM in a competition ELISA.
   E21. The antibody of E1-E20, wherein the TL1A IC₅₀ is less than 50 nM in a TF-1 NFκB reporter cell bioassay.
   E22. The antibody of E1-E21, wherein the TL1A IC₅₀ is less than 30 nM in a TF-1 NFκB reporter cell bioassay.
   E23. The antibody of E1-E22, wherein the TL1A IC₅₀ is less than 1 nM in a whole blood IFNγ assay.
   E24. The antibody of E1-E23, wherein the TL1A IC₅₀ is less than 30 nM in a TF-1 NFκB reporter cell bioassay.
   E25. The antibody of E1-E24, further comprising a constant heavy domain (TL1A-CH1) and a constant light domain (TL1A-CL).
   E26. The antibody of E25, wherein the TL1A-CH1 comprises a sequence selected from the group consisting of SEQ ID NO: 6, SEQ ID NO: 195, and SEQ ID NO: 199.
   E27. The antibody of E25-E26, wherein the TL1A-CH1 comprises a sequence according to SEQ ID NO: 6.
   E28. The antibody of E25-E27, wherein the TL1A-CL comprises a sequence selected from the group consisting of SEQ ID NO: 16, and SEQ ID NO: 201.
   E29. The antibody of E25-E28, wherein the TL1A-CL comprises a sequence according to SEQ ID NO:16.
   E30. The antibody of E25-E29, wherein the TL1A-CH1 is connected to the TL1A-VH, and the TL1A-CL is connected to the TL1A-VL forming a TL1A-binding Fab domain (TL1A-Fab).
   E31. The antibody of any one of E1-E30, comprising an antibody Fc domain comprising a first Fc chain and a second Fc chain.
   E32. The antibody of E31, wherein the Fc domain is the Fc domain of an IgA (for example IgA₁ or IgA₂), IgD, IgE, IgM, or IgG (for example IgG₁, IgG₂, IgG₃, or IgG₄).
   E33. The antibody of E32, wherein the Fc domain is the Fc domain of an IgG₁.
   E34. The antibody of E31-E34, wherein the first Fc chain comprises, from N-terminus to C-terminus: a first hinge region, a first CH2 region, and a first CH3 region, and the second Fc chain comprises, from N-terminus to C-terminus: a second hinge region, a second CH2 region, and a second CH3 region.
   E35. The antibody of E33, comprising at least a first TL1A-CH1 domain, wherein the N-terminus of the first Fc chain is connected to the C-terminus of the first TL1A-CH1 domain.
   E36. The antibody of E35-E37, wherein the first CH2 region and the second CH2 region each comprise a sequence according to SEQ ID NO: 8.
   E37. The antibody of E35, wherein either or both of the first hinge region comprises a sequence selected from the group consisting of SEQ ID NO: 7, SEQ ID NO: 157, SEQ ID NO: 179, SEQ ID NO: 182, and SEQ ID NO: 189, SEQ ID NO: 191, SEQ ID NO: 204, and SEQ ID NO: 206.
   E38. The antibody of E44, wherein the hinge region comprises a sequence according to SEQ ID NO: 7.
   E39. The antibody of E35-E38, wherein the CH3 region on the first Fc chain and the CH3 region on the second Fc chain each comprise a sequence according to SEQ ID NO: 9.
   E40. The antibody of any one of E35-E39, comprising a light chain (TL1A-LC) comprising the sequence according to SEQ ID NO: 19, and a heavy chain (TL1A-HC) comprising a sequence according to a sequence selected from the group consisting of SEQ ID NO: 43, SEQ ID NO: 45, SEQ ID NO: 48, SEQ ID NO: 50, SEQ ID NO: 53, SEQ ID NO: 55, SEQ ID NO: 58, SEQ ID NO: 71, SEQ ID NO: 73, SEQ ID NO: 79, SEQ ID NO: 81, SEQ ID NO: 90, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 111, and SEQ ID NO:121.
   E41. The antibody of any one of E35-E40, comprising a light chain (TL1A-LC) comprising the sequence according to SEQ ID NO: 19, and a heavy chain (TL1A-HC) comprising a sequence according to SEQ ID NO: 43.
   E42. The antibody of any one of E34-E41, comprising two identical TL1A-Fab domains.
   E43. The antibody as any one of E34-E37, comprising a single TL1A-Fab domain.
   E44. The antibody of E43, wherein the first Fc chain and the second Fc chain each contain one or more amino acid modifications that promote the association of the first Fc chain with the second Fc chain.
   E45. The antibody of E44, wherein the first Fc chain comprises a first CH3 region, and the second Fc chain comprises a second CH3 region, and the first CH3 region and the second CH3 region each comprise a different and complementary sequence, and the different and complementary sequences are selected from one of the following pairs of different and complementary sequences:
(i) SEQ ID NO: 160 and SEQ ID NO: 162;
(ii) SEQ ID NO: 180, and SEQ ID NO: 183;
(iii) SEQ ID NO: 155 and SEQ ID NO: 158; and
(iv) SEQ ID NO: 185 and SEQ ID NO: SEQ ID NO: 187
   E46. The antibody of E45, wherein the first CH3 region and the second CH3 region comprise SEQ ID NO: 160 and SEQ ID NO: 162.
   E47. The antibody of E44-E45, wherein the first hinge region and the second hinge region each contain one or more amino acid modifications that promote the association of the first hinge region with the second hinge region.
   E48. The antibody of E47, wherein the first hinge region and the second hinge region each comprise a different and complementary sequence, and the different and complementary sequences are selected from one of the following pairs of different and complementary sequences:
(i) SEQ ID NO: 179 and SEQ ID NO: 182; and
(ii) SEQ ID NO: 189 and SEQ ID NO: 191.
   E49. The antibody of any one of E1-E48, characterized by a score of less than 4 in an affinity-capture self-interaction nanoparticle spectroscopy (AC SINS) assay.
   E50. The antibody of E43-E49, wherein the TL1A-Fab domain is fused to the first Fc chain, and the second Fc chain is not fused to Fab domain.
   E51. The antibody of E43-E50, comprising a first heavy chain and a first light chain, and a second heavy chain, wherein the first heavy chain and the first light chain comprise a TL1A binding site that binds to TL1A, wherein the first antibody heavy chain comprises an amino acid sequence selected from the group consisting of SEQ ID NO: 161, SEQ ID NO: 164, and SEQ ID NO: 165, the first antibody light chain comprises the amino acid sequence of SEQ ID NO: 19, and the second antibody heavy chain comprises the amino acid sequence of SEQ ID NO: 163.
   E52. The antibody of E43-E51, comprising a first heavy chain and a first light chain, and a second heavy chain, wherein the first heavy chain and the first light chain comprise a TL1A binding site that binds to TL1A, wherein the first antibody heavy chain comprises the amino acid sequence of SEQ ID NO: 161, the first antibody light chain comprises the amino acid sequence of SEQ ID NO: 19, and the second antibody heavy chain comprises the amino acid sequence of SEQ ID NO: 163.
   E52. The antibody of E43-E51, comprising a first heavy chain and a first light chain, and a second heavy chain, wherein the first heavy chain and the first light chain comprise a TL1A binding site that binds to TL1A, wherein the first antibody heavy chain comprises the amino acid sequence of SEQ ID NO: 164, the first antibody light chain comprises the amino acid sequence of SEQ ID NO: 19, and the second antibody heavy chain comprises the amino acid sequence of SEQ ID NO: 163.
   E54. The antibody of E43-E51, comprising a first heavy chain and a first light chain, and a second heavy chain, wherein the first heavy chain and the first light chain comprise a TL1A binding site that binds to TL1A, wherein the first antibody heavy chain comprises the amino acid sequence of SEQ ID NO: 165, the first antibody light chain comprises the amino acid sequence of SEQ ID NO: 19, and the second antibody heavy chain comprises the amino acid sequence of SEQ ID NO: 163.
   E55. The antibody of E43-E49, comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target, (xTL1A-Fab), and wherein the first and second light chain are identical.
   E56. The antibody of E55, wherein the second Fab domain comprises a non-binding TL1A heavy chain variable region (xTL1A-VH) and a non-binding TL1A light chain variable region (xTL1A-VL), wherein a CDR-L1 sequence according to SEQ ID NO: 11; a CDR-L2 sequence according to SEQ ID NO: 12, and a CDR-L3 sequence according to SEQ ID NO: 13, and wherein
(i) a CDR-H1 sequence according to SEQ ID NO: 139; a CDR-H2 sequence according to SEQ ID NO: 140; a CDR-H3 sequence according to SEQ ID NO: 133;
(ii) a CDR-H1 sequence according to SEQ ID NO: 128; a CDR-H2 sequence according to SEQ ID NO: 129; a CDR-H3 sequence according to SEQ ID NO: 130;
(iii) a CDR-H1 sequence according to SEQ ID NO: 128; a CDR-H2 sequence according to SEQ ID NO: 129; a CDR-H3 sequence according to SEQ ID NO: 133;
(iv) a CDR-H1 sequence according to SEQ ID NO: 128; a CDR-H2 sequence according to SEQ ID NO: 129; a CDR-H3 sequence according to SEQ ID NO: 136;
(v) a CDR-H1 sequence according to SEQ ID NO: 139; a CDR-H2 sequence according to SEQ ID NO: 140; a CDR-H3 sequence according to SEQ ID NO: 130;
(vi) a CDR-H1 sequence according to SEQ ID NO: 139; a CDR-H2 sequence according to SEQ ID NO: 140; a CDR-H3 sequence according to SEQ ID NO: 136;
(vii) a CDR-H1 sequence according to SEQ ID NO: 147; a CDR-H2 sequence according to SEQ ID NO: 148; a CDR-H3 sequence according to SEQ ID NO: 130;
(viii) a CDR-H1 sequence according to SEQ ID NO: 147; a CDR-H2 sequence according to SEQ ID NO: 148; a CDR-H3 sequence according to SEQ ID NO: 133; or
(ix) a CDR-H1 sequence according to SEQ ID NO: 147; a CDR-H2 sequence according to SEQ ID NO: 148; a CDR-H3 sequence according to SEQ ID NO: 136.
   E57. The antibody of E55-E56, wherein the second Fab domain comprises a heavy chain variable region (xTL1A-VH) and a light chain variable region (xTL1A-VL), wherein a CDR-L1 sequence according to SEQ ID NO: 11; a CDR-L2 sequence according to SEQ ID NO: 12, and a CDR-L3 sequence according to SEQ ID NO: 13, and wherein a CDR-H1 sequence according to SEQ ID NO: 139; a CDR-H2 sequence according to SEQ ID NO: 140; a CDR-H3 sequence according to SEQ ID NO: 133.
   E58. The antibody of any one of E55-E56, wherein the second Fab domain comprises a light chain variable amino acid sequence according to SEQ ID NO: 18, and wherein the second Fab domain comprises a heavy chain variable amino acid sequence according to a sequence selected from the group consisting of SEQ ID NO: 143, SEQ ID NO: 131, SEQ ID NO: 134, SEQ ID NO: 137, SEQ ID NO: 141, SEQ ID NO: 145, SEQ ID NO: 57, SEQ ID NO: 70, SEQ ID NO: 72, SEQ ID NO: 78, SEQ ID NO: 80, SEQ ID NO: 89, SEQ ID NO: 149, SEQ ID NO: 151, and SEQ ID NO: 153.
   E59. The antibody of E58, wherein the second Fab domain comprises a light chain variable amino acid sequence according to SEQ ID NO: 18, and wherein the second Fab domain comprises a heavy chain variable amino acid sequence according to SEQ ID NO: 143.
   E60. The antibody of E55-E59, comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target (xTL1A-Fab), wherein the first antibody heavy chain comprises an amino acid sequence according to SEQ ID NO: 161, the first antibody light chain comprises an amino acid sequence according to SEQ ID NO: 19, the second antibody light chain comprises an amino acid sequence according to SEQ ID NO: 19, and the second antibody heavy chain comprises an amino acid sequence according to a sequence selected from the group consisting of SEQ ID NO: 166.
   E61. The antibody of E55-E60, comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target (xTL1A-Fab), wherein the first antibody heavy chain comprises an amino acid sequence according to SEQ ID NO: 161, the first antibody light chain comprises an amino acid sequence according to SEQ ID NO: 19, the second antibody light chain comprises an amino acid sequence according to SEQ ID NO: 19, and the second antibody heavy chain comprises an amino acid sequence according to SEQ ID NO: 166.
   E62. The antibody of any one of E1-E61, wherein the antibody has a terminal half-life of less than 7 days in cynomolgus monkeys.
   E63. The antibody of any one of E1-E63, characterized by a score of less than 2% high molecular mass species when determined by analytical size-exclusion chromatography (aSEC).
   E64. The antibody of any one of E1-E63, characterized by a score of less than 7 in an affinity-capture self-interaction nanoparticle spectroscopy (AC SINS) assay.
   E65. The antibody of any one of E1-E64, characterized by a score of less than 6 in an affinity-capture self-interaction nanoparticle spectroscopy (AC SINS) assay.
   E66. The antibody of any one of E1-E65, characterized by a score of less than 5 in an affinity-capture self-interaction nanoparticle spectroscopy (AC SINS) assay.
   E67. The antibody of any one of E1-E66, characterized by a score of less than 4 in an affinity-capture self-interaction nanoparticle spectroscopy (AC SINS) assay.
   E68. The antibody of any one of E1-E67, characterized by a score of less than 3 in an affinity-capture self-interaction nanoparticle spectroscopy (AC SINS) assay.
   E69. The antibody of any one of E1-E68, characterized by IC50 of less than 2 nM in an NFκB TL1A neutralization bioassay.
   E70. The antibody of any one of E1-E69, characterized by a KD of less than 500 pM as measured by SPR.
   E71. The antibody of any one of E1-E70, characterized by a KD of less than 320 pM as measured by SPR.
   E72. The antibody of any one of E1-E71, characterized by a KD of less than 300 pM as measured by SPR.
   E73. The antibody of any one of E1-E72, wherein the antibody has a terminal half-life of at least 21 days in cynomolgus monkeys.
   E74. The antibody of any one of E1-E73, wherein the antibody has a terminal half-life of at least 25 days in cynomolgus monkeys.
   E75. The antibody of any one of E1-E74, wherein the antibody has a terminal half-life of at least 28 days in cynomolgus monkeys.
   E76. The antibody of any one of E1-E75, wherein the antibody binds cynomologus TL1A within one order of magnitude of human TL1A as measured by SPR.
   E77. The antibody of any one of E1-E76, wherein antibody has a binding affinity at least 500× higher for human TL1A than for mouse T1A as measured by SPR.
   E78. The antibody of any one of E1-E77, wherein the antibody has a binding affinity at least 2000× higher for human TL1A than for mouse TL1A as measured by SPR.
   E79. The antibody of E1-E38, wherein the antibody specifically binds to p40 through a p40 binding domain, comprising a heavy chain variable region (p40-VH) and a light chain variable region (p40-VL), comprising the CDR-H1, CDR-H2, and CDR-H3 sequences of a p40-VH sequence according to SEQ ID NO: 171, and the CDR-L1, CDR-L2, and CDR-L3 sequences of a p40-VL sequence according to SEQ ID NO: 177.
   E80. The antibody of E1-E38, wherein the antibody specifically binds to p40 through a p40 binding domain, comprising a p40-binding heavy chain variable region (p40-VH) and a p40-binding light chain variable region (p40-VL), wherein a CDR-L1 sequence according to SEQ ID NO: 173; a CDR-L2 sequence according to SEQ ID NO: 174, and a CDR-L3 sequence according to SEQ ID NO: 175, and wherein a CDR-H1 sequence according to SEQ ID NO: 167; a CDR-H2 sequence according to SEQ ID NO: 168; a CDR-H3 sequence according to SEQ ID NO: 169.
   E81. An isolated antibody comprising a first antigen binding site that binds to TL1A and a second antigen binding site that binds to p40, wherein the first antigen binding site comprises a TL1A-binding heavy chain variable region (TL1A-VH) and a TL1A-binding light chain variable region (TL1A-VL), wherein the second antigen binding site comprises a p40-binding heavy chain variable region (p40-VH) and a p40-binding light chain variable region (p40-VL), and wherein the p40 binding domain comprises a CDR-L1 sequence according to SEQ ID NO:
   173; a CDR-L2 sequence according to SEQ ID NO: 174, and a CDR-L3 sequence according to SEQ ID NO: 175, a CDR-H1 sequence according to SEQ ID NO: 167, a CDR-H2 sequence according to SEQ ID NO: 168, and a CDR-H3 sequence according to SEQ ID NO: 169, and wherein the TL1A binding domain comprises a CDR-L1 sequence according to SEQ ID NO: 11; a CDR-L2 sequence according to SEQ ID NO: 12, and a CDR-L3 sequence according to SEQ ID NO: 13, and further comprises a set of heavy chain CDRs selected from the list:
(i) a CDR-H1 sequence according to SEQ ID NO: 30; a CDR-H2 sequence according to SEQ ID NO: 41; a CDR-H3 sequence according to SEQ ID NO: 32;
(ii) a CDR-H1 sequence according to SEQ ID NO: 30; a CDR-H2 sequence according to SEQ ID NO: 41; a CDR-H3 sequence according to SEQ ID NO: 35;
(iii) a CDR-H1 sequence according to SEQ ID NO: 30; a CDR-H2 sequence according to SEQ ID NO: 46; a CDR-H3 sequence according to SEQ ID NO: 32;
(iv) a CDR-H1 sequence according to SEQ ID NO: 30; a CDR-H2 sequence according to SEQ ID NO: 46; a CDR-H3 sequence according to SEQ ID NO: 35;
(v) a CDR-H1 sequence according to SEQ ID NO: 30; a CDR-H2 sequence according to SEQ ID NO: 51; a CDR-H3 sequence according to SEQ ID NO: 32;
(vi) a CDR-H1 sequence according to SEQ ID NO: 30; a CDR-H2 sequence according to SEQ ID NO: 51; a CDR-H3 sequence according to SEQ ID NO: 35;
(vii) a CDR-H1 sequence according to SEQ ID NO: 30; a CDR-H2 sequence according to SEQ ID NO: 56; a CDR-H3 sequence according to SEQ ID NO: 32;
(viii) a CDR-H1 sequence according to SEQ ID NO: 30; a CDR-H2 sequence according to SEQ ID NO: 56; a CDR-H3 sequence according to SEQ ID NO: 35;
(ix) a CDR-H1 sequence according to SEQ ID NO: 61; a CDR-H2 sequence according to SEQ ID NO: 41; a CDR-H3 sequence according to SEQ ID NO: 32;
(x) a CDR-H1 sequence according to SEQ ID NO: 61; a CDR-H2 sequence according to SEQ ID NO: 41; a CDR-H3 sequence according to SEQ ID NO: 35;
(xi) a CDR-H1 sequence according to SEQ ID NO: 61; a CDR-H2 sequence according to SEQ ID NO: 51; a CDR-H3 sequence according to SEQ ID NO: 32;
(xii) a CDR-H1 sequence according to SEQ ID NO: 61; a CDR-H2 sequence according to SEQ ID NO: 56; a CDR-H3 sequence according to SEQ ID NO: 32;
(xiii) a CDR-H1 sequence according to SEQ ID NO: 86; a CDR-H2 sequence according to SEQ ID NO: 31; a CDR-H3 sequence according to SEQ ID NO: 35;
(xiv) a CDR-H1 sequence according to SEQ ID NO: 86; a CDR-H2 sequence according to SEQ ID NO: 38; a CDR-H3 sequence according to SEQ ID NO: 35;
(xv) a CDR-H1 sequence according to SEQ ID NO: 86; a CDR-H2 sequence according to SEQ ID NO: 41; a CDR-H3 sequence according to SEQ ID NO: 32;
(xvi) a CDR-H1 sequence according to SEQ ID NO: 86; a CDR-H2 sequence according to SEQ ID NO: 41; a CDR-H3 sequence according to SEQ ID NO: 35;
(xvii) a CDR-H1 sequence according to SEQ ID NO: 86; a CDR-H2 sequence according to SEQ ID NO: 46; a CDR-H3 sequence according to SEQ ID NO: 35;
(xviii) a CDR-H1 sequence according to SEQ ID NO: 86; a CDR-H2 sequence according to SEQ ID NO: 51; a CDR-H3 sequence according to SEQ ID NO: 32;
(xix) a CDR-H1 sequence according to SEQ ID NO: 86; a CDR-H2 sequence according to SEQ ID NO: 51; a CDR-H3 sequence according to SEQ ID NO: 35;
(xx) a CDR-H1 sequence according to SEQ ID NO: 86; a CDR-H2 sequence according to SEQ ID NO: 56; a CDR-H3 sequence according to SEQ ID NO: 32;
(xxi) a CDR-H1 sequence according to SEQ ID NO: 109; a CDR-H2 sequence according to SEQ ID NO: 41; a CDR-H3 sequence according to SEQ ID NO: 35; and
(xxii) a CDR-H1 sequence according to SEQ ID NO: 109; a CDR-H2 sequence according to SEQ ID NO: 56; a CDR-H3 sequence according to SEQ ID NO: 32.
   E82. An isolated antibody comprising a first antigen binding site that binds to TL1A and a second antigen binding site that binds to p40, wherein the first antigen binding site comprises a TL1A-binding heavy chain variable region (TL1A-VH) and a TL1A-binding light chain variable region (TL1A-VL), wherein the second antigen binding site comprises a p40-binding heavy chain variable region (p40-VH) and a p40-binding light chain variable region (p40-VL), and wherein
(i) the p40 binding domain comprises a CDR-L1 sequence according to SEQ ID NO: 173; a CDR-L2 sequence according to SEQ ID NO: 174, and a CDR-L3 sequence according to SEQ ID NO: 175, a CDR-H1 sequence according to SEQ ID NO: 167, a CDR-H2 sequence according to SEQ ID NO: 168, and a CDR-H3 sequence according to SEQ ID NO: 169, and
(ii) the TL1A binding domain comprises a CDR-L1 sequence according to SEQ ID NO: 11; a CDR-L2 sequence according to SEQ ID NO: 12, and a CDR-L3 sequence according to SEQ ID NO: 13, a CDR-H1 sequence according to SEQ ID NO: 30; a CDR-H2 sequence according to SEQ ID NO: 41; and a CDR-H3 sequence according to SEQ ID NO: 32.
   E83. The antibody of any one of E79-E82, comprising a TL1A-VH framework sequence derived from a human germline VH sequence selected from the group consisting of IGHV1-18*01, IGHV1-18*01, IGHV1-18*01, IGHV1-46*01, IGHV3-7*01, IGHV1-2*02, IGHV1-46*01, IGHV3-7*01, IGHV1-2*02, IGHV1-46*01, IGHV3-7*01, IGHV1-2*02.
   E84. The antibody of any one of E79-E83, comprising a TL1A-VH framework sequence derived from a human IGHV1-18*01 germline sequence.
   E85. The antibody of any one of E79-E84, comprising a TL1A-VL framework sequence derived from a human germline VL sequence selected from the group consisting of IGKV1-39*01, IGKV3-11*01, IGKV3D-7*01, IGKV3-11*01, IGKV3-11*01, IGKV3-11*01, IGKV1-39*01, IGKV1-39*01, IGKV1-39*01, IGKV3D-7*01, IGKV3D-7*01, IGKV3D-7*01.
   E86. The antibody of any one of E79-E85, comprising a TL1A-VL framework sequence derived from a human germline IGKV1-39*01, sequence.
   E87. The antibody of any one of E79-E86, comprising a TL1A-VL framework sequence and a TL1A-VH framework sequence wherein one or both of the TL1A-VL framework sequence and the TL1A-VH framework sequence is at least 90%, 91%, 92%, 93%, 94%, 95%, 96%, 97%, 98%, 99%, or 100% identical to the human germline sequence from which it was derived.
   E88. The antibody of any one of E79-E87, comprising a TL1A-VL framework sequence and a TL1A-VH framework sequence, and wherein one or both of the TL1A-VL framework sequence or the TL1A-VH framework sequence is identical to the human germline sequence from which it was derived.
   E89. The antibody of any one of E79-E88, wherein the TL1A-VL comprises an amino acid sequence according to SEQ ID NO: 18, and wherein the TL1A-VH comprises an amino acid sequence according to a sequence selected from the group consisting of SEQ ID NO: 42, SEQ ID NO: 44, SEQ ID NO: 47, SEQ ID NO:49, SEQ ID NO:52, SEQ ID NO: 54, SEQ ID NO: 57, SEQ ID NO: 70, SEQ ID NO: 72, SEQ ID NO: 78, SEQ ID NO: 80, SEQ ID NO: 89, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 110, and SEQ ID NO:120.
   E90. The antibody of any one of E79-E89, comprising a TL1A-VH sequence at least 90%, 91%, 92%, 93%, 94%, 95%, 96%, 97%, 98%, 99%, or 100% identical to SEQ ID NO: 42, and comprising a TL1A-VL sequence at least 90%, 91%, 92%, 93%, 94%, 95%, 96%, 97%, 98%, 99%, or 100% identical to SEQ ID NO: 18.
   E91. The antibody of any one of E79-E90, comprising the TL1A-VH sequence of SEQ ID NO: 42, and the TL1A-VL of SEQ ID NO: 18.
   E92. The antibody of any one of E79-E91, comprising a TL1A-VH sequence encoded by a nucleic acid sequence of SEQ ID NO: 228.
   E93. The antibody of any one of E79-E92, comprising a TL1A-VL sequence encoded by a nucleic acid sequence of SEQ ID NO: 229.
   E94. The antibody of any one of E1-E15, comprising a TL1A-VH sequence encoded by the plasmid deposited at the ATCC and having ATCC Accession No. PTA-127347
   E95. The antibody of any one of E1-E16, comprising a TL1A-VL sequence encoded by the plasmid deposited at the ATCC and having ATCC Accession No. PTA-127348
   E96. An antibody comprising a TL1A-VH sequence encoded by the plasmid deposited at the ATCC and having ATCC Accession No. PTA-127347 and a TL1A-VL sequence encoded by the plasmid deposited at the ATCC and having ATCC Accession No. PTA-127438.
   E97. The antibody of any one of E79-E96, comprising a p40-VH framework sequence derived from a human germline VH sequence selected from the group consisting of DP3, DP7, DP73, DP75, and DP88.
   E98. The antibody of any one of E79-E97, comprising a p40-VH framework sequence derived from a human DP73 germline sequence.
   E99. The antibody of any one of E79-E98, comprising a p40-VL framework sequence derived from a human germline VL sequence selected from the group consisting of DPK4, DPK5, DPK7, DPK8, and DPK9.
   E100. The antibody of any one of E79-E99, comprising a p40-VL framework sequence derived from a human germline DPK9, sequence.
   E101. The antibody of any one of E79-E100, comprising a p40-VL framework sequence and a p40-VH framework sequence wherein one or both of the p40-VL framework sequence and the p40-VH framework sequence is at least 90%, 91%, 92%, 93%, 94%, 95%, 96%, 97%, 98%, 99%, or 100% identical to the human germline sequence from which it was derived.
   E102. The antibody of any one of E79-E101, comprising a p40-VL framework sequence and a p40-VH framework sequence, and wherein one or both of the p40-VL framework sequence or the p40-VH framework sequence is identical to the human germline sequence from which it was derived.
   E103. The antibody of any one of E79-E102, comprising a p40-VH sequence at least 90%, 91%, 92%, 93%, 94%, 95%, 96%, 97%, 98%, 99%, or 100% identical to SEQ ID NO: 177, and comprising a p40-VL sequence at least 90%, 91%, 92%, 93%, 94%, 95%, 96%, 97%, 98%, 99%, or 100% identical to SEQ ID NO: 171.
   E104. The antibody of any one of E79-E103, wherein the p40-VL comprises an amino acid sequence according to SEQ ID NO: 177, and wherein the p40-VH comprises an amino acid sequence according to SEQ ID NO: 171.
   E105. The antibody of any one of E79-E104, comprising a p40-VH sequence encoded by a nucleic acid sequence of SEQ ID NO: 238.
   E106. The antibody of any one of E79-E105, comprising a p40-VL sequence encoded by a nucleic acid sequence of SEQ ID NO: 239.
   E107. The antibody of any one of E79-E106, comprising a p40-VH sequence encoded by the plasmid deposited at the ATCC and having ATCC Accession No. PTA-127206.
   E108. The antibody of any one of E79-E107, comprising a p40-VL sequence encoded by the plasmid deposited at the ATCC and having ATCC Accession No. PTA-127205
   E109. An antibody comprising a p40-VH sequence encoded by the plasmid deposited at the ATCC and having ATCC Accession No. PTA-127206 and a p40-VL sequence encoded by the plasmid deposited at the ATCC and having ATCC Accession No. PTA-127205.
   E110. The antibody of E79-E109, wherein the TL1A-VH is fused to a CH1 domain (TL1A-CH1) and the TL1A-VL is fused to a constant light domain (TL1A-CL) forming a TL1A binding Fab domain (TL1A-Fab).
   E111. The antibody of E110, wherein the p40-VH is fused to a CH1 domain (p40-CH1) and the p40-VL is fused to a constant light domain (p40-CL) forming a p40 binding Fab domain (p40-Fab).
   E112. The antibody of E110-E111, wherein the TL1A-CH1 comprises a sequence according to a sequence selected from the group consisting of SEQ ID NO: 6, and SEQ ID NO: 195, and SEQ ID NO: 199.
   E113. The antibody of E100-E102, wherein the TL1A-CH1 comprises a sequence according to SEQ ID NO: 6.
   E114. The antibody of E100-E113, wherein the TL1A-CL comprises a sequence selected from the group consisting of SEQ ID NO: 16, SEQ ID NO: 197, and SEQ ID NO: 201.
   E115. The antibody of E100-E114, wherein the TL1A-CL comprises a sequence according to SEQ ID NO: 16.
   E116. The antibody of E100-E115, wherein the p40-CH1 comprises a sequence according to a sequence selected from the group consisting of SEQ ID NO: 6, and SEQ ID NO: 195, and SEQ ID NO: 199.
   E117. The antibody of E100-E116, wherein the p40-CH1 comprises a sequence according to SEQ ID NO: 6.
   E118. The antibody of E100-E117, wherein the p40-CL comprises a sequence selected from the group consisting of SEQ ID NO: 16, SEQ ID NO: 197, and SEQ ID NO: 201.
   E119. The antibody of E100-E118, wherein the p40-CL comprises a sequence according to SEQ ID NO:16.
   E120. The antibody of any one of E111-E119, comprising an antibody Fc domain comprising a first Fc chain and a second Fc chain.
   E121. The antibody of E120, wherein the Fc domain is the Fc domain of an IgA (for example IgA₁ or IgA₂), IgD, IgE, IgM, or IgG (for example IgG₁, IgG₂, IgG₃, or IgG₄).
   E122. The antibody of E121, wherein the Fc domain is the Fc domain of an IgG₁.
   E123. The antibody of E120-E122, wherein the first Fc chain comprises, from N-terminus to C-terminus: a first hinge region, a first CH2 region, and a first CH3 region, and the second Fc chain comprises, from N-terminus to C-terminus: a second hinge region, a second CH2 region, and a second CH3 region.
   E124. The antibody of E123, wherein the N-terminus of the first Fc chain is connected to the C-terminus of the first TL1A-CL domain.
   E125. The antibody of E123-E124, wherein the N-terminus of the second Fc chain is connected to the C-terminus of the first p40-CH1 domain.
   E126. The antibody of E123-E125, wherein the first CH2 region and the second CH2 region each comprise a sequence according to SEQ ID NO: 8.
   E127. The antibody of E123-E126, wherein either or both of the first hinge region comprises a sequence selected from the group consisting of SEQ ID NO: 7, SEQ ID NO: 157, SEQ ID NO:

### 179, SEQ ID NO: 182, and SEQ ID NO: 189, SEQ ID NO: 191, SEQ ID NO: 204, and SEQ ID NO: 206.

E128. The antibody of E123-E127, wherein the first hinge region and the second hinge region each contain one or more amino acid modifications that promote the association of the first hinge region with the second hinge region.
E129. The antibody of E128, wherein the first hinge region and the second hinge region each comprise a different and complementary sequence, and the different and complementary sequences are selected from one of the following pairs of different and complementary sequences:
(i) SEQ ID NO: 179 and SEQ ID NO: 182;
(ii) SEQ ID NO: 189 and SEQ ID NO: 191.
   E130. The antibody of E123-E128, wherein the first hinge region comprises a sequence according to SEQ ID NO: 204.
   E131. The antibody of E123-E128, wherein the second hinge region comprises a sequence according to SEQ ID NO: 7.
   E132. The antibody of E123-E131, wherein the first Fc chain and the second Fc chain each contain one or more amino acid modifications that promote the association of the first Fc chain with the second Fc chain.
   E133. The antibody of E123-E132, wherein the first Fc chain comprises a first CH3 region, and the second Fc chain comprises a second CH3 region, and the first CH3 region and the second CH3 region each comprise a different and complementary sequence, and the different and complementary sequences are selected from one of the following pairs of different and complementary sequences:
(i) SEQ ID NO: 160 and SEQ ID NO: 162;
(ii) SEQ ID NO: 180, and SEQ ID NO: 183;
(iii) SEQ ID NO: 155 and SEQ ID NO: 158;
(iv) SEQ ID NO: 185 and SEQ ID NO: SEQ ID NO: 187.
   E134. The antibody of E123, wherein the first CH3 region and the second CH3 region comprise SEQ ID NO: 160 and SEQ ID NO: 162.
   E135. The antibody of any one of E79-E134, wherein the antibody comprises a first, second, third, and fourth polypeptide chain, such that the first and third polypeptide chains together form the TL1A-Fab, and the second and fourth polypeptide chains together form the p40-Fab.
   E136. The antibody of E135, wherein the first, second, third, and fourth polypeptide chains have a combination of sequences in accordance with one of the groups selected from the following list:
(i) the first polypeptide sequence is in accordance with SEQ ID NO: 205, the second polypeptide is in accordance with SEQ ID NO: 203, the third polypeptide is in accordance with SEQ ID NO: 207, and the fourth polypeptide is in accordance with SEQ ID NO: 178;
(ii) the first polypeptide sequence is in accordance with SEQ ID NO: 188, the second polypeptide is in accordance with SEQ ID NO: 186, the third polypeptide is in accordance with SEQ ID NO: 19, and the fourth polypeptide is in accordance with SEQ ID NO: 178;
(iii) the first polypeptide sequence is in accordance with SEQ ID NO: 192, the second polypeptide is in accordance with SEQ ID NO: 190, the third polypeptide is in accordance with SEQ ID NO: 19, and the fourth polypeptide is in accordance with SEQ ID NO: 178;
(iv) the first polypeptide sequence is in accordance with SEQ ID NO: 194, the second polypeptide is in accordance with SEQ ID NO: 193, the third polypeptide is in accordance with SEQ ID NO: 19, and the fourth polypeptide is in accordance with SEQ ID NO: 178; and
(v) the first polypeptide sequence is in accordance with SEQ ID NO: 200, the second polypeptide is in accordance with SEQ ID NO: 196, the third polypeptide is in accordance with SEQ ID NO: 202, and the fourth polypeptide is in accordance with SEQ ID NO: 198.
   E137. The antibody of E135-E136, wherein the first polypeptide sequence is in accordance with SEQ ID NO: 205, the second polypeptide is in accordance with SEQ ID NO: 203, the third polypeptide is in accordance with SEQ ID NO: 207, and the fourth polypeptide is in accordance with SEQ ID NO: 178.
   E138. An isolated antibody that specifically binds to TL1A and p40, wherein the antibody comprises a first, second, third, and fourth polypeptide chain, such that the first and third polypeptide chains together form a TL1A binding region, and the second and fourth polypeptide chains together form a p40 binding region, and wherein the first polypeptide sequence is in accordance with SEQ ID NO: 205, the second polypeptide is in accordance with SEQ ID NO: 203, the third polypeptide is in accordance with SEQ ID NO: 207, and the fourth polypeptide is in accordance with SEQ ID NO: 178.
   E139. An isolated antibody that specifically binds to TL1A and p40, wherein the antibody comprises a first, second, third, and fourth polypeptide chain, such that the first and third polypeptide chains together form a TL1A binding region, and the second and fourth polypeptide chains together form a p40 binding region, and wherein the first polypeptide sequence comprises a sequence encoded by the plasmid deposited at the ATCC and having ATCC Accession No. PTA-127349, the second polypeptide comprises a sequence encoded by the plasmid deposited at the ATCC and having ATCC Accession No. PTA-127346, the third polypeptide comprises a sequence encoded by the plasmid deposited at the ATCC and having ATCC Accession No. PTA-127350, and the fourth polypeptide comprises a sequence encoded by the plasmid deposited at the ATCC and having ATCC Accession No. PTA-127203.
   E140. An isolated antibody that specifically binds TL1A, comprising the CDRs of an antibody selected from one or more of Tables 30, 31, 32, and 33.
   E141. An isolated antibody that specifically binds TL1A, comprising the VH and VL of an antibody selected from one or more of Tables 30, 31, 32, and 33.
   E142. An isolated antibody that specifically binds TL1A, selected from one or more of Tables 30, 31, 32, and 33.
   E143. An isolated antibody that specifically binds TL1A and specifically binds p40, comprising the CDRs of an antibody selected from one or more of Tables 30, 31, 32, and 33.
   E144. An isolated antibody that specifically binds TL1A and specifically binds p40, comprising the VH and VL of an antibody selected from one or more of Tables 30, 31, 32, and 33.
   E145. An isolated antibody that specifically binds TL1A and specifically binds p40, selected from one or more of Tables 30, 31, 32, and 33.
   E146. The antibody of any one of E79-E145, characterized by a score of less than 2 in an affinity-capture self-interaction nanoparticle spectroscopy (AC SINS) assay.
   E147. The antibody of any one of E79-E146, characterized in that the charge heterogeneity of the antibody is at least 50% non-acid and non-basic as measured in an antibody imaged capillary electrophoresis (iCE) for an unstressed sample (T0) to evaluate.
   E148. The antibody of any one of E79-E147, wherein the antibody binds to immobilized human TL1A with a binding affinity of less than 1 nM, as measured by SPR.
   E149. The antibody of any one of E79-E148, wherein the antibody binds to immobilized human TL1A with a binding affinity of less than 500 pM, as measured by SPR.
   E150. The antibody of any one of E79-E149, wherein the antibody binds to immobilized human TL1A with a binding affinity of less than 300 pM, as measured by SPR.
   E151. The antibody of any one of E79-E150, wherein the immobilized antibody binds to human p40 with a binding affinity of less than 1 nM, as measured by SPR.
   E152. The antibody of any one of E79-E151, wherein the immobilized antibody binds to human p40 with a binding affinity of less than 900 pM, as measured by SPR.
   E153. The antibody of any one of E79-E152, wherein the immobilized antibody binds to human p40 with a binding affinity of less than 600 pM, as measured by SPR.
   E154. The antibody of any one of E79-E153, wherein Ab binds p40(IL-23) and TL1A simultaneously in an SPR assay wherein the antibody is injected over immobilized TL1A and the resulting complex subsequently treated with IL-23.
   E155. The antibody of E79-E154, wherein the antibody is characterized by an IC₅₀ of less than 5 nM in a human IL-12 neutralization Kit-225 assay.
   E156. The antibody of E79-E155, wherein the antibody is characterized by an IC₅₀ of less than 4 nM in a human IL-12 neutralization Kit-225 assay.
   E157. The antibody of E79-E156, wherein the antibody is characterized by an IC₅₀ of less than 2 pM in a human IL-12 neutralization Kit-225 assay.
   E158. The antibody of E79-E157, wherein the antibody is characterized by an IC₅₀ of less than 5 nM in a human IL-23 neutralization Kit-225 assay.
   E159. The antibody of E79-E158, wherein the antibody is characterized by an IC₅₀ of less than 4 nM, in a human IL-23 neutralization Kit-225 assay.
   E160. The antibody of E79-E159, wherein the antibody is characterized by an IC₅₀ of less than 1 nM in a human IL-23 neutralization Kit-225 assay in human whole blood.
   E161. The antibody of E79-E160, wherein the antibody is characterized by an IC₅₀ of less than 0.5 nM, in a human IL-23 neutralization Kit-225 assay in human whole blood.
   E162. The antibody of E155-E161, wherein the Kit-225 assay is in human whole blood.
   E163. The antibody of E79-E162, wherein the antibody is characterized by an IC₅₀ of less than 5 nM in a cynomolgus monkey IL-23 neutralization Kit-225 assay.
   E164. The antibody of E79-E163, wherein the antibody is characterized by an IC₅₀ of less than 2 nM in a cynomolgus monkey IL-12 neutralization Kit-225 assay.
   E165. The antibody of E79-E164, wherein the antibody is characterized by an IC₅₀ of less than 1 nM in a cynomolgus monkey IL-12 neutralization Kit-225 assay.
   E166. The antibody of E79-E165, wherein the antibody is characterized by an IC₅₀ of less than 2 nM in a human whole blood assay measuring inhibition of IFNγ.
   E167. The antibody of E79-E166, wherein the antibody is characterized by an IC₅₀ of less than 3 nM in a human whole blood assay measuring inhibition of IFNγ.
   E168. The antibody of E79-E167, wherein the antibody is characterized by an IC₅₀ of less than 2 nM in a human whole blood assay measuring inhibition of IFNγ.
   E169. The antibody of E79-E168, wherein the antibody is characterized by an IC₅₀ of less than 500 pM in a human isolated CD4+ cell assay measuring inhibition of IFNγ.
   E170. The antibody of E79-E169, wherein the antibody is characterized by an IC₅₀ of less than 100 pM in a human isolated CD4+ cell assay measuring inhibition of IFNγ.
   E171. The antibody of E79-E170, wherein the antibody is characterized by an IC₅₀ of less than 200 nM in a human NFκB neutralization assay in TF-1 reporter cells.
   E172. The antibody of E79-E171, wherein the antibody is characterized by an IC₅₀ of less than 150 nM in a human NFκB neutralization assay in TF-1 reporter cells.
   E173. The antibody of any one of E79-E172, wherein the antibody has a terminal half-life of at least 10 days in TG32 mice, based on a two-week analysis.
   E174. The antibody of any one of E79-E173, wherein the antibody has a terminal half-life of at least 14 days in TG32 mice, based on a two-week analysis.
   E175. The antibody of any one of E79-E174, wherein the antibody has a terminal half-life of at least 16 days in TG32 mice, based on a two-week analysis.
   E176. The antibody of any one of E79-E175, wherein the antibody has a terminal half-life of at least 17 days in TG32 mice, based on a two-week analysis.
   E177. The antibody of any one of E79-E176, wherein the antibody has a terminal half-life of at least 21 days in cynomolgus monkeys.
   E178. The antibody of any one of E79-E177, wherein the antibody has a terminal half-life of at least 25 days in cynomolgus monkeys.
   E179. The antibody of any one of E79-E178, wherein the antibody has a terminal half-life of at least 28 days in cynomolgus monkeys.
   E180. The antibody of any one of E1-179, for use as a medicament.
   E181. The antibody of E180, wherein the use is for the treatment of one or more selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis.
   E182. The antibody of any one of E180-E181, wherein the use is for inflammatory bowel disease (IBD).
   E183. The antibody of any one of E180-E182, wherein the use is for Crohn's disease.
   E184. The antibody of any one of E180-E183, wherein the use is for ulcerative colitis.
   E185. A pharmaceutical composition comprising a therapeutically effective amount of the antibody of E1-E184 and a pharmaceutically acceptable carrier.
   E186. A method of treating a medical condition, comprising administering to a subject in need thereof a therapeutically effective amount of the antibody of any one of E1-E184, or the pharmaceutical composition of E185.
   E187. The method of E186, wherein the condition is selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis.
   E188. The method of any one of E186-E87, comprising administering said antibody or pharmaceutical composition, subcutaneously.
   E189. The method of any one of E186-E188, wherein said antibody thereof, or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.
   E190. An isolated polynucleotide, comprising one or more nucleotide sequences encoding the antibody of any one of E1-184.
   E191. The polynucleotide of E190, wherein said polynucleotide is RNA.
   E192. The polynucleotide of E190-E191, wherein said polynucleotide comprises at least one chemical modification.
   E193. The polynucleotide of E192, wherein the chemical modification wherein is selected from pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine), 5-methoxyuridine and 2′-O-methyl uridine.
   E194. The polynucleotide of E190-E191, wherein said polynucleotide does not comprise a chemical modification.
   E195. An isolated polynucleotide encoding the VH, VL, or both, of an antibody that binds TL1A, wherein the nucleic acid comprises: the nucleic acid sequence of SEQ ID NO: 228, the nucleic acid sequence of SEQ ID NO: 229, or both.
   E196. An isolated polynucleotide encoding a VH bearing polypeptide and a VL bearing polypeptide, or both, of an antibody that binds to TL1A, wherein said nucleic acid comprises the nucleic acid sequence of SEQ ID NO: 231, the nucleic acid sequence of SEQ ID NO: 233, or both.
   E197. An isolated polynucleotide encoding the VH, VL, or both, of an antibody that binds to TL1A, wherein said nucleic acid comprises the nucleic acid sequence of the insert of the plasmid deposited with the ATCC and having the Accession Number PTA-127347 the nucleic acid sequence of the insert of the plasmid deposited with the ATCC and having the Accession Number PTA-127348 or both.
   E198. An isolated polynucleotide encoding a VH bearing polypeptide and a VL bearing polypeptide, or both, of an antibody that binds to TL1A, wherein said nucleic acid comprises the nucleic acid sequence of the insert of the plasmid deposited with the ATCC and having the Accession Number PTA-127347, the nucleic acid sequence of the insert of the plasmid deposited with the ATCC and having the Accession Number PTA-127348, or both.
   E199. An isolated polynucleotide comprising one or more nucleic acids encoding the one or more sequences of a null-arm antibody that binds TL1A, wherein null-arm antibody comprises a null-arm TL1A antibody comprising a first variable heavy chain and a first variable light chain, and a second variable heavy chain and second variable light chain, wherein the first variable heavy chain and the first variable light chain comprise a first Fab domain that binds to TL1A (TL1A-Fab) and the second variable heavy chain and the second variable light chain comprise a second Fab domain that comprises a non-binding TL1A binding domain that does not specifically bind to a target, (xTL1A-Fab), and wherein the first and second variable light chains are identical, and wherein said polynucleotide comprises the nucleic acid sequence of SEQ ID NO: 228, the nucleic acid sequence of SEQ ID NO: 230, and/or the nucleic acid sequence of SEQ ID NO: 260.
   E200. An isolated polynucleotide comprising one or more nucleic acids encoding the one or more sequences of a null-arm antibody that binds TL1A, wherein null-arm antibody comprises a null-arm TL1A antibody comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target, (xTL1A-Fab), and wherein the first and second light chain are identical, and wherein said polynucleotide comprises the nucleic acid sequence of SEQ ID NO: 231, the nucleic acid sequence of SEQ ID NO: 232, and/or the nucleic acid sequence of SEQ ID NO: 233.
   E201. An isolated polynucleotide comprising one or more nucleic acids encoding the one or more sequences of a null-arm antibody that binds TL1A, wherein null-arm antibody comprises a null-arm TL1A antibody comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target, (xTL1A-Fab), and wherein the first and second light chain are identical, and wherein said polynucleotide comprises one or more of the nucleic acid sequence of the insert of the plasmid deposited with the ATCC and having the Accession Number PTA-127347, the nucleic acid sequence of the insert of the plasmid deposited with the ATCC and having the Accession Number PTA-127348, and the nucleic acid sequence of the insert of the plasmid deposited with the ATCC and having the Accession Number PTA-127351.
   E202. An isolated polynucleotide encoding one or more of the first, second, third, and fourth polypeptides of an antibody that binds to both TL1A and p40, comprising
(i) an TL1A-VH sequence encoded by a nucleic acid comprising the nucleic acid sequence of SEQ ID NO: 228, and an TL1A-VL encoded by a nucleic acid comprising the nucleic acid sequence of SEQ ID NO: 229; and
(ii) an p40-VH sequence encoded by a nucleic acid comprising the nucleic acid sequence of SEQ ID NO: 238, and an p40-VL sequence encoded by a nucleic acid comprising the nucleic acid sequence of SEQ ID NO: 239.
   E203. An isolated polynucleotide encoding one or more of the first, second, third, and fourth polypeptides of an antibody that binds to both TL1A and p40, comprising
(i) an TL1A-VH bearing polypeptide sequence encoded by a nucleic acid comprising the nucleic acid sequence of SEQ ID NO: 234, and an TL1A-VL bearing polypeptide encoded by a nucleic acid comprising the nucleic acid sequence of SEQ ID NO: 235; and
(ii) an p40-HC sequence encoded by a nucleic acid comprising the nucleic acid sequence of SEQ ID NO: 236, and an p40-LC sequence encoded by a nucleic acid comprising the nucleic acid sequence of SEQ ID NO: 237.
   E204. An isolated polynucleotide encoding one or more of the first, second, third, and fourth polypeptides of an antibody that binds to both TL1A and p40, comprising
(i) an TL1A-VH sequence encoded by the plasmid deposited at the ATCC and having ATCC Accession No. PTA-127347, and an TL1A-VL sequence encoded by the plasmid deposited at the ATCC and having ATCC Accession No. PTA-127348; and
(ii) an p40-VH sequence encoded by the plasmid deposited at the ATCC and having ATCC Accession No. PTA-127206, and an p40-VL sequence encoded by the plasmid deposited at the ATCC and having ATCC Accession No. PTA-127205.
   E205. An isolated polynucleotide encoding one or more of the first, second, third, and fourth polypeptides of an antibody that binds to both TL1A and p40, comprising
(i) an TL1A-VHbearing polypeptide sequence encoded by the plasmid deposited at the ATCC and having ATCC Accession No. PTA-127350, and an TL1A-VL bearing polypeptide sequence encoded by the plasmid deposited at the ATCC and having ATCC Accession No. PTA-127349; and
(ii) an p40-HC sequence encoded by the plasmid deposited at the ATCC and having ATCC Accession No. PTA-127346, and an p40-LC sequence encoded by the plasmid deposited at the ATCC and having ATCC Accession No. PTA-127203.
   E206. An isolated polynucleotide encoding a TL1A antibody VH domain, comprising the nucleic acid sequence of SEQ ID NO: 228.
   E207. An isolated polynucleotide encoding a TL1A antibody VL domain, comprising the nucleic acid sequence of SEQ ID NO: 229.
   E208. An isolated polynucleotide encoding a TL1A antibody VH domain engineered to eliminate TL1A binding, comprising the nucleic acid sequence of SEQ ID NO: 230.
   E209. An isolated polynucleotide encoding a TL1A antibody HC, comprising the nucleic acid sequence of SEQ ID NO: 231.
   E210. An isolated polynucleotide encoding a TL1A antibody HC engineered to eliminate TL1A binding, comprising the nucleic acid sequence of SEQ ID NO: 232.
   E211. An isolated polynucleotide encoding a TL1A antibody LC, comprising the nucleic acid sequence of SEQ ID NO: 233.
   E212. An isolated polynucleotide encoding a TL1A antibody mFd chain, comprising the nucleic acid sequence of SEQ ID NO: 234.
   E213. An isolated polynucleotide encoding a TL1A antibody LC-Fc chain comprising the nucleic acid sequence of SEQ ID NO: 235.
   E214. An isolated polynucleotide encoding a p40 antibody HC, comprising the nucleic acid sequence of SEQ ID NO: 236.
   E215. An isolated polynucleotide encoding a p40 antibody LC, comprising the nucleic acid sequence of SEQ ID NO: 237.
   E216. An isolated polynucleotide encoding a p40 antibody VH, comprising the nucleic acid sequence of SEQ ID NO: 238.
   E217. An isolated polynucleotide encoding a p40 antibody VL, comprising the nucleic acid sequence of SEQ ID NO: 239.
   E218. A vector comprising the polynucleotide of E190-E217.
   E219. An isolated host cell comprising the polynucleotide of E190-E217, or the vector of E218.
   E220. A method of producing an isolated antibody, comprising culturing the host cell of E219 under conditions that result in production of the antibody, and recovering the antibody.
   E221. A pharmaceutical composition comprising a therapeutically effective amount of the antibody of any one of E1-E189, and a pharmaceutically acceptable carrier.

### FIGURES

FIG. 1. Schematic of the “one-armed” antibody. This format includes one anti-TL1A binding Fab and a truncated Fc. Fc heterodimerization is driven through KiH mutations engineered in the CH3 domain.

FIG. 2. Schematic of the monovalent bispecific antibody. This format includes one anti-TL1A binding Fab and a “disabled” Fab (also known as a null-arm) with mutations that ablate TL1A binding. Fc pairing is driven through the introduction of KiH mutations in both CH3 domains.

FIGS. 3A-3C. The Majority of [xTL1A] Bivalent Antibody Variants Screened Demonstrate No binding to recombinant human TL1A. All [xTL1A] bivalent antibody variants are plotted in graphs FIG. 3A-FIG. 3C. [xTL1A] bivalent antibody variants that do not bind recombinant human TL1A are plotted with solid black lines and variants that show some binding are plotted with patterned lines. Binding of positive control TL1A-0260 to TL1A is plotted with dashed lines in all three graphs.

FIGS. 4A-4C. High Concentrations of [xTL1A] Bivalent Antibody Variants Do Not Bind to TL1A Except [xTL1A-0322]. Sensorgrams of select [xTL1A] bivalent antibody variants binding TL1A. FIG. 4A. Binding of positive control TL1A-0260 binding to TL1A at 196 nM concentration. FIG. 4B. Binding of [xTL1A]-0322 bivalent antibody to TL1A at 1 μM concentration; this is the only variant that showed binding to TL1A. FIG. 4C. Representative sensorgram demonstrating that [xTL1A]-0349 does not bind TL1A at 1 μM concentration. The remaining [xTL1A] bivalent variants had similar sensorgrams at 1 μM concentration.

FIG. 5. High concentrations of [xTL1A] Bivalent Antibody Variants Mostly Exhibited No Binding to Human TL1A Expressed on Cells. The binding of [xTL1A] bivalent antibody variants, isotype control and positive control TL1A-0260 to cells over-expressing human TL1A was plotted above. The black bars show binding at 1 μg/mL concentration and the grey bars show binding at 20 μg/mL concentration. The vertical black and grey lines were drawn at the level of isotype control binding for 1 μg/mL and 20 μg/mL concentrations, respectively. [xTL1A] variants that showed binding greater than the isotype control were deemed to bind TL1A expressing cells.

FIGS. 6A-6D. [xTL1A] Bivalent Antibody Variants Screened in a Competition ELISA Did Not Compete with Bio-TL1A-0260 for Binding to Recombinant Human TL1A Protein Indicating TL1A Binding Properties were Compromised. All [xTL1A] bivalent antibody variants are plotted in graphs A-D. [xTL1A] bivalent antibody variants that do not compete with bio-TL1A-0260 for recombinant human TL1A binding are plotted with solid black lines and variants that show some competition with bio-TL1A-0260 are plotted with patterned lines. Competition of positive control TL1A-0260 with bio-TL1A-0260 for binding to TL1A is plotted with dashed lines in all four graphs.

FIG. 7. Monovalent Bispecific Variants do not Present Binding to BSA or HSA.

FIGS. 8A-8F. General schematics and nomenclature of bispecific molecules and Fab domains engineered with Fc heterodimerization mutations for single cell line production method.

FIGS. 9A-9D. General schematics and nomenclature of bispecific molecules engineered with Knob-into-Home and Charged-based Fc heterodimerization mutations for dual cell line production method.

FIG. 10A. P40TL1A-0033 TL1A mFd sequence and numbering (SEQ ID NO: 207). FIG. 10B. P40TL1A-0033 TL1A LC Fc Sequence and numbering (SE ID NO: 205). FIG. 10C. P40TL1A-0033 p40 HC sequence and numbering (SEQ ID NO: 203). FIG. 10D. P40TL1A-0033 p40 LC sequence and numbering (SEQ ID NO: 178).

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of ***) means that the parameter may vary by as much as 10% below or above the stated numerical value for that parameter. For example, a dose of about 5 mg means 5%±10%, i.e. it may vary between 4.5 mg and 5.5 mg.

### Antibody

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g. monospecific, bispecific), and includes portions of intact antibodies that retain the ability to bind to a given antigen (e.g. an “antigen-binding fragment”), and any other modified configuration of an immunoglobulin molecule that comprises an antigen binding site.

An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

### Variable Region

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The extended definition is the combination of the Kabat and Chothia definitions. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions.

### Pfabat Numbering Method Developed for Consistent Antibody Numbering

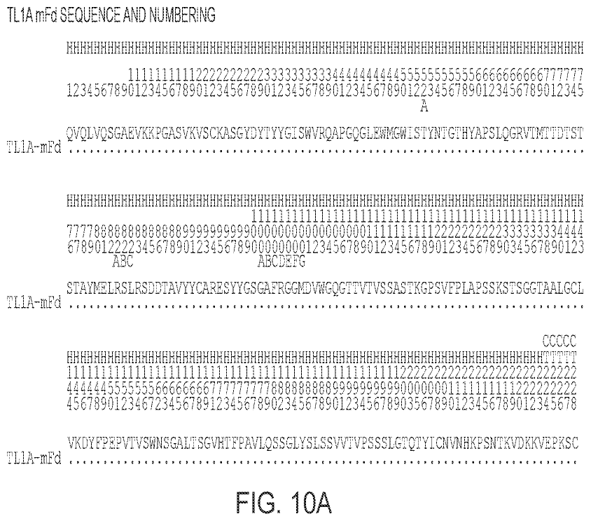

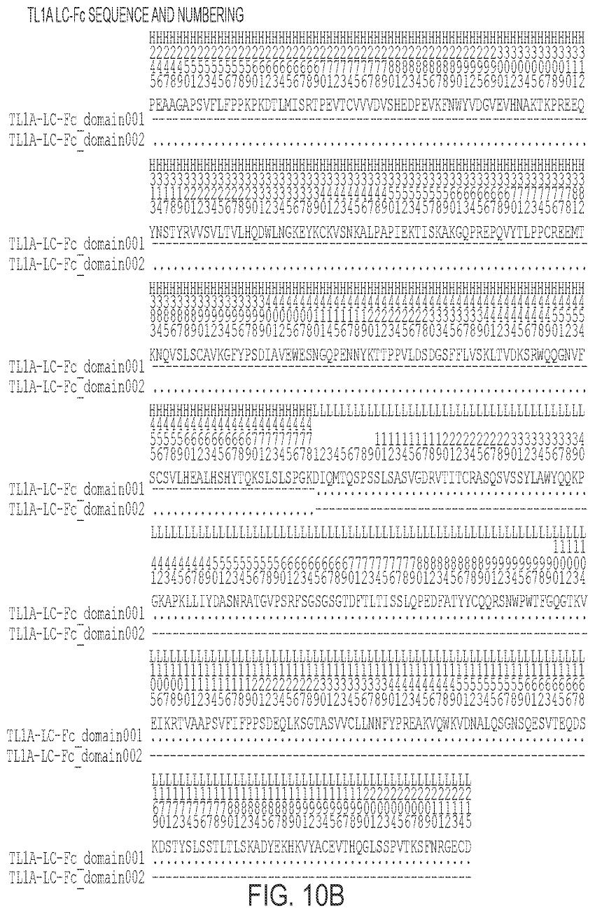

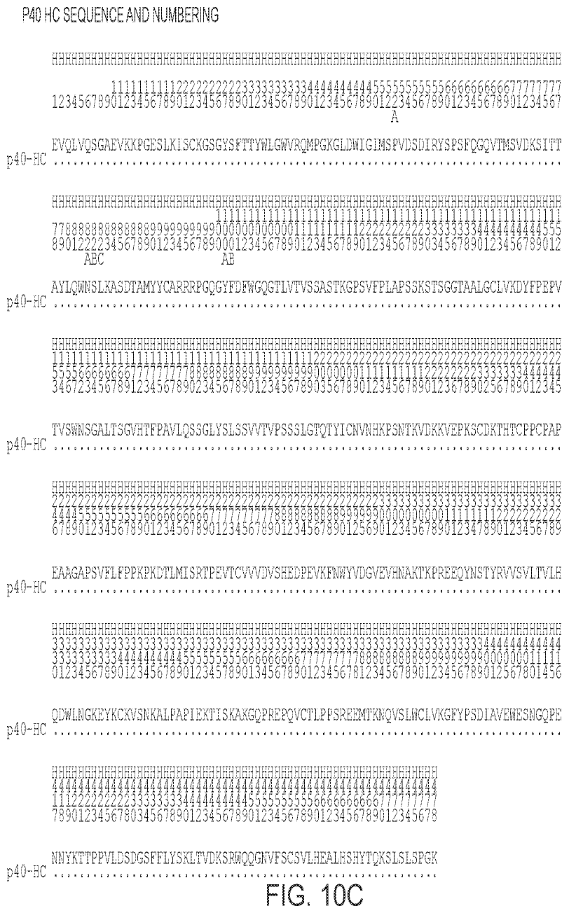

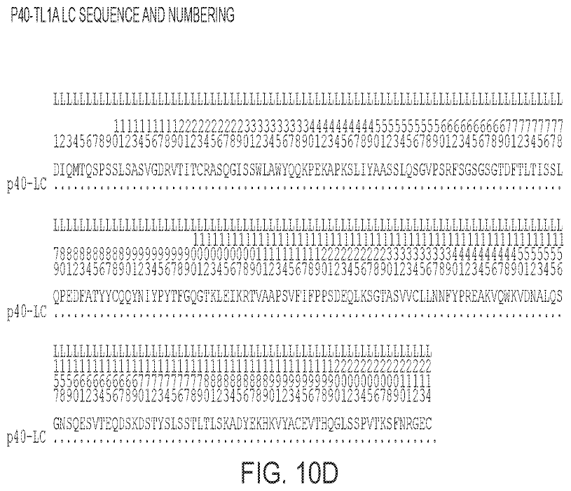

The Pfabat numbering method is a defined algorithm for consistent antibody numbering, based on the Kabat numbering system (Sequences of Proteins of Immunological Interest, Fifth Edition by Kabat et al., NIH Publication NO: 91-3242, 1991). Unlike many other computational implementations of Kabat numbering, Pfabat numbers entire human IgG1 heavy and light chains, including the constant (C) regions and heavy chain hinge. FIGS. 10A-10D show example numbering, with complementarity-determining regions (CDR) residues underlined in bold. For the light chain, the CDR definitions are: CDRL1 from residues L24-L34; CDRL2 from residues L50-L56; CDRL3 from residue L89-L97; CDRH1 from residues H26-H35 (including insertion positions such as H35B); CDRH2 from residues H50-H65; and CDRH3 from residues H95-H102. Note that the definition of CDRH1 used herein includes positions H26-H29, which are not included in some other interpretations of Kabat numbering.

### Constant Region

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL)

### Fc Domain and Fc Chain

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification

### Monoclonal Antibody

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

### Human Antibody

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

### Chimeric Antibody

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

### Humanized Antibody

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

### Antigen

An “antigen” refers to the molecular entity used for immunization of an immunocompetent vertebrate to produce the antibody that recognizes the antigen or to screen an expression library (e.g., phage, yeast or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

### Epitope

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition or alternatively, during the discovery process, the generation and characterization of antibodies may elucidate information about desirable epitopes. From this information, it is then possible to competitively screen antibodies for binding to the same epitope.

### Binding Affinity

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (k_{off})” or “k_{d}” to the association rate, or “on-rate (kₒₙ)” or “kₐ”. Thus, K_{D} equals k_{off}/kₒₙ (or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (Octet QK^{e} system, ForteBio). Alternatively, or in addition, a KinExA (Kinetic Exclusion Assay) assay, available from Sapidyne Instruments (Boise, ID) can also be used.

### Monospecific Antibody

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

### Bispecific Antibody

A “bispecific antibody” refers to a molecule that has binding specificity for at least two different epitopes. In some embodiments, bispecific antibodies can bind simultaneously two different antigens. In other embodiments, the two different epitopes may reside on the same antigen.

### One-Armed Antibody

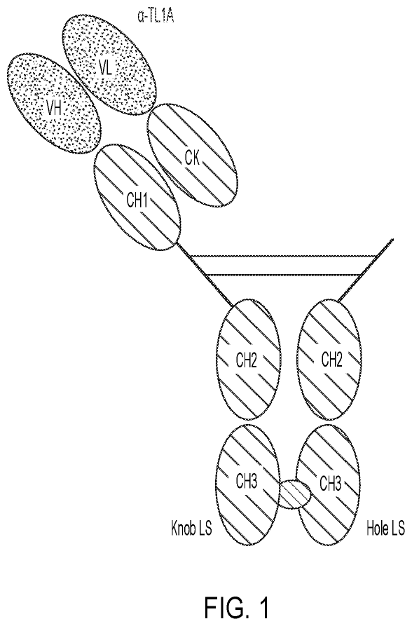

A one-armed antibody is an IgG antibody comprising an intact Fc domain of two hinge domains, two CH2 domain, and two CH3 domains, but only one Fab region. A one-armed antibody is monovalent and monospecific due to it possessing only a single Fab domain. FIG. 1 depicts a schematic of a one-armed antibody.

### Null-Armed Antibody or Disabled Antibody

An antibody comprising a null arm (also termed “disabled”) is a monovalent monospecific antibody, wherein one of the antigen binding sites has been modified (synonymous with engineered) to eliminate binding to the target. One or more of the heavy chain CDRs may have been modified to eliminate binding to the target and favorably, the light chain CDRs may not have been modified, so as to facilitate antibody production and minimize mispairing. Modification, or engineering, may be shown by a documented process wherein at least one structural feature is retained at each process step. FIG. 1 depicts a schematic of a null-armed antibody.

### Half Maximal Effective Concentration (EC₅₀)

The term “half maximal effective concentration (EC₅₀)” refers to the concentration of a therapeutic agent which causes a response halfway between the baseline and maximum after a specified exposure time. The therapeutic agent may cause inhibition or stimulation. The EC₅₀ value is commonly used, and is used herein, as a measure of potency.

### Agonist

An “agonist” refers to a substance which promotes (i.e., induces, causes, enhances, or increases) the biological activity or effect of another molecule. The term agonist encompasses substances (such as an antibody) which bind to a molecule to promote the activity of that molecule.

### Antagonist

An “antagonist” refers to a substance that prevents, blocks, inhibits, neutralizes, or reduces a biological activity or effect of another molecule, such as a receptor. The term antagonist encompasses substances (such as an antibody) which bind to a molecule to prevent or reduce the activity of that molecule.

### Compete

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

### Host Cell

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

### Vector

A “vector” refers to a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc). Vectors may include DNA or RNA. An “expression vector” as used herein refers to a vector that includes at least one polypeptide-encoding gene, at least one regulatory element (e.g. promoter sequence, poly(A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers and terminator). For translation, one or more translational controlling elements may also be included such as ribosome binding sites, translation initiation sites, and stop codons.

### Isolated

An “isolated” molecule (e.g. antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art.

### Polypeptide/Protein

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

### Polynucleotide/Nucleic Acid

A “polynucleotide” or “nucleic acid,” (used interchangeably herein) refers to a chain of nucleotides of any length, and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside.

### Conservative Substitution

A “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine.

### Identity

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g. algorithms), which are well known in the art.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

### Excipient

The term ‘excipient’ refers to any material which, which combined with an active ingredient of interest (e.g. antibody), allow the active ingredient to retain biological activity. The choice of excipient will to a large extent depend on factors such as the mode of administration, the effect of the excipient on solubility and stability, and the nature of the dosage form. As used herein, “excipient” “includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

### Treating

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder or condition is one or more selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis.

### Prevent

The terms “prevent” or “prevention” refer to one or more of delay of onset, reduction in frequency, or reduction in severity of at least one sign or symptom (e.g., ***specific for particular application) of a particular disease, disorder or condition (e.g., ***). In some embodiments, prevention is assessed on a population basis such that an agent is considered to “prevent” a particular disease, disorder or condition if a statistically significant decrease in the development, frequency or intensity of one or more symptoms of the disease, disorder or condition is observed in a population susceptible to the disease, disorder or condition. Prevention may be considered complete when onset of disease, disorder or condition has been delayed for a predefined period of time.

### Subject

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development. In some embodiments, a subject is a patient with one or more diseases or disorders selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis.

### Therapeutically Effective Amount

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following:

(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and
(3) ameliorating the disease; for example, ameliorating a disease, condition or disorder in an individual that is experiencing or displaying the pathology or symptomatology of the disease, condition or disorder (i.e., reversing the pathology or symptomatology).

### Antibodies to TL1A

The disclosure provides antibodies that bind to tumor necrosis factor like ligand 1A (TL1A). TL1A is also known as vascular endothelial growth inhibitor (VEGI) and TNF superfamily member 15 (TNFSF15).

As used herein, the term “TL1A” includes variants, isoforms, homologs, orthologs and paralogs of TL1A. In some embodiments, an antibody disclosed herein cross-reacts with TL1A from species other than human, such as TL1A of cynomolgus monkey, as well as different forms of TL1A. In some embodiments, an antibody may be completely specific for human TL1A and may not exhibit species cross-reactivity (e.g., does not bind mouse TL1A) or other types of cross-reactivity. As used herein the term TL1A refers to naturally occurring human TL1A unless contextually dictated otherwise. Therefore, an “TL1A antibody” “anti-TL1A antibody” or other similar designation means any antibody (as defined herein) that binds or reacts with TL1A, an isoform, fragment or derivative thereof. The full length, mature form of TL1A, as represented by UniProtKB/Swiss-Prot accession number AA104463.1 is herein provided as SEQ ID NO: 208. The full length, mature form of mouse TL1A, as represented by UniProtKB/Swiss-Prot accession number Q5UBV8 is herein provided as SEQ ID NO: 211. The full length, mature form of cynomolgus TL1A, as represented by UniProtKB/Swiss-Prot accession number XP_005581018.1 is herein provided as SEQ ID NO: 209.

TL1A, a member of the TNF superfamily, is a homotrimeric cytokine expressed by innate immune cells, including monocytes, macrophages and dendritic cells upon induction through Fc-receptor cross-linking by IC or stimulation of TLRs. Small subsets of activated T cells, endothelial cells, and fibroblasts also express TL1A^{20,21,22}. TL1A engages with its receptor DR3 expressed under inflammatory conditions on lymphocytes, NK cells, NKT cells, ILCs and epithelial cells to enhance production of pro-inflammatory and pro-fibrotic cytokines and can also bind decoy receptor DcR3, which functions as a negative regulator. TL1A can synergize with IL-12 and IL-23 to enhance the activity of Th1 and Th17 cells by increasing cell proliferation and cytokine secretion. Importantly, TL1A can promote cytokine production in memory T cells in the presence of IL-15 and IL-18 independently of IL-12 and IL-23. In addition to direct activation of immune cells, recent studies have identified additional roles for TL1A in promoting intestinal fibrosis through exacerbation of myofibroblast activity,³⁰ and inducing epithelial damage responses including IL-8 secretion and reduced barrier function through negative impacts on tight junction proteins. Multiple lines of evidence suggest a prominent role for TL1A in IBD, including numerous genome wide association studies pointing to several polymorphisms in the TL1A gene (TNFSF15) identified as IBD susceptibility loci in both European and Asian patient populations.^{30,33} In addition, increased expression of TL1A has been reported in inflamed tissues of IBD patients.^{21, 34}

Without wishing to be bound by any particular theory, blockade of the TL1A cellular ligand interaction inhibits direct activation of immune cells and intestinal fibrosis.

A neutralizing or “blocking” antibody refers to an antibody whose binding to TL1A one or both of (i) interferes with, limits, or inhibits the interaction between TL1A and a its ligand, such as DR3 receptor; or (ii) results in inhibition of at least one biological function of ligand binding. Assays to determine neutralization by an antibody of the disclosure are well-known in the art.

The biological function or biological activity of TL1A can, but need not be, mediated by the interaction between TL1A and its ligands.

Anti-TL1A antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-TL1A antibody is a monoclonal antibody. In some embodiments, an anti-TL1A antibody is a human or humanized antibody. In some embodiments, an anti-TL1A antibody is a chimeric antibody.

In some embodiments, the invention provides a TL1A antibody having a light chain variable region (VL) sequence and a heavy chain variable region (VH) sequence as found in one or more of Table 30, 31, 32, and 33, or variants thereof.

The invention also provides CDR portions of antibodies to TL1A. Determination of CDR regions is defined in Example 1. In some embodiments, the antibody comprises three CDRs of any one of the heavy chain variable regions shown in one or more of Table 30, 31, 32, and 33. In some embodiments, the antibody comprises three CDRs of any one of the light chain variable regions shown in one or more of Table 30, 31, 32, and 33. In some embodiments, the antibody comprises three CDRs of any one of the heavy chain variable regions and three CDRs of any one of the light chain variable regions each shown in one or more of Table 30, 31, 32, and 33.

In some embodiments, the antibody comprises the six CDRs of a TL1A antibody selected from one or more of Table 30, 31, 32, and 33. In some embodiments, the antibody comprises the VH and VL of a TL1A antibody each selected from one or more of Table 83, 84, 85, 86, and 87. In some embodiments, the antibody comprises the HC and LC of a TL1A antibody each selected from one or more of Table 30, 31, 32, and 33.

In some embodiments, the disclosure provides anti-TL1A antibodies containing variations of the CDRs, VH, VL, HC, and LC regions shown in one or more of Table 30, 31, 32, and 33, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 30, 31, 32, and 33, These amounts are not meant to be limiting and increments between the recited percentages are specifically envisioned as part of the disclosure.

In some aspects, the disclosure provides an isolated antibody that specifically binds to TL1A, comprising a heavy chain variable region (TL1A-VH) and a light chain variable region (TL1A-VL), comprising sequences selected from the group consisting of
(i) the CDR-H1, CDR-H2, and CDR-H3 sequences of SEQ ID NO: 42, and the CDR-L1, CDR-L2, and CDR-L3 sequences of SEQ ID NO: 18;
(ii) the CDR-H1, CDR-H2, and CDR-H3 sequences of SEQ ID NO: 44, and the CDR-L1, CDR-L2, and CDR-L3 sequences of SEQ ID NO: 18;
(iii) the CDR-H1, CDR-H2, and CDR-H3 sequences of SEQ ID NO: 47, and the CDR-L1, CDR-L2, and CDR-L3 sequences of SEQ ID NO: 18;
(iv) the CDR-H1, CDR-H2, and CDR-H3 sequences of SEQ ID NO: 49, and the CDR-L1, CDR-L2, and CDR-L3 sequences of SEQ ID NO: 18;
(v) the CDR-H1, CDR-H2, and CDR-H3 sequences of SEQ ID NO: 52, and the CDR-L1, CDR-L2, and CDR-L3 sequences of SEQ ID NO: 18;
(vi) the CDR-H1, CDR-H2, and CDR-H3 sequences of SEQ ID NO: 54, and the CDR-L1, CDR-L2, and CDR-L3 sequences of SEQ ID NO: 18;
(vii) the CDR-H1, CDR-H2, and CDR-H3 sequences of SEQ ID NO: 57, and the CDR-L1, CDR-L2, and CDR-L3 sequences of SEQ ID NO: 18;
(viii) the CDR-H1, CDR-H2, and CDR-H3 sequences of SEQ ID NO: 70, and the CDR-L1, CDR-L2, and CDR-L3 sequences of SEQ ID NO: 18;
(ix) the CDR-H1, CDR-H2, and CDR-H3 sequences of SEQ ID NO: 72, and the CDR-L1, CDR-L2, and CDR-L3 sequences of SEQ ID NO: 18;
(x) the CDR-H1, CDR-H2, and CDR-H3 sequences of SEQ ID NO: 78, and the CDR-L1, CDR-L2, and CDR-L3 sequences of SEQ ID NO: 18;
(xi) the CDR-H1, CDR-H2, and CDR-H3 sequences of SEQ ID NO: 80, and the CDR-L1, CDR-L2, and CDR-L3 sequences of SEQ ID NO: 18;
(xii) the CDR-H1, CDR-H2, and CDR-H3 sequences of SEQ ID NO: 89, and the CDR-L1, CDR-L2, and CDR-L3 sequences of SEQ ID NO: 18;
(xiii) the CDR-H1, CDR-H2, and CDR-H3 sequences of SEQ ID NO: 93, and the CDR-L1, CDR-L2, and CDR-L3 sequences of SEQ ID NO: 18;
(xiv) the CDR-H1, CDR-H2, and CDR-H3 sequences of SEQ ID NO: 95, and the CDR-L1, CDR-L2, and CDR-L3 sequences of SEQ ID NO: 18;
(xv) the CDR-H1, CDR-H2, and CDR-H3 sequences of SEQ ID NO: 97, and the CDR-L1, CDR-L2, and CDR-L3 sequences of SEQ ID NO: 18;
(xvi) the CDR-H1, CDR-H2, and CDR-H3 sequences of SEQ ID NO: 99, and the CDR-L1, CDR-L2, and CDR-L3 sequences of SEQ ID NO: 18;
(xvii) the CDR-H1, CDR-H2, and CDR-H3 sequences of SEQ ID NO: 101, and the CDR-L1, CDR-L2, and CDR-L3 sequences of SEQ ID NO: 18;
(xviii) the CDR-H1, CDR-H2, and CDR-H3 sequences of SEQ ID NO: 103, and the CDR-L1, CDR-L2, and CDR-L3 sequences of SEQ ID NO: 18;
(xix) the CDR-H1, CDR-H2, and CDR-H3 sequences of SEQ ID NO: 105, and the CDR-L1, CDR-L2, and CDR-L3 sequences of SEQ ID NO: 18;
(xx) the CDR-H1, CDR-H2, and CDR-H3 sequences of SEQ ID NO: 110, and the CDR-L1, CDR-L2, and CDR-L3 sequences of SEQ ID NO: 18; and
(xxi) the CDR-H1, CDR-H2, and CDR-H3 sequences of SEQ ID NO: 120, and the CDR-L1, CDR-L2, and CDR-L3 sequences of SEQ ID NO: 18.

In some aspects, the disclosure provides an isolated antibody that specifically binds to TL1A, comprising a heavy chain variable region (TL1A-VH) and a light chain variable region (TL1A-VL), comprising the CDR-H1, CDR-H2, and CDR-H3 sequences of SEQ ID NO: 42, and the CDR-L1, CDR-L2, and CDR-L3 sequences of SEQ ID NO: 18.

In some aspects, the disclosure provides an isolated antibody that specifically binds to TL1A, comprising a heavy chain variable region (TL1A-VH) and a light chain variable region (TL1A-VL), comprising a CDR-L1 sequence according to SEQ ID NO: 11; a CDR-L2 sequence according to SEQ ID NO: 12, and a CDR-L3 sequence according to SEQ ID NO: 13, and comprising a set of heavy chain CDRs selected from the list:
(i) a CDR-H1 sequence according to SEQ ID NO: 30; a CDR-H2 sequence according to SEQ ID NO: 41; a CDR-H3 sequence according to SEQ ID NO: 32;
(ii) a CDR-H1 sequence according to SEQ ID NO: 30; a CDR-H2 sequence according to SEQ ID NO: 41; a CDR-H3 sequence according to SEQ ID NO: 35;
(iii) a CDR-H1 sequence according to SEQ ID NO: 30; a CDR-H2 sequence according to SEQ ID NO: 46; a CDR-H3 sequence according to SEQ ID NO: 32;
(iv) a CDR-H1 sequence according to SEQ ID NO: 30; a CDR-H2 sequence according to SEQ ID NO: 46; a CDR-H3 sequence according to SEQ ID NO: 35;
(v) a CDR-H1 sequence according to SEQ ID NO: 30; a CDR-H2 sequence according to SEQ ID NO: 51; a CDR-H3 sequence according to SEQ ID NO: 32;
(vi) a CDR-H1 sequence according to SEQ ID NO: 30; a CDR-H2 sequence according to SEQ ID NO: 51; a CDR-H3 sequence according to SEQ ID NO: 35;
(vii) a CDR-H1 sequence according to SEQ ID NO: 30; a CDR-H2 sequence according to SEQ ID NO: 56; a CDR-H3 sequence according to SEQ ID NO: 32;
(viii) a CDR-H1 sequence according to SEQ ID NO: 30; a CDR-H2 sequence according to SEQ ID NO: 56; a CDR-H3 sequence according to SEQ ID NO: 35;
(ix) a CDR-H1 sequence according to SEQ ID NO: 61; a CDR-H2 sequence according to SEQ ID NO: 41; a CDR-H3 sequence according to SEQ ID NO: 32;
(x) a CDR-H1 sequence according to SEQ ID NO: 61; a CDR-H2 sequence according to SEQ ID NO: 41; a CDR-H3 sequence according to SEQ ID NO: 35;
(xi) a CDR-H1 sequence according to SEQ ID NO: 61; a CDR-H2 sequence according to SEQ ID NO: 51; a CDR-H3 sequence according to SEQ ID NO: 32;
(xii) a CDR-H1 sequence according to SEQ ID NO: 61; a CDR-H2 sequence according to SEQ ID NO: 56; a CDR-H3 sequence according to SEQ ID NO: 32;
(xiii) a CDR-H1 sequence according to SEQ ID NO: 86; a CDR-H2 sequence according to SEQ ID NO: 31; a CDR-H3 sequence according to SEQ ID NO: 35;
(xiv) a CDR-H1 sequence according to SEQ ID NO: 86; a CDR-H2 sequence according to SEQ ID NO: 38; a CDR-H3 sequence according to SEQ ID NO: 35;
(xv) a CDR-H1 sequence according to SEQ ID NO: 86; a CDR-H2 sequence according to SEQ ID NO: 41; a CDR-H3 sequence according to SEQ ID NO: 32;
(xvi) a CDR-H1 sequence according to SEQ ID NO: 86; a CDR-H2 sequence according to SEQ ID NO: 41; a CDR-H3 sequence according to SEQ ID NO: 35;
(xvii) a CDR-H1 sequence according to SEQ ID NO: 86; a CDR-H2 sequence according to SEQ ID NO: 46; a CDR-H3 sequence according to SEQ ID NO: 35;
(xviii) a CDR-H1 sequence according to SEQ ID NO: 86; a CDR-H2 sequence according to SEQ ID NO: 51; a CDR-H3 sequence according to SEQ ID NO: 32;
(xix) a CDR-H1 sequence according to SEQ ID NO: 86; a CDR-H2 sequence according to SEQ ID NO: 51; a CDR-H3 sequence according to SEQ ID NO: 35;
(xx) a CDR-H1 sequence according to SEQ ID NO: 86; a CDR-H2 sequence according to SEQ ID NO: 56; a CDR-H3 sequence according to SEQ ID NO: 32;
(xxi) a CDR-H1 sequence according to SEQ ID NO: 109; a CDR-H2 sequence according to SEQ ID NO: 41; a CDR-H3 sequence according to SEQ ID NO: 35; or
(xxii) a CDR-H1 sequence according to SEQ ID NO: 109; a CDR-H2 sequence according to SEQ ID NO: 56; a CDR-H3 sequence according to SEQ ID NO: 32.

In some aspects, the disclosure provides an isolated antibody that specifically binds to TL1A, comprising a heavy chain variable region (TL1A-VH) and a light chain variable region (TL1A-VL), wherein a CDR-L1 sequence according to SEQ ID NO: 11; a CDR-L2 sequence according to SEQ ID NO: 12, and a CDR-L3 sequence according to SEQ ID NO: 13, and wherein a CDR-H1 sequence according to SEQ ID NO: 30; a CDR-H2 sequence according to SEQ ID NO: 41; a CDR-H3 sequence according to SEQ ID NO: 32.

The TL1A antibody may comprise an TL1A-VH framework sequence comprising a human germline VH framework sequence. The TL1A-VH framework sequence may comprise one or more amino acid substitutions, additions, or deletions, while still retaining functional and structural similarity with the germline from which it was derived. In some aspects, the VH framework is at least 90%, at least 91%, at least 92%, at least 93%, at least 94%, at least 95%, at least 96%, at least 97%, at least 98%, at least 99%, or 100% identical to a human germline VH framework sequence. In some aspects, the TL1A antibody comprises an TL1A-VH framework sequence comprising 1, 2, 3, 4, 5, 6, 7, 8, 9, 10 amino acid substitutions, additions or deletions relative to the human germline VH framework sequence. In some aspects, the 1, 2, 3, 4, 5, 6, 7, 8, 9, or 10 amino acid substitutions, additions or deletions are only in the framework regions. In some aspects, the % identity is based on similarity with VH excluding those portions herein defined as CDRs.

In some aspects, the TL1A-VH framework sequence may be derived from a human germline VH sequence selected from the group consisting of IGHV1-18*01, IGHV1-18*01, IGHV1-18*01, IGHV1-46*01, IGHV3-7*01, IGHV1-2*02, and IGHV1-2*02. The foregoing frameworks are modelled to be compatible with TL1A-VH CDRs of the invention. The invention has identified the human germline VH framework IGHV1-18*01 as highly advantageous with TL1A-VLH CDRs of the invention. In some aspects, the TL1A-VH framework sequence is derived from IGHV1-18*01.

The TL1A antibody may comprise an TL1A-VL framework sequence comprising a human germline VL framework sequence. The TL1A-VL framework sequence may comprise one or more amino acid substitutions, additions, or deletions, while still retaining functional and structural similarity with the germline from which it was derived. In some aspects, the VL framework is at least 90%, at least 91%, at least 92%, at least 93%, at least 94%, at least 95%, at least 96%, at least 97%, at least 98%, at least 99%, or 100% identical to a human germline VL framework sequence. In some aspects, the TL1A antibody comprises an TL1A-VL framework sequence comprising 1, 2, 3, 4, 5, 6, 7, 8, 9, 10 amino acid substitutions, additions or deletions relative to the human germline VL framework sequence. In some aspects, the 1, 2, 3, 4, 5, 6, 7, 8, 9, or 10 amino acid substitutions, additions or deletions are only in the framework regions. In some aspects, the % identity is based on similarity with VL excluding those portions herein defined as CDRs.

The TL1A-VL framework sequence may be derived from a human germline VH sequence selected from the group consisting of IGKV1-39*01, IGKV3-11*01, IGKV3D-7*01, and IGKV3-11*01. The foregoing frameworks are modelled to be compatible with TL1A-VL CDRs of the invention. The invention has identified the human germline VL framework IGKV1-39*01 as highly advantageous with TL1A-VL CDRs of the invention. In some aspects, the TL1A-VH framework sequence is derived from IGKV1-39*01.

The TL1A-VL may comprise an amino acid sequence according to SEQ ID NO: 18, and the TL1A-VH may comprise an amino acid sequence according to a sequence selected from the group consisting of SEQ ID NO: 42, SEQ ID NO: 44, SEQ ID NO: 47, SEQ ID NO:49, SEQ ID NO:52, SEQ ID NO: 54, SEQ ID NO: 57, SEQ ID NO: 70, SEQ ID NO: 72, SEQ ID NO: 78, SEQ ID NO: 80, SEQ ID NO: 89, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 110, and SEQ ID NO:120.

The TL1A antibody may comprise a TL1A-VH sequence at least 90%, 91%, 92%, 93%, 94%, 95%, 96%, 97%, 98%, 99%, or 100% identical to SEQ ID NO: 42, and comprise a TL1A-VL sequence at least 90%, 91%, 92%, 93%, 94%, 95%, 96%, 97%, 98%, 99%, or 100% identical to SEQ ID NO: 18. The TL1A antibody may comprise the TL1A-VH sequence of SEQ ID NO: 42, and the TL1A-VL sequence of SEQ ID NO: 18.

The TL1A antibody may further comprise a constant heavy domain (TL1A-CH1) and a constant light domain (TL1A-CL). In some aspects of the disclosure, the TL1A-CH1 of the antibody comprises a sequence selected from the group consisting of SEQ ID NO: 6, SEQ ID NO: 195, and SEQ ID NO: 199. In some aspects of the disclosure, the TL1A-CL of the antibody comprises a sequence selected from the group consisting of SEQ ID NO: 16, and SEQ ID NO: 201. The TL1A-CH1 may comprise a sequence according to SEQ ID NO: 6. The TL1A-CL may comprise a sequence according to SEQ ID NO: 16. The TL1A-CH1 and TL1A-CL may each be part of a multispecific antibody.

The TL1A-CH1 may be connected to the TL1A-VH, and the TL1A-CL may be connected to the TL1A-VL forming a TL1A-binding Fab domain (TL1A-Fab).

In some aspects, the antibody comprises a single TL1A-Fab domain. Examples of single TL1A-Fab domain antibodies are one-armed antibodies and bispecific antibodies.

TL1A antibodies of the invention may comprise an antibody Fc domain comprising a first Fc chain and a second Fc chain. The Fc domain may be the Fc domain of an IgA (for example IgA₁ or IgA₂), IgD, IgE, IgM, or IgG (for example IgG₁, IgG₂, IgG₃, or IgG₁). The Fc domain may be the Fc domain of an IgG₁. The first Fc chain may comprise, from N-terminus to C-terminus: a first hinge region, a first CH2 region, and a first CH3 region, and the second Fc chain may comprise, from N-terminus to C-terminus: a second hinge region, a second CH2 region, and a second CH3 region. In some aspects, the first Fc chain and the second Fc chain each contain one or more amino acid modifications that promote the association of the first Fc chain with the second Fc chain.

Antibodies of the invention may comprise a hinge region. The hinge region may be selected from any suitable sequence, including a sequence selected from any of Tables 30, 31, 32, and 33. In some aspects, the hinge region is selected from the group consisting of SEQ ID NO: 7, SEQ ID NO: 157, SEQ ID NO: 179, SEQ ID NO: 182, and SEQ ID NO: 189, SEQ ID NO: 191, SEQ ID NO: 204, and SEQ ID NO: 206.

In some aspects, the first hinge region and the second hinge region each contain one or more amino acid modifications that promote the association of the first hinge region with the second hinge region. The first hinge region and the second hinge region may each comprise a different and complementary sequence, and the different and complementary sequences may be selected from one of the following pairs of different and complementary sequences:
(i) SEQ ID NO: 179 and SEQ ID NO: 182; and
(ii) SEQ ID NO: 189 and SEQ ID NO: 191.

In some aspects particularly suitable for one-armed antibody variant, antibodies may comprise a modified full-length hinge for the truncated Fc that include a C(H230)S mutation (SEQ ID NO: 157). SEQ ID NO: 179 is a full-length hinge suitable for the charge mutation variant RRR/EEE with D(H232)R and P(H241)R mutations. SEQ ID NO: 182 is a full-length hinge suitable for the charge mutation variant RRR/EEE with D(H232)E and P(H241)E mutations. Accordingly, SEQ ID NO: 179 and SEQ ID NO: 182 are a pair of different and complimentary hinge sequences. SEQ ID NO: 189 is a full-length hinge suitable for the charge mutation variant RR/EE with the D(H232)R mutation. SEQ ID NO: 191 is a full-length hinge suitable for the charge mutation variant RR/EE with the D(H232)E mutation. Accordingly, SEQ ID NO: 189 and SEQ ID NO: 191 are a pair of different and complimentary hinge sequences. SEQ ID NO: 204 is the lower hinge region starting with D(H232) up to and including P(H243). SEQ ID NO: 206 is the upper hinge region starting with E(H226) up to and including C(H230) (note that SID NO: 204 and 206 are not complete hinge regions and are part of the mFd KiH bispecific antibody format).

TL1A antibodies of the invention may comprise at least a first TL1A-CH1 domain, wherein the N-terminus of the first Fc chain is connected to the C-terminus of the first TL1A-CH1 domain. The TL1A-CL may be connected to a hinge region which is then connected to a CH2 domain. Alternatively, the TL1A-CH1 may be connected to a hinge region which is then connected to a CH2 domain.

The CH2 region may comprise a sequence selected from any one of Tables 30, 31, 32, and 33. The CH2 domain may comprise SEQ ID NO: 8. The first CH2 region and the second CH2 region may each comprise a sequence according to SEQ ID NO: 8. The CH2 region may be connected to a CH3 region.

The CH3 region may comprise a sequence selected from any one of Tables 30, 31, 32, and 33. The CH3 region may comprise a sequence selected from the group consisting of SEQ ID NO: 9, SEQ ID NO: 160, SEQ ID NO: 162, SEQ ID NO: 180, SEQ ID NO: 183, SEQ ID NO: 155, SEQ ID NO: 158, SEQ ID NO: 185, and SEQ ID NO: 187. In some aspects, the CH3 region on the first Fc chain and the CH3 region on the second Fc chain each comprise a sequence according to SEQ ID NO: 9. In some aspects, the first Fc chain comprises a first CH3 region, and the second Fc chain comprises a second CH3 region, and the first CH3 region and the second CH3 region each comprise a different and complementary sequence, and the different and complementary sequences are selected from one of the following pairs of different and complementary sequences:
(i) SEQ ID NO: 160 and SEQ ID NO: 162;
(ii) SEQ ID NO: 180, and SEQ ID NO: 183;
(iii) SEQ ID NO: 155 and SEQ ID NO: 158; and
(iv) SEQ ID NO: 185 and SEQ ID NO: SEQ ID NO: 187.

In some embodiments employing a Knob-in-Hole (KiH) method of heterodimerization, the first CH3 region and the second CH3 region comprise SEQ ID NO: 160 and SEQ ID NO: 162 (including bispecific antibodies targeting TL1A and p40). In some embodiments employing a Knob-in-Hole (KiH) method of heterodimerization the first CH3 region and the second CH3 region comprise SEQ ID NO: 185 and SEQ ID NO: 187 (The 185/187 pair differs from the 160/162 pair by the absence of Cys residues). In some embodiments employing the EEE/RRR, EE/RR, or E/R method of heterodimerization, the first CH3 region and the second CH3 region comprise SEQ ID NO: 180 and SEQ ID NO: 183 (including bispecific antibodies targeting TL1A and p40).

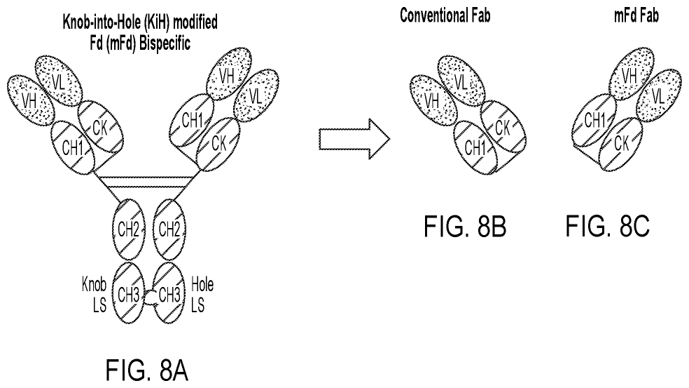

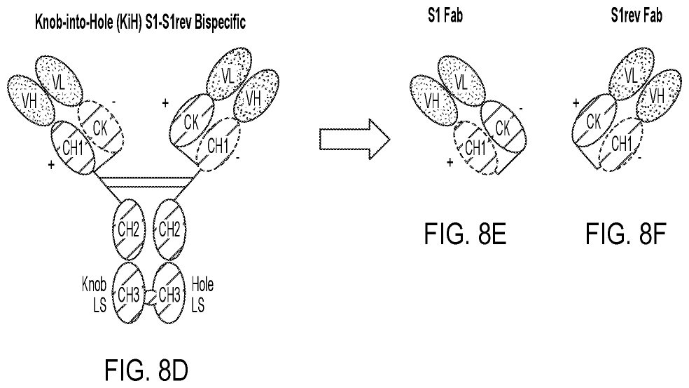

The TL1A antibody may comprise a TL1A-VL bearing polypeptide. A TL1A-VL bearing polypeptide is a polypeptide that comprises a TL1A-VL, and one or more additional peptide regions, such as one or more additional antibody domains. A standard IgG light chain (LC), comprising a TL1A-VL and CL is an example of a TL1A-VL bearing polypeptide. Exemplary TL1A-VL bearing polypeptides that consist essentially of a TL1A-VL and CL include SEQ ID NO: 19 and SEQ ID NO: 202. The term TL1A-VL bearing polypeptide also includes a polypeptides comprising TL1A-VL and other protein domains/peptides (such as a hinge, CH2 and/or CH3 domain). A TL1A LC-Fc (see Table 31, FIG. 8A and p40TL1A-0033) is an example of a TL1A-VL bearing polypeptide comprising a TL1A-VL fused to a CL, fused in turn to a Hinge domain, a CH2 domain, and a CH3 domain. Exemplary TL1A-VL bearing polypeptides that comprise a TL1A-VL, CL, Hinge domain, CH2 and CH3 include SEQ ID NO: 205 of p40TL1A-0033.

The TL1A antibody may comprise a TL1A-VH bearing polypeptide. A TL1A-VH bearing polypeptide is a polypeptide that comprises a TL1-VH, and one or more additional peptide regions, such as one or more additional antibody domains. A standard IgG heavy chain (HC), comprising a TL1A-VH, CH1, Hinge domain, CH2 and CH3 is an example of a TL1A-VH bearing polypeptide. Exemplary TL1A-VH bearing polypeptides that consist essentially of a TL1A-VH, CH1, Hinge, CH2, and CH3 include the HC sequences from Table 30, such as SEQ ID NOs: 10, 23, 25, 27, 29, 34, 37, 40, 43, 45, 48, 50, 53, 55, 58, 60, 63, 65, 67, 69, 71, 73, 75, 77, 79, 81, 83, 85, 88, 90, 92, 94, 96, 98, 100, 102, 104, 106, 108, 111, 113, 115, 117, 119, 121, 123, 127, 132, 135, 138, 142, 144, 146, 150, 152, 154, and 172; the TL1A HC sequences from Table 31: SEQ ID NOs: 184, 188, 192, 194, and 200; the TL1A HC sequences from Table 32: SEQ ID NOs: 156, 161, 164, 165, and 166. The term TL1A-VH bearing polypeptide also includes a polypeptides comprising TL1A-VH and other domains/peptides (such as CH1 and/or short peptides only). A TL1A mFd (see Table 31, FIG. 8A and p40TL1A-0033) is an example of a TL1A-HL bearing polypeptide comprising a TL1A-VH fused to a CH1, fused in turn to a short peptide derived from the upper IgG1 human hinge EPKSC and comprising Cys at position H230 (SEQ ID NO: 206). Exemplary TL1A-VH bearing polypeptides that comprise a TL1A-VH, CH1 and SEQ ID NO: 206 include SEQ ID NO: 207 of p40TL1A-0033.

The TL1A antibody may comprise two identical light chains (two identical TL1A-LC) and two identical heavy chains (two identical TL1A HC). The TL1A-LCs may comprise a TL1A-VL fused to a CL domain. The TL1A-HCs may comprise The TL1A-VH may comprise a sequence selected from any one of Tables 30, 31, 32, and 33. The TL1A-VH may comprise a sequence at least 90%, 91%, 92%, 93%, 94%, 95%, 96%, 97%, 98%, 99%, 100% identical to a sequence selected from the group consisting of SEQ ID NO: 43, SEQ ID NO: 45, SEQ ID NO: 48, SEQ ID NO:50, SEQ ID NO:53, SEQ ID NO: 55, SEQ ID NO: 58, SEQ ID NO: 71, SEQ ID NO: 73, SEQ ID NO: 79, SEQ ID NO: 81, SEQ ID NO: 90, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 111, and SEQ ID NO:121.

The TL1A-VL bearing polypeptide may comprise a sequence selected from any one of Tables 30, 31, 32, and 33. The TL1A-VL bearing polypeptide may comprise a sequence at least 90%, 91%, 92%, 93%, 94%, 95%, 96%, 97%, 98%, 99%, or 100% identical to a sequence according to SEQ ID NO: 19.

In some aspects, the TL1A antibody comprises two identical TL1A-Fab domains. In some aspects of this invention, the TL1A antibody comprises two identical light chains and two identical heavy chains. In some aspects the TL1A antibody comprises two identical light chains comprising a sequence in accordance with SEQ ID NO: 19. In some aspects, the TL1A antibody comprises two identical heavy chains comprising a sequence in accordance with SEQ ID NO: 43.

In some aspects, the TL1A antibodies are characterized by a TL1A IC₅₀ of less than 20 nM in a competition ELISA. The TL1A antibodies may be characterized by a TL1A IC₅₀ of less than 10 nM in a competition ELISA. The TL1A antibodies may be characterized by a TL1A IC₅₀ of less than 50 nM in a TF-1 NFκB reporter cell bioassay. The TL1A antibodies may be characterized by a TL1A IC₅₀ of less than 30 nM in a TF-1 NFκB reporter cell bioassay. The TL1A antibodies may be characterized by a TL1A IC₅₀ of less than 1 nM in a whole blood IFNγ assay.

In some aspects, the TL1A antibody is bivalent and monospecific. Exemplary bivalent and monospecific TI1A antibodies comprise a standard IgG format of two identical heave chains and two identical light chains. Bivalent and monospecific TL1A antibodies are exemplified in Table 30. In some aspects, the antibody is characterized by a score of less than 4 in an affinity-capture self-interaction nanoparticle spectroscopy (AC SINS) assay.

In some aspects, the TL1A antibody is monovalent monospecific antibody. Such an antibody is monospecific in that it can only bind to a single epitope or target, and monovalent in that it only has a single binding domain.

The present disclosure provides examples of two different formats of monospecific monovalent TL1A antibodies. The first format is a one-armed TL1A antibody, which comprises a single Fab domain fused to a functional Fc domain, and the second format is an antibody comprising a null arm, which is a TL1A antibody comprising a TL1A binding Fab domain and a second Fab domain that does not bind TL1A, or any other known target.

In some aspects, the TL1A antibody comprises a one-armed antibody. A one-armed antibody is an IgG antibody comprising an intact Fc domain of two hinge domains, two CH2 domain, and two CH3 domains, but only one Fab region. A one-armed TL1A antibody is an IgG TL1A antibody comprising an intact Fc domain of two hinge domains, two CH2 domain, and two CH3 domains, but only one TL1A-Fab region. In some aspects, the TL1A antibody comprises a monovalent monospecific antibody. A one-armed TL1A antibody is monovalent and monospecific due to its single TL1A Fab domain.

The one-armed antibody may comprise a first heavy chain and a first light chain, and a second heavy chain, wherein the first heavy chain is a heavy chain as herein described, and the first light chain is as herein described, and the second heavy chain does not comprise a VH domain or CH1 domain.

The one-armed antibody may comprise a first heavy chain and a first light chain, and a second heavy chain, wherein the first heavy chain and the first light chain comprise a TL1A binding site that binds to TL1A, and wherein the first antibody heavy chain comprises an amino acid sequence selected from the group consisting of SEQ ID NO: 161, SEQ ID NO: 164, and SEQ ID NO: 165, the first antibody light chain comprises the amino acid sequence of SEQ ID NO: 19, and the second antibody heavy chain comprises the amino acid sequence of SEQ ID NO: 163.

The one-armed antibody may comprise a first heavy chain and a first light chain, and a second heavy chain, wherein the first heavy chain and the first light chain comprise a TL1A binding site that binds to TL1A, wherein the first antibody heavy chain comprises the amino acid sequence of SEQ ID NO: 161, the first antibody light chain comprises the amino acid sequence of SEQ ID NO: 19, and the second antibody heavy chain comprises the amino acid sequence of SEQ ID NO: 163.

The one-armed antibody may comprise a first heavy chain and a first light chain, and a second heavy chain, wherein the first heavy chain and the first light chain comprise a TL1A binding site that binds to TL1A, wherein the first antibody heavy chain comprises the amino acid sequence of SEQ ID NO: 164, the first antibody light chain comprises the amino acid sequence of SEQ ID NO: 19, and the second antibody heavy chain comprises the amino acid sequence of SEQ ID NO: 163.

The one-armed antibody may comprise a first heavy chain and a first light chain, and a second heavy chain, wherein the first heavy chain and the first light chain comprise a TL1A binding site that binds to TL1A, wherein the first antibody heavy chain comprises the amino acid sequence of SEQ ID NO: 165, the first antibody light chain comprises the amino acid sequence of SEQ ID NO: 19, and the second antibody heavy chain comprises the amino acid sequence of SEQ ID NO: 163.

In some aspects, the antibody comprises a one-armed TL1A antibody wherein the antibody has a terminal half-life of less than 7 days in cynomolgus monkeys. Such an antibody can be advantageous in applications where a shorter half-life is useful, such as certain oncology applications and applications in discrete organs or compartments, such as the eye.

In some aspects, the TL1A antibody comprises a null arm. An antibody comprising a null arm is a monovalent monospecific antibody, wherein one of the antigen binding sites has been modified to eliminate binding to the target. In some aspects, the heavy chain CDRs have been modified to eliminate binding to the target. In some aspects, the light chain CDRs have not been modified. A null-arm TL1A antibody is a monovalent monospecific TL1A antibody, wherein one of the antigen binding sites has been modified to eliminate binding to TL1A. In some aspects, one or more of the TL1A VH CDRs have been modified to eliminate binding to TL1A. A TL1A-VH comprising one or more CDRs modified so as to eliminate TL1A binding may be referred to as a xTL1A-VH. In some aspects, the TL1A VL CDRs have not been modified.

A null-arm TL1A antibody may comprise a single TL1A-VH and a single xTL1A-VH, and two identical TL1A-VLs.

In some aspects, the CDR-H1, CDR-H2, and CDR-H3 are each derived from a TL1A-VH. A CDR is derived from a TL1A-binding VH if the CDR was made or produced by modifying a TL1A-binding VH by changing its amino acid sequence by a documented process wherein at least one structural feature is retained at each process step.

In some aspects, the null-arm TL1A antibody comprises a xTL1A-VH comprising a sequence corresponding to a sequence selected from the group consisting of SEQ ID NO: 131, SEQ ID NO: 134, SEQ ID NO: 137, SEQ ID NO: 141, SEQ ID NO: 145, SEQ ID NO: 149, SEQ ID NO: 151, SEQ ID NO: 153.

In some aspects, the antibody comprises a null-arm TL1A antibody comprising a first heavy chain and a first light chain, and a second heavy chain and second light chain, wherein the first heavy chain and the first light chain comprise a first Fab domain that binds to TL1A (TL1A-Fab) (as herein described) and the second heavy chain and the second light chain comprise a second Fab domain that comprises a non-binding TL1A binding domain that does not specifically bind to a target, (xTL1A-Fab), and wherein the first and second light chain are identical.

In some aspects, the xTL1A-Fab comprises a VH comprising a sequence corresponding to a sequence selected from the group consisting of SEQ ID NO: 131, SEQ ID NO: 134, SEQ ID NO: 137, SEQ ID NO: 141, SEQ ID NO: 145, SEQ ID NO: 149, SEQ ID NO: 151, SEQ ID NO: 153. xTL1A-Fabs can be combined with any of the TL1A-VH described herein to generate a null-arm TL1A antibody.

In some aspects, an antibody comprising a TL1A-Fab and an xTL1A-Fab may be referred to as a null-arm TL1A antibody.

In some aspects, the null-arm TL1A antibody comprises the sequences as set forth in Table 30. In some aspects, the null-arm TL1A antibody is selected from the group consisting of [xTL1A]-0336; [xTL1A]-0338; [xTL1A]-0341; [xTL1A]-0347; [xTL1A]-0349; [xTL1A]-0352; [xTL1A]-0358; [xTL1A]-0360; [xTL1A]-0363, and as set forth in Table 30. Any of these antibodies may be generated using modified Fc domains, and may, for example, incorporate LS mutations or alternative heterodimerization strategies such as KiH.

In some aspects, the antibody comprises a null-arm TL1A antibody wherein the second Fab domain comprises a non-binding TL1A heavy chain variable region (xTL1A-VH) and a non-binding TL1A light chain variable region (xTL1A-VL), wherein a CDR-L1 sequence according to SEQ ID NO: 11; a CDR-L2 sequence according to SEQ ID NO: 12, and a CDR-L3 sequence according to SEQ ID NO: 13, and wherein
(i) a CDR-H1 sequence according to SEQ ID NO: 139; a CDR-H2 sequence according to SEQ ID NO: 140; a CDR-H3 sequence according to SEQ ID NO: 133;
(ii) a CDR-H1 sequence according to SEQ ID NO: 128; a CDR-H2 sequence according to SEQ ID NO: 129; a CDR-H3 sequence according to SEQ ID NO: 130;
(iii) a CDR-H1 sequence according to SEQ ID NO: 128; a CDR-H2 sequence according to SEQ ID NO: 129; a CDR-H3 sequence according to SEQ ID NO: 133;
(iv) a CDR-H1 sequence according to SEQ ID NO: 128; a CDR-H2 sequence according to SEQ ID NO: 129; a CDR-H3 sequence according to SEQ ID NO: 136;
(v) a CDR-H1 sequence according to SEQ ID NO: 139; a CDR-H2 sequence according to SEQ ID NO: 140; a CDR-H3 sequence according to SEQ ID NO: 130;
(vi) a CDR-H1 sequence according to SEQ ID NO: 139; a CDR-H2 sequence according to SEQ ID NO: 140; a CDR-H3 sequence according to SEQ ID NO: 136;
(vii) a CDR-H1 sequence according to SEQ ID NO: 147; a CDR-H2 sequence according to SEQ ID NO: 148; a CDR-H3 sequence according to SEQ ID NO: 130;
(viii) a CDR-H1 sequence according to SEQ ID NO: 147; a CDR-H2 sequence according to SEQ ID NO: 148; a CDR-H3 sequence according to SEQ ID NO: 133; or
(ix) a CDR-H1 sequence according to SEQ ID NO: 147; a CDR-H2 sequence according to SEQ ID NO: 148; a CDR-H3 sequence according to SEQ ID NO: 136.

In some aspects, the antibody comprises a null-arm TL1A antibody wherein the second Fab domain comprises a heavy chain variable region (xTL1A-VH) and a light chain variable region (xTL1A-VL), wherein a CDR-L1 sequence according to SEQ ID NO: 11; a CDR-L2 sequence according to SEQ ID NO: 12, and a CDR-L3 sequence according to SEQ ID NO: 13, and wherein a CDR-H1 sequence according to SEQ ID NO: 139; a CDR-H2 sequence according to SEQ ID NO: 140; a CDR-H3 sequence according to SEQ ID NO: 133.

In some aspects, the antibody comprises a null-arm TL1A antibody wherein the second Fab domain comprises a light chain variable amino acid sequence according to SEQ ID NO: 18, and wherein the second Fab domain comprises a heavy chain variable amino acid sequence according to a sequence selected from the group consisting of SEQ ID NO: 143, SEQ ID NO: 131, SEQ ID NO: 134, SEQ ID NO: 137, SEQ ID NO: 141, SEQ ID NO: 145, SEQ ID NO: 57, SEQ ID NO: 70, SEQ ID NO: 72, SEQ ID NO: 78, SEQ ID NO: 80, SEQ ID NO: 89, SEQ ID NO: 149, SEQ ID NO: 151, and SEQ ID NO: 153.

In some aspects, the antibody comprises a null-arm TL1A antibody wherein the second Fab domain comprises a light chain variable amino acid sequence according to SEQ ID NO: 18, and wherein the second Fab domain comprises a heavy chain variable amino acid sequence according to SEQ ID NO: 143.

In some aspects, the antibody comprises a null-arm TL1A antibody comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target (xTL1A-Fab), wherein the first antibody heavy chain comprises an amino acid sequence according to SEQ ID NO: 161, the first antibody light chain comprises an amino acid sequence according to SEQ ID NO: 19, the second antibody light chain comprises an amino acid sequence according to SEQ ID NO: 19, and the second antibody heavy chain comprises an amino acid sequence according to a sequence selected from the group consisting of SEQ ID NO: 166.

In some aspects, the antibody comprises a null-arm TL1A antibody comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target (xTL1A-Fab), wherein the first antibody heavy chain comprises an amino acid sequence according to SEQ ID NO: 161, the first antibody light chain comprises an amino acid sequence according to SEQ ID NO: 19, the second antibody light chain comprises an amino acid sequence according to SEQ ID NO: 19, and the second antibody heavy chain comprises an amino acid sequence according to a sequence selected from the group consisting of SEQ ID NO: 166.

In some aspects, the antibody comprises a null-arm TL1A antibody comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target (xTL1A-Fab), wherein the first antibody heavy chain comprises an amino acid sequence according to SEQ ID NO: 161, the first antibody light chain comprises an amino acid sequence according to SEQ ID NO: 19, the second antibody light chain comprises an amino acid sequence according to SEQ ID NO: 19, and the second antibody heavy chain comprises an amino acid sequence according to SEQ ID NO: 166.

The null-arm TL1A antibody may be characterized by a score of less than 2% high molecular mass species when determined by analytical size-exclusion chromatography (aSEC). The aSEC assay may be conducted as herein described in the Examples.

The null-arm TL1A antibody may be characterized by a score of less than 7 in an affinity-capture self-interaction nanoparticle spectroscopy (AC SINS) assay. The null-arm TL1A antibody may be characterized by a score of less than 6 in an affinity-capture self-interaction nanoparticle spectroscopy (AC SINS) assay. The null-arm TL1A antibody may be characterized by a score of less than 5 in an affinity-capture self-interaction nanoparticle spectroscopy (AC SINS) assay.

The null-arm TL1A antibody may be characterized by a score of less than 4 in an affinity-capture self-interaction nanoparticle spectroscopy (AC SINS) assay. The null-arm TL1A antibody may be characterized by a score of less than 3 in an affinity-capture self-interaction nanoparticle spectroscopy (AC SINS) assay. The AC SINS assay may be conducted as herein described in the Examples.

The null-arm TL1A antibody may be characterized by IC50 of less than 2 nM in an NFκB TL1A neutralization bioassay. The calculation of IC50 in an NFκB TL1A neutralization bioassay may be conducted as herein described in the Examples.

In some aspects, the null-arm TL1A antibody binds cynomologus TL1A within one order of magnitude of human TL1A as measured by SPR. In some aspects, the Null-arm TL1A antibody has a binding affinity at least 500× higher for human TL1A than for mouse T1A as measured by SPR. In some aspects, the null-arm TL1A antibody has a binding affinity at least 2000× higher for human TL1A than for mouse TL1A as measured by SPR. The null-arm TL1A antibody may be characterized by a KD of less than 500 pM as measured by SPR. The null-arm TL1A antibody may be characterized by a KD of less than 320 pM as measured by SPR. The null-arm TL1A antibody may be characterized by a KD of less than 300 pM as measured by SPR. The respective SPR assay may be conducted as herein described in the Examples.

The null-arm TL1A antibody may be characterized by a terminal half-life of at least 21 days in cynomolgus monkeys. The null-arm TL1A antibody may be characterized by a terminal half-life of at least 25 days in cynomolgus monkeys. The null-arm TL1A antibody may be characterized by a terminal half-life of at least 28 days in cynomolgus monkeys.

The null-arm TL1A antibody may be characterized by a terminal half-life of at between 21 days and 45 days in cynomolgus monkeys. The null-arm TL1A antibody may be characterized by a terminal half-life of between 25 days and 35 days in cynomolgus monkeys. The null-arm TL1A antibody may be characterized by a terminal half-life of between 28 days and 30 days in cynomolgus monkeys.

Antibodies to p40 The disclosure provides antibodies that bind to p40. Anti-p40 antibodies of the invention may bind one or more additional targets. P40 is also known as IL12B, CLMF, CLMF2, IL-12B, IMD28, NKSF, NKSF2, and IMD29. As used herein, the term “p40” includes variants, isoforms, homologs, orthologs and paralogs of one or more of p40. In some embodiments, an antibody disclosed herein cross-reacts with p40 from species other than human, such as p40 of cynomolgus monkey, as well as different forms of p40. In some embodiments, an antibody may be completely specific for human p40 and may not exhibit species cross-reactivity or other types of cross-reactivity. As used herein the term p40 refers to naturally occurring human p40 unless contextually dictated otherwise. A “p40 antibody” “anti-p40 antibody” or other similar designation means any antibody (as defined herein) that binds or reacts with p40, an isoform, fragment or derivative thereof. The full length, mature form of p40 is represented by UniProtKB/Swiss-Prot accession number P29460. The full length, mature form of cynomolgus monkey p40, is represented by UniProtKB/Swiss-Prot accession number G7P6S2.

The p40 antibody may comprise a p40-VH framework sequence comprising a human germline VH framework sequence. The p40-VH framework sequence may comprise one or more amino acid substitutions, additions, or deletions, while still retaining functional and structural similarity with the germline from which it was derived. In some aspects, the VH framework sequence is at least 90%, at least 91%, at least 92%, at least 93%, at least 94%, at least 95%, at least 96%, at least 97%, at least 98%, at least 99%, or 100% identical to a human germline VH framework sequence. In some aspects, the p40 antibody comprises an p40-VH framework sequence comprising 1, 2, 3, 4, 5, 6, 7, 8, 9, 10 amino acid substitutions, additions or deletions relative to the human germline VH framework sequence. In some aspects, the 1, 2, 3, 4, 5, 6, 7, 8, 9, or 10 amino acid substitutions, additions or deletions are only in the framework regions. In some aspects, the % identity is based on similarity with VH excluding those portions herein defined as CDRs.

The disclosure provides for an isolated antibody that specifically binds to p40 through a p40 binding domain and wherein the antibody comprises at least one additional antigen binding domain that specifically binds TL1A, wherein the p40 binding domain comprises a heavy chain variable region (p40-VH) and a light chain variable region (p40-VL), comprising the CDR-H1, CDR-H2, and CDR-H3 sequence of SEQ ID NO: 171, and the CDR-L1, CDR-L2, and CDR-L3 sequences of SEQ ID NO: 177.

The disclosure provides for an isolated antibody that specifically binds to p40 through a p40 binding domain and wherein the antibody comprises a TL1A antigen binding domain that specifically binds to aTL1A, and wherein the p40 binding domain comprises a heavy chain variable region (p40-VH) and a light chain variable region (p40-VL), wherein the CDR-H1 of the p40 binding domain comprises the amino acid sequence of SEQ ID NO: 167; the CDR-H2 of the p40 binding domain comprises the amino acid sequence of SEQ ID NO: 168; the CDR-H3 of the p40 binding domain comprises the amino acid sequence of SEQ ID NO: 169; the CDR-L1 of the p40 binding domain comprises the amino acid sequence of SEQ ID NO: 173; the CDR-L2 of the p40 binding domain comprises the amino acid sequence of SEQ ID NO: 174, and the CDR-L3 of the p40 binding domain comprises the amino acid sequence of SEQ ID NO: 175.

In some aspects, the p40-VH framework sequence may be derived from a human germline VH sequence selected from the group consisting of DP3, DP7, DP73, DP75, and DP88. In some aspects, the p40-VH framework sequence is derived from DP73.

The p40-VH framework sequence may be derived from a human germline VH sequence selected from the group consisting of IGHV5-51*01/5-51*03, IGHV1-46*01/1-46*03, IGKV1-39*01, IGHV5-51*02, IGHV5-51*04, IGHV1-46*02, IGHV1-69-2*01, IGHV1-69*08, IGHV1-69*02, IGHV1-69*06/1-69*14, IGHV1-69*04/1-69*09 and IGHV1-2*02. In some aspects, the p40-VH framework sequence is derived from IGHV5-51*01/5-51*03.

The invention has identified the human germline to IGHV5-51*01/5-51*03 (DP-73) as highly advantageous for p40-VH CDRs of the invention. Experimental data demonstrates that IGHV1-46*01/1-46*03 (DP-7) and IGKV1-39*01 or DPK9 (with L465 mutation) for V_{L} and VK1-33 (with L465 mutation) were advantageously able to retain binding within ˜3-4 fold after grafting. Other possible germlines suitable for use with p40-VH CDRs of the invention include other IGHV5-51 loci germlines IGHV5-51*02 and IGHV5-51*04, other IGHV1-46 loci germlines such as IGHV1-46*02, IGHV1-69-2*01 (DP-3) and other IGHV1-69 loci germlines IGHV1-69*08, IGHV1-69*02, IGHV1-69*06/1-69*14 (DP-88), IGHV1-69*04/1-69*09 and IGHV1-2*02 (DP-75). The foregoing frameworks are modelled to be compatible with p40-VH CDRs of the invention.

The p40 antibody may comprise a p40-VL framework sequence comprising a human germline VL framework sequence. The p40-VL framework sequence may comprise one or more amino acid substitutions, additions, or deletions, while still retaining functional and structural similarity with the germline from which it was derived. In some aspects, the VL framework is at least 90%, at least 91%, at least 92%, at least 93%, at least 94%, at least 95%, at least 96%, at least 97%, at least 98%, at least 99%, or 100% identical to a human germline VL framework sequence. In some aspects, the p40 antibody comprises a p40-VL framework sequence comprising 1, 2, 3, 4, 5, 6, 7, 8, 9, 10 amino acid substitutions, additions or deletions relative to the human germline VL framework sequence. In some aspects, the 1, 2, 3, 4, 5, 6, 7, 8, 9, or 10 amino acid substitutions, additions or deletions are only in the framework regions. In some aspects, the % identity is based on similarity with VL excluding those portions herein defined as CDRs.

In some aspects, the p40-VL framework sequence may be derived from a human germline VL sequence selected from the group consisting of DPK4, DPK5, DPK7, DPK8, and DPK9. In some aspects, the p40-VL framework sequence is derived from DPK7.

The p40-VL framework sequence may be derived from a human germline VL sequence selected from the group consisting of IGKV1D-16*01, IGKV1D-16*02, IGKV1-16*01, IGKV1-16*02, IGKV1-39*01, IGKV1-12*01/1-12*02/1D-12*01/1D-12*02, IGKV1-9*01, IGKV1-5*03, IGKV1-5*01, and IGKV1-27*01. In some aspects, the p40-VL framework sequence is derived from IGKV1D-16*01.

The invention has identified the human germline VL framework IGKV1D-16*01 (DPK7) as highly advantageous with p40-VL CDRs of the invention. Advantageously, other VL germlines that may be used with p40-VL regions of the invention include other IGKV1D-16 loci germlines IGKV1D-16*02, IGKV1-16*01, IGKV1-16*02, IGKV1-39*01 (DPK9), IGKV1-12*01/1-12*02/1D-12*01/1D-12*02 (DPK5), IGKV1-9*01 (DPK8), IGKV1-5*03, IGKV1-5*01, IGKV1-27*01 (DPK4). The foregoing frameworks are modelled to be compatible with p40-VL CDRs of the invention.

In some aspects of the disclosure, the p40-CH1 of the antibody comprises a sequence selected from the group consisting of SEQ ID NO: 6, SEQ ID NO: 195, and SEQ ID NO: 199. In some aspects of the disclosure, the p40-CL of the antibody comprises a sequence selected from the group consisting of SEQ ID NO: 16, SEQ ID NO: 197, and SEQ ID NO: 201. The p40-CH1 may comprise a sequence according to SEQ ID NO:6. The p40-CL may comprise a sequence according to SEQ ID NO: 16. The p40-CH1 and p40-CL may each be part of a bispecific antibody.

The p40-CH1 may be connected to the p40-VH, and the p40-CL may be connected to the p40-VL forming an p40-binding Fab domain (p40-Fab).

p40 antibodies of the invention may comprise a hinge region. The hinge region may be selected from any suitable sequence, including a sequence selected from any of Tables 31 and 33. In some aspects, the hinge region is selected from the group consisting of SEQ ID NO: 7, SEQ ID NO: 157, SEQ ID NO: 179, SEQ ID NO: 182, and SEQ ID NO: 189, SEQ ID NO: 191, SEQ ID NO: 204, and SEQ ID NO: 206.

The p40-CL may be connected to a hinge region which is then connected to a CH2 domain. Alternatively, the p40-CH1 may be connected to a hinge region which is then connected to a CH2 domain. The CH2 domain may comprise SEQ ID NO: 8. The CH2 region may be connected to a CH3 region. The CH3 region may comprise a sequence selected from any one of Tables 31, and 33. The CH3 region may comprise a sequence selected from the group consisting of SEQ ID NO: 9, SEQ ID NO: 160, SEQ ID NO: 162, SEQ ID NO: 180, SEQ ID NO: 183, SEQ ID NO: 155, SEQ ID NO: 158, SEQ ID NO: 185, and SEQ ID NO: 187. In some aspects, the first Fc chain comprises a first CH3 region, and the second Fc chain comprises a second CH3 region, and the first CH3 region and the second CH3 region each comprise a different and complementary sequence, and the different and complementary sequences are selected from one of the following pairs of different and complementary sequences:
(i) SEQ ID NO: 160 and SEQ ID NO: 162;
(ii) SEQ ID NO: 180, and SEQ ID NO: 183;
(iii) SEQ ID NO: 155 and SEQ ID NO: 158;
(iv) SEQ ID NO: 185 and SEQ ID NO: SEQ ID NO: 187.

In some aspects, the first CH3 region and the second CH3 region comprise SEQ ID NO: 160 and SEQ ID NO: 162.

The p40-VH bearing polypeptide may comprise a sequence selected from any one of Tables 31, and 33. The p40-VH bearing polypeptide may comprise a sequence at least 90%, 91%, 92%, 93%, 94%, 95%, 96%, 97%, 98%, 99%, or 100% identical to a sequence selected from the group consisting of SEQ ID NO: 181, SEQ ID NO: 186, SEQ ID NO: 190, SEQ ID NO: 193, SEQ ID NO: 196, and SEQ ID NO: 203.

The p40-VL bearing polypeptide may comprise a sequence selected from any one of Tables 31, and 33. The p40-VL bearing polypeptide may comprise a sequence at least 90%, 91%, 92%, 93%, 94%, 95%, 96%, 97%, 98%, 99%, or 100% identical to a sequence selected from the group consisting of SEQ ID NO: 178, and SEQ ID NO: 198.

In some aspects, the disclosure provides an isolated bispecific antibody that specifically binds p40 through a p40 binding domain and specifically binds TL1A through a TL1A binding domain, wherein the p40 binding domain comprises a heavy chain variable region (p40-VH) and a light chain variable region (p40-VL), wherein the p40-VH comprises the CDR-H1, CDR-H2, and CDR-H3 sequences of encoded by the plasmid deposited at the ATCC and having ATCC Accession No. PTA-127206, and the p40-VL comprises the CDR-L1, CDR-L2, and CDR-L3 sequences encoded by the plasmid deposited at the ATCC and having ATCC Accession No. PTA-127205.

In some aspects, the disclosure provides p40 antibodies comprising a p40-VH sequence encoded by the plasmid deposited at the ATCC and having ATCC Accession No. PTA-127206, and a p40-VL sequence encoded by the plasmid deposited at the ATCC and having ATCC Accession No. PTA-127205.

In some aspects, the disclosure provides p40 antibodies comprising a p40-VH bearing polypeptide sequence encoded by the plasmid deposited at the ATCC and having ATCC Accession No. PTA-127204. In some aspects, the disclosure provides p40 antibodies comprising a p40-VL bearing polypeptide sequence encoded by the plasmid deposited at the ATCC and having ATCC Accession No. PTA-127203.

Antibodies to TL1A and p40

The disclosure provides antibodies that bind to TL1A and p40. As used herein, the terms TL1A and p40 include variants, isoforms, homologs, orthologs and paralogs of TL1A and p40 respectively. In some embodiments, an antibody disclosed herein cross-reacts with one or more of TL1A and p40 from species other than human, such as TL1A and p40 of cynomolgus monkey. In some embodiments, an antibody may be completely specific for TL1A and p40 and may not exhibit species cross-reactivity or other types of cross-reactivity. As used herein the term TL1A and p40 refers to naturally occurring human TL1A and p40 unless contextually dictated otherwise. A “TL1A/p40 antibody” “anti-TL1A/p40 antibody” or other similar designation means any antibody (as defined herein) that binds or reacts with TL1A and p40, an isoform, fragment or derivative thereof.

In some embodiments, the invention provides a TL1A/p40 antibody having a light chain variable region (VL) sequence and a heavy chain variable region (VH) sequence as found in one or more of Tables 30, 31, 32, and 33, or variants thereof.

The invention also provides CDR portions of TL1A/p40 antibodies. Determination of CDR regions is defined in FIGS. 10A-10D. In some embodiments, the TL1A/p40 antibody comprises the six CDRs of a TL1A antibody selected from one of Tables 30, 31, 32, and 33. In some embodiments, the antibody comprises the VH and VL of a TL1A antibody selected from one of Tables 30, 31, 32, and 33, In some embodiments, the TL1A/p40 antibody comprises sequences selected from one of Tables 30, 31, 32, and 33.

In some embodiments, the disclosure provides anti-TL1A/p40 antibodies containing variations of the CDRs, VH, VL, HC, and LC regions shown in one or more of Table 30, 31, 32, and 33, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 30, 31, 32, and 33. These amounts are not meant to be limiting and increments between the recited percentages are specifically envisioned as part of the disclosure.

The present disclosure provides an isolated antibody comprising a first antigen binding site that binds to TL1A and a second antigen binding site that binds to p40, wherein the first antigen binding site comprises a TL1A-binding heavy chain variable region (TL1A-VH) and a TL1A-binding light chain variable region (TL1A-VL), wherein the second antigen binding site comprises a p40-binding heavy chain variable region (p40-VH) and a p40-binding light chain variable region (p40-VL), and wherein the p40 binding domain comprises a CDR-L1 sequence according to SEQ ID NO: 173; a CDR-L2 sequence according to SEQ ID NO: 174, and a CDR-L3 sequence according to SEQ ID NO: 175, a CDR-H1 sequence according to SEQ ID NO: 167, a CDR-H2 sequence according to SEQ ID NO: 168, and a CDR-H3 sequence according to SEQ ID NO: 169, and wherein the TL1A binding domain comprises a CDR-L1 sequence according to SEQ ID NO: 11; a CDR-L2 sequence according to SEQ ID NO: 12, and a CDR-L3 sequence according to SEQ ID NO: 13, and further comprises a set of heavy chain CDRs selected from the list:
(i) a CDR-H1 sequence according to SEQ ID NO: 30; a CDR-H2 sequence according to SEQ ID NO: 41; a CDR-H3 sequence according to SEQ ID NO: 32;
(ii) a CDR-H1 sequence according to SEQ ID NO: 30; a CDR-H2 sequence according to SEQ ID NO: 41; a CDR-H3 sequence according to SEQ ID NO: 35;
(iii) a CDR-H1 sequence according to SEQ ID NO: 30; a CDR-H2 sequence according to SEQ ID NO: 46; a CDR-H3 sequence according to SEQ ID NO: 32;
(iv) a CDR-H1 sequence according to SEQ ID NO: 30; a CDR-H2 sequence according to SEQ ID NO: 46; a CDR-H3 sequence according to SEQ ID NO: 35;
(v) a CDR-H1 sequence according to SEQ ID NO: 30; a CDR-H2 sequence according to SEQ ID NO: 51; a CDR-H3 sequence according to SEQ ID NO: 32;
(vi) a CDR-H1 sequence according to SEQ ID NO: 30; a CDR-H2 sequence according to SEQ ID NO: 51; a CDR-H3 sequence according to SEQ ID NO: 35;
(vii) a CDR-H1 sequence according to SEQ ID NO: 30; a CDR-H2 sequence according to SEQ ID NO: 56; a CDR-H3 sequence according to SEQ ID NO: 32;
(viii) a CDR-H1 sequence according to SEQ ID NO: 30; a CDR-H2 sequence according to SEQ ID NO: 56; a CDR-H3 sequence according to SEQ ID NO: 35;
(ix) a CDR-H1 sequence according to SEQ ID NO: 61; a CDR-H2 sequence according to SEQ ID NO: 41; a CDR-H3 sequence according to SEQ ID NO: 32;
(x) a CDR-H1 sequence according to SEQ ID NO: 61; a CDR-H2 sequence according to SEQ ID NO: 41; a CDR-H3 sequence according to SEQ ID NO: 35;
(xi) a CDR-H1 sequence according to SEQ ID NO: 61; a CDR-H2 sequence according to SEQ ID NO: 51; a CDR-H3 sequence according to SEQ ID NO: 32;
(xii) a CDR-H1 sequence according to SEQ ID NO: 61; a CDR-H2 sequence according to SEQ ID NO: 56; a CDR-H3 sequence according to SEQ ID NO: 32;
(xiii) a CDR-H1 sequence according to SEQ ID NO: 86; a CDR-H2 sequence according to SEQ ID NO: 31; a CDR-H3 sequence according to SEQ ID NO: 35;
(xiv) a CDR-H1 sequence according to SEQ ID NO: 86; a CDR-H2 sequence according to SEQ ID NO: 38; a CDR-H3 sequence according to SEQ ID NO: 35;
(xv) a CDR-H1 sequence according to SEQ ID NO: 86; a CDR-H2 sequence according to SEQ ID NO: 41; a CDR-H3 sequence according to SEQ ID NO: 32;
(xvi) a CDR-H1 sequence according to SEQ ID NO: 86; a CDR-H2 sequence according to SEQ ID NO: 41; a CDR-H3 sequence according to SEQ ID NO: 35;
(xvii) a CDR-H1 sequence according to SEQ ID NO: 86; a CDR-H2 sequence according to SEQ ID NO: 46; a CDR-H3 sequence according to SEQ ID NO: 35;
(xviii) a CDR-H1 sequence according to SEQ ID NO: 86; a CDR-H2 sequence according to SEQ ID NO: 51; a CDR-H3 sequence according to SEQ ID NO: 32;
(xix) a CDR-H1 sequence according to SEQ ID NO: 86; a CDR-H2 sequence according to SEQ ID NO: 51; a CDR-H3 sequence according to SEQ ID NO: 35;
(xx) a CDR-H1 sequence according to SEQ ID NO: 86; a CDR-H2 sequence according to SEQ ID NO: 56; a CDR-H3 sequence according to SEQ ID NO: 32;
(xxi) a CDR-H1 sequence according to SEQ ID NO: 109; a CDR-H2 sequence according to SEQ ID NO: 41; a CDR-H3 sequence according to SEQ ID NO: 35; and
(xxii) a CDR-H1 sequence according to SEQ ID NO: 109; a CDR-H2 sequence according to SEQ ID NO: 56; a CDR-H3 sequence according to SEQ ID NO: 32;

In some aspects, the present disclosure provides an isolated antibody comprising a first antigen binding site that binds to TL1A and a second antigen binding site that binds to p40, wherein the first antigen binding site comprises a TL1A-binding heavy chain variable region (TL1A-VH) and a TL1A-binding light chain variable region (TL1A-VL), wherein the second antigen binding site comprises a p40-binding heavy chain variable region (p40-VH) and a p40-binding light chain variable region (p40-VL), and wherein
(i) the p40 binding domain comprises a CDR-L1 sequence according to SEQ ID NO: 173; a CDR-L2 sequence according to SEQ ID NO: 174, and a CDR-L3 sequence according to SEQ ID NO: 175, a CDR-H1 sequence according to SEQ ID NO: 167, a CDR-H2 sequence according to SEQ ID NO: 168, and a CDR-H3 sequence according to SEQ ID NO: 169, and
(ii) the TL1A binding domain comprises a CDR-L1 sequence according to SEQ ID NO: 11; a CDR-L2 sequence according to SEQ ID NO: 12, and a CDR-L3 sequence according to SEQ ID NO: 13, a CDR-H1 sequence according to SEQ ID NO: 30; a CDR-H2 sequence according to SEQ ID NO: 41; and a CDR-H3 sequence according to SEQ ID NO: 32.

In some aspects, the TL1A/p40 antibody comprises a TL1A-VH framework sequence derived from a human germline VH sequence selected from the group consisting of IGHV1-18*01, IGHV1-18*01, IGHV1-18*01, IGHV1-46*01, IGHV3-7*01, IGHV1-2*02, IGHV1-46*01, IGHV3-7*01, IGHV1-2*02, IGHV1-46*01, IGHV3-7*01, IGHV1-2*02. In some aspects, the TL1A/p40 antibody comprises a TL1A-VH framework sequence derived from a human IGHV1-18*01 germline sequence.

In some aspects, the TL1A/p40 antibody comprises a TL1A-VL framework sequence derived from a human germline VL sequence selected from the group consisting of IGKV1-39*01, IGKV3-11*01, IGKV3D-7*01, IGKV3-11*01, IGKV3-11*01, IGKV3-11*01, IGKV1-39*01, IGKV1-39*01, IGKV1-39*01, IGKV3D-7*01, IGKV3D-7*01, IGKV3D-7*01. In some aspects, the TL1A/p40 antibody comprises a TL1A-VL framework sequence derived from a human germline IGKV1-39*01, sequence.

In some aspects, the TL1A/p40 antibody comprises a TL1A-VL framework sequence and a TL1A-VH framework sequence wherein one or both of the TL1A-VL framework sequence and the TL1A-VH framework sequence is at least 90%, 91%, 92%, 93%, 94%, 95%, 96%, 97%, 98%, 99%, or 100% identical to the human germline sequence from which it was derived.

In some aspects, the TL1A/p40 antibody comprises a TL1A-VL framework sequence and a TL1A-VH framework sequence, and wherein one or both of the TL1A-VL framework sequence or the TL1A-VH framework sequence is identical to the human germline sequence from which it was derived.

In some aspects, the TL1A-VL comprises an amino acid sequence according to SEQ ID NO: 18, and wherein the TL1A-VH comprises an amino acid sequence according to a sequence selected from the group consisting of SEQ ID NO: 42, SEQ ID NO: 44, SEQ ID NO: 47, SEQ ID NO:49, SEQ ID NO:52, SEQ ID NO: 54, SEQ ID NO: 57, SEQ ID NO: 70, SEQ ID NO: 72, SEQ ID NO: 78, SEQ ID NO: 80, SEQ ID NO: 89, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 110, and SEQ ID NO:120.

In some aspects, the TL1A/p40 antibody comprises a TL1A-VH sequence at least 90%, 91%, 92%, 93%, 94%, 95%, 96%, 97%, 98%, 99%, or 100% identical to SEQ ID NO: 42, and comprising a TL1A-VL sequence at least 90%, 91%, 92%, 93%, 94%, 95%, 96%, 97%, 98%, 99%, or 100% identical to SEQ ID NO: 18.

In some aspects, the TL1A/p40 antibody comprises the TL1A-VH sequence of SEQ ID NO: 42, and the TL1A-VL of SEQ ID NO: 18.

In some aspects, the TL1A/p40 antibody comprises a TL1A-VH sequence encoded by a nucleic acid sequence of SEQ ID NO: 228. In some aspects, the TL1A/p40 antibody comprises a TL1A-VL sequence encoded by a nucleic acid sequence of SEQ ID NO: 229.

In some aspects, the TL1A/p40 antibody comprises a TL1A-VH sequence encoded by the plasmid deposited at the ATCC and having ATCC Accession No. PTA-127347. In some aspects, the TL1A/p40 antibody comprises a TL1A-VL sequence encoded by the plasmid deposited at the ATCC and having ATCC Accession No. PTA-127348. In some aspects, the TL1A/p40 antibody comprises a TL1A-VH sequence encoded by the plasmid deposited at the ATCC and having ATCC Accession No. PTA-127347 and a TL1A-VL sequence encoded by the plasmid deposited at the ATCC and having ATCC Accession No. PTA-127348.

In some aspects, the TL1A/p40 antibody comprises a p40-VH framework sequence derived from a human germline VH sequence selected from the group consisting of DP3, DP7, DP73, DP75, and DP88. In some aspects, the TL1A/p40 antibody comprises a p40-VH framework sequence derived from a human DP73 germline sequence. In some aspects, the TL1A/p40 antibody comprises a p40-VL framework sequence derived from a human germline VL sequence selected from the group consisting of DPK4, DPK5, DPK7, DPK8, and DPK9. In some aspects, the TL1A/p40 antibody comprises a p40-VL framework sequence derived from a human germline DPK9, sequence.

In some aspects, the TL1A/p40 antibody comprises a p40-VL framework sequence and a p40-VH framework sequence wherein one or both of the p40-VL framework sequence and the p40-VH framework sequence is at least 90%, 91%, 92%, 93%, 94%, 95%, 96%, 97%, 98%, 99%, or 100% identical to the human germline sequence from which it was derived. In some aspects, the TL1A/p40 antibody comprises a p40-VL framework sequence and a p40-VH framework sequence, and wherein one or both of the p40-VL framework sequence or the p40-VH framework sequence is identical to the human germline sequence from which it was derived.

In some aspects, the TL1A/p40 antibody comprises a p40-VH sequence at least 90%, 91%, 92%, 93%, 94%, 95%, 96%, 97%, 98%, 99%, or 100% identical to SEQ ID NO: 177, and comprising a p40-VL sequence at least 90%, 91%, 92%, 93%, 94%, 95%, 96%, 97%, 98%, 99%, or 100% identical to SEQ ID NO: 171.

In some aspects, the p40-VL comprises an amino acid sequence according to SEQ ID NO: 177, and the p40-VH comprises an amino acid sequence according to SEQ ID NO: 171.

In some aspects, the TL1A/p40 antibody comprises a p40-VH sequence encoded by a nucleic acid sequence of SEQ ID NO: 238.

In some aspects, the TL1A/p40 antibody comprises a p40-VL sequence encoded by a nucleic acid sequence of SEQ ID NO: 239.

In some aspects, the TL1A/p40 antibody comprises a p40-VH sequence encoded by the plasmid deposited at the ATCC and having ATCC Accession No. PTA-127206. In some aspects, the TL1A/p40 antibody comprises a p40-VL sequence encoded by the plasmid deposited at the ATCC and having ATCC Accession No. PTA-127205. In some aspects, the TL1A/p40 antibody comprises a p40-VH sequence encoded by the plasmid deposited at the ATCC and having ATCC Accession No. PTA-127206 and a p40-VL sequence encoded by the plasmid deposited at the ATCC and having ATCC Accession No. PTA-127205.

In some aspects, the TL1A/p40 antibody comprises a TL1A-VH fused to a CH1 domain (TL1A-CH1) and a TL1A-VL is fused to a constant light domain (TL1A-CL) forming a TL1A binding Fab domain (TL1A-Fab). The TL1A-CH1 may comprises a sequence according to a sequence selected from the group consisting of SEQ ID NO: 6, and SEQ ID NO: 195, and SEQ ID NO: 199. The TL1A-CH1 may comprise a sequence according to SEQ ID NO: 6. The TL1A-CL may comprise a sequence selected from the group consisting of SEQ ID NO: 16, SEQ ID NO: 197, and SEQ ID NO: 201. The TL1A-CL may comprise a sequence according to SEQ ID NO: 16.

In some aspects, the TL1A/p40 antibody comprises a p40-VH fused to a CH1 domain (p40-CH1) and a p40-VL fused to a constant light domain (p40-CL) forming a p40 binding Fab domain (p40-Fab). The p40-CH1 may comprise a sequence according to a sequence selected from the group consisting of SEQ ID NO: 6, and SEQ ID NO: 195, and SEQ ID NO: 199. The p40-CH1 may comprise a sequence according to SEQ ID NO: 6. The p40-CL may comprise a sequence selected from the group consisting of SEQ ID NO: 16, SEQ ID NO: 197, and SEQ ID NO: 201. The p40-CL may comprise a sequence according to SEQ ID NO:16.

In some aspects, the TL1A/p40 antibody comprises an antibody Fc domain comprising a first Fc chain and a second Fc chain. The Fc domain may be the Fc domain of an IgA (for example IgA₁ or IgA₂), IgD, IgE, IgM, or IgG (for example IgG₁, IgG₂, IgG₃, or IgG₄). In some aspects, the Fc domain is the Fc domain of an IgG₁.

In some aspects, the TL1A/p40 antibody comprises a first Fc chain comprising, from N-terminus to C-terminus: a first hinge region, a first CH2 region, and a first CH3 region, and a second Fc chain comprising, from N-terminus to C-terminus: a second hinge region, a second CH2 region, and a second CH3 region. In some aspects, the N-terminus of the first Fc chain is connected to the C-terminus of the first TL1A-CL domain. In some aspects, the N-terminus of the second Fc chain is connected to the C-terminus of the first p40-CH1 domain. In some aspects, the first CH2 region and the second CH2 region each comprise a sequence according to SEQ ID NO: 8.

In some aspects, either or both of the first hinge region comprises a sequence selected from the group consisting of SEQ ID NO: 7, SEQ ID NO: 157, SEQ ID NO: 179, SEQ ID NO: 182, and SEQ ID NO: 189, SEQ ID NO: 191, SEQ ID NO: 204, and SEQ ID NO: 206. In some aspects, the first hinge region and the second hinge region each contain one or more amino acid modifications that promote the association of the first hinge region with the second hinge region.

In some aspects, the first hinge region and the second hinge region each comprise a different and complementary sequence, and the different and complementary sequences are selected from one of the following pairs of different and complementary sequences:
(i) SEQ ID NO: 179 and SEQ ID NO: 182; and (EEE)
(ii) SEQ ID NO: 189 and SEQ ID NO: 191 (EE)

The first hinge region may comprise a sequence according to SEQ ID NO: 204. The second hinge region may comprise a sequence according to SEQ ID NO: 7.

In some aspects, the first Fc chain and the second Fc chain each contain one or more amino acid modifications that promote the association of the first Fc chain with the second Fc chain. The first Fc chain may comprises a first CH3 region, and the second Fc chain may comprise a second CH3 region, and the first CH3 region and the second CH3 region each comprise a different and complementary sequence, and the different and complementary sequences are selected from one of the following pairs of different and complementary sequences:
(i) SEQ ID NO: 160 and SEQ ID NO: 162;
(ii) SEQ ID NO: 180, and SEQ ID NO: 183; (EEE, EE, E)
(iii) SEQ ID NO: 155 and SEQ ID NO: 158; and
(iv) SEQ ID NO: 185 and SEQ ID NO: SEQ ID NO: 187.

Where the first and second CH3 domains comprise SEQ ID NO: 160 and SEQ ID NO: 162, these CH3 domains function well with a complimentary hinge pair of either SEQ ID NO: 179 and SEQ ID NO: 182 (EEE/RRR heterodimerization strategy) or SEQ ID NO: 189 and SEQ ID NO: 191 (EE/RR heterodimerization strategy).

In some aspects, the TL1A/p40 antibody comprises a TL1A-Fab wherein the TL1A-CL is covalently fused to the first hinge region. In some aspects, the TL1A-CL is covalently fused to the first Fc chain. In some aspects, the TL1A-CL is covalently fused to the first Fc Chain through the first Hinge region.

In some aspects, the TL1A/p40 antibody comprises a first, second, third, and fourth polypeptide chain, such that the first and third polypeptide chains together form the TL1A-Fab, and the second and fourth polypeptide chains together form the p40-Fab. In some aspects, the first, second, third, and fourth polypeptide chains have a combination of sequences in accordance with one of the groups selected from the following list:
(i) the first polypeptide sequence is in accordance with SEQ ID NO: 205, the second polypeptide is in accordance with SEQ ID NO: 203, the third polypeptide is in accordance with SEQ ID NO: 207, and the fourth polypeptide is in accordance with SEQ ID NO: 178;
(ii) the first polypeptide sequence is in accordance with SEQ ID NO: 188, the second polypeptide is in accordance with SEQ ID NO: 186, the third polypeptide is in accordance with SEQ ID NO: 19, and the fourth polypeptide is in accordance with SEQ ID NO: 178;
(iii) the first polypeptide sequence is in accordance with SEQ ID NO: 192, the second polypeptide is in accordance with SEQ ID NO: 190, the third polypeptide is in accordance with SEQ ID NO: 19, and the fourth polypeptide is in accordance with SEQ ID NO: 178;
(iv) the first polypeptide sequence is in accordance with SEQ ID NO: 194, the second polypeptide is in accordance with SEQ ID NO: 193, the third polypeptide is in accordance with SEQ ID NO: 19, and the fourth polypeptide is in accordance with SEQ ID NO: 178; or
(v) the first polypeptide sequence is in accordance with SEQ ID NO: 200, the second polypeptide is in accordance with SEQ ID NO: 196, the third polypeptide is in accordance with SEQ ID NO: 202, and the fourth polypeptide is in accordance with SEQ ID NO: 198.

In some aspects, the TL1A/p40 antibody comprises a first polypeptide sequence in accordance with SEQ ID NO: 205, a second polypeptide in accordance with SEQ ID NO: 203, a third polypeptide in accordance with SEQ ID NO: 207, and a fourth polypeptide in accordance with SEQ ID NO: 178.

In some aspects, the disclosure provides an isolated TL1A/p40 antibody that specifically binds to TL1A and p40, wherein the antibody comprises a first, second, third, and fourth polypeptide chain, such that the first and third polypeptide chains together form a TL1A binding region, and the second and fourth polypeptide chains together form a p40 binding region, and wherein the first polypeptide sequence is in accordance with SEQ ID NO: 205, the second polypeptide is in accordance with SEQ ID NO: 203, the third polypeptide is in accordance with SEQ ID NO: 207, and the fourth polypeptide is in accordance with SEQ ID NO: 178.

In some aspects, the disclosure provides an isolated TL1A/p40 antibody that specifically binds to TL1A and p40, wherein the antibody comprises a first, second, third, and fourth polypeptide chain, such that the first and third polypeptide chains together form a TL1A binding region, and the second and fourth polypeptide chains together form a p40 binding region, and wherein the first polypeptide sequence comprises a sequence encoded by the plasmid deposited at the ATCC and having ATCC Accession No. PTA-127349, the second polypeptide comprises a sequence encoded by the plasmid deposited at the ATCC and having ATCC Accession No. PTA-127346, the third polypeptide comprises a sequence encoded by the plasmid deposited at the ATCC and having ATCC Accession No. PTA-127350, and the fourth polypeptide comprises a sequence encoded by the plasmid deposited at the ATCC and having ATCC Accession No. PTA-127203.

In some aspects, the disclosure provides an isolated TL1A/p40 antibody that specifically binds to TL1A and p40, comprising the CDRs of an antibody selected from one or more of Tables 30, 31, 32, and 33. In some aspects, the disclosure provides an isolated TL1A/p40 antibody that specifically binds to TL1A and p40, comprising the VH and VL of an antibody that binds TL1A selected from one or more of Tables 30, 31, 32, and 33. In some aspects, the disclosure provides an isolated TL1A/p40 antibody that specifically binds to TL1A and p40, selected from one or more of Tables 30, 31, 32, and 33. In some aspects, the disclosure provides an isolated TL1A/p40 antibody that specifically binds to TL1A and p40, comprising the CDRs of an antibody selected from one or more of Tables 30, 31, 32, and 33. In some aspects, the disclosure provides an isolated TL1A/p40 antibody that specifically binds to TL1A and p40, comprising the VH and VL of an antibody selected from one or more of Tables 30, 31, 32, and 33.

In some aspects, the TL1A/p40 antibody is characterized by a score of less than 2 in an affinity-capture self-interaction nanoparticle spectroscopy (AC SINS) assay. The AC SINS assay may be conducted as herein described in the Examples.

In some aspects, the TL1A/p40 antibody is characterized in that the charge heterogeneity of the antibody is at least 50% non-acid and non-basic as measured in an antibody imaged capillary electrophoresis (iCE) for an unstressed sample (T0) to evaluate. The measurement of non-acid and non-basic species by iCE may be conducted as herein described in the Examples.

In some aspects, the TL1A/p40 antibody binds to immobilized human TL1A with a binding affinity of less than 1 nM, as measured by SPR. In some aspects, the TL1A/p40 antibody binds to immobilized human TL1A with a binding affinity of less than 500 pM, as measured by SPR. In some aspects, the TL1A/p40 antibody binds to immobilized human TL1A with a binding affinity of less than 300 pM, as measured by SPR. In some aspects, the TL1A/p40 antibody binds to human p40 with a binding affinity of less than 1 nM, as measured by SPR. In some aspects, the TL1A/p40 antibody binds to human p40 with a binding affinity of less than 900 pM, as measured by SPR. In some aspects, the TL1A/p40 antibody binds to human p40 with a binding affinity of less than 600 pM, as measured by SPR. In some aspects, the TL1A/p40 antibody binds p40(IL-23) and TL1A simultaneously in an SPR assay wherein the antibody is injected over immobilized TL1A and the resulting complex subsequently treated with IL-23. The SPR may be conducted as herein described in the Examples.

In some aspects, the TL1A/p40 antibody is characterized by an IC₅₀ of less than 5 nM in a human IL-12 neutralization Kit-225 assay. The antibody may be characterized by an IC₅₀ of less than 4 nM in a human IL-12 neutralization Kit-225 assay. The antibody may be characterized by an IC₅₀ of less than 2 pM in a human IL-12 neutralization Kit-225 assay. The antibody may be characterized by an IC₅₀ of less than 5 nM in a human IL-23 neutralization Kit-225 assay. The antibody may be characterized by an IC₅₀ of less than 4 nM, in a human IL-23 neutralization Kit-225 assay. The antibody may be characterized by an IC₅₀ of less than 1 nM in a human IL-23 neutralization Kit-225 assay in human whole blood. The antibody may be characterized by an IC₅₀ of less than 0.5 nM, in a human IL-23 neutralization Kit-225 assay in human whole blood. In some aspects, the Kit-225 assay is in human whole blood. The respective Kit-225 assays may be conducted as herein described in the Examples for that respective assay.

In some aspects, the TL1A/p40 antibody is characterized by an IC₅₀ of less than 5 nM in a cynomolgus monkey IL-23 neutralization Kit-225 assay. The antibody may be characterized by an IC₅₀ of less than 2 nM in a cynomolgus monkey IL-12 neutralization Kit-225 assay. The antibody may be characterized by an IC₅₀ of less than 1 nM in a cynomolgus monkey IL-12 neutralization Kit-225 assay. The respective Kit-225 assays may be conducted as herein described in the Examples for that respective assay.

The antibody may be characterized by an IC₅₀ of less than 2 nM in a human whole blood assay measuring inhibition of IFNγ. The antibody may be is characterized by an IC₅₀ of less than 3 nM in a human whole blood assay measuring inhibition of IFNγ. The antibody may be characterized by an IC₅₀ of less than 2 nM in a human whole blood assay measuring inhibition of IFNγ. The antibody may be characterized by an IC₅₀ of less than 500 pM in a human isolated CD4+ cell assay measuring inhibition of IFNγ. The antibody may be characterized by an IC₅₀ of less than 100 pM in a human isolated CD4+ cell assay measuring inhibition of IFNγ. The antibody may be characterized by an IC₅₀ of less than 200 nM in a human NFκB neutralization assay in TF-1 reporter cells. The antibody may be characterized by an IC₅₀ of less than 150 nM in a human NFκB neutralization assay in TF-1 reporter cells. The respective IC-50 assays referred to in this paragraph may be conducted as herein described in the Examples for that respective assay.

In some aspects, the TL1A/p40 antibody is characterized by a terminal half-life of at least 10 days in TG32 mice, based on a two-week analysis. In some aspects, the terminal half-life is at least 14 days in TG32 mice, based on a two-week analysis. In some aspects, the terminal half-life is at least 16 days in TG32 mice, based on a two-week analysis. In some aspects, the terminal half-life is at least 17 days in TG32 mice, based on a two-week analysis. In some aspects, the TL1A/p40 antibody is characterized by a terminal half-life of at least 21 days in cynomolgus monkeys. In some aspects, the terminal half-life is at least 25 days in cynomolgus monkeys. In some aspects, the terminal half-life is at least 28 days in cynomolgus monkeys. The respective half-life determining assays in TG32 mice and cynomolgus monkeys may be conducted as herein described in the Examples for that respective assay.

In some aspects, the disclosure provides an isolated anti-TL1A/p40 antibody that specifically binds TL1A through a TL1A heavy chain variable region (TL1A-VH) and a TL1A light chain variable region (TL1A-VL); and that specifically binds p40 through a p40 heavy chain variable region (p40-VH) and an p40 light chain variable region (p40-VL); wherein the TL1A-VH comprises the CDR-H1, CDR-H2, and CDR-H3 sequences encoded by the plasmid deposited at the ATCC and having ATCC Accession No. PTA-127347, and the TL1A-VL comprises the CDR-L1, CDR-L2, and CDR-L3 sequences encoded by the plasmid deposited at the ATCC and having ATCC Accession No. PTA-127348; and the p40-VH comprises the CDR-H1, CDR-H2, and CDR-H3 sequences encoded by the plasmid deposited at the ATCC and having ATCC Accession No. PTA-127206, and the p40-VL comprises the CDR-L1, CDR-L2, and CDR-L3 sequences encoded by the plasmid deposited at the ATCC and having ATCC Accession No. PTA-127205.

In some aspects, the disclosure provides an isolated anti-TL1A/p40 antibody that specifically binds TL1A through a TL1A heavy chain variable region (TL1A-VH) and a TL1A light chain variable region (TL1A-VL); and that specifically binds p40 through a p40 heavy chain variable region (p40-VH) and an p40 light chain variable region (p40-VL); comprising a TL1A-VH sequence encoded by the plasmid deposited at the ATCC and having ATCC Accession No. PTA-127347, and a TL1A-VL sequence encoded by the plasmid deposited at the ATCC and having ATCC Accession No. PTA-127348; and a p40-VH sequence encoded by the plasmid deposited at the ATCC and having ATCC Accession No. PTA-127206, and a p40-VL sequence encoded by the plasmid deposited at the ATCC and having ATCC Accession No. PTA-127205.

In some aspects, the disclosure provides an isolated anti-TL1A/p40 antibody comprising the sequence encoded by the plasmid deposited at the ATCC and having ATCC Accession No. PTA-127203; the sequence encoded by the plasmid deposited at the ATCC and having ATCC Accession No. PTA-127346; the sequence encoded by the plasmid deposited at the ATCC and having ATCC Accession No. PTA-127349; and the sequence encoded by the plasmid deposited at the ATCC and having ATCC Accession No. PTA-127350.

### Nucleic Acids

The disclosure also provides polynucleotides encoding any of the antibodies of the invention, including antibody portions and modified antibodies described herein. The invention also provides a method of making any of the antibodies and polynucleotides described herein. Polynucleotides can be made and the proteins expressed by procedures known in the art.

If desired, an antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide comprising a sequence encoding one or both of the heavy chain or the light chain variable regions of an antibody provided herein. The sequence encoding the antibody of interest may be maintained in a vector in a host cell and the host cell can then be expanded and frozen for future use. Vectors (including expression vectors) and host cells are further described herein.

In some embodiments, the disclosure provides polynucleotides encoding the amino acid sequences of any of the antibodies listed in one or more of Tables 30, 31, 32, and 33. The polynucleotide may be RNA. The polynucleotide may comprise at least one chemical modification. The chemical modification may be selected from the group consisting of pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine), 5-methoxyuridine, and 2′-O-methyl uridine.

In some aspects, the polynucleotide does not comprise a chemical modification.

In some aspects, there is provided an isolated polynucleotide encoding the VH, VL, or both, of an antibody that binds TL1A, wherein the nucleic acid comprises: the nucleic acid sequence of SEQ ID NO: 228, the nucleic acid sequence of SEQ ID NO: 229, or both. E196. An isolated polynucleotide encoding a VH bearing polypeptide and a VL bearing polypeptide, or both, of an antibody that binds to TL1A, wherein said nucleic acid comprises the nucleic acid sequence of SEQ ID NO: 231, the nucleic acid sequence of SEQ ID NO: 233, or both.

In some aspects, there is provided an isolated polynucleotide encoding the VH, VL, or both, of an antibody that binds to TL1A, wherein said nucleic acid comprises the nucleic acid sequence of the insert of the plasmid deposited with the ATCC and having the Accession Number PTA-127347 the nucleic acid sequence of the insert of the plasmid deposited with the ATCC and having the Accession Number PTA-127348 or both.

In some aspects, there is provided a VH bearing polypeptide and a VL bearing polypeptide, or both, of an antibody that binds to TL1A, wherein said nucleic acid comprises the nucleic acid sequence of the insert of the plasmid deposited with the ATCC and having the Accession Number PTA-127347, the nucleic acid sequence of the insert of the plasmid deposited with the ATCC and having the Accession Number PTA-127348, or both.

In some aspects, there is provided an isolated polynucleotide comprising one or more nucleic acids encoding the one or more sequences of a null-arm antibody that binds TL1A, wherein null-arm antibody comprises a null-arm TL1A antibody comprising a first variable heavy chain and a first variable light chain, and a second variable heavy chain and second variable light chain, wherein the first variable heavy chain and the first variable light chain comprise a first Fab domain that binds to TL1A (TL1A-Fab) and the second variable heavy chain and the second variable light chain comprise a second Fab domain that comprises a non-binding TL1A binding domain that does not specifically bind to a target, (xTL1A-Fab), and wherein the first and second variable light chains are identical, and wherein said polynucleotide comprises the nucleic acid sequence of SEQ ID NO: 228, the nucleic acid sequence of SEQ ID NO: 230, and/or the nucleic acid sequence of SEQ ID NO: 260.

In some aspects, there is provided an isolated polynucleotide comprising one or more nucleic acids encoding the one or more sequences of a null-arm antibody that binds TL1A, wherein null-arm antibody comprises a null-arm TL1A antibody comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target, (xTL1A-Fab), and wherein the first and second light chain are identical, and wherein said polynucleotide comprises the nucleic acid sequence of SEQ ID NO: 231, the nucleic acid sequence of SEQ ID NO: 232, and/or the nucleic acid sequence of SEQ ID NO: 233.

In some aspects, there is provided an isolated polynucleotide comprising one or more nucleic acids encoding the one or more sequences of a null-arm antibody that binds TL1A, wherein null-arm antibody comprises a null-arm TL1A antibody comprising a first heavy chain and a first light chain, and a second heavy chain and second light chain, wherein the first heavy chain and the first light chain comprise a first Fab domain that binds to TL1A (TL1A-Fab) and the second heavy chain and the second light chain comprise a second Fab domain that comprises a non-binding TL1A binding domain that does not specifically bind to a target, (xTL1A-Fab), and wherein the first and second light chain are identical, and wherein said polynucleotide comprises one or more of the nucleic acid sequence of the insert of the plasmid deposited with the ATCC and having the Accession Number PTA-127347, the nucleic acid sequence of the insert of the plasmid deposited with the ATCC and having the Accession Number PTA-127348, and the nucleic acid sequence of the insert of the plasmid deposited with the ATCC and having the Accession Number PTA-127351.

In some aspects, there is provided an isolated polynucleotide encoding one or more of the first, second, third, and fourth polypeptides of an antibody that binds to both TL1A and p40, comprising
(iii) an TL1A-VH sequence encoded by a nucleic acid comprising the nucleic acid sequence of SEQ ID NO: 228, and an TL1A-VL encoded by a nucleic acid comprising the nucleic acid sequence of SEQ ID NO: 229; and
(iv) an p40-VH sequence encoded by a nucleic acid comprising the nucleic acid sequence of SEQ ID NO: 238, and an p40-VL sequence encoded by a nucleic acid comprising the nucleic acid sequence of SEQ ID NO: 239.

In some aspects, there is provided an isolated polynucleotide encoding one or more of the first, second, third, and fourth polypeptides of an antibody that binds to both TL1A and p40, comprising
(i) an TL1A-VH bearing polypeptide sequence encoded by a nucleic acid comprising the nucleic acid sequence of SEQ ID NO: 234, and an TL1A-VL bearing polypeptide encoded by a nucleic acid comprising the nucleic acid sequence of SEQ ID NO: 235; and
(ii) an p40-HC sequence encoded by a nucleic acid comprising the nucleic acid sequence of SEQ ID NO: 236, and an p40-LC sequence encoded by a nucleic acid comprising the nucleic acid sequence of SEQ ID NO: 237.

In some aspects, there is provided an isolated polynucleotide encoding one or more of the first, second, third, and fourth polypeptides of an antibody that binds to both TL1A and p40, comprising
(i) an TL1A-VH sequence encoded by the plasmid deposited at the ATCC and having ATCC Accession No. PTA-127347, and an TL1A-VL sequence encoded by the plasmid deposited at the ATCC and having ATCC Accession No. PTA-127348; and
(ii) an p40-VH sequence encoded by the plasmid deposited at the ATCC and having ATCC Accession No. PTA-127206, and an p40-VL sequence encoded by the plasmid deposited at the ATCC and having ATCC Accession No. PTA-127205.

In some aspects, there is provided an isolated polynucleotide encoding one or more of the first, second, third, and fourth polypeptides of an antibody that binds to both TL1A and p40, comprising
(i) an TL1A-VHbearing polypeptide sequence encoded by the plasmid deposited at the ATCC and having ATCC Accession No. PTA-127350, and an TL1A-VL bearing polypeptide sequence encoded by the plasmid deposited at the ATCC and having ATCC Accession No. PTA-127349; and
(ii) an p40-HC sequence encoded by the plasmid deposited at the ATCC and having ATCC Accession No. PTA-127346, and an p40-LC sequence encoded by the plasmid deposited at the ATCC and having ATCC Accession No. PTA-127203.

In some aspects, there is provided an isolated polynucleotide encoding a TL1A antibody VH domain, comprising the nucleic acid sequence of SEQ ID NO: 228. In some aspects, there is provided an isolated polynucleotide encoding a TL1A antibody VL domain, comprising the nucleic acid sequence of SEQ ID NO: 229. In some aspects, there is provided an isolated polynucleotide encoding a TL1A antibody VH domain engineered to eliminate TL1A binding, comprising the nucleic acid sequence of SEQ ID NO: 230. In some aspects, there is provided an isolated polynucleotide encoding a TL1A antibody HC, comprising the nucleic acid sequence of SEQ ID NO: 231. In some aspects, there is provided an isolated polynucleotide encoding a TL1A antibody HC engineered to eliminate TL1A binding, comprising the nucleic acid sequence of SEQ ID NO: 232. In some aspects, there is provided an isolated polynucleotide encoding a TL1A antibody LC, comprising the nucleic acid sequence of SEQ ID NO: 233. In some aspects, there is provided an isolated polynucleotide encoding a TL1A antibody mFd chain, comprising the nucleic acid sequence of SEQ ID NO: 234. In some aspects, there is provided an isolated polynucleotide encoding a TL1A antibody LC-Fc chain comprising the nucleic acid sequence of SEQ ID NO: 235. In some aspects, there is provided an isolated polynucleotide encoding a p40 antibody HC, comprising the nucleic acid sequence of SEQ ID NO: 236. In some aspects, there is provided an isolated polynucleotide encoding a p40 antibody LC, comprising the nucleic acid sequence of SEQ ID NO: 237. In some aspects, there is provided an isolated polynucleotide encoding a p40 antibody VH, comprising the nucleic acid sequence of SEQ ID NO: 238. In some aspects, there is provided an isolated polynucleotide encoding a p40 antibody VL, comprising the nucleic acid sequence of SEQ ID NO: 239.

In some aspects, there is provided a vector comprising one or more of the polynucleotides herein provided.

In some aspects, there is provided an isolated host cell comprising one or more the polynucleotides herein provided, or the vectors herein provided.

In some aspects, there is provided a method of producing an isolated antibody, comprising culturing the host cell of as herein provided under conditions that result in production of the antibody, and recovering the antibody.

In some aspects, there is provided a pharmaceutical composition comprising a therapeutically effective amount of the antibody as herein provided, and a pharmaceutically acceptable carrier.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

### Pharmaceutical Compositions

In other embodiments, the invention comprises pharmaceutical compositions.

A “pharmaceutical composition” refers to a mixture of an antibody the invention and one or excipient. As used herein, pharmaceutical compositions may comprise one or more antibodies that bind to TL1A, and/or TL1A and p40, or one or more polynucleotides comprising sequences encoding one or more these antibodies. These compositions may further comprise suitable excipients, such as pharmaceutically acceptable excipients including buffers, which are well known in the art.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

The invention provided herein further encompasses methods and compositions for treatment, prevention or management of one or more disorders or diseases selected from the group consisting of a TL1A related disorder, a p40 related disorder, a TL1A and p40 related disorder, inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis.

In one aspect, the invention provides a method for treating a condition associated with one or more of TL1A and p40 expression in a subject. In some embodiments, the method of treating a condition associated with one or more of TL1A and p40 expression in a subject comprises administering to the subject in need thereof an effective amount of a composition (e.g., pharmaceutical composition) comprising the respective anti-TL1A, anti-p40, or TL1A/p40 multispecific antibodies as described herein. The conditions associated with TL1A and p40 expression include, but are not limited to, abnormal expression of one or more of TL1A and p40 expression, altered or aberrant TL1A or p40 expression.

In one aspect, the present invention provides one or more selected from the group consisting of anti-TL1A, anti-p40, and anti-TL1A/p40 antibodies described herein, or a pharmaceutical composition comprising such antibody for use in therapy. In a particular embodiment, the invention also provides one or more of anti-TL1A, anti-p40, anti-TL1A/p40 antibodies for use in treating a disorder associated with one more of TL1A and p40 related disorders.

The present invention further provides one or more selected from the group consisting of anti-TL1A, anti-p40, and anti-TL1A/p40 antibodies as described herein, or a pharmaceutical composition comprising such antibody for use in the manufacture of a medicament for use in therapy. In some embodiments, the therapy is a treatment of a disorder associated with one or more of TL1A, p40, and TL1A/p40.

### Therapeutic, Diagnostic, and Other Methods

The antibodies and the antibody conjugates of the present invention are useful in various applications including, but are not limited to, therapeutic treatment methods and diagnostic treatment methods.

TL1A antibodies of the invention may inhibit the activity of TL1A and may be useful in the treatment, prevention, suppression and amelioration of TL1A related diseases. The invention provides a method for treating disorders associated with TL1A expression. The invention provides a method of treating one or more selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis in a subject comprising administering to the subject in need thereof an effective amount of a pharmaceutical composition comprising any of the TL1A antibodies as described herein. The preceding sentence provides a list of disorders associated with TL1A expression.

In some aspects, TL1A antibodies of the invention may inhibit the activity of TL1A and may be useful in the treatment, prevention, suppression and amelioration of one or more diseases selected from the group consisting inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis. In some aspects, TL1A antibodies of the invention may be useful in the treatment, prevention, suppression and amelioration of asthma. In some aspects, TL1A antibodies of the invention may be useful in the treatment, prevention, suppression and amelioration of NASH.

In another aspect, the invention further provides an antibody or pharmaceutical composition as described herein for use in the described method of treating one or more of the disorders associated with TL1A expression. The invention also provides the use of an antibody as described herein in the manufacture of a medicament for treating one or more disorders associated with TL1A expression. In another aspect, provided is a method of one or more of detecting, diagnosing, or monitoring one or more of the disorders associate with TL1A expression. For example, the anti-TL1A antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent.

With respect to all methods described herein, reference to anti-TL1A antibodies also includes pharmaceutical compositions comprising the anti-TL1A antibodies and one or more additional agents.

TL1A/p40 antibodies of the invention may inhibit the activity of TL1A and or p40 and may be useful in the treatment, prevention, suppression and amelioration of TL1A and or p40 related diseases. The invention provides a method for treating disorders associated with TL1A expression. The invention provides a method of treating one or more selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis in a subject comprising administering to the subject in need thereof an effective amount of a pharmaceutical composition comprising any of the TL1A/p40 antibodies as described herein. The preceding sentence provides a list of disorders associated with TL1A/p40 expression.

In some aspects, TL1A/p40 antibodies of the invention may inhibit the activity of TL1A and or p40 and may be useful in the treatment, prevention, suppression and amelioration of one or more diseases selected from the group consisting inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis. In some aspects, TL1A antibodies of the invention may be useful in the treatment, prevention, suppression and amelioration of asthma. In some aspects, TL1A/p40 antibodies of the invention may be useful in the treatment, prevention, suppression and amelioration of NASH.

In another aspect, the invention further provides an antibody or pharmaceutical composition as described herein for use in the described method of treating one or more of the disorders associated with TL1A and or p40 expression. The invention also provides the use of an antibody as described herein in the manufacture of a medicament for treating one or more disorders associated with TL1A and or p40 expression. In another aspect, provided is a method of one or more of detecting, diagnosing, or monitoring one or more of the disorders associate with TL1A and or p40 expression. For example, the anti-TL1A/p40 antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent.

With respect to all methods described herein, reference to anti-TL1A/p40 antibodies also includes pharmaceutical compositions comprising the anti-TL1A/p40 antibodies and one or more additional agents.

### Administration and Dosing

Typically, an antibody of the invention is administered in an amount effective to treat a condition as described herein. The antibodies the invention can be administered as an antibody per se, or alternatively, as a pharmaceutical composition containing the antibody.

The antibodies of the invention are administered by any suitable route in the form of a pharmaceutical composition adapted to such a route, and in a dose effective for the treatment intended.

In some embodiments, the antibodies may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In some aspects, TL1A antibodies of the invention are administered subcutaneously. In some aspects, TL1A/p40 antibodies of the invention are administered subcutaneously.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the antibodies of the invention or compositions containing said antibodies is based on a variety of factors, including the type, age, weight, sex and medical condition of the subject; the severity of the condition; the route of administration; and the activity of the particular antibody employed. Thus, the dosage regimen may vary widely. In one embodiment, the total daily dose of an antibody of the invention is typically from about 0.01 to about 100 mg/kg (i.e., mg antibody of the invention per kg body weight) for the treatment of the indicated conditions discussed herein. In another embodiment, total daily dose of the antibody of the invention is from about 0.1 to about 50 mg/kg, and in another embodiment, from about 0.5 to about 30 mg/kg.

### Co-Administration

The antibodies of the invention can be used alone, or in combination with one or more other therapeutic agents. The invention provides any of the uses, methods or compositions as defined herein wherein an antibody of the invention is used in combination with one or more other therapeutic agent discussed herein.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

Various formulations of the antibodies of the present invention (e.g., one or more of anti-TL1A and anti-TL1A/p40 antibodies) may be used for administration. In some embodiments, the antibodies may be administered neat. In some embodiments, the antibody and a pharmaceutically acceptable excipient may be in various formulations. Pharmaceutically acceptable excipients are known in the art and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 21st Ed. Mack Publishing, 2005.

In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

The antibodies (e.g., one or more of TL1A and TL1A/p40 antibodies) as described herein can be administered using any suitable method, including by injection (e.g., intraperitoneally, intravenously, subcutaneously, intramuscularly, etc.). The antibody, e.g., monoclonal antibody or multispecific antibody, also be administered via inhalation, as described herein. Generally, for administration of the antibody of the present, the dosage depends upon the host treated and the particular mode of administration. In one embodiment, the dose range of the antibody of the present invention will be about 0.001 μg/kg body weight to about 20,000 μg/kg body weight. The term “body weight” is applicable when a patient is being treated. When isolated cells are being treated, “body weight” as used herein refers to a “total cell body weight”. The term “total body weight” may be used to apply to both isolated cell and patient treatment. All concentrations and treatment levels are expressed as “body weight” or simply “kg” in this application are also considered to cover the analogous “total cell body weight” and “total body weight” concentrations. However, those of ordinary skill in the art will recognize the utility of a variety of dosage range, for example, 0.01 μg/kg body weight to 20,000 μg/kg body weight, 0.02 μg/kg body weight to 15,000 μg/kg body weight, 0.03 μg/kg body weight to 10,000 μg/kg body weight, 0.04 μg/kg body weight to 5,000 μg/kg body weight, 0.05 μg/kg body weight to 2,500 μg/kg body weight, 0.06 μg/kg body weight to 1,000 μg/kg body weight, 0.07 μg/kg body weight to 500 μg/kg body weight, 0.08 μg/kg body weight to 400 μg/kg body weight, 0.09 μg/kg body weight to 200 μg/kg body weight or 0.1 μg/kg body weight to 100 μg/kg body weight. Further, those of skill will recognize that a variety of different dosage levels will be of use, for example, one or more selected from the group consisting of 0.0001 μg/kg, 0.0002 μg/kg, 0.0003 μg/kg, 0.0004 μg/kg, 0.005 μg/kg, 0.0007 μg/kg, 0.001 μg/kg, 0.1 μg/kg, 1.0 μg/kg, 1.5 μg/kg, 2.0 μg/kg, 5.0 μg/kg, 10.0 μg/kg, 15.0 μg/kg, 30.0 μg/kg, 50 μg/kg, 75 μg/kg, 80 μg/kg, 90 μg/kg, 100 μg/kg, 120 μg/kg, 140 μg/kg, 150 μg/kg, 160 μg/kg, 180 μg/kg, 200 μg/kg, 225 μg/kg, 250 μg/kg, 275 μg/kg, 300 μg/kg, 325 μg/kg, 350 μg/kg, 375 μg/kg, 400 μg/kg, 450 μg/kg, 500 μg/kg, 550 μg/kg, 600 μg/kg, 700 μg/kg, 750 μg/kg, 800 μg/kg, 900 μg/kg, 1 μg/kg, 5 μg/kg, 10 μg/kg, 12 μg/kg, 15 mg/kg, 20 mg/kg, and 30 mg/kg. All of these dosages are exemplary, and any dosage in-between these points is also expected to be of use in the invention. Any of the above dosage ranges or dosage levels may be employed for an antibody of the present invention. For repeated administrations over several days or longer, depending on the condition, the treatment is sustained until a desired suppression of symptoms occurs or until sufficient therapeutic levels are achieved.

Generally, for administration of antibodies provided herein, the candidate dosage can be administered daily, every week, every other week, every three weeks, every four weeks, every five weeks, every six weeks, every seven weeks, every eight weeks, every ten weeks, every twelve weeks, or more than every twelve weeks.

In some embodiments, the candidate dosage is administered daily with the dosage ranging from about any of 1 μg/kg, to 30 μg/kg, to 300 μg/kg, to 3 mg/kg, to 30 mg/kg, to 100 mg/kg or more, depending on the factors mentioned above. For example, daily dosage of about 0.01 mg/kg, about 0.03 mg/kg, about 0.1 mg/kg, about 0.3 mg/kg, about 1 mg/kg, about 2.5 mg/kg, about 3 mg/kg, about 5 mg/kg, about 10 mg/kg, about 15 mg/kg, and about 25 mg/kg may be used.

In some embodiments, the candidate dosage is administered every week with the dosage ranging from about any of 1 μg/kg, to 30 μg/kg, to 300 μg/kg, to 3 mg/kg, to 30 mg/kg, to 100 mg/kg or more, depending on the factors mentioned above. For example, a weekly dosage of about 0.01 mg/kg, about 0.03 mg/kg, about 0.1 mg/kg, about 0.3 mg/kg, about 0.5 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every two weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a bi-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every three weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a tri-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/k may be used.

In some embodiments, the candidate dosage is administered every month or every four weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a monthly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In other embodiments, the candidate dosage is administered daily with the dosage ranging from about 0.01 mg to about 1200 mg or more, depending on the factors mentioned above. For example, daily dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, or about 1200 mg may be used.

In other embodiments, the candidate dosage is administered every week with the dosage ranging from about 0.01 mg to about 2000 mg or more, depending on the factors mentioned above. For example, weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other embodiments, the candidate dosage is administered every two weeks with the dosage ranging from about 0.01 mg to about 2000 mg or more, depending on the factors mentioned above. For example, bi-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other embodiments, the candidate dosage is administered every three weeks with the dosage ranging from about 0.01 mg to about 2500 mg or more, depending on the factors mentioned above. For example, tri-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or about 2500 mg may be used.

In other embodiments, the candidate dosage is administered every four weeks or month with the dosage ranging from about 0.01 mg to about 3000 mg or more, depending on the factors mentioned above. For example, monthly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500, about 2600 mg, about 2700 mg, about 2800 mg, about 2900 mg, or about 3000 mg may be used.

Other dosage regimens may also be useful, depending on the pattern of pharmacokinetic decay that the practitioner wishes to achieve. In one embodiment, the antibody of the present invention is administered in an initial priming dose followed by a higher and/or continuous, substantially constant dosage. In some embodiments, dosing from one to four times a week is contemplated. In other embodiments, dosing once a month or once every other month or every three months is contemplated. The progress of this therapy is easily monitored by conventional techniques and assays. The dosing regimen can vary over time.

For the purpose of the present invention, the appropriate dosage of an antibody (e.g., one or more selected from the group consisting of TL1A and TL1A/p40 antibodies) will depend on the antibody or compositions thereof employed, the type and severity of symptoms to be treated, whether the agent is administered for therapeutic purposes, previous therapy, the patient's clinical history and response to the agent, the patient's clearance rate for the administered agent, and the discretion of the attending physician. Typically, the clinician will administer an antibody until a dosage is reached that achieves the desired result. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of symptoms. Alternatively, sustained continuous release formulations of antibodies may be appropriate. Various formulations and devices for achieving sustained release are known in the art.

In one embodiment, dosages for an antibody (e.g., one or more selected from the group consisting of TL1A and TL1A/p40 antibodies) may be determined empirically in individuals who have been given one or more administration(s) of the antibody. Individuals are given incremental dosages of an antibody. To assess efficacy, an indicator of the disease can be followed.

In some embodiments, an antibody provided herein (e.g., one or more selected from the group consisting of TL1A and TL1A/p40 antibodies) may be administered to a subject that has previously received one or more antibodies selected from the group consisting of TL1A and TL1A/p40 antibody therapeutic for treatment of a disease. In some embodiments, an antibody provided herein may be an administered to a subject that has previously received an antibody selected from the group consisting of TL1A and TL1A/p40 antibody therapeutic for treatment of a disease, and for which the previous TL1A or TL1A/p40 antibody therapeutic is of limited or no efficacy in the subject (e.g. for which the subject's disease is resistant to treatment with the prior therapeutic).

Administration of an antibody in accordance with the method in the present invention can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced doses.

Therapeutic formulations of the antibody used in accordance with the present invention are prepared for storage by mixing an antibody having the desired degree of purity with optional pharmaceutically acceptable carriers, excipients or stabilizers (Remington, The Science and Practice of Pharmacy 21st Ed. Mack Publishing, 2005), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### Kits

Another aspect of the invention provides kits comprising the antibody of the invention or pharmaceutical compositions comprising the antibody. A kit may include, in addition to the antibody of the invention or pharmaceutical composition thereof, diagnostic or therapeutic agents. A kit may also include instructions for use in a diagnostic or therapeutic method. In some embodiments, the kit includes the antibody or a pharmaceutical composition thereof and a diagnostic agent. In other embodiments, the kit includes the antibody or a pharmaceutical composition thereof and one or more therapeutic agents.

A further aspect of the invention is a kit comprising one or more selected from the group consisting of TL1A and TL1A/p40 antibodies as disclosed herein above and instructions for use in accordance with any of the methods of the invention described herein. Generally, these instructions comprise a description of administration one or more selected from the group consisting of TL1A and TL1A/p40 antibodies for the above described therapeutic treatments.

In yet another embodiment, the invention comprises kits that are suitable for use in performing the methods of treatment described herein. In one embodiment, the kit contains a first dosage form comprising one or more of the antibodies of the invention in quantities sufficient to carry out the methods of the invention. In another embodiment, the kit comprises one or more antibodies of the invention in quantities sufficient to carry out the methods of the invention and at least a first container for a first dosage and a second container for a second dosage.

Several aspects of the pharmaceutical compositions, prophylactic, or therapeutic agents of the invention are preferably tested in vitro, in a cell culture system, and in an animal model organism, such as a rodent animal model system, for the desired therapeutic activity prior to use in humans.

Toxicity and efficacy of the prophylactic and/or therapeutic protocols of the instant invention maybe determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it may be expressed as the ratio LD₅₀/ED₅₀. Prophylactic and/or therapeutic agents that exhibit large therapeutic indices are preferred.

Further, any assays known to those skilled in the art may be used to evaluate the prophylactic and/or therapeutic utility of the therapies or combinatorial therapies disclosed herein for treatment or prevention of cancer.

The instructions relating to the use of one or more selected from the group consisting of TL1A and TL1A/p40 antibodies as described herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, ampules, tubes, bottles, jars, flexible packaging (e.g., sealed Mylar or plastic bags), and the like for each pharmaceutical composition and other included reagents, e.g., buffers, balanced salt solutions, etc., for use in administering the pharmaceutical compositions to subjects. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition selected from the group consisting of TL1A and TL1A/p40 antibodies. The container may further comprise a second pharmaceutically active agent.

Normally, the kit comprises a container and a label or package insert(s) on or associated with the container.

Incorporated by reference herein for all purposes is the content of U.S. Provisional Patent Application Nos. 62/949,120 (filed Dec. 17, 2019) and 63/110,693 (Filed Nov. 6, 2020).

As used herein, “mammalian cells” include reference to cells derived from mammals including humans, rats, mice, hamsters, guinea pigs, chimpanzees, or macaques. The cells may be cultured in vivo or in vitro.

As used herein, the term “purified product” refers to a preparation of the product which has been isolated from the cellular constituents with which the product is normally associated or from other types of cells that may be present in the sample of interest.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure. The term “non-human animals” of the invention includes all non-human vertebrates, e.g., non-human mammals and non-mammals, such as non-human primates, sheep, dog, cow, chickens, amphibians, reptiles, mouse, rat, rabbit or goat etc., unless otherwise noted.

As used herein, the term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

As used herein, the terms “pharmaceutically acceptable excipient, carrier or adjuvant” or “acceptable pharmaceutical carrier” refer to an excipient, carrier or adjuvant that can be administered to a subject, together with at least one antibody of the present disclosure, and which does not destroy the activity of the antibody. The excipient, carrier or adjuvant should be nontoxic when administered with an antibody in doses sufficient to deliver a therapeutic effect.

As used herein, the term “ameliorating” means a lessening or improvement of one or more symptoms as compared to not administering an antibody molecule of the invention. “Ameliorating” also includes shortening or reduction in duration of a symptom.

As used herein, the terms “prevent”, “preventing” and “prevention” refer to the prevention of the recurrence or onset of one or more symptoms of a disorder in a subject as result of the administration of a prophylactic or therapeutic agent.

Potency is a measure of the activity of a therapeutic agent expressed in terms of the amount required to produce an effect of given intensity. A highly potent agent evokes a greater response at low concentrations compared to an agent of lower potency that evokes a smaller response at low concentrations. Potency is a function of affinity and efficacy. Efficacy refers to the ability of therapeutic agent to produce a biological response upon binding to a target ligand and the quantitative magnitude of this response.

### Biological Deposits

Representative materials of the present invention were deposited in the American Type Culture Collection, 10801 University Boulevard, Manassas, VA 20110-2209, USA, on Dec. 17, 2021.

Vector “SFab p40-LC” having ATCC Accession No. PTA-127203 comprises a DNA insert encoding the “SFab p40-LC” and comprises SEQ ID NO: 178. SFab p40-LC is also referred to herein as “p40-LC.” Vector “p40-0003 VL” having ATCC Accession No. PTA-127205 comprises a DNA insert encoding the “p40-0003 VL” and comprises SEQ ID NO: 177. “p40-0003 VL” is referred to herein as “p40 VL.” Vector “p40-0003 VH” having ATCC Accession No. PTA-127206 comprises a DNA insert encoding the “p40-0003 VH” and comprises SEQ ID NO: 171. “p40-0003 VH” is referred to herein as “p40 VH.” Vector “p40-HC” having ATCC Accession No. PTA-127346 comprises a DNA insert encoding the “p40-HC” and comprises SEQ ID NO: 203. Vector “TL1A-0260-HC” having ATCC Accession No. PTA-127347 comprises a DNA insert encoding the “TL1A-0260-HC” and comprises SEQ ID NO: 161. “TL1A-0260-HC” is also referred to herein as TL1A HC (Functional). Vector “TL1A-LC” having ATCC Accession No. PTA-127348 comprises a DNA insert encoding the “TL1A-LC” and comprises SEQ ID NO: 19. Vector “TL1A-LC-Fc” having ATCC Accession No. PTA-127349 comprises a DNA insert encoding the “TL1A-LC-Fc” and comprises SEQ ID NO: 205. Vector “TL1AmFd” having ATCC Accession No. PTA-127350 comprises a DNA insert encoding the “TL1AmFd” and comprises SEQ ID NO: 207. Vector “[xTL1A]-0349-HC” having ATCC Accession No. PTA-127351 comprises a DNA insert encoding the “[xTL1A]-0349-HC” and comprises SEQ ID NO: 166.

| | | ATCC Accession | | |
|---|---|---|---|---|
| Antibody | Description | No. | SEQ ID NO: | DNA SEQ ID NO: |
| P40TL1A-0033 | SFab p40-LC | PTA-127203 | SEQ ID NO: 178 | SEQ ID NO: 237 |
| P40TL1A-0033 | p40-0003 VL | PTA-127205 | SEQ ID NO: 177 | SEQ ID NO: 239 |
| P40TL1A-0033 | p40-0003 VH | PTA-127206 | SEQ ID NO: 171 | SEQ ID NO: 238 |
| P40TL1A-0033 | P40-HC | PTA-127346 | SEQ ID NO: 203 | SEQ ID NO: 236 |
| TL1A-0260 | TL1A-0260-HC | PTA-127347 | SEQ ID NO: 161 | SEQ ID NO: 231 |
| TL1A-0260 | TL1A-LC | PTA-127348 | SEQ ID NO: 19 | SEQ ID NO: 233 |
| P40TL1A-0033 | TL1A-LC-Fc | PTA-127349 | SEQ ID NO: 205 | SEQ ID NO: 235 |
| P40TL1A-0033 | TL1AmFd | PTA-127350 | SEQ ID NO: 207 | SEQ ID NO: 234 |
| TL1A-0372 | [xTL1A]-0349-HC | PTA-127351 | SEQ ID NO: 166 | SEQ ID NO: 232 |

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions; the materials will be promptly replaced on notification with another of the same. Availability of the deposited material is not to be construed as a license to practice the invention in contravention of the rights granted under the authority of any government in accordance with its patent laws.

### Materials and Methods

Various techniques for the production of antibodies have been described which include the traditional hybridoma method for making monoclonal antibodies, recombinant techniques for making antibodies (including chimeric antibodies, e.g., humanized antibodies), antibody production in transgenic animals and the recently described phage display technology for preparing “fully human” antibodies.

Provided herein are methods of making any of the antibodies provided herein. The antibodies of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

Any suitable method for preparing multispecific antibodies may be used to prepare multispecific antibodies provided herein (e.g. depending on the choice of antibody features and components).

According to one approach to making multispecific antibodies, antibody variable domains with the desired binding specificities are fused to immunoglobulin constant region sequences. The fusion preferably is with an immunoglobulin heavy chain constant region, comprising at least part of the hinge, CH2 and CH3 regions. In some embodiments, the first heavy chain constant region (CH1), containing the site for light chain binding can be present in at least one of the fusions. In some embodiments, polynucleotides encoding the immunoglobulin heavy chain fusions and, if desired, the immunoglobulin light chain, may be inserted into separate expression vectors, and may be cotransfected into a suitable host organism. In other embodiments the coding sequences for two or all three polypeptide chains may be inserted into one expression vector when the expression of at least two polypeptide chains in equal ratios results in high yields or when the ratios are of no particular significance.

In one approach, the multispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the multispecific molecule, facilitates the separation of the desired multispecific compound from unwanted immunoglobulin chain combinations. This approach is described in PCT Publication No. WO 94/04690.

In another approach, the multispecific antibodies are composed of amino acid modification in the first hinge region in one arm, and the substituted amino acid in the first hinge region has an opposite charge to the corresponding amino acid in the second hinge region in another arm. This approach is described in International Patent Application No. PCT/US2011/036419 (WO2011/143545).

In another approach, the formation of a desired heteromultimeric or heterodimeric protein (e.g., bispecific antibody) is enhanced by altering or engineering an interface between a first and a second Fc chain. In this approach, the multispecific antibodies may be composed of a CH3 region, wherein the CH3 region comprises a first CH3 polypeptide and a second CH3 polypeptide which interact together to form a CH3 interface, wherein one or more amino acids within the CH3 interface destabilize homodimer formation and are not electrostatically unfavorable to homodimer formation. This approach is described in International Patent Application No. PCT/US2011/036419 (WO2011/143545). In some embodiments, one Fc chain of a bispecific antibody can comprise amino acid modifications at positions 223 and 228 (e.g., (C223E or C223R), and (P228E or P228R)) in the hinge region and at position 409 (e.g., K409R (EU numbering scheme)) in the CH3 region of human IgG2, and the other Fc chain of the bispecific antibody can comprise amino acid modifications at positions 223, 225 and 228 (e.g., (C223E or C223R), (E225R), and (P228E or P228R)) in the hinge region and at position 368 (e.g., L368E (EU numbering scheme)) in the CH3 region of human IgG2. In other embodiments, one Fc chain of a bispecific antibody can comprise amino acid modifications at positions 223 and 228 (e.g., (C223E or C223R) and (P228E or P228R)) in the hinge region and at position 368 (e.g., L368E (EU numbering scheme)) in the CH3 region of human IgG2, and the other Fc chain of the bispecific antibody can comprise amino acid modifications at positions 223, 225 and 228 (e.g., (C223E or C223R), (E225R), and (P228E or P228R)) in the hinge region and at position 409 (e.g., K409R (EU numbering scheme)) in the CH3 region of human IgG2. In some embodiments, a bispecific antibody can comprise amino acid modifications at positions 221 and 228 (e.g., (D221R or D221E) and (P228R or P228E)) in the hinge region and at position 409 or 368 (e.g., K409R or L368E (EU numbering scheme)) in the CH3 region of human IgG1. In some embodiments, a bispecific antibody can comprise amino acid modifications at positions 228 (e.g., (P228E or P228R)) in the hinge region and at position 409 or 368 (e.g., R409 or L368E (EU numbering scheme)) in the CH3 region of human IgG4.

In some embodiments, a multispecific antibody may have knob-in-hole mutations in the Fc chains. For example, in some embodiments, in a bispecific antibody having knob-in-hole mutations, the first Fc chain of the antibody Fc domain has one or more mutations to form a “knob”, and the second Fc chain of the antibody Fc domain has one or more mutations to form a “hole” (or vice-versa). Exemplary knob-in-hole engineering of antibodies is described in U.S. Pat. No. 5,731,168, PCT Publication No. WO2009089004, U.S. Publication No. 20090182127, Marvin and Zhu, Acta Pharmacologica Sincia (2005) 26(6):649-658 and Kontermann (2005) Acta Pharacol. Sin., 26:1-9.

A “knob” refers to at least one amino acid side chain which projects from the interface of a first polypeptide (e.g. first Fc chain) and is therefore positionable in a compensatory hole in an adjacent second polypeptide (e.g. second Fc chain) so as to stabilize a heterodimer, and thereby favor heterodimer formation over homodimer formation. The knob may exist in the original interface or may be introduced synthetically (e.g., by altering a nucleic acid encoding the interface). Normally, nucleic acid encoding the interface of the first polypeptide is altered to encode the knob. To achieve this, the nucleic acid encoding at least one original amino acid residue in the first polypeptide is replaced with nucleic acid encoding at least one “import” amino acid residue which has a larger side chain volume than the original amino acid residue. Certain import residues for the formation of a knob are generally naturally occurring amino acid residues and are preferably selected from arginine (R), phenylalanine (F), tyrosine (Y) and tryptophan (W).

A “hole” refers to at least one amino acid side chain which is recessed from the interface of a second polypeptide (e.g. second Fc chain) and therefore accommodates a corresponding knob in an adjacent first polypeptide (e.g. first Fc chain). The hole may exist in the original interface or may be introduced synthetically (e.g., by altering a nucleic acid encoding the interface). Normally, nucleic acid encoding the interface of the second polypeptide is altered to encode the hole. To achieve this, the nucleic acid encoding at least one original amino acid residue of the second polypeptide is replaced with DNA encoding at least one “import” amino acid residue which has a smaller side chain volume than the original amino acid residue. Certain import residues for the formation of a hole are usually naturally occurring amino acid residues and are preferably selected from alanine (A), serine (S), threonine (T) and valine (V).

The term “interface,” as used herein typically refers to any amino acid residue present in the domain that can be involved in first polypeptide and second polypeptide contacts. An “original amino acid” residue is one which is replaced by an “import amino acid” residue which can have a smaller or larger side chain volume than the original residue. The import amino acid residue can be a naturally occurring or non-naturally occurring amino acid residue, but preferably is the former. “Naturally occurring” amino acid residues are those residues encoded by the genetic code. By “non-naturally occurring” amino acid residue is meant a residue which is not encoded by the genetic code, but which is able to covalently bind adjacent amino acid residue(s) in the polypeptide chain. Examples of non-naturally occurring amino acid residues are norleucine, ornithine, norvaline, homoserine and other amino acid residue analogues such as those described in Ellman et al., Meth. Enzym. 202:301-336 (1991).

Once a nucleic acid sequence encoding molecules of the invention (i.e., binding domains) has been obtained, the vector for the production of the molecules may be produced by recombinant DNA technology using techniques well known in the art.

The polynucleotides encoding the antibody (binding domains of the present invention may include an expression control polynucleotide sequence operably linked to the antibody coding sequences, including naturally-associated or heterologous promoter regions known in the art. The expression control sequences may be eukaryotic promoter systems in vectors capable of transforming or transfecting eukaryotic host cells, but control sequences for prokaryotic hosts may also be used. Once the vector has been incorporated into the appropriate host cell line, the host cell is propagated under conditions suitable for expressing the nucleotide sequences, and, as desired, for the collection and purification of the antibodies. Eukaryotic cell lines include the CHO cell lines, various COS cell lines, HeLa cells, myeloma cell lines, transformed B-cells, or human embryonic kidney cell lines.

In one embodiment, the DNA encoding the antibodies of the invention is isolated and sequenced using conventional procedures (e.g., by using oligonucleotide probes that are capable of binding specifically to genes encoding the heavy and light chains of antibodies). Once isolated, the DNA may be placed into expression vectors, which are then transfected into host cells such as simian COS cells, CHO cells, or myeloma cells that do not otherwise produce immunoglobulin protein, to obtain the synthesis of monoclonal antibodies in the recombinant host cells. The DNA also may be modified, for example, to improve one or more properties of the corresponding antibody (e.g. binding affinity, immunogenicity, etc.).

In one aspect, the invention provides a method of making any of the polynucleotides described herein. For example, the polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art (e.g., Sambrook et al., 1989).

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cells capable of over-expressing heterologous DNAs can be used for the purpose expressing genes encoding the antibody, polypeptide or protein of interest. Non-limiting examples of mammalian host cells include but not limited to COS, HeLa, and CHO cells. See also PCT Publication No. WO87/04462. Suitable non-mammalian host cells include prokaryotes (such as E. coli or B. subtillis) and yeast (such as S. cerevisae, S. pombe; or K. lactis). A cell overexpressing the antibody or protein of interest can be identified by known screening methods.

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

In some embodiments of the invention, the antibody comprises a modified constant region, such as a constant region that has increased affinity to a human Fc gamma receptor, is immunologically inert or partially inert, e.g., does not trigger complement mediated lysis, does not stimulate antibody-dependent cell mediated cytotoxicity (ADCC), or does not activate macrophages; or has reduced activities (compared to the unmodified antibody) in any one or more of the following: triggering complement mediated lysis, stimulating antibody-dependent cell mediated cytotoxicity (ADCC), or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 29:2613-2624, 1999; PCT Application No. PCT/GB99/01441; and/or UK Application No. 9809951.8. In still other embodiments, the constant region is aglycosylated for N-linked glycosylation. In some embodiments, the constant region is aglycosylated for N-linked glycosylation by mutating the glycosylated amino acid residue or flanking residues that are part of the N-glycosylation recognition sequence in the constant region. For example, N-glycosylation site N297 may be mutated to A, Q, K, or H. See, Tao et al., J. Immunology 143: 2595-2601, 1989; and Jefferis et al., Immunological Reviews 163:59-76, 1998. In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications include antibodies that have been modified as described in PCT Publication No. WO99/58572. These antibodies comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain is capable of specifically binding either or both of the FcRn or the FcγRIIb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy.

In some embodiments, the Fc chain of an antibody provided herein may be modified to ablate effector function. For example, the Fc chain of human IgG1 may be modified to introduce mutations L234A, L235A and G237A using standard primer-directed PCR mutagenesis to oblate effector function due to binding to FcγRIII, providing for an effector function null phenotype (Canfield et al., J. Exp. Med (1991) 173: 1483-1491; Shields et al., J. Biol. Chem. (2001) 276:6591-604).

In some embodiments, a multispecific antibody provided herein may be engineered to comprise at least one cysteine residue that may interact with a counterpart cysteine residue on another polypeptide chain of the invention to form an inter-chain disulfide bond. The inter-chain disulfide bonds may serve to stabilize the multispecific antibody, improving expression and recovery in recombinant systems, resulting in a stable and consistent formulation, as well as, improving the stability of the isolated and/or purified product in vivo. The cysteine residue or residues may be introduced as a single amino acid or as part of larger amino-acid sequence, e.g., hinge region, in any portion of the polypeptide chain. In a specific aspect, at least one cysteine residue is engineered to occur at the C-terminus of the polypeptide chain.

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### EXAMPLES

The following examples of specific aspects for carrying out the present invention are offered for illustrative purposes only and are not intended to limit the scope of the present invention in any way.

### Example 1. Generation of Anti-TL1A Antibody TL1A-0002

Fully human affinity optimized anti-TL1A heavy and light chain amino acid sequences were previously described in Arch et al. U.S. Pat. No. 9,683,998 Pfizer Inc. and Bristol-Myers Squibb Company, Jun. 19, 2017 (SEQ ID NO: 228 and SEQ ID NO: 106)¹. 1D1 1.31 is a human IgG1/Kappa neutralizing antibody harboring L(247)A, L(248)A and G(250)A mutations (Pfabat numbering) within the fragment crystallizable region (Fc region) to minimize effector function and will be referred to herein as TL1A-0002. Heavy and light chain amino acid sequences comprising TL1A-0002 are depicted in SEQ ID NO: 10 and SEQ ID NO: 17, respectively. TL1A-0002 antibody demonstrated efficacy in a Phase 2a trial for moderate-to-severe ulcerative colitis (UC) but yielded a high frequency of positive anti-drug antibodies (ADA) subjects, 41 of 50 dosed, or 82%, with anti-drug antibodies (ADA) and 5 of 50 dosed, or 10%, of subjects with neutralizing antibodies (https://clinicaltrials.gov, Identification No. NCT02840721). The development and maturation of neutralizing anti-drug antibodies in patients could limit the long-term benefits of the drug. We sought to develop a biobetter antibody that maintains the efficacy of TL1A-0002 while de-risking immunogenicity to result in a therapeutic with low ADA.

### Example 2. Derivation of Anti-TL1A Binding Domains Engineered to Reduce Immunogenicity and Eliminate Post-Translational Asparagine Deamidation Modifications

Fully human anti-TL1A antibody TL1A-0002 underwent engineering efforts to reduce immunogenicity and remove post-translational asparagine deamidation modifications as a follow-on biotherapeutic intended for chronic treatment of inflammatory bowel disease (IBD). Predictive immunogenicity of the amino acid sequence of TL1A-0002 was analyzed using the two methods described below to identify in silico predicted T cell epitopes. Any sequence flagged by the rules described herein for either protocol was considered an epitope. Sequences were submitted for EpiMatrix analysis in the ISPRI software package (ISPRI v 1.8.0, EpiVax Inc., Providence, RI; Schafer et al., 1998). The raw results provide rankings of likelihood of binding of each 9-mer amino acid fragment against 8 different HLA types. 9-mer amino acid peptides that are identified as natural T regulatory cell epitopes (Tregitopes)² that bind different HLA types are excluded as EpiVax hits. Sequences were also submitted for analysis using the MHC-II binding consensus method (Wang et al, 2010; Wang et al, 2008) in IEDB (IEDB MHC-II Binding Predictions, Vita et al., 2015). The raw results provide rankings of likelihood of binding of each 9-mers amino acid fragments against the same 8 different HLA types. Each epitope is classified as a germline or non-germline epitope. For antibodies, each epitope is further classified based on its location within the antibody complementary determining region (CDR) or non-CDR location. The analysis of TL1A-0002 using these two methods identified a total of five predicted non-germline T cell epitopes in the antibody heavy chain variable region (V_{H}), one in CDR-H1 (SEQ ID NO: 1), three in CDR-H2 (SEQ ID NO: 2) and one in CDR-H3 (SEQ ID NO: 3), reported in Table 1. To minimize the risk of immunogenicity with TL1A-0002, amino acid substitutions were introduced at the highlighted residues to eliminate putative T cell epitopes, without affecting binding to TL1A, as described below.

TABLE 1
Location of in silico Predicted Non-Germline T Cell Epitopes in CDRs of TL1A-0002V_{H}.

| Non- | | Hits | | | |
|---|---|---|---|---|---|
| germline | Predicted T | EpiVax | | IEDB | |
| CDR | cell epitope | Number of | | Number of | |
| epitope | fragment | HLA hits | Allele List | HLA hits | Allele List |
| H29 | FTYYGISWV | NA | N/A | 3 | HLA- |
| | | | | | DRB1*04:01 |
| | | | | | HLA- |
| | | | | | DRB1*08:01 |
| | | | | | HLA- |
| | | | | | DRB1*15:01 |
| H50 | WISTYNGNT | 4 | HLA- | NA | N/A |
| | | | DRB1*01:01 | | |
| | | | HLA- | | |
| | | | DRB1*04:01 | | |
| | | | HLA- | | |
| | | | DRB1*07:01 | | |
| | | | HLA- | | |
| | | | DRB1*08:01 | | |
| H53 | YNGNTHYAR | 4 | HLA- | NA | N/A |
| | | | DRB1*03:01 | | |
| | | | HLA- | | |
| | | | DRB1*08:01 | | |
| | | | HLA- | | |
| | | | DRB1*11:01 | | |
| | | | HLA- | | |
| | | | DRB1*13:01 | | |
| H59 | YARMLQGRV | NA | N/A | 4 | HLA- |
| | | | | | DRB1*01:01 |
| | | | | | HLA- |
| | | | | | DRB1*08:01 |
| | | | | | HLA- |
| | | | | | DRB1*11:01 |
| | | | | | HLA- |
| | | | | | DRB1*15:01 |
| H97 | YYGSGAYRG | 4 | HLA- | NA | N/A |
| | | | DRB1*01:01 | | |
| | | | HLA- | | |
| | | | DRB1*04:01 | | |
| | | | HLA- | | |
| | | | DRB1*08:01 | | |
| | | | HLA- | | |
| | | | DRB1*11:01 | | |

NA indicates no 9mers were identified, or the sequence did not meet cutoffs for inclusion in the report for that particular piece of software. No EpiVax results are returned for sequences flagged as T-regitopes (because these sequences do not contribute to the EpiVax score), even if the sequence is also flagged by IEDB.
N/A = not applicable

Manual inspection of the TL1A-0002 heavy and light chain variable regions identified two putative deamidation hot spots within the V_{H}, Asn (H54) and Asn (H96). Forced degradation analysis was performed on TL1A-0002 antibody to understand if these potential deamidation hot spots were being chemically modified. For this stressed protein analysis, TL1A-0002 antibody was formulated to 5 mg/mL into three standard formulation buffers minus excipients (20 mM Tris pH 7.5, 20 mM Histidine pH 5.8, and 20 mM Glutamic acid pH 4.5), incubated at 40° C. and samples removed at 0, 2, and 4 weeks for evaluation. Analytical Imaged Capillary

Electrophoresis (iCE) was used to assess chemical post-translational deamidation which frequently correlates with an increased level of acidic species that corresponds with a lower level of the main isoform. iCE analysis of anti-TL1A antibody TL1A-0002 forced degradation samples showed elevated levels of acidic charged species upon storage at 40° C. in Tris pH 7.5 buffer and amount of acidic charged species increased over the two- and four-week time intervals. Mass spectrometric analyses (LC-MS/MS) were performed to assess thermal and pH stress on TL1A-0002 antibody and evaluate level of each modification within the CDR regions at initial conditions (T=0) and stressed conditions (T=4w). For this analysis, TL1A-0002 formulated into 20 mM Histidine, 85 mg/mL sucrose, 0.05 mg/mL EDTA, 0.2 mg/mL PS80, pH 5.8 (T=0) was buffer exchanged into: Tris pH 7.5, Histidine pH 5.8, and Glutamic acid pH 4.5 buffers and then subjected to thermal stress (40° C.) for 4 weeks. Next, a low-artifact Lys-C/trypsin (LATD) peptide mapping LC-MS/MS method was performed at pH 6.0 and pH 8.2 with a 0.5, 1- and 2-hour time course at 37° C. The extracted ion chromatograms for modified and unmodified peptides were integrated and normalized to 100% and the percent modified peptide is reported (Table 2). Evaluation of data indicated that it was Asn residues at both heavy chain positions H54 (Pfabat numbering) in the V_{H} CDR2 (SEQ ID NO: 2) and H96 (Pfabat numbering) in the V_{H} CDR3 (SEQ ID NO: 3) that was partially deamidated. Aspartate (Asp)-Isoaspartate (IsoAsp) deamidation was elevated after thermal stress at both pH 5.8 and pH 7.5 which is consistent with forced degradation analysis results.

TABLE 2
Summary of Asn Deamidation Liability Data Identified
in Parent TL1A-0002 Antibody by LC-MS/MS analysis.

| Material | N(H54) in CDR-H2 | N(H96) in CDR-H3 |
|---|---|---|
| Reference material | 96.2/3.7*/<0.1 | 85.4/14.0*/0.6 |
| 4 w, pH 7.5 | 87.1/11.5*/1.4 | 16.4/81.8*/1.8 |
| 4 w, pH 5.8 | 93.7/5.1*/1.2 | 75.7/21.5*/2.8 |
| 4 w, pH 4.5 | 94.6/4.2*/1.2 | 84.7/13.5*/1.8 |

Values are reported as percent Asn/Asp-IsoAsp/Succinimide, respectively.
*The total percent deamidation is reported as Asp-IsoAsp.

Amino acid substitutions for reducing T cell epitope content and eliminating deamidation liabilities in TL1A-0002 antibody CDRs were guided by computational and manual inspection of the binding interface in the co-crystal structure of the parental 1D1 antigen-binding fragment (Fab) with human TL1A (Arch et al. U.S. Pat. No. 9,683,998 Pfizer Inc. and Bristol-Myers Squibb Company, Jun. 19, 2017, EXAMPLE 6) to aide in maintaining TL1A binding properties. Using a structure guided approach, variants with amino acid substitutions in TL1A-0002 V_{H} CDRs that remove in silico predicted T cell epitopes, non-germline framework residue and post-translational deamidation modifications without altering the binding to TL1A were generated. Table 3 lists the amino acids that were substituted and their substitution(s).

TABLE 3
Summary of Recommended Structure Guided Amino Acid
Substitutions in TL1A-0002 V_{H}.

| CDR-H1 | | CDR-H2 | | CDR-H3 | | VH Framework | |
|---|---|---|---|---|---|---|---|
| in | | In | | In | | In | |
| TL1A- | | TL1A- | | TL1A- | | TL1A- | |
| 0002 | Variant | 0002 | Variant | 0002 | Variant | 0002 | Variant |
| (SEQ ID | Amino | (SEQ ID | Amino | (SEQ ID | Amino | (SEQ ID | Amino |
| NO: 1) | Acids | NO: 2) | Acids | NO: 3) | Acids | NO: 5) | Acids |
| G26 | | W50 | | E95 | | R76 | S |
| Y27 | F | I51 | A | N96 | E, Q, | | |
| | | | | | S, T, V | | |
| D28 | | S52 | A | Y97 | L | | |
| F29 | Y | T52a | A | Y98 | W | | |
| T30 | A, S | Y53 | W | G99 | | | |
| Y31 | F, K, | N54 | G, Q, | S100 | | | |
| | R, W | | S, T | | | | |
| Y32 | H | G55 | A, D, | G100a | E, H, | | |
| | | | T | | P, W | | |
| G33 | A | N56 | G, V | A100b | D, E, | | |
| | | | | | L | | |
| I34 | V | T57 | D, E | Y100c | F, H | | |
| S35 | G | H58 | N | R100d | T, V, | | |
| | | | | | Y | | |
| | | Y59 | H | G100e | A, H, | | |
| | | | | | V | | |
| | | A60 | | G100f | | | |
| | | R61 | D, P, | M100g | | | |
| | | | Q | | | | |
| | | M62 | K, S | D101 | | | |
| | | L63 | | V102 | | | |
| | | Q64 | | | | | |
| | | G65 | | | | | |

Panels of variants based on TL1A-0002 were designed one V_{H} CDR at a time as human IgG1-effector function-minimized/human Kappa antibodies and produced transiently in Expi293F™ cells. TL1A binding properties were assessed for the TL1A-0002 antibody variants using a competition enzyme-linked immunosorbent assay (ELISA) assay with biotinylated TL1A-0002 (bioTL1A-0002) as the reporter antibody to determine if variants could effectively compete with the parental TL1A-0002 antibody for binding to TL1A antigen. The parental TL1A-0002 reporter antibody was biotinylated using EZ-link™ Sulfo-NHS-Biotin Sulfosuccinimidobiotin at a molar coupling ratio of 20:1 according to the manufacturer's protocols. For this competition ELISA assay procedure, 96-well high binding plates (Corning, product number 3590) were coated with 1 μg/mL recombinant TL1A in 100 μL/well in Phospate Buffered Saline (PBS) overnight at 4° C. Plates were then blocked in PBS+0.02% casein solution and washed with PBS+0.05% Tween-20 following standard ELISA protocol. Biotinylated TL1A-0002 antibody diluted to the EC₅₀ (5 ng/mL) in assay buffer (PBS+0.5% BSA+0.02% Tween-20) was mixed with a 0.015-300 nM concentration range of the variants or parental TL1A-0002 antibody as the positive control, using a singleton sample for each antibody concentration evaluated. Plates were washed with PBS+0.05% Tween-20 following standard ELISA protocol, and Streptavidin-HRP (Southern Biotech, product number 7100-05) diluted to 1:10,000 in assay buffer was added to plates and incubated for 25-30 minutes at room temperature. Next, the plates were washed with PBS+0.05% Tween-20 following standard ELISA protocol, and then TMB substrate (BioFX Laboratories, catalog number TMBW-0100-01) was added to plates. Reactions were stopped after approximately 5 minutes by adding 0.18 M ortho-phosphoric acid to each well and absorbance was read at 450 nm. Variants harboring mutations that retained TL1A binding properties in the bioTL1A-0002 competition ELISA within 3-fold of the parental TL1A-0002 antibody were considered tolerated and are summarized in Table 4.

The TL1A-0002 variants were additionally subjected to DNA/insulin polyreactivity³ and AC-SINS self-association⁴ biophysical characterization assays to evaluate non-specificity properties. DNA and insulin direct binding ELISAs were performed to assess polyreactivity of the variants using the following method adapted from Tiller et. al using a PerkinElmer Janus Automated Workstation liquid handling robot. DNA (Sigma-Aldrich, D1626) or insulin (Sigma-Aldrich, 19278-5 mL) diluted in PBS-CMF pH 7.2 to 10 μg/mL or 5 μg/mL, respectively, was pipetted into 384-well ELISA plates (Nunc Maxisorp) and incubated at 4° C. overnight. The plates were then washed with water, blocked with 50 μl of Polyreactivity ELISA Buffer (PBS containing 0.05% Tween-20, 1 mM EDTA) for 1 hour at room temperature, and rinsed three times with water. Serially diluted samples were added in quadruplicate to the wells and incubated for 1 hour at room temperature. Plates were washed three times with water, and goat anti-human IgG conjugated to horseradish peroxidase (Jackson ImmunoResearch, 109-035-008) diluted to 10 ng/mL was added to plates and incubated for 1 hour at room temperature. Next, the plates were washed three times with water and then TMB substrate (Sigma-Aldrich, T-0440) was added to plates. Reactions were stopped after approximately 7 minutes by adding 0.18 M ortho-phosphoric acid to each well and absorbance read at 450 nm. The DNA and insulin binding scores were calculated as the ratio of the ELISA signal of the antibody at 10 μg/mL versus the signal of control wells containing buffer instead of the primary antibody. Ranking of polyreactivity scores are the following: Good 0-5, Moderate >5 and <10, High >10.

The AC-SINS assay was adapted to a 384-well format using the Perkin-Elmer Janus liquid handling robot. Specifically, 20 nm gold nanoparticles (Ted Pella, Inc., catalog #15705) were coated with a mixture of 80% goat anti-human Fc (Jackson ImmunoResearch Laboratories, Inc. catalog #109-005-098) and 20% non-specific goat polyclonal antibodies (Jackson ImmunoResearch Laboratories, Inc. catalog #005-000-003) that were buffer exchanged into 20 mM sodium acetate pH 4.3 and diluted to 0.4 mg/mL. The plate was incubated for 1 hour at room temperature. Next, unoccupied sites on the gold nanoparticles were blocked with thiolated polyethylene glycol (2 kD). The coated/blocked nanoparticles were then concentrated 10-fold using a syringe filter and 10 μL were added to 100 μL of the antibody at 0.05 mg/mL in PBS pH 7.2. The coated/blocked nanoparticles were incubated with the sample of interest for 2 hours in a 96-well polypropylene plate, transferred to a 384-well polystyrene plate and the absorbance was read on a Tecan M1000 spectrophotometer from 450-650 in 2 nm increments. A Microsoft Excel macro was used to identify the max absorbance, and smooth and fit the data using a second-order polynomial. The smoothed maximum absorbance of the average blank (PBS buffer alone) was subtracted from the smoothed maximum absorbance of the sample to determine the AC-SINS score. Ranking of AC-SINS scores is the following: Good 0-5, Moderate >5 and <10, High >10.

T-reg Adjusted Pfizer scores were calculated for each antibody by utilizing the raw per-allele, per-9mer amino acid binding scores against 8 different HLA types in the EpiMatix and IEDB analysis (EXAMPLE 2). This score reflects an overall in silico immunogenicity score for each antibody. Ranking of scores are the following: Good ≤−50, Moderate ≤−30 and ≥−50, Poor ≥−30.

Characterization of TL1A binding properties using the bioTL1A-0002 antibody competition ELISA identified 56 variants from the 101 antibodies that retain TL1A binding properties within 3-fold of parental antibody TL1A-0002 (Table 4). Non-specificity evaluation showed that these variants maintained similar or improved AC-SINS, DNA, and Insulin scores compared to those of parental TL1A-0002 (Table 4). The T-reg Adjusted Pfizer score improves for antibody variants where amino acid substitutions were introduced for reduction of T cell epitope content (Table 4).

TABLE 4
Properties of TL1A-0002 and Variants with Rational Mutations to Remove Predicted T
Cell Epitopes and Deamidation Liabilities Introduced into Individual CDRs.

| | | Competition ELISA | | | | | |
|---|---|---|---|---|---|---|---|
| | | | Bioactivity | | | | T-reg |
| | V_{H} mutation(s) | | relative to | Non-specificity score | | | Adjusted |
| Name | Pfabat numbering | IC₅₀ [nM] | TL1A-0002 | AC-SINS | DNA | Insulin | Pfizer score |
| TL1A-0002 | Parental | 4.5 | 1.0 | 7 | 8 | 4 | −18.5 |
| TL1A-0012 | R(61)D, M(62)S | 5.0 | 1.1 | 3 | 1 | 1 | −26.3 |
| TL1A-0013 | R(61)P, M(62)S | 5.5 | 1.2 | 2 | 6 | 3 | −24.52 |
| TL1A-0014 | R(61)Q, M(62)K | 5.1 | 1.1 | 5 | 4 | 3 | −21.58 |
| TL1A-0019 | Y(59)H | 5.0 | 1.1 | 8 | 6 | 3 | −23.21 |
| TL1A-0021 | T(57)D | 6.0 | 1.3 | 5 | 3 | 3 | −24.04 |
| TL1A-0022 | T(57)E | 6.9 | 1.5 | 4 | 4 | 3 | −22.91 |
| TL1A-0023 | T(57)D, R(61)P, | 5.6 | 1.3 | 3 | 3 | 3 | −28.76 |
| | M(62)S | | | | | | |
| TL1A-0024 | T(57)E, R(61)P, | 5.4 | 1.2 | 2 | 3 | 2 | −28.85 |
| | M(62)S | | | | | | |
| TL1A-0025 | T(57(D, R(61)D, | 7.2 | 1.6 | 3 | 2 | 1 | −28.68 |
| | M(62)S | | | | | | |
| TL1A-0026 | T(57)E, R(61)D, | 8.9 | 2.0 | 2 | 2 | 1 | −29.65 |
| | M(62)S | | | | | | |
| TL1A-0027 | T(52a)A, H(58)N, | 8.3 | 1.9 | 4 | 2 | 2 | −16.11 |
| | R(61)Q, M(62)K | | | | | | |
| TL1A-0028 | Y(98)W | 5.4 | 1.2 | 8 | 6 | 2 | −19.52 |
| TL1A-0029 | Y(97)L | 7.8 | 1.7 | 4 | 6 | 3 | −22.63 |
| TL1A-0030 | A(100b)E | 6.1 | 1.4 | 2 | 3 | 3 | −22.79 |
| TL1A-0031 | A(100b)D | 7.4 | 1.6 | 4 | 3 | 3 | −22.79 |
| TL1A-0033 | Y(100c)F | 3.6 | 0.8 | 6 | 6 | 3 | −20.59 |
| TL1A-0038 | A(100b)D, | 7.2 | 1.6 | 2 | 6 | 2 | −22.79 |
| | Y(100c)F | | | | | | |
| TL1A-0040 | A(100b)D, | 4.7 | 1.0 | 3 | 6 | 2 | −21.83 |
| | Y(100c)H | | | | | | |
| TL1A-0061 | Y(27)F | 4.6 | 1.0 | 6 | 5 | 4 | −18.5 |
| TL1A-0062 | F(29)Y | 4.0 | 0.9 | 7 | 3 | 3 | −18.51 |
| TL1A-0063 | T(30)S | 4.1 | 0.9 | 7 | 3 | 2 | −19.47 |
| TL1A-0067 | Y(31)F | 5.1 | 1.1 | 5 | 1 | 1 | −18.37 |
| TL1A-0068 | Y(31)K | 4.6 | 1.0 | 3 | 1 | 1 | −20.43 |
| TL1A-0069 | Y(32)H | 4.1 | 0.9 | 5 | 1 | 1 | −18.74 |
| TL1A-0071 | I(34)V | 3.9 | 0.9 | 7 | 2 | 1 | −21.44 |
| TL1A-0073 | G(55)T, R(61)Q, | 5.0 | 1.1 | 4 | 5 | 3 | −21.84 |
| | M(62)K | | | | | | |
| TL1A-0074 | G(55)T, R(61)P, | 4.5 | 1.0 | 3 | 5 | 5 | −25.52 |
| | M(62)S | | | | | | |
| TL1A-0076 | G(55)D, R(61)P, | 5.4 | 1.2 | 3 | 2 | 2 | −28.07 |
| | M(62)S | | | | | | |
| TL1A-0079 | N(54)Q | 5.8 | 1.3 | 7 | 9 | 3 | −20.31 |
| TL1A-0080 | N(54)G | 5.0 | 1.1 | 8 | 2 | 2 | −23.82 |
| TL1A-0081 | T(52a)A | 4.1 | 0.9 | 7 | 2 | 3 | −14.73 |
| TL1A-0082 | T(52a)A, R(61)Q, | 5.2 | 1.2 | 6 | 3 | 3 | −17.81 |
| | M(62)K | | | | | | |
| TL1A-0083 | N(54)G, R(61)Q, | 6.8 | 1.5 | 6 | 2 | 2 | −25.19 |
| | M(62)K | | | | | | |
| TL1A-0084 | I(51)A | 4.5 | 1.0 | 6 | 5 | 4 | −22.01 |
| TL1A-0085 | I(51)A, R(61)Q, | 4.7 | 1.0 | 5 | 4 | 2 | −25.09 |
| | M(62)K | | | | | | |
| TL1A-0086 | I(51)A, R(61)P, | 5.0 | 1.1 | 4 | 1 | 2 | −28.03 |
| | M(62)S | | | | | | |
| TL1A-0087 | T(52a)A, R(61)P, | 4.8 | 1.1 | 4 | 1 | 2 | 20.75 |
| | M(62)S | | | | | | |
| TL1A-0088 | N(54)G, R(61)P, | 6.5 | 1.4 | 4 | 2 | 1 | −28.54 |
| | M(62)S | | | | | | |
| TL1A-0089 | G(55)T, N(56)G, | 5.4 | 1.2 | 2 | 1 | 1 | −25.63 |
| | R(61)Q, M(62)K | | | | | | |
| TL1A-0090 | G(55)T, N(56)G, | 4.5 | 1.0 | 2 | 1 | 2 | −27.58 |
| | R(61)P, M(62)S | | | | | | |
| TL1A-0091 | G(55)T, N(56)V, | 5.4 | 1.2 | 4 | 1 | 1 | −22.32 |
| | R(61)Q, M(62)K | | | | | | |
| TL1A-0092 | G(55)T, N(56)V, | 5.2 | 1.2 | 4 | 1 | 1 | −23.49 |
| | R(61)P, M(62)S | | | | | | |
| TL1A-0093 | T(52a)A, G(55)A, | 5.9 | 1.3 | 2 | 4 | 4 | −20.97 |
| | R(61)P, M(62)S | | | | | | |
| TL1A-0094 | N(96)S | 5.5 | 1.2 | 4 | 4 | 4 | −19.52 |
| TL1A-0095 | N(96)T | 8.8 | 2.0 | 4 | 2 | 3 | −19.55 |
| TL1A-0096 | N(96)V | 10.3 | 2.3 | 3 | 3 | 3 | −19.67 |
| TL1A-0097 | N(96)Q | 10.0 | 2.2 | 4 | 9 | 6 | −20.65 |
| TL1A-0098 | N(96)E | 6.6 | 1.5 | 4 | 3 | 3 | −20.65 |
| TL1A-0147 | T(57)E, R(61)Q, | 7.2 | 1.6 | 3 | 2 | 1 | −25.01 |
| | M(62)K | | | | | | |
| TL1A-0160 | T(57)E, R(61)P, | 9.1 | 2.0 | 7 | 7 | 2 | −32.12 |
| | M(62)S, A(100b)L | | | | | | |
| TL1A-0167 | R(76)S | 4.2 | 0.9 | 6 | 5 | 2 | −18.5 |
| TL1A-0193 | N(96)Q, A(100b)E | 12.0 | 2.7 | 3 | 2 | 3 | −24.94 |
| TL1A-0194 | N(96)Q, Y(100c)F | 8.3 | 1.8 | 6 | 4 | 4 | −22.75 |
| TL1A-0195 | N(96)Q, Y(100c)H | 8.2 | 1.8 | 7 | 7 | 4 | −22.64 |
| TL1A-0196 | N(96)S, A(100b)E | 11.9 | 2.7 | 5 | 4 | 3 | −23.82 |
| TL1A-0197 | N(96)S, Y(100c)F | 8.7 | 1.9 | 7 | 7 | 4 | −21.62 |

In addition to reducing in silico immunogenicity assessment scores of the TL1A-0002 and variant amino acid sequences by removal of predicted T cell epitopes as described above, another approach taken to reduce the in silico predicted immunogenicity score was to re-humanize or graft TL1A-0002 CDRs onto alternate heavy and light chain variable region human germline frameworks. These specific human germline frameworks were chosen because they have lower risk T-reg Adjusted Scores than the germline usage for TL1A-0002, IGHV1-18*01 and IGKV3-11*01, and thus lower the overall in silico immunogenicity score. Antibody binding properties and/or non-specificity scores were evaluated because re-humanization or grafting CDRs onto alternate germline frameworks may change presentation of the CDR loops. Screening of the 11 of 14 re-humanized variants retained TL1A binding properties in the competition ELISA within 1.2-fold of parental antibody TL1A-0002 (Table 5). TL1A binding properties, non-specificity scores and the T-reg Adjusted Pfizer score for all re-humanized variants relative to parental TL1A-0002 are summarized in Table 5.

TABLE 5
Comparison of Properties for Re-humanized Anti-TL1A Variants versus Parental TL1A-0002 Antibody.

| | | | Competition ELISA | | | | | T-reg |
|---|---|---|---|---|---|---|---|---|
| | V_{H} | V_{L} | | Bioactivity | Non-specificity scores | | | Adjusted |
| | germline | germline | IC₅₀ | relative to | AC- | | | Pfizer |
| Name | framework | framework | [nM] | TL1A-0002 | SINS | DNA | Insulin | score |
| TL1A- | IGHV1- | IGKV3- | 4.5 | 1.0 | 7 | 8 | 4 | −18.50 |
| 0002 | 18*01 | 11*01 | | | | | | |
| TL1A- | IGHV1- | IGKV1- | 4.2 | 0.9 | 7 | 11 | 5 | −37.28 |
| 0042 | 18*01 | 39*01 | | | | | | |
| TL1A- | IGHV1- | IGKV3D- | 3.9 | 0.9 | 3 | 5 | 5 | −26.47 |
| 0043 | 18*01 | 7*01 | | | | | | |
| TL1A- | IGHV1- | IGKV3- | 5.5 | 1.2 | 3 | 4 | 5 | −18.16 |
| 0045 | 46*01 | 11*01 | | | | | | |
| TL1A- | IGHV3- | IGKV3- | 27.3 | 6.1 | 1 | 9 | 9 | −24.66 |
| 0046 | 23*01 | 11*01 | | | | | | |
| TL1A- | IGHV3- | IGKV3- | 5.3 | 1.2 | 2 | 12 | 10 | −28.48 |
| 0047 | 7*01 | 11*01 | | | | | | |
| TL1A- | IGHV1- | IGKV3- | 4.1 | 0.9 | 3 | 13 | 6 | −23.48 |
| 0048 | 2*02 | 11*01 | | | | | | |
| TL1A- | IGHV1- | IGKV1- | 4.9 | 1.1 | 3 | 6 | 6 | −36.95 |
| 0049 | 46*01 | 39*01 | | | | | | |
| TL1A- | IGHV3- | IGKV1- | 19.6 | 4.4 | 1 | 13 | 12 | −43.45 |
| 0050 | 23*01 | 39*01 | | | | | | |
| TL1A- | IGHV3- | IGKV1- | 4.5 | 1.0 | 3 | 17 | 10 | −47.27 |
| 0051 | 7*01 | 39*01 | | | | | | |
| TL1A- | IGHV1- | IGKV1- | 3.5 | 0.8 | 4 | 15 | 7 | −42.26 |
| 0052 | 2*02 | 39*01 | | | | | | |
| TL1A- | IGHV1- | IGKV3D- | 4.5 | 1.0 | 4 | 6 | 6 | −26.14 |
| 0053 | 46*01 | 7*01 | | | | | | |
| TL1A- | IGHV3- | IGKV3D- | 20.4 | 4.6 | 1 | 12 | 13 | −32.64 |
| 0054 | 23*01 | 7*01 | | | | | | |
| TL1A- | IGHV3- | IGKV3D- | 4.4 | 1.0 | 4 | 9 | 11 | −36.46 |
| 0055 | 7*01 | 7*01 | | | | | | |
| TL1A- | IGHV1- | IGKV3D- | 3.8 | 0.8 | 5 | 6 | 6 | −31.45 |
| 0056 | 2*02 | 7*01 | | | | | | |

These combined efforts identified TL1A-0042 antibody which is comprised of the parental TL1A-0002 heavy chain combined with the re-humanized light chain where CDRs were grafted onto the IGKV1-39*01 (DPK9) human germline framework. TL1A-0042 antibody had the most significant improvement in the T-reg adjusted Pfizer risk score compared to any other combination of V_{H} or V_{L} human germline framework CDR graft when combined with parental TL1A-0002 light or heavy chain (Table 5). TL1A-0042 scored at −37.28 in the moderate risk category, an improvement from parental TL1A-0002 with a T-reg adjusted Pfizer score of −18.5 in the poor risk category. Additionally, TL1A-0042 antibody retained TL1A binding properties and had similar acceptable non-specificity scores compared to parental TL1A-0002. Because of the unexpected and marked improvement in the T-reg adjusted Pfizer risk score, retained TL1A binding properties and similar non-specificity scores for TL1A-0042, the IGKV1-39*01 germline framework light chain (SEQ ID NO: 132) was utilized in all subsequent engineered variants.

Parental TL1A-0002 antibody also contains a non-human IGHV1-18*01 germline somatic mutation, S(H76)R (Pfabat numbering), in framework 3 (FW3) of the V_{H}. Variants based on TL1A-0002 heavy chain were generated where R(H76) was replaced with Serine to fully germline FW3 to IGHV1-18*01 and then combined with either TL1A-0002 light chain (SEQ ID NO: 17) or TL1A-0042 light chain (SEQ ID NO: 132) to generate TL1A-0167 and TL1A-0189 respectively. Characterization of TL1A-0167 and TL1A-0189 variants demonstrate that germlining the V_{H} FW3 did not affect TL1A binding properties in the competition ELISA, alter non-specificity scores or change the T-reg adjusted Pfizer score (Table 4 and Table 6, respectively), and therefore inclusion of R(H76)S was utilized in all subsequent variants (Table 6).

Variants harboring mutations that remove in silico predicted T cell epitopes and post-translational deamidation modifications in individual V_{H} CDRs and retain TL1A binding properties (Table 4) were next combined with tolerated mutations in other V_{H} CDRs to generate 75 variants that remove in silico predicted T cell epitopes and post-translational deamidation modifications in either CDR-H2 & CDR-H3 (TL1A-0183-187 and TL1A-0242-255) or all three V_{H} CDRs (TL1A-0256-311). In addition, 10 variant antibodies were generated to remove one or both deamidation hot spots (TL1A-0120-123, TL1A-0137-141 and TL1A-0324) and one variant antibody was generated remove the non-germline residue R(H76) in the V_{H} FW3 (TL1A-0189). Antibodies were produced transiently in Expi293F™ cells as human IgG1-effector function-minimized/human Kappa all utilizing the IGKV1-39*01 germline framework light chain as described above, SEQ ID NO: 132. All variants were screened for retention of TL1A binding properties in a bio-TL1A-0002 competition ELISA, assayed for non-specificity properties and T-reg adjusted Pfizer scores were determined as described above.

The collective results from these assays used to triage variants designed to remove in silico predicted T cell epitopes and post-translational deamidation modifications in TL1A-0002 heavy chain CDR regions show that 27 of 86 variants retained TL1A binding properties within 2-fold of the parental TL1A-0002 antibody, maintain similar or improved non-specificity scores and present an improved T-reg adjusted Pfizer score (Table 6). TL1A-0189 variant, containing the R(H76)S mutation that germlines V_{H} FW3, retained TL1A binding properties within 1-fold of parental TL1A-0002 confirming inclusion of this mutation did not affect TL1A binding or alter non-specificity or T-reg adjusted Pfizer score (Table 6). Of the variants that remove one or both deamidation hot spots, 5 of 10 retained TL1A binding properties within 2-fold of the parental TL1A-0002 antibody, maintain similar or improved non-specificity scores and present an improved T-reg adjusted Pfizer score (Table 6). For the variants that remove in silico predicted T cell epitopes and post-translational deamidation modifications in TL1A-0002 heavy chain CDRs 21 of 75 retained TL1A binding properties within 2-fold of the parental TL1A-0002 antibody, maintain similar or improved non-specificity scores and present an improved T-reg adjusted Pfizer score (Table 6). Further examination of the variants revealed that mutations at both V_{H} N(H54) and G(H55) were tolerated to remove the deamidation site in CDR-H2, however, when mutations at N(H54) were included in combination with mutations to remove in silico predicted T cell epitopes in either CDR-H2 and CDR-H3 or all three CDRs, those variants have reduced TL1A binding properties of 6-fold or less than compared to parental TL1A-0002 antibody (TL1A-0255, 254, 311, 269, 268, 282, 297, 310, 296, and 283). Also of note was the observation that a set of mutations to remove in silico predicted T cell epitopes and deamidation liabilities in CDR-H2 and CDR-H3 in TL1A-0250, G(55)T, N(56)V, R(61)P, M(62)S, R(76)S, N(96)S, Y(100c)F reduced TL1A binding properties >300-fold compared to parental TL1A-0002 antibody (TL1A-0250). Surprisingly, when this set of mutations was combined with mutations to remove the predicted T cell epitope in CDR-H1, utilizing either F(29)Y, T(30)S, I(34)V, or S(35)G in TL1A-0264, 278, 292, or 306, respectively, the TL1A-binding properties of the resulting combinations was restored to within 1.8-3.1-fold of the parental TL1A-0002 antibody.

TABLE 6
Summary of Analysis used to Triage Variants Engineered to Remove in silico Predicted T Cell Epitopes and
Deamidation Post−translational Modifications Present in TL1A−0002 Antibody.

| | | Competition ELISA | | | | | |
|---|---|---|---|---|---|---|---|
| | | | Bioactivity | | | | T−reg |
| | | | relative to | Non−specificity score | | | Adjusted |
| | | IC₅₀ | TL1A− | AC− | | | Pfizer |
| Name | V_{H} mutation(s) Pfabat numbering | [nM] | 0002 | SINS | DNA | Insulin | score |
| TL1A-0002 | Parental | 5.0 | 1.0 | 7 | 8 | 4 | −18.50 |
| TL1A-0042 | Parental | 4.2 | 0.8 | 7 | 11 | 5 | −37.28 |
| TL1A-0120 | N(54)S | 4.3 | 0.9 | 9 | 7 | 5 | −39.02 |
| TL1A-0121 | N(54)T | 8.0 | 1.6 | 11 | 8 | 5 | −37.32 |
| TL1A-0122 | N(54)Q | 4.8 | 1.0 | 7 | 5 | 5 | −39.09 |
| TL1A-0123 | N(54)G | 5.5 | 1.1 | 8 | 3 | 3 | −42.61 |
| TL1A-0137 | N(96)S | 5.4 | 1.1 | 7 | 1 | 2 | −38.30 |
| TL1A-0138 | N(96)T | 6.2 | 1.2 | 7 | 1 | 2 | −38.34 |
| TL1A-0139 | N(96)V | 24.0 | 4.8 | 6 | 1 | 1 | −38.46 |
| TL1A-0140 | N(96)Q | 11.4 | 2.3 | 6 | 1 | 2 | −39.43 |
| TL1A-0141 | N(96)E | 31.7 | 6.3 | 5 | 1 | 1 | −39.43 |
| TL1A-0183 | T(57E), R(61)P, M(62)S, N(96)S, A(100b)L | 13.2 | 2.6 | 3 | 15 | 2 | −51.93 |
| TL1A-0184 | T(57)E, R(61)P, M(62)S, N(96)T, A(100b)L | 61.8 | 12.3 | 3 | 16 | 2 | −51.97 |
| TL1A-0185 | T(57)E, R(61)P, M(62)S, N(96)V, A(100b)L | 33.8 | 6.7 | 2 | 3 | 2 | −52.08 |
| TL1A-0186 | T(57)E, R(61)P, M(62)S, N(96)Q, A(100b)L | 12.1 | 2.4 | 4 | 2 | 2 | −53.06 |
| TL1A-0187 | T(57)E, R(61)P, M(62)S, N(96)E, A(100b)L | 24.2 | 4.8 | 2 | 2 | 2 | −53.06 |
| TL1A-0189 | R(76)S | 3.8 | 0.8 | 6 | 7 | 3 | −37.28 |
| TL1A-0242 | G(55)D, R(61)P, M(62)S, R(76)S, N(96)S, Y(100c)F | 16.2 | 3.2 | 1 | 4 | 4 | −49.97 |
| TL1A-0243 | G(55)D, R(61)P, M(62)S, R(76)S, N(96)S, Y(100c)H | 14.5 | 2.9 | 1 | 5 | 5 | −49.86 |
| TL1A-0244 | G(55)D, R(61)Q, M(62)K, R(76)S, N(96)S, Y(100c)F | 17.6 | 3.5 | 0 | 5 | 4 | −47.88 |
| TL1A-0245 | G(55)D, R(61)Q, M(62)K, R(76)S, N(96)S, Y(100c)H | 16.2 | 3.2 | 1 | 6 | 5 | −47.77 |
| TL1A-0246 | G(55)T, N(56)G, R(61)P, M(62)S, R(76)S, N(96)S, Y(100c)F | 13.3 | 2.6 | 1 | 5 | 5 | −49.48 |
| TL1A-0247 | G(55)T, N(56)G, R(61)P, M(62)S, R(76)S, N(96)S, Y(100c)H | 12.2 | 2.4 | 1 | 5 | 5 | −49.37 |
| TL1A-0248 | G(55)T, N(56)G, R(61)Q, M(62)K, R(76)S, N(96)S, Y(100c)F | 16.0 | 3.2 | 0 | 6 | 6 | −47.54 |
| TL1A-0249 | G(55)T, N(56)G, R(61)Q, M(62)K, R(76)S, N(96)S, Y(100c)H | 15.7 | 3.1 | 1 | 6 | 6 | −47.43 |
| TL1A-0250 | G(55)T, N(56)V, R(61)P, M(62)S, R(76)S, N(96)S, Y(100c)F | >300 | N/A | 2 | 1 | 2 | −45.40 |
| TL1A-0251 | G(55)T, N(56)V, R(61)P, M(62)S, R(76)S, N(96)S, Y(100c)H | 16.2 | 3.2 | 2 | 4 | 5 | −45.29 |
| TL1A-0252 | G(55)T, R(61)P, M(62)S, R(76)S, N(96)S, Y(100c)F | 13.5 | 2.7 | 1 | 4 | 5 | −47.43 |
| TL1A-0253 | G(55)T, R(61)P, M(62)S, R(76)S, N(96)S, Y(100c)H | 13.4 | 2.7 | 1 | 5 | 6 | −47.32 |
| TL1A-0254 | N(54)G, R(61)P, M(62)S, R(76)S, N(96)S, Y(100c)F | 46.3 | 9.2 | 2 | 5 | 5 | −50.44 |
| TL1A-0255 | N(54)G, R(61)P, M(62)S, R(76)S, N(96)S, Y(100c)H | 38.4 | 7.6 | 2 | 7 | 6 | −50.33 |
| TL1A-0256 | F(29)Y, G(55)D, R(61)P, M(62)S, R(76)S, N(96)S, Y(100c)F | 11.6 | 2.3 | 1 | 4 | 4 | −49.98 |
| TL1A-0257 | F(29)Y, G(55)D, R(61)P, M(62)S, R(76)S, N(96)S, Y(100c)H | 9.5 | 1.9 | 1 | 4 | 4 | −49.87 |
| TL1A-0258 | F(29)Y, G(55)D, R(61)Q, M(62)K, R(76)S, N(96)S, Y(100c)F | 10.9 | 2.2 | 1 | 4 | 5 | −47.89 |
| TL1A-0259 | F(29)Y, G(55)D, R(61)Q, M(62)K, R(76)S, N(96)S, Y(100c)H | 18.4 | 3.7 | 2 | 2 | 2 | −47.78 |
| TL1A-0260 | F(29)Y, G(55)T, N(56)G, R(61)P, M(62)S, R(76)S, N(96)S, | 6.8 | 1.3 | 3 | 3 | 4 | −49.50 |
| | Y(100c)F | | | | | | |
| TL1A-0261 | F(29)Y, G(55)T, N(56)G, R(61)P, M(62)S, R(76)S, N(96)S, | 5.6 | 1.1 | 1 | 6 | 5 | −49.39 |
| | Y(100c)H | | | | | | |
| TL1A-0262 | F(29)Y, G(55)T, N(56)G, R(61)Q, M(62)K, R(76)S, N(96)S, | 7.3 | 1.4 | 2 | 6 | 6 | −47.55 |
| | Y(100c)F | | | | | | |
| TL1A-0263 | F(29)Y, G(55)T, N(56)G, R(61)Q, M(62)K, R(76)S, N(96)S, | 6.2 | 1.2 | 2 | 10 | 6 | −47.44 |
| | Y(100c)H | | | | | | |
| TL1A-0264 | F(29)Y, G(55)T, N(56)V, R(61)P, M(62)S, R(76)S, N(96)S, | 15.8 | 3.1 | 3 | 3 | 5 | −45.41 |
| | Y(100c)F | | | | | | |
| TL1A-0265 | F(29)Y, G(55)T, N(56)V, R(61)P, M(62)S, R(76)S, N(96)S, | 8.7 | 1.7 | 2 | 4 | 5 | −45.30 |
| | Y(100c)H | | | | | | |
| TL1A-0266 | F(29)Y, G(55)T, R(61)P, M(62)S, R(76)S, N(96)S, Y(100c)F | 6.6 | 1.3 | 1 | 4 | 5 | −47.44 |
| TL1A-0267 | F(29)Y, G(55)T, R(61)P, M(62)S, R(76)S, N(96)S, Y(100c)H | 6.8 | 1.3 | 1 | 6 | 6 | −47.33 |
| TL1A-0268 | F(29)Y, N(54)G, R(61)P, M(62)S, R(76)S, N(96)S, Y(100c)F | 48.2 | 9.6 | 1 | 4 | 6 | −50.46 |
| TL1A-0269 | F(29)Y, N(54)G, R(61)P, M(62)S, R(76)S, N(96)S, Y(100c)H | 37.7 | 7.5 | 2 | 4 | 5 | −50.34 |
| TL1A-0270 | T(30)S, G(55)D, R(61)P, M(62)S, R(76)S, N(96)S, Y(100c)F | 11.5 | 2.3 | 1 | 4 | 5 | −50.94 |
| TL1A-0271 | T(30)S, G(55)D, R(61)P, M(62)S, R(76)S, N(96)S, Y(100c)H | 10.6 | 2.1 | 1 | 4 | 4 | −50.83 |
| TL1A-0272 | T(30)S, G(55)D, R(61)Q, M(62)K, R(76)S, N(96)S, Y(100c)F | 11.6 | 2.3 | 1 | 5 | 5 | −48.85 |
| TL1A-0273 | T(30)S, G(55)D, R(61)Q, M(62)K, R(76)S, N(96)S, Y(100c)H | 10.9 | 2.2 | 1 | 4 | 4 | −48.74 |
| TL1A-0274 | T(30)S, G(55)T, N(56)G, R(61)P, M(62)S, R(76)S, N(96)S, | 10.1 | 2.0 | 0 | 4 | 4 | −50.46 |
| | Y(100c)F | | | | | | |
| TL1A-0275 | T(30)S, G(55)T, N(56)G, R(61)P, M(62)S, R(76)S, N(96)S, | 9.0 | 1.8 | 1 | 4 | 5 | −50.34 |
| | Y(100c)H | | | | | | |
| TL1A-0276 | T(30)S, G(55)T, N(56)G, R(61)Q, M(62)K, R(76)S, N(96)S, | 10.6 | 2.1 | 1 | 5 | 6 | −48.51 |
| | Y(100c)F | | | | | | |
| TL1A-0277 | T(30)S, G(55)T, N(56)G, R(61)Q, M(62)K, R(76)S, N(96)S, | 10.2 | 2.0 | 1 | 5 | 6 | −48.40 |
| | Y(100c)H | | | | | | |
| TL1A-0278 | T(30)S, G(55)T, N(56)V, R(61)P, M(62)S, R(76)S, N(96)S, | 9.1 | 1.8 | 1 | 4 | 5 | −46.37 |
| | Y(100c)F | | | | | | |
| TL1A-0279 | T(30)S, G(55)T, N(56)V, R(61)P, M(62)S, R(76)S, N(96)S, | 10.5 | 2.1 | 1 | 5 | 5 | −46.26 |
| | Y(100c)H | | | | | | |
| TL1A-0280 | T(30)S, G(55)T, R(61)P, M(62)S, R(76)S, N(96)S, Y(100c)F | 8.2 | 1.6 | 1 | 3 | 6 | −48.40 |
| TL1A-0281 | T(30)S, G(55)T, R(61)P, M(62)S, R(76)S, N(96)S, Y(100c)H | 13.1 | 2.6 | 2 | 2 | 2 | −48.29 |
| TL1A-0282 | T(30)S, N(54)G, R(61)P, M(62)S, R(76)S, N(96)S, Y(100c)F | 52.1 | 10.3 | 2 | 4 | 5 | −51.41 |
| TL1A-0283 | T(30)S, N(54)G, R(61)P, M(62)S, R(76)S, N(96)S, Y(100c)H | 246.5 | 48.9 | 3 | 2 | 2 | −51.30 |
| TL1A-0284 | I(34)V, G(55)D, R(61)P, M(62)S, R(76)S, N(96)S, Y(100c)F | 10.3 | 2.0 | 1 | 4 | 5 | −52.92 |
| TL1A-0285 | I(34)V, G(55)D, R(61)P, M(62)S, R(76)S, N(96)S, Y(100c)H | 8.6 | 1.7 | 1 | 4 | 4 | −52.81 |
| TL1A-0286 | I(34)V, G(55)D, R(61)Q, M(62)K, R(76)S, N(96)S, Y(100c)F | 11.6 | 2.3 | 1 | 4 | 5 | −50.83 |
| TL1A-0287 | I(34)V, G(55)D, R(61)Q, M(62)K, R(76)S, N(96)S, Y(100c)H | 10.1 | 2.0 | 2 | 5 | 4 | −50.72 |
| TL1A-0288 | I(34)V, G(55)T, N(56)G, R(61)P, M(62)S, R(76)S, N(96)S, | 8.0 | 1.6 | 1 | 5 | 5 | −52.43 |
| | Y(100c)F | | | | | | |
| TL1A-0289 | I(34)V, G(55)T, N(56)G, R(61)P, M(62)S, R(76)S, N(96)S, | 7.9 | 1.6 | 1 | 5 | 5 | −52.32 |
| | Y(100c)H | | | | | | |
| TL1A-0290 | I(34)V, G(55)T, N(56)G, R(61)Q, M(62)K, R(76)S, N(96)S, | 21.0 | 4.2 | ND | ND | ND | −50.48 |
| | Y(100c)F | | | | | | |
| TL1A-0291 | I(34)V, G(55)T, N(56)G, R(61)Q, M(62)K, R(76)S, N(96)S, | 9.0 | 1.8 | 1 | 5 | 6 | −50.37 |
| | Y(100c)H | | | | | | |
| TL1A-0292 | I(34)V, G(55)T, N(56)V, R(61)P, M(62)S, R(76)S, N(96)S, | 9.6 | 1.9 | 1 | 3 | 6 | −48.34 |
| | Y(100c)F | | | | | | |
| TL1A-0293 | I(34)V, G(55)T, N(56)V, R(61)P, M(62)S, R(76)S, N(96)S, | 9.7 | 1.9 | 2 | 4 | 6 | −48.23 |
| | Y(100c)H | | | | | | |
| TL1A-0294 | I(34)V, G(55)T, R(61)P, M(62)S, R(76)S, N(96)S, Y(100c)F | 8.0 | 1.6 | 1 | 5 | 5 | −50.38 |
| TL1A-0295 | I(34)V, G(55)T, R(61)P, M(62)S, R(76)S, N(96)S, Y(100c)H | 12.3 | 2.4 | 2 | 2 | 2 | −50.27 |
| TL1A-0296 | I(34)V, N(54)G, R(61)P, M(62)S, R(76)S, N(96)S, Y(100c)F | 87.3 | 17.3 | 3 | 1 | 2 | −53.39 |
| TL1A-0297 | I(34)V, N(54)G, R(61)P, M(62)S, R(76)S, N(96)S, Y(100c)H | 73.1 | 14.5 | 2 | 1 | 2 | −53.28 |
| TL1A-0298 | S(35)G, G(55)D, R(61)P, M(62)S, R(76)S, N(96)S, Y(100c)F | 25.3 | 5.0 | 1 | 2 | 2 | −53.04 |
| TL1A-0299 | S(35)G, G(55)D, R(61)P, M(62)S, R(76)S, N(96)S, Y(100c)H | 22.9 | 4.5 | 2 | 2 | 2 | −52.93 |
| TL1A-0300 | S(35)G, G(55)D, R(61)Q, M(62)K, R(76)S, N(96)S, Y(100c)F | 23.8 | 4.7 | ND | ND | ND | −50.94 |
| TL1A-0301 | S(35)G, G(55)D, R(61)Q, M(62)K, R(76)S, N(96)S, Y(100c)H | 24.7 | 4.9 | 2 | 2 | 2 | −50.83 |
| TL1A-0302 | S(35)G, G(55)T, N(56)G, R(61)P, M(62)S, R(76)S, N(96)S, | 20.4 | 4.0 | 1 | 2 | 2 | −52.55 |
| | Y(100c)F | | | | | | |
| TL1A-0303 | S(35)G, G(55)T, N(56)G, R(61)P, M(62)S, R(76)S, N(96)S, | 15.0 | 3.0 | 1 | 2 | 2 | −52.44 |
| | Y(100c)H | | | | | | |
| TL1A-0304 | S(35)G, G(55)T, N(56)G, R(61)Q, M(62)K, R(76)S, N(96)S, | 14.9 | 3.0 | 0 | 7 | 6 | −50.60 |
| | Y(100c)F | | | | | | |
| TL1A-0305 | S(35)G, G(55)T, N(56)G, R(61)Q, M(62)K, R(76)S, N(96)S, | 14.5 | 2.9 | 1 | 7 | 5 | −50.49 |
| | Y(100c)H | | | | | | |
| TL1A-0306 | S(35)G, G(55)T, N(56)V, R(61)P, M(62)S, R(76)S, N(96)S, | 15.8 | 3.1 | 1 | 5 | 5 | −48.46 |
| | Y(100c)F | | | | | | |
| TL1A-0307 | S(35)G, G(55)T, N(56)V, R(61)P, M(62)S, R(76)S, N(96)S, | 17.2 | 3.4 | 2 | 2 | 3 | −48.35 |
| | Y(100c)H | | | | | | |
| TL1A-0308 | S(35)G, G(55)T, R(61)P, M(62)S, R(76)S, N(96)S, Y(100c)F | 14.2 | 2.8 | 2 | 2 | 3 | −50.50 |
| TL1A-0309 | S(35)G, G(55)T, R(61)P, M(62)S, R(76)S, N(96)S, Y(100c)H | 16.8 | 3.3 | 2 | 2 | 2 | −50.39 |
| TL1A-0310 | S(35)G, N(54)G, R(61)P, M(62)S, R(76)S, N(96)S, Y(100c)F | 74.7 | 14.8 | 1 | 2 | 3 | −53.51 |
| TL1A-0311 | S(35)G, N(54)G, R(61)P, M(62)S, R(76)S, N(96)S, Y100cH | 30.2 | 6.0 | 3 | 2 | 2 | −53.40 |
| TL1A-0324 | G(55)T, N(96)S | 6.1 | 1.2 | 7 | 11 | 5 | −40.51 |

ND = not done.

Select variants harboring mutations that (1) removed all five in silico predicted T cell epitopes and post-translational deamidation modifications in V_{H} CDRs, (2) retained TL1A binding properties within two-fold of parental antibody in the competition ELISA, (3) maintained or improved non-specificity scores compared to parental TL1A-0002 and (4) had improved T-reg Adjusted Pfizer scores compared to parental TL1A-0002 were selected for further analysis. Functional bioactivity of these optimized variants relative to the parental TL1A-0002 antibody was measured in two different in vitro cell based assays, one assessed the inhibition of TL1A induced nuclear factor kappa-light-chain-enhancer of activated B cells (NF-κB) activity in cells and the other measured the inhibition of interferon gamma (IFN-γ) secretion in whole blood. To determine whether the optimized antibody variants neutralize TL1A signaling through Death Receptor 3 (DR3), TF-1 human erythroleukemic cells, which endogenously expresses DR3, were utilized. DR3 signals through the Tumor Necrosis Factor Receptor (TNFR)-associated DEATH domain protein (TRADD), which in turn recruits TNFR-associated factor (TRAF) containing complexes resulting in activation of proinflammatory NF-κB and mitogen-activated protein kinase (MAPK) pathways. TF-1 human erythroleukemic cells (ATCC CRL-2003) were transduced with a pCignal Lenti-TRE-Reporter gene (Qiagen catalog number 336851) and used for this bioassay. The human erythroleukemia TF-1 cells transduced with pCignal Lenti-TRE-Reporter gene (TF-1 reporter cells) were cultured in RPMI-1640 media (Gibco catalog number 11875-093) plus 10% heat-inactivated fetal bovine serum (ATCC catalog number 30-2020), 10 mM 4-(2-hydroxyethyl)-1-piperazineethanesulfonic acid (HEPES, Gibco catalog number 15630), 10,000 units/mL Penicillin-Streptomycin (Gibco catalog number 15140122), 1 mM sodium pyruvate (Gibco catalog number 11360-070), 200 mM L-Glutamine (Gibco catalog number 25036-081), 2 ng/mL recombinant human granulocyte-macrophage colony-stimulating factor (R&D Systems catalog number 215-GM) and 1 μg/mL puromycin (Invitrogen catalog number A11138-03). The TF-1 reporter cells were added to a 96-well plate (50,000 cells per well) and stimulated with a fixed concentration of recombinant human TL1A (200 pM) in the presence of either the optimized variants or parental anti-TL1A-0002 antibody titrated to a range of 1.37-3000 pM in a total volume of 100 μL and then incubated at 37° C. for 24 hours. Following this incubation, 100 μL of Beetle Luciferin (Promega E1601) was added to the wells for a final luciferin concentration of 150 μg/mL and incubated at 37° C. for 30 minutes. Luminescence was read on Envision (Perkin-Elmer) for NF-κB activity. Optimized variants that exhibit half-maximal inhibitory concentration (IC₅₀) within a 3-fold range of parental TL1A-0002 were considered to have retained the potent neutralization ability.

To evaluate the functional inhibitory potency of variants to inhibit IFN-γ secretion in whole blood, a 0.167-5 nM titration range of each antibody was evaluated in duplicate in a whole blood assay where human blood was stimulated overnight with a cytokine cocktail including recombinant TL1A (861 pM), IL-12 (0.5 ng/ml, BD Pharmingen Catalog number 554613), IL-18 (5 ng/ml, Gibco Catalog number PHC0186), IL-15 (5 ng/ml, R&D Systems Catalog number 247-ILB-025) and α-CD3 antibody (50 ng/ml, Invitrogen Catalog number 16-0037-85) in a 96-well plate. Following this incubation, IFN-γ was measured in the plasma from the centrifuged blood, with an IFN-γ meso scale discovery (MSD) assay (Catalog number L451AEB-1). The design of this study mimics the mechanism of action of TL1A which is co-stimulatory and augments the activity of other cytokines. Variants that retained inhibitory potency within a 2-fold range decreased potency (IC₅₀) relative to parental TL1A-0002 antibody and were considered to have retained potent inhibition of TL1A-mediated IFN-γ secretion.

The collective results from these assays used to triage variants designed to remove in silico predicted T cell epitopes and post-translational deamidation modifications in TL1A-0002 heavy chain CDR regions that retained TL1A binding properties within 2-fold of the parental TL1A-0002 antibody in the bio-TL1A-0002 competition ELISA show that 13 of 15 variants tested in the TF-1 NF-κB reporter cell bioassay retained TL1A binding properties within 2-fold of the parental TL1A-0002 antibody, and all 19 of 19 variants tested in the IFNγ assay retained TL1A binding properties within 1.5-fold of the parental TL1A-0002 antibody (Table 7). As reported above, the 22 selected variants remove all five in silico predicted T cell epitopes and post-translational deamidation modifications in V_{H} CDRs, maintain DNA and Insulin non-specificity scores and improve AC-SINS non-specificity scores compared to parental TL1A-0002, and have improved T-reg Adjusted Pfizer scores compared to parental TL1A-0002.

TABLE 7
Summary of Analysis used to Further Triage Variants Engineered to Remove in silico Predicted T Cell Epitopes and
Deamidation Post-translational Modifications Present in TL1A-0002 Antibody.

| | | | | TF-1 NF-κB | | | | | | | |
|---|---|---|---|---|---|---|---|---|---|---|---|
| | | Competition | | reporter cell | | Whole blood IFNγ | | | | | |
| | | ELISA | | bioassay | | assay | | | | | |
| | | | Bioactivity | | Bioactivity | | Bioactivity | | | | T-reg |
| | VH mutation(s) | | relative to | | relative to | | relative to | Non-specificity score | | | Adjusted |
| | Pfabat | IC₅₀ | TL1A- | IC₅₀ | TL1A- | IC₅₀ | TL1A- | AC- | | | Pfizer |
| Name | numbering | [nM] | 0002 | [nM] | 0002 | [nM] | 0002 | SINS | DNA | Insulin | score |
| TL1A- | Parental | 5.0 | 1.0 | 34.7 | 1.0 | 0.8302 | 1.0 | 7 | 8 | 4 | −18.50 |
| 0002 | | | | | | | | | | | |
| TL1A- | Parental | 4.2 | 0.8 | 25.9 | 0.7 | 0.7480 | 0.9 | 7 | 11 | 5 | −37.28 |
| 0042 | | | | | | | | | | | |
| TL1A- | F(29)Y, G(55)T, | 6.8 | 1.3 | 29.2 | 0.8 | 0.7548 | 0.9 | 3 | 3 | 4 | −49.50 |
| 0260 | N(56)G, R(61)P, | | | | | | | | | | |
| | M(62)S, R(76)S, | | | | | | | | | | |
| | N(96)S, Y(100c)F | | | | | | | | | | |
| TL1A- | F(29)Y, G(55)T, | 5.6 | 1.1 | 29.5 | 0.8 | 0.9226 | 1.1 | 1 | 6 | 5 | −49.39 |
| 0261 | N(56)G, R(61)P, | | | | | | | | | | |
| | M(62)S, R(76)S, | | | | | | | | | | |
| | N(96)S, Y(100c)H | | | | | | | | | | |
| TL1A- | F(29)Y, G(55)T, | 7.3 | 1.4 | 43.9 | 1.3 | 1.028 | 1.2 | 2 | 6 | 6 | −47.55 |
| 0262 | N(56)G, R(61)Q, | | | | | | | | | | |
| | M(62)K, R(76)S, | | | | | | | | | | |
| | N(96)S, Y(100c)F | | | | | | | | | | |
| TL1A- | F(29)Y, G(55)T, | 6.2 | 1.2 | 40.4 | 1.2 | 0.9323 | 1.1 | 2 | 10 | 6 | −47.44 |
| 0263 | N(56)G, R(61)Q, | | | | | | | | | | |
| | M(62)K, R(76)S, | | | | | | | | | | |
| | N(96)S, Y(100c)H | | | | | | | | | | |
| TL1A- | F(29)Y, G(55)T, | 15.8 | 3.1 | 30.0 | 0.9 | 1.064 | 1.3 | 3 | 3 | 5 | −45.41 |
| 0264 | N(56)V, R(61)P, | | | | | | | | | | |
| | M(62)S, R(76)S, | | | | | | | | | | |
| | N(96)S, Y(100c)F | | | | | | | | | | |
| TL1A- | F(29)Y, G(55)T, | 8.7 | 1.7 | 45.1 | 1.3 | 1.012 | 1.2 | 2 | 4 | 5 | −45.30 |
| 0265 | N(56)V, R(61)P, | | | | | | | | | | |
| | M(62)S, R(76)S, | | | | | | | | | | |
| | N(96)S, Y(100c)H | | | | | | | | | | |
| TL1A- | F(29)Y, G(55)T, | 6.6 | 1.3 | 37.0 | 1.1 | 0.7450 | 0.9 | 1 | 4 | 5 | −47.44 |
| 0266 | R(61)P, M(62)S, | | | | | | | | | | |
| | R(76)S, N(96)S, | | | | | | | | | | |
| | Y(100c)F | | | | | | | | | | |
| TL1A- | F(29)Y, G(55)T, | 6.8 | 1.3 | 42.2 | 1.2 | 0.9398 | 1.1 | 1 | 6 | 6 | −47.33 |
| 0267 | R(61)P, M(62)S, | | | | | | | | | | |
| | R(76)S, N(96)S, | | | | | | | | | | |
| | Y(100c)H | | | | | | | | | | |
| TL1A- | T(30)S, G(55)T, | 10.1 | 2.0 | 19.1 | 0.6 | ND | N/A | 0 | 4 | 4 | −50.46 |
| 0274 | N(56)G, R(61)P, | | | | | | | | | | |
| | M(62)S, R(76)S, | | | | | | | | | | |
| | N(96)S, Y(100c)F | | | | | | | | | | |
| TL1A- | T(30)S, G(55)T, | 9.0 | 1.8 | 24.7 | 0.7 | ND | N/A | 1 | 4 | 5 | −50.34 |
| 0275 | N(56)G, R(61)P, | | | | | | | | | | |
| | M(62)S, R(76)S, | | | | | | | | | | |
| | N(96)S, Y(100c)H | | | | | | | | | | |
| TL1A- | T(30)S, G(55)T, | 9.1 | 1.8 | ND | N/A | 0.8198 | 1.0 | 1 | 4 | 5 | −46.37 |
| 0278 | N(56)V, R(61)P, | | | | | | | | | | |
| | M(62)S, R(76)S, | | | | | | | | | | |
| | N(96)S, Y(100c)F | | | | | | | | | | |
| TL1A- | T(30)S, G(55)T, | 8.2 | 1.6 | ND | N/A | 0.9139 | 1.1 | 1 | 3 | 6 | −48.40 |
| 0280 | R(61)P, M(62)S, | | | | | | | | | | |
| | R(76)S, N(96)S, | | | | | | | | | | |
| | Y(100c)F | | | | | | | | | | |
| TL1A- | I(34)V, G(55)D, | 8.6 | 1.7 | ND | N/A | 0.9(55)0 | 1.2 | 1 | 4 | 4 | −52.81 |
| 0285 | R(61)P, M(62)S, | | | | | | | | | | |
| | R(76)S, N(96)S, | | | | | | | | | | |
| | Y(100c)H | | | | | | | | | | |
| TL1A- | I(34)V, G(55)D, | 10.1 | 2.0 | ND | N/A | 0.9933 | 1.2 | 2 | 5 | 4 | −50.72 |
| 0287 | R(61)Q, M(62)K, | | | | | | | | | | |
| | R(76)S, N(96)S, | | | | | | | | | | |
| | Y(100c)H | | | | | | | | | | |
| TL1A- | I(34)V, G(55)T, | 8.0 | 1.6 | 96.0 | 2.8 | ND | N/A | 1 | 5 | 5 | −52.43 |
| 0288 | N(56)G, R(61)P, | | | | | | | | | | |
| | M(62)S, R(76)S, | | | | | | | | | | |
| | N(96)S, Y(100c)F | | | | | | | | | | |
| TL1A- | I(34)V, G(55)T, | 7.9 | 1.6 | ND | N/A | 0.7992 | 1.0 | 1 | 5 | 5 | −52.32 |
| 0289 | N(56)G, R(61)P, | | | | | | | | | | |
| | M(62)S, R(76)S, | | | | | | | | | | |
| | N(96)S, Y(100c)H | | | | | | | | | | |
| TL1A- | I(34)V, G(55)T, | 9.0 | 1.8 | ND | N/A | 0.9289 | 1.1 | 1 | 5 | 6 | −50.37 |
| 0291 | N(56)G, R(61)Q, | | | | | | | | | | |
| | M(62)K, R(76)S, | | | | | | | | | | |
| | N(96)S, Y(100c)H | | | | | | | | | | |
| TL1A- | I(34)V, G(55)T, | 9.6 | 1.9 | 77.9 | 2.2 | 0.9498 | 1.1 | 1 | 3 | 6 | −48.34 |
| 0292 | N(56)V, R(61)P, | | | | | | | | | | |
| | M(62)S, R(76)S, | | | | | | | | | | |
| | N(96)S, Y(100c)F | | | | | | | | | | |
| TL1A- | I(34)V, G(55)T, | 9.7 | 1.9 | ND | N/A | 0.9856 | 1.2 | 2 | 4 | 6 | −48.23 |
| 0293 | N(56)V, R(61)P, | | | | | | | | | | |
| | M(62)S, R(76)S, | | | | | | | | | | |
| | N(96)S, Y(100c)H | | | | | | | | | | |
| TL1A- | I(34)V, G(55)T, | 8.0 | 1.6 | 56.5 | 1.6 | 0.9416 | 1.1 | 1 | 5 | 5 | −50.38 |
| 0294 | R(61)P, M(62)S, | | | | | | | | | | |
| | R(76)S, N(96)S, | | | | | | | | | | |
| | Y(100c)F | | | | | | | | | | |
| TL1A- | S(35)G, G(55)T, | 15.0 | 3.0 | 49.4 | 1.4 | 1.228 | 1.5 | 1 | 2 | 2 | −52.44 |
| 0303 | N(56)G, R(61)P, | | | | | | | | | | |
| | M(62)S, R(76)S, | | | | | | | | | | |
| | N(96)S, Y(100c)H | | | | | | | | | | |
| TL1A- | S(35)G, G(55)T, | 14.2 | 2.8 | 43.0 | 1.2 | 0.8099 | 1.0 | 2 | 2 | 3 | −50.50 |
| 0308 | R(61)P, M(62)S, | | | | | | | | | | |
| | R(76)S, N(96)S, | | | | | | | | | | |
| | Y(100c)F | | | | | | | | | | |

ND = not done; N/A = not applicable.

### Example 3. Engineering of Therapeutic Format to Reduce Immunogenicity

Another approach employed to reduce observed ADA with anti-TL1A therapy was to engineer the antibody into a monovalent molecular format based on a hypothesis that the TL1A-0002 antibody formed high order complexes that could be presented to the immune system.

The co-crystal structure of anti-TL1A 1D1 parental Fab in complex with TL1A homotrimer indicates that a single Fab arm binds TL1A in the groove between two monomers thus stabilizing the TL1A trimer and allowing for the other Fab arm, present in the bivalent IgG, to bind to another TL1A trimer (Arch et al. U.S. Pat. No. 9,683,998 Pfizer Inc. and Bristol-Myers Squibb Company, Jun. 19, 2017, Example 6)¹. Pharmacokinetic/pharmacodynamic (PK/PD) modeling suggests that bivalent anti-TL1A antibodies, such as TL1A-0002, can induce high molecular weight (HMW) complex formation at sites of elevated TL1A expression. HMW complex formation of TL1A trimers bound to anti-TL1A antibodies poses an immunogenic risk as Fc-gamma receptors (FcγRs) may recognize these IgG-coated TL1A complexes⁵. Increased formation and uptake of such HMW complexes may serve to amplify any immunogenic response as broad presentation that may increase the likelihood of T cells initiating an immunological response.

To avert the risk of immunogenicity due to HMW complex formation, TL1A-0002 antibody was engineered into a monovalent binding modality that should be incapable of forming high molecular weight complexes between soluble TL1A and TL1A-0002. The monovalent TL1A antagonist or “one-armed” antibody is composed of a functional anti-TL1A heavy chain, functional anti-TL1A LC, and a truncated heavy chain which lacks the anti-TL1A Fd and includes the C(H230)S mutation (Pfabat numbering) within the hinge region to disrupt interchain disulfide bond formation with the light chain (FIG. 1). Heterodimerization of the functional and truncated heavy chains was driven by engineering a protuberance (‘bump’ or ‘knob’) at the interface of one of the heavy chain CH3 domains and a corresponding cavity (hole) in the interface of the other heavy chain CH3 domain, such that the protuberance can be positioned in the cavity as to promote heterodimer formation and hinder homodimer formation (FIG. 1). This latter Fc heterodimerization approach will be referred to herein as Knob-into-Hole (KiH)⁶. Specifically, the Knob mutation T(389)W with Y(370)C (Pfabat numbering) was engineered into the IgG1-CH3 domain on the functional anti-TL1A heavy chain and the Hole mutations T(389)S, L(391)A and Y(438)V with S(375)C (Pfabat numbering) were introduced into the IgG1-CH3 domain of the truncated heavy chain. In some instances, the Fc region also contained mutations for half-life extension, specifically M(H459)L and N(H465)S (Pfabat numbering) referred to herein as LS mutations' that were incorporated to increase serum half-life of the therapeutic to offer increased dosing flexibility. The functional TL1A binding domain was comprised of either TL1A-0002 Fab to produce one-armed TL1A-0057, or of Fabs derived from variants designed to reduce in silico predicted immunogenicity and remove the deamidation sequence liabilities as described above. Specifically, TL1A-0260, TL1A-0266 and TL1A-0308 were used to generate the one-armed antibodies TL1A-0326, TL1A-0327, and TL1A-0328, respectively.

To assess whether the monovalent and bivalent anti-TL1A antibodies can form high order molecular complexes, recombinant human TL1A was incubated with the parental TL1A-0002 bivalent antibody or its one-armed format, TL1A-0057, at a 1:1 molar ratio of antibody and recombinant soluble human TL1A trimer (both at 0.015 mM) and the average radius of high molecular mass species (HMMS) was measured with Dynamic Light Scattering (DLS). The results illustrate that HMMS are formed only when the bivalent TL1A-0002 antibody is combined with TL1A, however no HMMS are observed when the one-armed TL1A-0057 antibody is incubated with TL1A indicating that the one-armed antibody does not form high order molecular complexes with recombinant human TL1A in vitro (Table 8).

TABLE 8
One-armed Anti-TL1A antibody Does Not Form
High Molecular Mass Complexes when Incubated
with Recombinant Human TL1A in vitro.

| | Radius ± SD (nm) |
|---|---|
| Sample | Measured by DLS |
| Bivalent TL1A-0002 | 5.7 ± 0.0 |
| Bivalent TL1A-0002 + TL1A | 108.5 ± 6.3 |
| One-armed TL1A-0057 | 5.1 ± 0.7 |
| One-armed TL1A-0057 + TL1A | 7.8 ± 0.4 |

The human FcRn (hFcRn) transgenic 32 (Tg32) homozygous mouse model was utilized to evaluate the pharmacokinetics (PK) of the anti-TL1A one-armed antibodies. The Tg32 mouse model has been established as an in vivo tool for the prediction of antibody human clearance (CL)⁸. This assessment was conducted to gauge how similar the anti-TL1A one-armed variants were relative to the TL1A-0002 bivalent antibody and to an LS containing antibody, both have established human PK and known PK in the Tg32 model. In brief, 6-10-week-old mice (Jackson Labs, #014565) received a single 5 mg/kg IV dose, and plasma samples were collected out to 8 weeks (4 mice/group). The study was conducted at Pfizer, Inc., and was designed and executed within accordance of the Animal Use Protocol and adherence to the Pfizer institutional animal care and use committee regulations. Quantitative analysis of plasma samples was conducted using a ligand binding assay (total human IgG assay format) developed on the Gyrolab® platform. Precisely, the total human IgG assay format uses reagents specific for human IgG and will quantify the total amount of drug present regardless of if it is bound to target. Results from the Tg32 transgenic PK model show that the serum half-life was notably shorter for the one-armed variant TL1A-0057 compared to the parental TL1A-0002 antibody in a typical IgG format (Table 9). Further, variants TL1A-0326, TL1A-0327, and TL1A-0328 that harbor LS mutations to extend the serum half-life also exhibited markedly shorter half-lives when compared to an anti-TL1A antibody containing LS mutations (Table 9). These combined findings indicate that anti-TL1A antibodies engineered into a one-armed antibody modality exhibit unexpected rapid clearance in vivo and that this monovalent binding modality would not be suitable for chronic administration.

TABLE 9
Terminal Half-lives Determined for LS Engineered Anti-TL1A One-
Armed Variants as Compared to Bivalent Antibody TL1A-0002 and
a Reference Antibody Harboring LS Mutations in Tg32 Mice.

| | | Terminal |
|---|---|---|
| | | Half-life |
| Sample | Fc format | (Days) |
| Reference | Bivalent IgG, LS | 19.1 |
| Antibody | | |
| TL1A-0002 | Bivalent IgG | 13.4 |
| TL1A-0057 | One-armed IgG, KiH | ~5 |
| TL1A-0326 | One-armed IgG, KiH, LS | ~6 |
| TL1A-0327 | One-armed IgG, KiH, LS | ~6 |
| TL1A-0328 | One-armed IgG, KiH, LS | ~6 |

### Example 4. Engineering Monovalent TL1A Binding Modality to Restore PK Properties

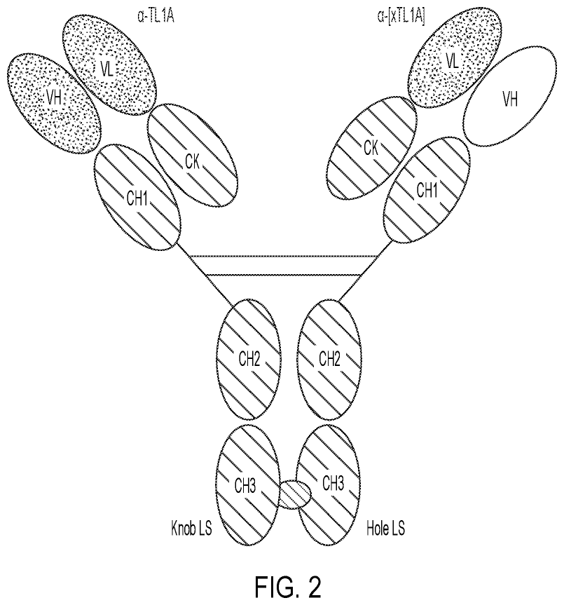

An alternate monovalent TL1A antagonist modality was employed to overcome the short serum half-life observed for the one-armed antibody variants. This modality was designed to resemble a bivalent antibody with only one antigen-binding fragment (Fab) permitting only monovalent binding to TL1A and is referred to herein as a monovalent bispecific antibody. The TL1A-0326 one-armed variant derived from optimized bivalent TL1A-0260 antibody was selected to engineer into a monovalent bispecific format. The monovalent bispecific antibody is assembled from three independent protein chains: the functional TL1A-0326 HC; a “disabled” or “null” heavy chain, noted as [xTL1A], which has ablated binding to TL1A; and the TL1A-0326 LC, which can pair with both functional and disabled heavy chains (FIG. 2). KiH mutations were used to drive heterodimerization of the functional and disabled heavy chains, as described above. The monovalent bispecific Fc was also engineered with mutations to minimize effector function and extend serum half-life to offer increased dosing flexibility. X-ray crystallography studies of parental 1D1 antibody bound to human TL1A trimer indicated that the V_{L} was not heavily involved in the binding interaction (Arch et al. U.S. Pat. No. 9,683,998 Pfizer Inc. and Bristol-Myers Squibb Company, Jun. 19, 2017, EXAMPLE 6)¹, therefore the V_{L} region of the LC was not mutagenized to ablate TL1A binding. The [xTL1A] V_{H} was generated by fusing three different human heavy chain variable region germline sequences, IGHV1-18*01, IGHV3-23*01 or IGHV1-2*02, to the TL1A-0326 CDRH3 (SEQ ID NO: 128) and J-segment (JH, SEQ ID NO: 4). Next, using computational and manual inspection of the binding interface in the co-crystal structure of the parental 1D1 Fab with human TL1A¹ mutations were identified in CDRH3 to diminish TL1A binding (Table 10).

TABLE 10
Summary of Structure Guided Amino Acid
Substitutions in TL1A-0326 CDRH3.

| Anti-TL1A CDRH3 Position | |
|---|---|
| (Pfabat numbering) | Substituted Amino Acids |
| Y97 | S |
| G100a | E |
| G100e | A, H |

A panel of [xTL1A] bivalent antibody variants were designed as human IgG1-effector function-minimized/human Kappa antibodies and produced transiently in Expi293F™ cells. The following in vitro assays were used to determine whether TL1A binding was ablated for [xTL1A] bivalent antibody variants: human TL1A direct binding ELISA, Surface Plasmon Resonance (SPR) assessment of binding to recombinant human TL1A, binding to cells over-expressing human TL1A, and evaluation of TL1A binding properties in a competition ELISA with biotinylated TL1A-0260 antibody (bio-TL1A-0260) as the reporter.

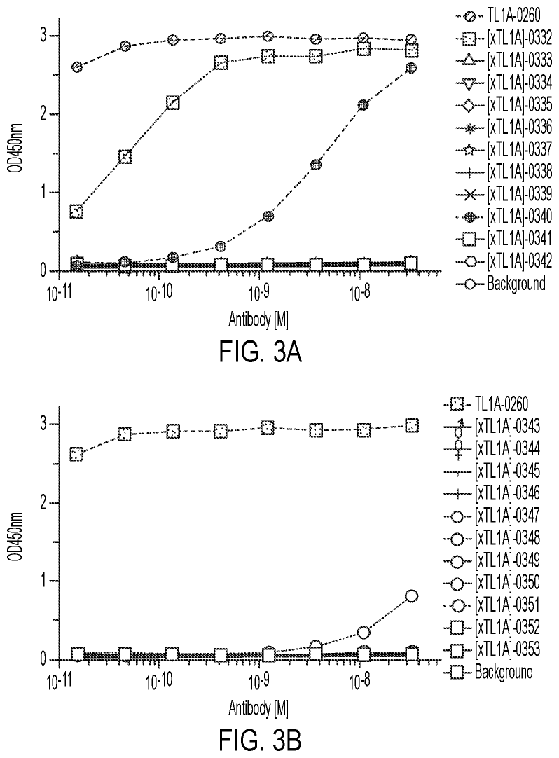

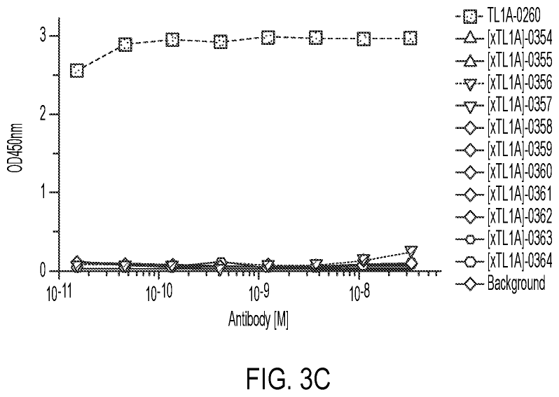

For the direct binding ELISA, 96-well high binding plates (Corning, product number 3590) were coated with 1 μg/mL recombinant TL1A in 100 μL/well in PBS overnight at 4° C. Plates were then blocked in PBS+0.02% casein solution and washed with PBS+0.05% Tween-20 following standard ELISA protocol. Assay buffer (PBS+0.5% BSA+0.02% Tween-20) was mixed with a 0.015-33.3 nM concentration range of the [xTL1A] antibody variants or optimized TL1A-0260 antibody as the positive control, using a singleton sample for each antibody concentration evaluated. TL1A-0260 is a bivalent antibody variant that includes mutations to remove all in silico predicted T cell epitopes and post-translational deamidation modifications in the individual V_{H} CDR regions, maintains TL1A binding properties within 2-fold of parental TL1A-0002 in a competition ELISA, has similar or improved non-specificity scores and has an improved T-reg adjusted Pfizer score as described above (Table 7). Plates were washed with PBS+0.05% Tween-20 following a standard ELISA protocol, and goat anti-human IgG-Fc HRP conjugated secondary antibody (Pierce, catalog number 31413) diluted to 1:10,000 in assay buffer was added to plates and incubated for 1 hour at room temperature. Next, the plates were washed with PBS+0.05% Tween-20 following a standard ELISA protocol, and then TMB substrate (BioFX Laboratories, catalog number TMBW-0100-01) was added to the plates. Reactions were stopped after approximately 5 minutes by adding 0.18 M ortho-phosphoric acid to each well and absorbance was read at 450 nm. Output from the direct binding ELISA was assessed and scored as binding or no binding (NB), binding curves are shown in FIGS. 3A-3C. The results show that 29 of 33 [xTL1A] bivalent antibodies screened demonstrated no binding to recombinant human TL1A signifying binding to TL1A was compromised (FIGS. 3A-3C and Table 11).

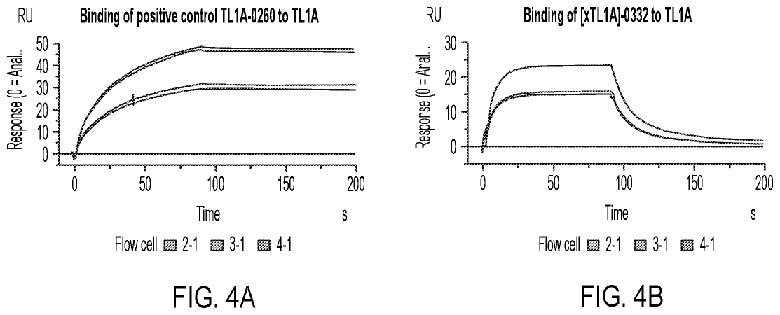

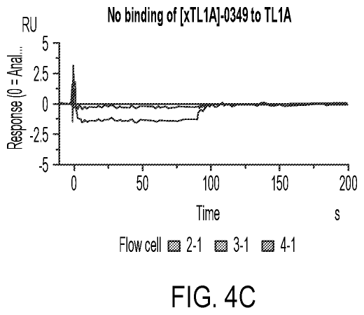

An SPR assay was also used to determine whether the [xTL1A] bivalent antibody variants could bind recombinant human TL1A. Recombinant human TL1A was biotinylated using EZ-link™ Sulfo-NHS-Biotin Sulfosuccinimidobiotin at a molar coupling ratio of 20:1 according to the manufacturer's protocols. Biotinylated recombinant TL1A was captured on the surface of a Biotin CAPture Sensorchip (GE Healthcare, 28-9202-34) to a density of about 30-50 resonance units (RU) following the manufacturer's protocol and conducted at 37° C. using a collection rate of 10 Hz on a Biacore™ T200 instrument (GE Healthcare). A high concentration (1 μM) of [xTL1A] variant antibodies were injected in assay buffer (HBS-EP+, pH 7.4, Cytiva, product number BR100669). Binding to TL1A was assessed and scored as binding or no binding (NB), representative binding sensorgrams are shown in FIGS. 4A-4C. Results from this SPR binding evaluation show that 32 of 33 [xTL1A] bivalent antibodies demonstrated no binding to recombinant human TL1A despite the very high concentration of variant antibodies injected over immobilized TL1A showing binding to TL1A was compromised (FIGS. 4A-4C and Table 11).

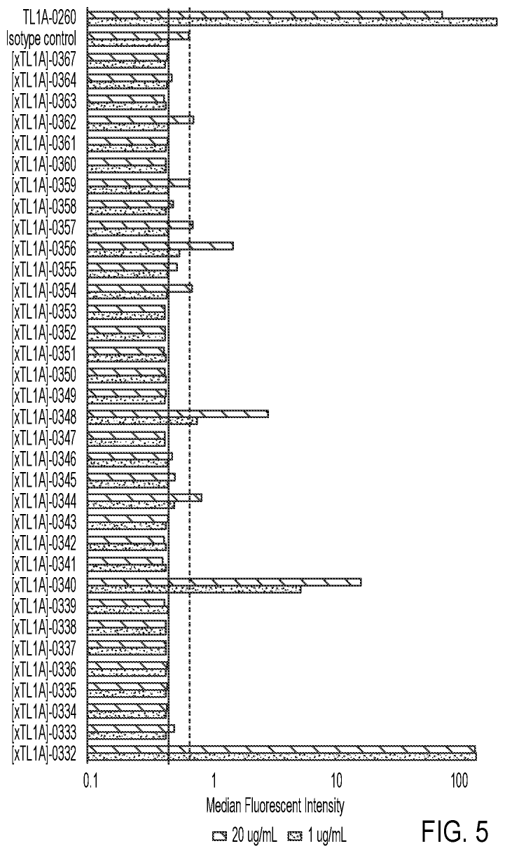

A cell binding assay was performed to examine if the panel of [xTL1A] bivalent antibody variants could bind to human TL1A over-expressed on the cell surface. Briefly, CHO-K1 cells stably expressing full-length human TL1A were resuspended into Dulbecco's phosphate-buffered saline (DPBS) (Gibco 14190-144) supplemented with 4% FBS (Gibco 10082-147) and plated at 100,000 cells per well in a 96-well round bottomed plate (Falcon #353077). [xTL1A] bivalent antibody variants were incubated with cells on ice for 30-60 minutes at a concentration of either 1 μg/mL or 20 μg/mL. Cells were pelleted by centrifugation at 1500 RPM for 5 minutes at 4° C. and supernatant was discarded by aspiration. The cells were washed three times with DPBS with 4% FBS buffer as described. Cells were resuspended in 100 μL of PE-anti-human IgG (Life technologies Catalog number H10104) diluted 1:500 in DPBS with 4% FBS buffer and incubated on ice for 15 minutes. Cells were washed three times as described and resuspended in 200 μL of DPBS with 4% FBS buffer, then run on a flow cytometry instrument. [xTL1A] variants that showed binding greater than the isotype control were deemed to bind TL1A expressing cells. Evaluation of cell binding demonstrated that 26 of the 33 [xTL1A] bivalent antibodies screened exhibited no binding to human TL1A over-expressed on cells demonstrating that both binding to TL1A was compromised and non-specific binding to cells was not observed (FIG. 5 and Table 11).

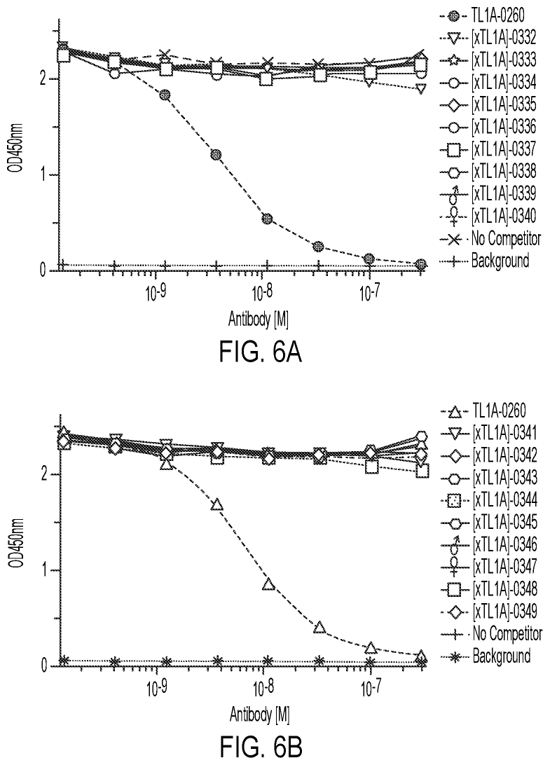

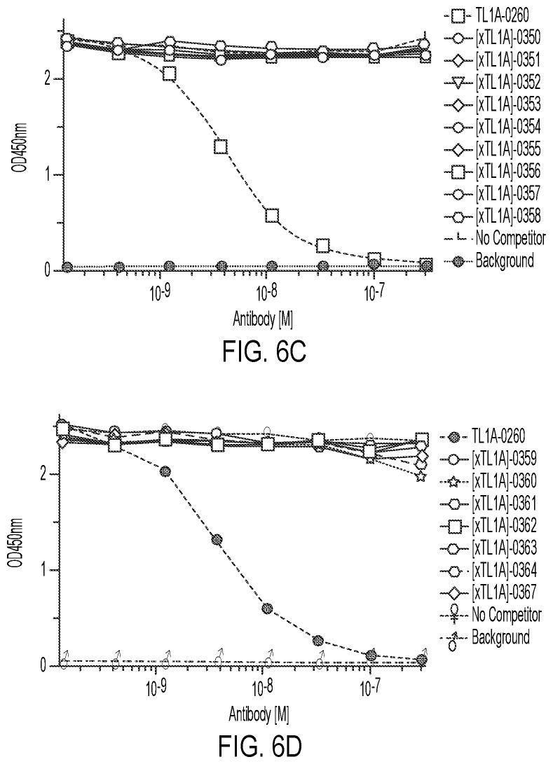

For the bio-TL1A-0260 competition ELISA, TL1A-0260 reporter antibody was biotinylated using EZ-link™ Sulfo-NHS-Biotin Sulfosuccinimidobiotin at a molar coupling ratio of 20:1 according to the manufacturer's protocols. For this competition ELISA assay procedure, 96-well high binding plates (Corning, product number 3590) were coated with 1 μg/mL recombinant TL1A in 100 μL/well in Phospate Buffered Saline (PBS) overnight at 4° C. Plates were then blocked in PBS+0.02% casein solution and washed with PBS+0.05% Tween-20 following standard ELISA protocol. Biotinylated TL1A-0260 (bioTL1A-0260) antibody diluted to the EC₇₀ (30 ng/mL) in assay buffer (PBS+0.5% BSA+0.02% Tween-20) was mixed with a 0.137-300 nM concentration range of the variants or optimized TL1A-0260 antibody as the positive control, using a singleton sample for each antibody concentration evaluated. Plates were washed with PBS+0.05% Tween-20 following standard ELISA protocol, and Streptavidin-HRP (Southern Biotech, product number 7100-05) diluted to 1:10,000 in assay buffer was added to plates and incubated for 25-30 minutes at room temperature. Next, the plates were washed with PBS+0.05% Tween-20 following standard ELISA protocol, and then TMB substrate (BioFX Laboratories Catalog number TMBW-0100-01) was added to plates. Reactions were stopped after approximately 5 minutes by adding 0.18 M ortho-phosphoric acid to each well and absorbance was read at 450 nm. Competition with bio-TL1A-0260 antibody for binding to TL1A was assessed and scored as weak competition, or no competition (NC), competition binding curves are shown in FIGS. 6A-6D. The results showed that 29 of 33 [xTL1A] bivalent antibodies did not compete with bio-TL1A-0260 for binding to recombinant human TL1A protein indicating TL1A binding properties were compromised (FIGS. 6A-6D and Table 11).

The [xTL1A] variants were additionally subjected to assessment for polyreactivity to DNA and insulin and AC-SINS self-association biophysical characterization assays to evaluate non-specificity properties and T-reg adjusted Pfizer scores were calculated as previously described above. Engineering of the disabled or null arm did not introduce any in silico predicted T cell epitopes. From this non-specificity analysis, all [xTL1A] variants exhibit comparable or improved non-specificity scores and T-reg Adjusted Pfizer score (Table 11).

The panel of [xTL1A] variants were screened using the in vitro assays described above to identify those that do not bind TL1A and retain the favorable non-specificity properties of TL1A-0326 one-armed antibody that these were derived from in order to select “disabled” heavy chains for engineering monovalent bispecific antibody variants. Characterization of the 33 disabled [xTL1A] bivalent antibody variants identified 26 variants with undetectable binding to recombinant TL1A and TL1A presented on the cell surface, and seven were selected to re-format into the monovalent bispecific antibody modality with the functional TL1A-0326 HC and functional TL1A-0326 LC.

TABLE 11
Summary of in vitro Assay Results Used to Triage [xTL1A] Variants.

| | | | | Assessment of binding to Human TL1A | | | | | | | |
|---|---|---|---|---|---|---|---|---|---|---|---|
| | | | | | | | Bio-TL1A- | Non-specificity | | | T-reg |
| | Human | Mutation(s) | | | Cells | | 0260 | (Antibody) | | | Adjusted |
| | Germline | CDRH3 (Pfabat | | | 6.7 | 133 | Competition | AC- | | | Pfizer |
| Name | FW | numbering) | ELISA | SPR | [nM] | [nM] | ELISA | SINS | DNA | Insulin | score |
| [xTL1A]- | IGHV1- | None | binding | binding | binding | binding | weak | 2 | 2 | 4 | −39.92 |
| 0332 | 18*01 | | | | | | competition | | | | |
| [xTL1A]- | IGHV1- | G(100a)E | NB | NB | NB | NB | NC | 0 | 2 | 4 | −38.89 |
| 0333 | 18*01 | | | | | | | | | | |
| [xTL1A]- | IGHV1- | G(100e)A | NB | NB | NB | NB | NC | 2 | 4 | 4 | −37.93 |
| 0334 | 18*01 | | | | | | | | | | |
| [xTL1A]- | IGHV1- | G(100e)H | NB | NB | NB | NB | NC | 2 | 4 | 5 | −40.11 |
| 0335 | 18*01 | | | | | | | | | | |
| [xTL1A]- | IGHV1- | G(100a)E, | NB | NB | NB | NB | NC | 2 | 3 | 4 | −37.84 |
| 0336 | 18*01 | G(100e)A | | | | | | | | | |
| [xTL1A]- | IGHV1- | G(100a)E, | NB | NB | NB | NB | NC | 1 | 3 | 5 | −40.08 |
| 0337 | 18*01 | G(100e)H | | | | | | | | | |
| [xTL1A]- | IGHV1- | Y(97)S, | NB | NB | NB | NB | NC | 0 | 2 | 4 | −40.15 |
| 0338 | 18*01 | G(100a)E | | | | | | | | | |
| [xTL1A]- | IGHV1- | Y(97)S, | NB | NB | NB | NB | NC | 2 | 3 | 5 | −39.16 |
| 0339 | 18*01 | G(100e)A | | | | | | | | | |
| [xTL1A]- | IGHV1- | Y(97)S, | binding | NB | binding | binding | NC | 2 | 4 | 5 | −39.16 |
| 0340 | 18*01 | G(100e)H | | | | | | | | | |
| [xTL1A]- | GHV1 | Y(97)S, | NB | NB | NB | NB | NC | 1 | 2 | 4 | −39.16 |
| 0341 | 18*01 | G(100a)E, | | | | | | | | | |
| | | G(100e)A | | | | | | | | | |
| [xTL1A]- | IGHV1- | Y(97)S, | NB | NB | NB | NB | NC | 1 | 2 | 5 | −39.16 |
| 0342 | 18*01 | G(100a)E, | | | | | | | | | |
| | | G(100e)H | | | | | | | | | |
| [xTL1A]- | IGHV3- | None | NB | NB | NB | NB | NC | 1 | 3 | 4 | −54.22 |
| 0343 | 23*01 | | | | | | | | | | |
| [xTL1A]- | IGHV3- | G(100a)E | NB | NB | binding | binding | weak | 0 | 2 | 3 | −53.18 |
| 0344 | 23*01 | | | | | | competition | | | | |
| [xTL1A]- | IGHV3- | G(100e)A | NB | NB | NB | NB | NC | 1 | 4 | 4 | −52.23 |
| 0345 | 23*01 | | | | | | | | | | |
| [xTL1A]- | IGHV3- | G(100e)H | NB | NB | NB | NB | NC | 1 | 3 | 4 | −54.41 |
| 0346 | 23*01 | | | | | | | | | | |
| [xTL1A]- | IGHV3- | G(100a)E, | NB | NB | NB | NB | NC | 0 | 2 | 4 | −52.14 |
| 0347 | 23*01 | G(100e)A | | | | | | | | | |
| [xTL1A]- | IGHV3- | G(100a)E, | binding | NB | binding | binding | NC | 0 | 2 | 3 | −54.38 |
| 0348 | 23*01 | G(100e)H | | | | | | | | | |
| [xTL1A]- | IGHV3- | Y(97)S, | NB | NB | NB | NB | NC | 1 | 2 | 4 | −54.45 |
| 0349 | 23*01 | G(100a)E | | | | | | | | | |
| [xTL1A]- | IGHV3- | Y(97)S, | NB | NB | NB | NB | NC | 1 | 2 | 4 | −53.46 |
| 0350 | 23*01 | G(100e)A | | | | | | | | | |
| [xTL1A]- | IGHV3- | Y(97)S, | NB | NB | NB | NB | NC | 2 | 2 | 4 | −53.46 |
| 0351 | 23*01 | G(100e)H | | | | | | | | | |
| [xTL1A]- | IGHV3- | Y(97)S, | NB | NB | NB | NB | NC | 0 | 2 | 4 | −53.46 |
| 0352 | 23*01 | G(100a)E, | | | | | | | | | |
| | | G(100e)A | | | | | | | | | |
| [xTL1A]- | IGHV3- | Y(97)S, | NB | NB | NB | NB | NC | 0 | 2 | 4 | −53.46 |
| 0353 | 23*01 | G(100a)E, | | | | | | | | | |
| | | G(100e)H | | | | | | | | | |
| [xTL1A]- | IGHV1- | None | NB | NB | NB | NB | NC | 3 | 4 | 6 | −59.21 |
| 0354 | 2*02 | | | | | | | | | | |
| [xTL1A]- | IGHV1- | G(100a)E | NB | NB | NB | NB | NC | 2 | 4 | 5 | −58.17 |
| 0355 | 2*02 | | | | | | | | | | |
| [xTL1A]- | IGHV1- | G(100e)A | binding | NB | binding | binding | NC | 10 | 6 | 6 | −57.22 |
| 0356 | 2*02 | | | | | | | | | | |
| [xTL1A]- | IGHV1- | G(100e)H | NB | NB | NB | binding | NC | 5 | 5 | 4 | −59.40 |
| 0357 | 2*02 | | | | | | | | | | |
| [xTL1A]- | IGHV1- | G(100a)E, | NB | NB | NB | NB | NC | 3 | 4 | 6 | −57.13 |
| 0358 | 2*02 | G(100e)A | | | | | | | | | |
| [xTL1A]- | IGHV1- | G(100a)E, | NB | NB | NB | NB | NC | 3 | 4 | 5 | −59.37 |
| 0359 | 2*02 | G(100e)H | | | | | | | | | |
| [xTL1A]- | IGHV1- | Y(97)S, | NB | NB | NB | NB | weak | 1 | 4 | 5 | −59.44 |
| 0360 | 2*02 | G(100a)E | | | | | competition | | | | |
| [xTL1A]- | IGHV1- | Y(97)S, | NB | NB | NB | NB | NC | 13 | 5 | 6 | −58.45 |
| 0361 | 2*02 | G(100e)A | | | | | | | | | |
| [xTL1A]- | IGHV1- | Y(97)S, | NB | NB | NB | binding | NC | 3 | 7 | 6 | −58.44 |
| 0362 | 2*02 | G(100e)H | | | | | | | | | |
| [xTL1A]- | IGHV1- | Y(97)S, | NB | NB | NB | NB | NC | 1 | 4 | 6 | −58.45 |
| 0363 | 2*02 | G(100a)E, | | | | | | | | | |
| | | G(100e)A | | | | | | | | | |
| [xTL1A]- | IGHV1- | Y(97)S, | NB | NB | NB | NB | weak | 2 | 4 | 6 | −58.44 |
| 0364 | 2*02 | G(100a)E, | | | | | competition | | | | |
| | | G(100e)H | | | | | | | | | |
| TL1A- | IGHV1- | N/A | maximal | maximal | NB | maximal | maximal | 7 | 8 | 4 | −18.50 |
| 0002 | 18*01 | | binding | binding | | binding | competition | | | | |
| TL1A- | IGHV1- | N/A | maximal | maximal | NB | maximal | maximal | 7 | 11 | 5 | −37.28 |
| 0042 | 18*01 | | binding | binding | | binding | competition | | | | |

N/A = Not Applicable; NB = no binding; NC = no competition

A panel of anti-TL1A monovalent bispecific antibody (MBsAb) variants was constructed by pairing select “disabled” [xTL1A] heavy chains with the functional TL1A-0326 binding domain that was engineered to reduce predicted in silico immunogenicity and eliminate confirmed deamidation liabilities. Specifically, the monovalent bispecific antibody variants are composed of three independent protein chains: the functional TL1A-0326 HC, functional TL1A-0326 LC and a “disabled” [xTL1A] heavy chain designed to eliminate binding to TL1A (FIG. 2). KiH mutations described in EXAMPLE 3 were used to drive heterodimerization of the functional and disabled heavy chains. The monovalent bispecific variants were produced by transient co-transfection of the three independent protein chains in Expi293F™ cells using the manufacturer's recommended protocol and resultant protein was purified using a standard 2-step method: Protein A followed by preparative size-exclusion chromatography (SEC). The monovalent bispecific variants were evaluated for purity using non-reduced capillary gel electrophoresis (cGE) and quantified for presence of HMMS by analytical size exclusion chromatography (aSEC). Affinity constants were calculated for the variants binding recombinant human TL1A using SPR. Ability of MBsAb molecules to neutralize TL1A in the TF-1 NF-κB reporter cell bioassay (described in EXAMPLE 2) was determined. The anti-TL1A monovalent bispecific molecules were also scored in the AC-SINS, DNA and insulin non-specificity assays (EXAMPLE 2) and additionally screened at high concentrations for off-target binding to human serum albumin (HSA) and bovine serum albumin (BSA) to ensure presence of the disabled [xTL1A] Fab arm does not introduce off-target binding. Evaluation of binding to HSA and BSA was included since serum albumin can bind a remarkable variety of proteins, including exogenous pharmacological drugs.

For the direct binding ELISA method to HSA or BSA, 96-well high binding plates (Corning, product number 3590) were coated with 1 μg/mL recombinant HSA or BSA in 100 μL/well in PBS overnight at 4° C. Plates were then blocked in PBS+0.02% casein solution and washed with PBS+0.05% Tween-20 following standard ELISA protocol. Assay buffer (PBS+0.5% BSA+0.02% Tween-20) was mixed with a 0.457-1000 nM concentration range of the [xTL1A] antibody variants, optimized TL1A-0260 antibody, a positive control antibody with known high binding to HSA or BSA and a negative control antibody with known low binding to HSA or BSA, using a singleton sample for each antibody concentration evaluated. Plates were washed with PBS+0.05% Tween-20 following standard ELISA protocol, and goat anti-human IgG-Fc HRP conjugated secondary antibody (Pierce, #31413) diluted to 1:10,000 in assay buffer was added to plates and incubated for 1 hour at room temperature. Next, the plates were washed with PBS+0.05% Tween-20 following standard ELISA protocol, and then TMB substrate (BioFX Laboratories, catalog number TMBW-0100-01) was added to plates.

Reactions were stopped after approximately 5 minutes by adding 0.18 M ortho-phosphoric acid to each well and absorbance was read at 450 nm. Results demonstrated that all 33 [xTL1A] bivalent antibodies screened demonstrated very low binding to HSA or BSA protein, below that of the negative control. Differential scanning calorimetry (DSC) was used to monitor thermal stability of the MBsAb variants relative to the one-armed antibody TL1A-0326. For this method, samples diluted to 0.3 mg/mL were dispensed into the sample tray of a MicroCal VP-Capillary DSC with Autosampler (Malvern Instruments, Inc.), equilibrated for 5 minutes at 10° C. and then scanned up to 110° C. at a rate of 100° C. per/hour. A filtering period of 16 seconds was selected. Raw data was baseline corrected and the protein concentration was normalized. Origin Software 7.0 (Origin Lab Corporation, Northampton, MA) was used to fit the data to an MN2-State Model with an appropriate number of transitions.

Non-reduced capillary gel electrophoresis (cGE) was performed to examine purity of desired intact MBsAb molecule in the purified preparations using the Caliper LabChip GXII (PerkinElmer Inc., Hopkinton, MA) according to manufacturer's recommended protocol. MBsAb samples were analyzed by HPLC analytical SEC (aSEC) with a YMC-Pack Diol-200 SEC column using a buffer containing 20 mM sodium phosphate and 400 mM NaCl at pH 7.2. Injection volumes of 5 μL molecular weight standards, 25 μL AAB001, and 50 μg per sample were used, drawing and ejecting both at 150 μL/minute. The retention time and peak width of the main peak as well as the areas and percent areas of the main, low-molecular mass species (LMMS), and high-molecular mass species (HMMS) peaks were recorded.

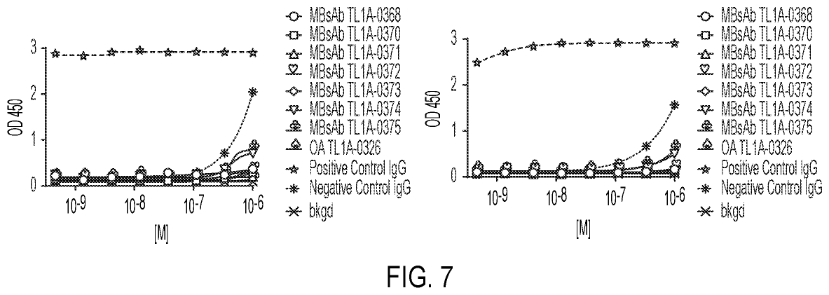

The panel of MBsAb variants was produced using the transient Expi293F™ expression system and resultant titers ranged from 183-224 mg/L which are comparable to that of antibodies in a standard IgG format. Variants purified by Protein A followed by a preparative size-exclusion chromatography (SEC) step were evaluated for purity of intact MBsAb using non-reducing cGE and monitored for amount of HMMS present in the final preparation. Characterization of the MBsAB preparations by NR cGE show that they are of high purity at ˜97% intact molecule and in the expected range of a typical well-behaved antibody amenable to standard purification methods (Table 12). Further, the purified MBsAb preparations were shown to contain less than 1% HMMS (Table 12) indicating presence of the [xTL1A] Fab arm does not alter favorable biophysical characteristics of the one-armed antibody TL1A-0326. Standard non-specificity analysis, specifically assessing properties in the AC-SINS self-association assay and evaluation of binding to DNA or Insulin, show that presence of the various [xTL1A] Fab arms does not alter the favorable non-specificity properties of OA TL1A-0326 used to generate the MBsAB variants (Table 12). Additional screening assays were employed to ensure that [xTL1A] Fab arms do not contribute to off-target binding, precisely the MBsAb variants were screened at high concentrations (0.5-1000 nM) for off-target binding to human serum albumin (HSA) and bovine serum albumin (BSA) relative to positive and negative control antibodies. The positive control antibody has established off-target binding, while the negative control IgG exhibits minimal off-target binding. The results from the BSA and HSA binding ELISAs show that all the MBsAB variants screened exhibit significantly less binding than the positive control and less than the negative control antibody to BSA and HSA (FIG. 7). Neutralization ability of the MBsAb variants was evaluated using the TF-1 NF-κB reporter cell bioassay (EXAMPLE 2) and potency of neutralization for all MBsAB variants was comparable to the one-armed antibody TL1A-0326 indicating that the presence of the various [xTL1A] Fab arms screened does not compromise activity of the TL1A-0326 Fab (Table 13). Affinity constants calculated using SPR show that all MBsAB variants exhibit equivalent binding to recombinant TL1A as the one-armed antibody TL1A-0326 and suggests no additive binding was contributed by the different [xTL1A] Fab arms used to construct the MBsAB molecules (Table 13).

TABLE 12
Summary of Key Biophysical and Bioanalytical Properties Evaluated for Anti-TL1A Monovalent Bispecific Molecules.

| Monovalent | | | | | | | | |
|---|---|---|---|---|---|---|---|---|
| Bispecific | Disabled Fab | | | | Purity: | | | |
| (MBsAb) or One- | | Human | | | % Non- | | | |
| armed Antibody | | Germline | aSEC % | | reduced | Non-specificity Scores | | |
| (OA-Ab) | Variant | Usage | HMMS | DSC Tm1 (° C.) | cGE | AC-SINS | DNA | Insulin |
| TL1A-0368 MBsAb | TL1A-0336 | IGHV1-18*01 | 0.15 | 71.77 ± 0.02 | 97.5 | 2 | 3 | 3 |
| TL1A-0370 MBsAb | TL1A-0341 | IGHV1-18*01 | 0.10 | 71.41 ± 0.01 | 97.9 | 1 | 3 | 3 |
| TL1A-0371 MBsAb | TL1A-0347 | IGHV3-23*01 | 0.13 | 70.80 ± 0.01 | 96.2 | 1 | 2 | 2 |
| TL1A-0372 MBsAb | TL1A-0349 | IGHV3-23*01 | 0.09 | 70.72 ± 0.02 | 97.5 | 1 | 2 | 3 |
| TL1A-0373 MBsAb | TL1A-0352 | IGHV3-23*01 | 0.10 | 70.65 ± 0.01 | 97.2 | 1 | 2 | 3 |
| TL1A-0374 MBsAb | TL1A-0358 | IGHV1-2*02 | 0.11 | 72.13 ± 0.03 | 97.6 | 3 | 4 | 4 |
| TL1A-0375 MBsAb | TL1A-0360 | IGHV1-2*02 | 0.09 | 70.95 ± 0.02 | 97.5 | 1 | 4 | 3 |
| TL1A-0326 OA-Ab | N/A | N/A | 0.49* | 71.07 ± 0.006* | ND | 1* | 3* | 3* |

N/A = non-applicable; ND = not determined; * = calculated values for TL1A-0326 OA Ab were determined in separate experiments versus MBsAb variants.

TABLE 13
Calculated Affinity Constants for Monovalent Bispecific Variants Binding Human TL1A and
Potency of Neutralization in the NFκB Bioassay Relative to One-Armed Antibody TL1A-0326.

| Monovalent | Disabled Fab | | | | | |
|---|---|---|---|---|---|---|
| Bispecific (MBsAb) or | | Human | | | | NFκB |
| One-armed Antibody | | Germline | Binding to Human TL1A (SPR) | | | Bioassay |
| (OA-Ab) | Variant | Usage | ka (1/Ms) | kd (1/s) | K_{D} [pM] | IC₅₀ [nM] |
| TL1A-0368 MBsAb | TL1A-0336 | IGHV1-18*01 | 1.91E+05 | 4.61E−05 | 241.96 ± 11.29 | 1.56 |
| TL1A-0370 MBsAb | TL1A-0341 | IGHV1-18*01 | 1.90E+05 | 5.73E−05 | 302.56 ± 19.48 | 1.46 |
| TL1A-0371 MBsAb | TL1A-0347 | IGHV3-23*01 | 1.68E+05 | 4.36E−05 | 260.58 ± 12.39 | 1.37 |
| TL1A-0372 MBsAb | TL1A-0349 | IGHV3-23*01 | 1.74E+05 | 4.98E−05 | 285.96 ± 19.17 | 1.47 |
| TL1A-0373 MBsAb | TL1A-0352 | IGHV3-23*01 | 1.73E+05 | 4.95E−05 | 285.47 ± 15.99 | 1.66 |
| TL1A-0374 MBsAb | TL1A-0358 | IGHV1-2*02 | 2.09E+05 | 5.59E−05 | 267.84 ± 6.63 | 1.49 |
| TL1A-0375 MBsAb | TL1A-0360 | IGHV1-2*02 | 2.11E+05 | 4.90E−05 | 232.2 ± 11.79 | 1.35 |
| TL1A-0326 OA-Ab | N/A | N/A | 2.00E+05 | 5.84E−05 | 292.36 ± 18.64 | 1.28 |

N/A = non-applicable

The TL1A-0372 monovalent bispecific antibody which is comprised of the TL1A-0326 functional Fab paired with the [xTL1A]-0349 Fab, and the [xTL1A]-0349 bivalent antibody were further examined for antibody specificity and off-target binding by screening for interactions with cell surface receptors and secreted proteins (Retrogenix, Whaley Bridge, UK). For the primary screen, a pool containing the following, with final concentrations noted, TL1A-0372 (25 μg/mL), [xTL1A]-0349 (25 μg/mL), and the isotype control (20 μg/mL) was screened for binding against fixed HEK293 cells/slides expressing duplicate 5528 human plasma membrane proteins and cell surface tethered human secreted proteins (16 slide sets, n=2 slides per slide set). All transfection efficiencies exceeded the minimum threshold. An AlexaFluor647 anti-human IgG Fc detection antibody was used in the primary screen. In total, primary hits (duplicate spots) were identified by analyzing fluorescence (AF647 and ZsGreen1) on ImageQuant. There were a range of intensities (signal to background) from very weak to strong. For the confirmation/specificity screening, vectors encoding all primary hits, plus control vectors encoding CD20 and EGFR, were spotted in duplicate on new slides, and used to reverse transfect human HEK293 cells as before in the primary screen. All transfection efficiencies exceeded the minimum threshold. Slides were subsequently spotted with gelatin±human TL1A antigen. Identical fixed slides were treated with 25 μg/mL of TL1A-0372 monovalent bispecific, 25 μg/mL [xTL1A]-0349, and 20 μg/mL for the isotype control, or 1 μg/ml Rituximab biosimilar (positive control), or no test molecule (secondary only; negative control) (n=2 slides per treatment). Slides were analyzed as primary screen. Hits were categorized as specific, or non-specific (i.e., it also came up with at least one of the positive and/or negative controls). The summary of conclusions after screening the TL1A-0372 monovalent bispecific, [xTL1A]-0349 antibody and the isotype control for binding against 5528 human plasma membrane proteins and cell surface-tethered secreted proteins expressed in human HEK293 cells are the following. TL1A-0372 monovalent bispecific specifically interacted with TL1A, the primary target, with strong intensity and no other interactions were identified demonstrating high specificity for the primary target human TL1A. [xTL1A]-0349 antibody had no specific interactions, while the isotype control interacted with HAVCR2 only (medium intensity). These results demonstrated on-target binding only for TL1A-0372 to TL1A; [xTL1A]-0349 did not exhibit binding to any proteins. Thus, off-target binding was not observed for either the anti-TL1A or disabled binding domains in TL1A-0372 or [xTL1A]-0349 bivalent disabled antibody.

PK parameters for TL1A-0372 monovalent bispecific antibody and TL1A-0326 one-armed antibody were determined in cynomolgus monkeys. TL1A-0372 test article was administered to 4 cynomolgus monkeys as a single IV bolus dose of 50 mg/kg. Blood samples were collected pre-dose and post-dose to 2016 hours. The TL1A-0372 PK study was conducted at UL Lafayette-New Iberia Research Center (New Iberia, LA 70560) and executed within accordance of the Animal Use Protocol. Quantitative analysis of serum samples was conducted using a ligand binding assay (generic human IgG assay format) developed on the Gyrolab® platform. Non-compartmental analysis was performed using Phoenix 64 software (build 8.0.03176). In a separate study, TL1A-0326 test article was administered to 6 cynomolgus monkeys as a single IV bolus dose, with dose levels ranging from 0.01 mg/kg to 300 mg/kg. Blood samples were collected pre-dose and post-dose to 1344 hours. The study was conducted at Charles River Laboratories (Reno, NV) and executed within accordance of the Animal Use Protocol. Quantitative analysis of serum samples was conducted using a ligand binding assay (generic human IgG assay format) developed on the Gyrolab® platform. Non-compartmental analysis was performed using SimBiology® software in MATLAB® (MathWorks®). Results from these monkey PK studies show that despite inclusion of LS mutations intended to extend the half-life in the TL1A-0326 monospecific one-armed variant, the terminal half-life was notably short and is consistent with findings from the Tg32 study (Table 9). However, TL1A-0372 monovalent bispecific antibody engineered with a disabled TL1A binding arm, had a significantly longer serum terminal half-life tha TL1A-0326 one-armed antibody and indicates that the addition of the disabled Fab arm restored the serum half-life in cynomolgus monkeys to the range of an antibody harboring LS mutations in a typical IgG format of approximately 30 days^{7, 9, 10} (Table 13).

TABLE 13
Terminal Half-lives Determined for LS Engineered TL1A-0326
One-Armed Variant and TL1A-0372 Monovalent Bispecific Variant
as Compared to TL1A-0002 Antibody in Cynomolgus Monkey.

| | | | Dose level | Terminal Half- |
|---|---|---|---|---|
| Sample | Modality | Fc format | (mg/kg) | life (Days) |
| TL1A- | One-armed | IgG, KiH, | 0.01-300^{A} | 5.4 |
| 0326 | Antibody | LS | | |
| TL1A- | Monovalent | IgG, KiH, | 50^{B} | 28.0 |
| 0372 | bispecific | LS | | |
| | antibody | | | |

^{A}n = 1 animal/dose group
^{B}n = 4 animal/dose group

### Example 5: Kinetic Evaluation of Monovalent Bispecific Antibody TL1A-0372 Binding Human, Cynomolgus Monkey, Rabbit, Mouse and Rat TL1A Using Surface Plasmon Resonance

Surface plasmon resonance (SPR) was used to evaluate cross-species binding of the monovalent bispecific TL1A-0372 to recombinant human, cynomolgus monkey, rabbit, mouse, and rat TL1A (human SEQ ID NO: 208, Cynomolgus monkey SEQ ID NO: 209, Rabbit SEQ ID NO: 210, Mouse SEQ ID NO: 211 and Rat SEQ ID NO: 212). For these analyses, kinetic assays were conducted at 37° C. using a collection rate of 10 Hz on a Biacore™ T200 instrument (GE Healthcare). Recombinant human, cynomolgus monkey, rabbit, mouse, and rat TL1A were biotinylated using EZ-link™ Sulfo-NHS-Biotin Sulfosuccinimidobiotin at a molar coupling ratio of 20:1 according to the manufacturer's protocols. For this evaluation, biotinylated recombinant TL1A was captured on the surface of a Biotin CAPture Sensorchip (GE Healthcare, 28-9202-34) to a density of about 30-50 resonance units (RU) following the manufacturer's protocol. TL1A-0372 was injected for 90 seconds at a flow rate of 55 μL/minute at 11.1, 33.3, 100, 300 and 900 nM concentrations. The association phase was 90 seconds, and the dissociation phases were 1600 seconds over human, cynomolgus monkey and rabbit TL1A and 180 seconds for mouse and rat TL1A. The results from this SPR analysis show that monovalent bispecific TL1A-0372 exhibits ˜2-fold higher affinity to human TL1A versus cynomolgus monkey TL1A, has ˜48-fold higher affinity to human TL1A than for rabbit TL1A, and shows ˜546-fold higher affinity to human TL1A relative to mouse TL1A (Table 14). Affinity constants of monovalent bispecific TL1A-0372 binding to rat TL1A were approximated and are much weaker than those for human, cynomolgus monkey, rabbit or mouse TL1A (Table 14).

TABLE 14
Affinity Constants for Monovalent Bispecific Antibody TL1A-
0372 Binding Human, Cynomolgus Monkey, Rabbit, Mouse and
Rat TL1A Measured using Surface Plasmon Resonance.

| Species TL1A | ka (1/Ms) | kd (1/s) | K_{D} [nM] |
|---|---|---|---|
| Human | 1.14E+05 | 6.21E−05 | 0.54 ± 0.01 |
| Cynomolgus monkey | 9.64E+04 | 1.21E−04 | 1.27 ± 0.17 |
| Rabbit | 3.90E+04 | 1.01E−03 | 25.95 ± 1.42 |
| Mouse | 2.40E+04 | 7.08E−03 | 294.78 ± 3.93 |
| Rat | 3.22E+04 | 4.83E−02 | *1506.91 ± 71.76 |

*Approximate K_{D} value

### Example 6. Derivation of Anti-IL-12/23 p40 Antibody p40-0003

IL-12p40, or referred herein as p40, is a shared subunit of the heterodimeric cytokines IL-12 which is composed of p40 and p35 subunits and IL-23 which is composed of p40 and p19 subunits. The approach used to generate an anti-p40 binding domain was to obtain the heavy and light chain variable region (V_{H} and V_{L}) amino acid sequences comprising anti-IL-12p40 human monoclonal antibody C230 (ustekinumab, Stelara®) obtained from Giles-Komar et al. U.S. Pat. No. 6,902,734 (Centocor Inc., Jun. 7, 2005)¹¹. The C230 V_{L} (SEQ ID NO: 177) was fused to the human Kappa constant region (SEQ ID NO: 16) within the pTT5 expression vector to generate p40-0003 LC (SEQ ID NO: 178). The C230 V_{H} (SEQ ID NO: 171) was joined to the human IgG1 constant region harboring mutations in the CH2 domain (L(H247)A, L(H248)A, G(H250)A Pfabat numbering) to minimize effector function within the pTT5 expression vector to create p40-0003 HC (SEQ ID NO: 172). Expi293F™ HEK cells were transiently co-transfected with the expression vectors encoding p40-0003 LC and HC and the resultant p40-0003 antibody protein was purified using a MabSelect™ SuRe™ column followed by buffer exchange into PBS-CMF pH7.2.

### Example 7. Kinetic Evaluation of Anti-IL12p40 p40-0003 Antibody Binding Human and Cynomolgus Monkey IL-12 and IL-23 Cytokines Using Surface Plasmon Resonance

Surface plasmon resonance (SPR) was performed to determine the affinity constants for p40-0003 antibody binding human and cynomolgus monkey (cyno) IL-12 (human SEQ ID NO: 213 and SEQ ID NO: 215; cyno SEQ ID NO: 214 and SEQ ID NO. 216) and human and cyno IL-23 (human SEQ ID NO: 213 and SEQ ID NO: 217; cyno SEQ ID NO: 214 and SEQ ID NO: 218) that both contain the shared IL-12p40 subunit (human SEQ ID NO: 213 and cyno SEQ ID NO: 214). For these analyses, kinetic assays were conducted at 37° C. using a collection rate of 10 Hz on a BIAcore™ T200 instrument (GE Healthcare). An anti-human IgG antibody (anti-human Fc, catalog number 10-005-098, Jackson ImmunoResearch) was amine coupled to all four flow cells of a carboxymethylated dextran coated sensor chip (GE Healthcare) using the manufacturer's recommended protocol. Next, the p40-0003 antibody was immobilized to ˜100 RU and then various concentrations of human IL-12, cyno IL-12, human IL-23 or cyno IL-23 were injected over the surface. The measured affinity constants of p40-0003 antibody binding human and cynomolgus monkey IL-12, and IL-23 are presented in Table 15. The results show that anti-IL12p40 p40-0003 antibody binds to human IL-12 and IL-23 with low pM affinity (Table 15). Additionally, p40-0003 also exhibits ˜2-3 fold higher affinity to human IL-12 and IL-23 than to the respective cynomolgus monkey cytokines.

TABLE 15
Affinity Constants for p40-0003 Antibody Binding
Human and Cynomolgus Monkey IL-12 and IL-23
Measured using Surface Plasmon Resonance

| IL-12 | | IL-23 | |
|---|---|---|---|
| Human | Cyno | Human | Cyno |
| 156 + 7.07 pM | 368 + 12.0 pM | 159.5 + 0.71 pM | 316.5 + 40.3 pM |

### Example 8. Engineering Anti-p40/TL1A Bispecific Variants Using Different Molecular Formats and Heterodimerization Mutations to Minimize Byproducts

Analysis of RNA sequencing data generated from UC colon samples of patients treated with anti-TL1A mAb (PF-06480605) in the Ph2a TUSCANY trial (NCT02840721)¹², as reported in Hassan-Zahraee et al.¹³, identified molecular-level differences in tissue transcriptomes of responders and non-responders. While responders to treatment experienced a strong reversal of UC disease gene signature and inflammatory pathway activation, non-responders experienced only partial reversal, and a residual disease signature was observed. Importantly, remaining Th1 and Th17 inflammatory tone was detected in inflamed biopsies of non-responders, which could be effectively addressed with the direct neutralization of IL-12 and IL-23 by anti-p40. Furthermore, computational cell type deconvolution of inflamed biopsy transcriptomes pre and post anti-TL1A treatment demonstrated both a significant decrease in fibroblasts and an increase in epithelial cell contribution in tissues of responders, and partially in non-responders, consistent with an anti-fibrotic and pro tissue resolution effect. The ability of anti-TL1A to inhibit fibrosis and promote epithelial restoration will serve to augment the efficacy of the anti-inflammatory activity of anti-p40 therapy. In conclusion, the bispecific blockade of p40 and TL1A will provide optimal therapeutic coverage of multiple disease-modifying mechanisms along the IBD spectrum, resulting in maximized inhibition of pathogenic Th1 and Th17 inflammation, reduction in fibrotic tone, and repair of epithelial damage.

Several bifunctional format designs were engineered to identify molecules that could simultaneously bind and neutralize p40 and TL1A while conferring developability parameters of a standard monoclonal antibody. Design considerations for engineering the bispecific variants were to address multiple challenges: in addition to binding to and simultaneously blocking p40 and TL1A, the molecule needed efficient pairing of four different protein chains, high-level protein expression with minimal undesired byproducts, high transient and stable expression titers, efficient cell line generation and protein purification processes, and pharmacokinetic properties consistent with a standard monoclonal antibody.

Two approaches were used to confer heterodimerization of the human IgG1-effector function-minimized Fc region in the p40TL1A bispecific molecules. The first approach was done by pairing of two different heavy chains driven by engineering KiH mutations described above in EXAMPLE 3. The second Fc heterodimerization approach used was where two antibodies or antibody-based molecules are expressed separately in dual cell lines or dual transiently expressed pools, one engineered with excess positive charge and the other with excess negative charge in the complementary location at the dimer interface^{14,15}. The two antibodies are purified separately, reduced, and then mixed under appropriate conditions to allow for oxidation that will result in preferential formation of a heterodimer bispecific molecule. This latter approach will be referred to here on as charge-based (CB) mutations for dual cell production. One advantage for producing bispecific molecules using a post-expression chemical redox approach is that each heavy chain-light chain pair is expressed in a separate cell line, eliminating mispairing of cognate light chains. Bispecific molecules engineered with KiH Fc heterodimerization mutations can also be produced via a dual cell line post-expression chemical redox approach. The LS mutations, specifically M(H459)L and N(H465)S (Pfabat numbering), were incorporated into all p40TL1A bispecific molecules to increase serum half-life of the therapeutic to offer increased dosing flexibility.

Two separate strategies were employed to produce KiH engineered bispecific molecules for single cell expression and minimize light chain mispairing when all four protein chains were expressed in a single cell line. The first strategy was to introduce the electrostatic complementary S1 and S1-reverse (S1rev) mutations¹⁶ into the α-p40 Fab and α-TL1A Fab human Kappa and human IgG1-CH1 constant domains, respectively (FIG. 8D). For the S1 design, a negative charge was engineered into the human Kappa constant domain by replacing S(L176)D along with the supporting mutation V(L133)S (FIG. 8E, SEQ ID NO. 197). The complementary S1 design positive charge L(H124)K was introduced into the CH1 with the supporting mutation V(H190)S (FIG. 8E, SEQ ID NO. 195). For the S1-reverse design, a positive charge was engineered onto the human Kappa CL by replacing S(L176)K along with the supporting mutation V(L133)S (FIG. 8F, SEQ ID NO. 201). The complementary S1-reverse design negative charge L(H124)E was introduced into the CH1 with the supporting mutation S(H188)G (FIG. 8F, SEQ ID NO. 199). p40TL1A-0032 bispecific was constructed based on the KiH-S1-S1rev design (FIG. 8D) with the p40-0003 Fab containing the S1 mutations, the TL1A-260 Fab harboring the S1-rev mutations. The four unique amino acid sequences comprising p40TL1A-0032 bispecific are depicted as SEQ ID NO. 196, SEQ ID NO. 198, SEQ ID NO. 200 and SEQ ID NO. 202.

The other design strategy to enhance the fidelity of light chain pairing for p40-TL1A KiH bispecific molecules produced in a single cell line utilizes a Fab arrangement referred to as “modified Fd” (mFd) and was employed for the anti-TL1A Fab (FIG. 8A). In this arrangement, the anti-TL1A light chain (SEQ ID NO: 19) was joined to the lower hinge region (DKTHTCPPCP SEQ ID NO: 204) of the human IgG1-effector function-minimized Fc region to generate the VL-CL-Fc protein chain of this bispecific modality (FIG. 8A). A modified Fd chain was designed to pair with the anti-TL1A LC within the VL-CL-Fc chain, and it is composed of an anti-TL1A VH, human IgG1-CH1 (SEQ ID NO: 6) and the upper human IgG1 hinge amino acids EPKSC harboring Cys at H230 (SEQ ID. NO: 206) for interchain disulfide formation with Cys (L214) in anti-TL1A Fab Kappa constant domain (FIG. 8C). The p40TL1A-0033 bispecific was constructed using the KiH mFd-bispecific modality. Specifically, TL1A-260 LC (SEQ ID NO: 19) was joined to the to the lower hinge region (SEQ ID NO: 204) of an IgG1-effector function-minimized Fc engineered with the Hole heterodimerization mutations T(389)S, L(391)A and Y(438)V along with S(375)C to generate the p40TL1A-0033 VL-CL-Fc chain (SEQ ID NO: 205). The p40TL1A-0033 modified Fd chain (SEQ ID NO: 207) was constructed by fusing the TL1A-260 VH (SEQ ID NO: 42) to the IgG1 CH1 (SEQ ID NO: 6) and upper hinge region (SEQ ID NO: 206). The p40-0003 VH (SEQ ID NO: 171) was fused to an IgG1-effector function-minimized Fc harboring the Knob heterodimerization mutation T(389)W plus Y(370)C to generate the p40TL1A-0033 HC (SEQ ID NO: 203). The p40TL1A-0033 LC is identical to the p40-0003 LC and does not contain mutations in the CL to increase fidelity of chain pairing.

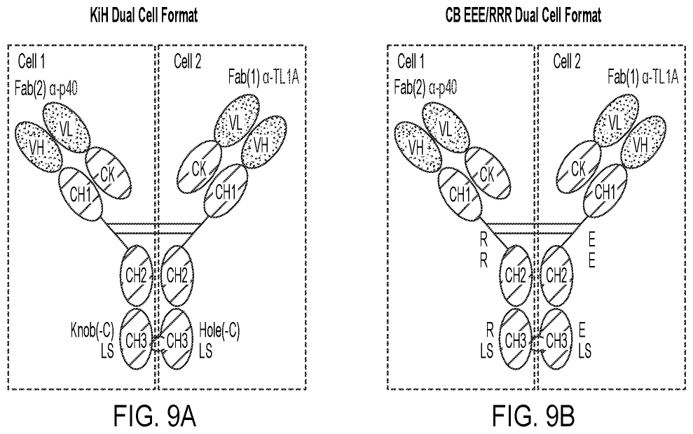

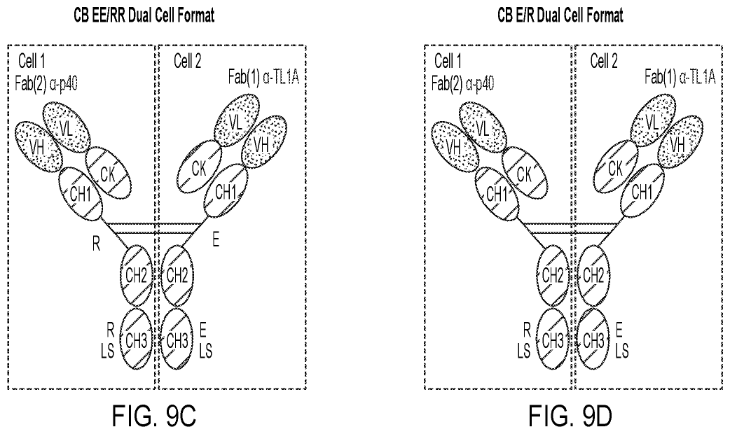

Anti-p40/TL1A bispecific molecules were also constructed for production in dual cell lines which eliminates mispairing of cognate light chains since each heavy chain-light chain pair is expressed in a separate cell line. One bispecific design used the KiH Fc-heterodimerization mutations (EXAMPLE 3) where the anti-p40 HC was engineered with the Knob mutation and anti-TL1A HC contains corresponding Hole mutations, but without the Y(H370)C and S(375)C supporting mutations (FIG. 9A). Bispecific p40TL1A-0018 was constructed with KiH mutations to drive Fc heterodimerization using p40-TL1A-0003 as Fab1 and TL1A-0260 in the Fab2 position (FIG. 9A) and the four unique protein chains are shown as SEQ ID NO. 178, SEQ ID NO. 186, SEQ ID NO: 188 and SEQ ID NO: 19. An alternate approach for dual cell line production of anti-p40/TL1A bispecific molecules utilizes a charge-based (CB) heterodimerization approach, described by Strop et al.¹⁵. The CB strategy, in the context of bispecific antibody generation, allows separate expression of the two portions of a heterodimeric molecule and subsequent joining to form the bispecific heterodimer. This technology relies on placement of oppositely charged residues, arginine and glutamic acid, in equivalent positions in the hinge and CH3 domains of two IgG molecules. These IgGs are expressed and purified separately as homodimers, but then are mixed and treated with a reducing agent to break the disulfide bonds between the two heavy chains. The positioning of opposite charges at the Fc interface leads to preferential pairing of one heavy chain from each of the two starting IgG molecules, and when the mixture is then re-oxidized, disulfide bonds re-form, yielding a mixture that is predominantly heterodimeric. For example, the Fab1 arm composed of the anti-p40 binding domain p40-0003 is expressed in one cell, while the second cell produces the anti-TL1A Fab2 arm TL1A-0260 (FIGS. 9A-9D). The bispecific p40TL1A-0001 (FIG. 9B) was engineered where three positively charged residues (referred to as RRR) were introduced into the human IgG1 constant region of the anti-p40 HC: two mutations D(H232)R and P(H241)R into the hinge (SEQ ID NO: 179) and K(H440)R into the CH3 region of the single Fab chain (SEQ ID NO. 180). The three opposite negative charge-paired mutations incorporated into the human IgG1 constant region of the anti-TL1A HC were D(H232)E and P(H241)E into the hinge region (SEQ ID NO: 182) and L(H391)E into the CH3 domain (SEQ ID NO: 183), referred to collectively as EEE (FIG. 9B). Variations of opposite charge-paired mutations were used to construct anti-p40/TL1A bispecific molecules where the amount of excess charge was reduced in the antibody hinge region. Bispecific p40TL1A-0021 was engineered to include only D(H232)R or D(H232)E in the hinge region and L(H391)E or K(H440)R only in the CH3 domain (“EE/RR;” FIG. 9C), while p40TL1A-0024 contains no charged mutations in the hinge region and harbors L(H391)E or K(H440)R only in the CH3 domain (“E/R;” FIG. 9D).

### Example 9: Transient Expression & Purification of Anti-p40/TL1A Bispecific Variants

The p40TL1A bispecific molecules described above were generated using both single cell and dual cell expression approaches and conditioned medium containing these p40TL1A bispecific proteins was produced using Expi293F™ host cells with the manufacturer's recommended protocol. The general method for purifying single cell derived bispecific variants uses a three-column step: MabSelect™ SuRe™ LX (Cytiva Life Sciences), followed by Mono-S Cation Exchange (Cytiva Life Sciences), and Superdex 200 gel filtration (Cytiva Life Sciences) with the final formulation in PBS-CMF pH 7.2 with details noted in Table 16. In some instance for the single cell produced bispecifics, the Superdex 200 gel filtration step can be eliminated after process development optimization. The general purification process for dual cell produced bispecific variants was the following, with molecule-specific details noted in Table 16; independent MabSelect™ SuRe™ LX capture of the p40 and TL1A homodimers, redox reaction that varies with choice of opposite charge-paired mutations in Fc region, followed by buffer exchange, Mono-S cation exchange, then final buffer exchange into PBS-CMF pH 7.2. These results support that methods can be developed to purify bispecific variants produced via the single cell or dual cell approach, which each have their own benefits and challenges. For the dual cell approach, redox conditions vary depending on which Fc heterodimerization mutations are utilized, and extensive screening for appropriate conditions is required. For the single cell approach, although the expression can be challenging since the four chains comprising the bispecific need to be produced in a single cell, the overall purification method is closer to a standard antibody process and does not require multiple purification steps to obtain the molecule of interest. The single cell approach has increased complexity from an analytical perspective, since production of components from each dual cell, specifically p40 homodimer and TL1A homodimer, would be required to analyze mispaired species.

TABLE 16
Purification Scheme for Single Cell and Dual Cell Expi293F ™ Produced Bispecific Variants.

| Bispecific | | | | | |
|---|---|---|---|---|---|
| Variant | Approach | ProA capture | Redox | Post-redox purification | Analytical |
| p40TL1A- | Dual Cell: | +Platform | +20x Cys at 25° C. | +2 column purification | +Methods established for |
| 0001 | EEE/RRR | | | steps (TMAE-CHA) | parental homodimer detection |
| p40TLIA- | Dual Cell: | −tris/arginine to | −high GHS molar | −limited prep. | +LC pairs correctly when |
| 0018 | KiH no Cys | stabilize KiK | excess | chromatography | expressing in dual cell format |
| | in CH3 | and HiH dimers | −overnight | method (poor separation | −Analytical method for |
| | | | reaction at 37° C. | on HIC and SEC) | mispaired species detection |
| | | | | | needs development |
| p40TL1A- | Dual Cell: | +Platform | −moderate | +good separation on | +LC pairs correctly when |
| 0021 | EE/RR | | cysteine molar | CEX (2 step | expressing in dual cell format |
| | | | excess | purification) | +Parental homodimer |
| | | | −overnight | | detection with aCEX (Agilent |
| | | | reaction at ambient | | 1290) |
| | | | temperature | | |
| p40TL1A- | Dual | +Platform | −high cysteine | −limited prep. | +LC pairs correctly when |
| 0024 | Cell: E/R | | molar excess | chromatography | expressing in dual cell format |
| | | | −overnight | method (poor separation | −Analytical method for |
| | | | reaction at ambient | on HIC and SEC) | parental homodimer detection |
| | | | temperature | | needs development |
| p40TL1A- | Single | +Platform | +No redox needed | +single-step SEC | −limited analytical methods |
| 0032 | Cell: KiH, | | | purification | to detect LC mispairing other |
| | S1-S1rev | | | | than LCMS |
| p40TL1A- | Single Cell: | +Platform | +No redox needed | +single-step SEC | +LC pairs correctly when |
| 0033 | KiH, modified Fd | | | purification | expressing |
| | | | | | +Mispaired species detection |
| | | | | | with aCEX (Agilent 1290) |

+ = favorable property; − = negative property; aCEX = analytical Cation Exchange; CEX = Mono-S Cation Exchange; TMAE-CHA = trimethylaminoethyl acrylamide-ceramic hydroxyapatite chromatography; HIC = hydrophobic interaction chromatography; SEC = size exclusion chromatography; Cys = Cysteine; GSH = Glutathione: KiH = Knob-into-Hole; KiK = Knob-into-Knob; HiH = Hole-into-Hole; Platform = according to manufacturer's recommended protocol.

### Example 10: Stable CHO Expression and Purification of a p40TL1A Bispecific Antibody Produced from a Single Cell Line

Stable CHO expression was evaluated for bispecific variant p40TL1A-0033 engineered using the modified Fd KiH modality single cell approach to gauge feasibility for stable cell line development and to evaluate whether expression of a bispecific molecule produced via transient Expi293F™ cells translated to stable CHO cells. The four unique chains comprising the p40TL1A-0033 bispecific were sub-cloned into a CHO SS1 vector (Lonza) using the following arrangements: (1) LC→HC→mFd→VL-CL-Fc and (2) LC→HC→VL-CL-Fc→mFd. Three independent stable CHO pools were generated for each bispecific variant, and expression titers were determined from 200 mL shake flask cultures (Table 17). Analytical Size Exclusion Chromatography (aSEC) results (Table 17) indicated ≥60% peak-of-interest (POI) was observed for p40TL1A-0033 variants that correlated with observations from non-reduced stain-free SDS-PAGE assessment of the Protein A purified protein. The expression titers and the POI were similar using both vector arrangements, but slightly higher with the arrangement (1) LC→HC→mFd→VL-CL-Fc (Table 17). Further, stable expression in CHO cells of p40TL1A-0033 bispecific molecule correlates with transient expression levels obtained in Expi293F™ cells.

TABLE 17
Stable CHO Expression Titers and Analytical SEC for Single Cell Produced Bispecific
Variants using Stable CHO SSI Expression System.

| | | | | | | % POI via |
|---|---|---|---|---|---|---|
| | Fc | SSI vector | Titer Pool A | Titer Pool B | Titer Pool C | analytical |
| Bispecific | format | Arrangement | [mg/L] | [mg/L] | [mg/L] | SEC |
| p40TL1A-0033 | KiH, LS | LC→HC→mFd→VL-CL-Fc | 1821.4 | 1666.5 | 1720.9 | 69 |
| p40TL1A-0033 | KiH, LS | LC→HC→VL-CL-Fc→mFd | 1449.9 | 1429.7 | 1394.8 | 60 |

### Example 11: Characterization of Biophysical Properties and Non-Specificity Scores Characterization of p40TL1A Bispecific Variants

Anti-p40/TL1A bispecific variants produced using either single or dual cell expression approaches described above were subjected to extensive characterization to evaluate bioanalytical and biophysical properties of these complex molecules prepared using different Fc heterodimerization strategies, electrostatic complementary mutations and mFd Fab format to limit undesired byproducts and encourage maximum yield of the intact bispecific molecule. In particular, the following methods were used to assess key molecular properties: analytical size-exclusion chromatography (aSEC) to determine percent high molecular mass species (HMMS) as an indicator of aggregation, thermal stability using Differential Scanning calorimetry (DSC), non-reduced cGE to assess percent peak of interest (POI), imaged capillary electrophoresis (iCE) for unstressed sample (T0) to evaluate charge heterogeneity, non-specificity evaluation and in silico immunogenicity assessment.

Analytical SEC was performed using a YMC-Pack Diol-200 SEC column in 20 mM Na₃PO₄, 400 mM NaCl, pH 7.2 buffer. Retention time and peak width of the main peak as well as the areas and percent areas of the main (POI), low molecular mass species (LMMS) and HMMS peaks were recorded and used to calculate percent main (POI), HMMS and LMMS.

For the DSC method, samples at 0.3 mg/mL were dispensed into the sample tray of a MicroCal VP-Capillary DSC with Autosampler (Malvern Instruments, Inc.), equilibrated for 5 minutes at 10° C. and then scanned up to 110° C. at a rate of 100° C. per/hour. A filtering period of 16 seconds was selected. Raw data was baseline corrected and the protein concentration was normalized. Origin Software 7.0 (Origin Lab Corporation, Northampton, MA) was used to fit the data to an MN2-State Model with an appropriate number of transitions.

Non-reduced cGE was performed using the Caliper LabChip GXII (PerkinElmer Inc., Hopkinton, MA) according to manufacturer's recommended protocol. Protein Simple iCE3 instrument with PrinCE Autosampler (ProteinSimple, San Jose, CA) was used to analyze charge heterogeneity for the unstressed (T0) Tri-Fab-Fc samples. Proteins were diluted to 2 mg/mL in water. Sample diluent is comprised of 0.01 mg/mL μl marker 4.65, 0.01 mg/mL μl marker 9.5, 4.0% Pharmalyte pH 3-10, 0.25% methyl cellulose, and 2.0 M urea. Samples contained 15 μL protein at 2 mg/mL and 85 μL sample diluent. Samples were focused for 1 minute at 1500 Volts and then 6 minutes at 3000 Volts. The anti-p40/anti-TL1A variant bispecific antibody variants were additionally subjected to DNA/insulin polyreactivity and AC-SINS self-association biophysical characterization assays to evaluate non-specificity properties and T-reg adjusted Pfizer scores were calculated as previously described above.

The summary of results for the biophysical and bioanalytical evaluation of six anti-p40/anti-TL1A variant bispecific antibody variants generated using either the single cell or dual cell approach and engineered with alternate Fc heterodimerization mutations indicate that all generally have favorable molecular properties comparable to standard well-behaved antibodies (Table 18). All six exhibit good thermal stability with Tm1 values >65° C. Integrity of intact bispecific is >97% for all six variants as determined with non-reducing cGE assay and propensity to aggregate was deemed low by analytical SEC analysis. All six bispecific variants present acceptable non-specificity scores and good in silico predicted immunogenicity T-reg adjusted Pfizer scores (Table 18).

TABLE 18
Summary of Key Biophysical and Bioanalytical Properties Evaluated for Bispecific Variants.

| | | | | cGE % | | | | Non-specificity | | | T-reg |
|---|---|---|---|---|---|---|---|---|---|---|---|
| Bispecific | | aSEC % | DSC | Intact | iCE T0 | | | Scores | | | Adjusted |
| Variant | Description | HMMS | Tm1 (° C.) | NR | % acidic | % main | % basic | AC-SINS | DNA | Insulin | Pfizer score |
| p40TL1A- | Dual Cell: | 0.37 | 71.26 | >99 | 22.4 | 72.4 | 5.2 | 1 | 5 | 6 | −31.39 |
| 0001 | EEE/RRR | | | | | | | | | | |
| p40TL1A- | Dual Cell: | 0.24 | 70.73 | 97.8 | 52.4 | 39.2 | 8.5 | 1 | 8 | 9 | −29.88 |
| 0018 | KiH no Cys | | | | | | | | | | |
| | in CH3 | | | | | | | | | | |
| p40TL1A- | Dual Cell: | 0.17 | 71.45 | 99.5 | 30.5 | 66.9 | 2.6 | 1 | 6 | 8 | −31.39 |
| 0021 | EE/RR | | | | | | | | | | |
| p40TL1A- | Dual | 0.04 | 70.76 | 98.4 | 23.3 | 72.9 | 3.9 | 1 | 5 | 7 | −31.39 |
| 0024 | Cell: E/R | | | | | | | | | | |
| p40TL1A- | Single | 0.32 | 71.65 | 97.3 | 28.6 | 48.9 | 22.5 | 1 | 13 | 8 | −29.96 |
| 0032 | Cell: KiH, | | | | | | | | | | |
| | S1-S1rev | | | | | | | | | | |
| p40TL1A- | Single | 0.78 | 71.92 | 97.8 | 33 | 61 | 6 | 1 | 7 | 7 | −30.01 |
| 0033 | Cell: KiH, | | | | | | | | | | |
| | modified | | | | | | | | | | |
| | Fd | | | | | | | | | | |

### Example 12: Kinetic Evaluation of p40/TL1A Bispecific Variant Antibodies Binding Human IL-23 and Human TL1A Using Surface Plasmon Resonance

SPR was utilized to determine if the affinity constants of each arm of the bispecific variants binding human IL-23, containing the p40 subunit, and human TL1A were the same for each bispecific approach. For the analysis of the p40 binding arm, kinetic assays were conducted at 37° C. using a collection rate of 10 Hz on a Biacore™ 8 K+ instrument (GE Healthcare). An anti-human IgG antibody (anti-human Fc, GE Healthcare, 22-0648-88) was amine coupled to all four flow cells of a carboxymethylated dextran coated sensor chip (GE Healthcare,) using the manufacturer's recommended protocol. Next, the bispecific variants were immobilized to ˜100 RU and then human IL-23, ranging in concentration from 1.67-45 nM, were injected over the surface. For the analysis of the TL1A binding arm, kinetic assays were conducted at 37° C. using a collection rate of 10 Hz on a Biacore™ T200 instrument (GE Healthcare). For this evaluation, biotinylated recombinant human TL1A was captured on the surface of a Biotin CAPture Sensorchip (GE Healthcare, 28-9202-34) by injecting a concentration of 50 μg/mL for 1 minute at a flow rate of 2 μL/minute to capture density of about 30-50 RU following the manufacturer's protocol. Next, various concentrations of the bispecific variants, ranging from 11.1-300 nM, were injected over the surface. The results show that all bispecific variants have similar affinity values for both cytokines (Table 19).

TABLE 19
Affinity Constants for Bispecific Variant Antibodies Binding Human IL-23 and TL1A
Measured using Surface Plasmon Resonance.

| Bispecific | Affinity constants binding IL-23 | | | Affinity constants binding TL1A | | |
|---|---|---|---|---|---|---|
| variant | ka (1/Ms) | kd (1/s) | K_{D} [pM] | ka (1/Ms) | kd (1/s) | K_{D} [pM] |
| p40TL1A- | 1.73E+06 | 1.56E−03 | 901 | 2.56E+05 | 4.28E−05 | 167.42 ± 8.42 |
| 0001 | | | | | | |
| p40TL1A- | 1.83E+06 | 1.21E−03 | 659 | 2.24E+5 ± 1.55E+3 | 4.87E-5 ± 4.20E−6 | 217.08 ± 17.84 |
| 0018 | | | | | | |
| p40TL1A- | 1.77E+06 | 1.52E−03 | 859 | 3.07E+5 ± 6.05E+3 | 5.60E-5 ± 2.24E−6 | 182.54 ± 4.95 |
| 0021 | | | | | | |
| p40TL1A- | 1.75E+06 | 1.57E−03 | 900 | 2.19E+5 ± 6.68E+3 | 5.99E-5 ± 3.40E−6 | 272.93 ± 7.30 |
| 0024 | | | | | | |
| p40TL1A- | 1.92E+06 | 1.09E−03 | 567 | 2.43E+5 ± 1.13E+4 | 6.15E-5 ± 2.13E−6 | 253.80 ± 19.63 |
| 0032 | | | | | | |
| p40TL1A- | 2.07E+06 | 1.15E−03 | 557 | 2.18E+5 ± 4.32E+3 | 5.52E-5 ± 4.22E−6 | 252.76 ± 15.05 |
| 0033 | | | | | | |

### Example 13: Simultaneous Binding of TL1A and IL-23 Cytokines by p40/TL1A Bispecific Variant Antibodies

SPR was used to determine if the bispecific variants could simultaneously bind TL1A and IL-23, containing the p40 domain. For this analysis, a semi-quantitative SPR method was developed using Biacore™ T200 (GE Healthcare) instrumentation. For this evaluation, biotinylated recombinant human TL1A was captured on the surface of a Biotin CAPture Sensorchip (GE Healthcare, 28-9202-34) to a density of about 30-50 RU following the manufacturer's protocol. The bispecific was injected for 90 seconds at a concentration of 20 μg/m L, a flow rate of 50 μL/minute, and captured to a level of approximately 40-150 RU. Next, recombinant human IL-23 was injected for 60 seconds and a flow rate of 50 μL/minute at a concentration range of 0.37-15 nM. The kinetics of binding of the IL-23 cytokine was calculated and data demonstrates that both cytokines can bind the bispecific simultaneously and to similar affinity values (Table 20).

TABLE 20
Anti-p40/TL1A Bispecific Variants Bind TL1A
and IL-23 Cytokines Simultaneously.

| Bispecific | | | |
|---|---|---|---|
| Variant | ka (1/Ms) | kd (1/s) | K_{D} [pM] |
| p40TL1A-0001 | 5.98E+06 | 5.84E−04 | 90.0 ± 10.7 |
| p40TL1A-0018 | 5.51E+06 | 5.21E−04 | 94.40 |
| p40TL1A-0021 | 7.11E+06 | 5.24E−04 | 73.80 |
| p40TL1A-0024 | 5.32E+06 | 5.71E−04 | 107.00 |
| p40TL1A-0032 | 6.93E+06 | 5.46E−04 | 78.80 |
| p40TL1A-0033 | 6.56E+06 | 5.19E−04 | 79.10 |

### Example 14: Bioactivity Assessment of p40/TL1A Bispecific Variant Antibodies Produced Using Different Bispecific Modalities

To determine whether the different bispecific modality or production method utilized for preparing bispecific molecules adversely impacted the potent cytokine neutralization ability of the binding domains, bioassays were performed to measure bioactivity against each cytokine.

The NF-κB assay, as described above in EXAMPLE 2, was utilized to determine neutralization of human TL1A cytokine activity in cells for bispecific variants. Bioactivity was compared to that of the monovalent bispecific antibody TL1A-0372 that utilizes the same anti-TL1A binding domain. All of the p40TL1A bispecific antibodies demonstrated bioactivity similar to TL1A-0372, within the range of 0.5-1.3-fold (Table 21).

For human IL-12 and IL-23 neutralization assays, a cell-based neutralization assay was utilized to determine neutralization of bispecific variants compared to that of the bivalent p40-0003 antibody. Neutralization activity of the individual Fab binding domain within the bispecific modality should be ˜50% less that the bivalent antibody variant in standard IgG format since binding reflects monovalent versus bivalent neutralization ability. The binding of IL-12 to the IL-12 receptor complex, composed of IL-12R131 and IL-12R132, and the binding of IL-23 to the IL-23 receptor complex, composed of IL-12R131 and IL-23R, lead to receptor complex activation and proximal signaling events that include phosphorylation of STAT4 and STAT3, respectively. Bispecific variants were evaluated for their ability to prevent IL12-induced STAT4 phosphorylation or IL23-induced STAT3 phosphorylation in the KIT-225 T-cell line (National Cancer Institute, NIH), which is an IL-2 dependent cell line derived from human chronic lymphocytic leukemia peripheral blood. KIT-225 cells were cultured in RPMI 1640 media (Invitrogen #21870-076) supplemented with 10% heat inactivated FBS (Gibco #10082-147), 10 mM HEPES (Gibco #15630-080), 1 mM sodium pyruvate (Gibco #11360-070), 1% penicillin/streptomycin (Gibco #15140-122), 100 uM non-essential amino acids (Gibco #11140-050) and 2 mM L-glutamine (Gibco #25030-081). For cytokine stimulations, 110 ng/mL (1.9 nM) of IL-12 was used to induce pSTAT4, and 80 ng/mL (1.5 nM) of IL-23 was used to induce pSTAT3. Cytokines were pre-incubated with a titration of bispecific variants or p40-0003 (0.026 100 nM) for 10 minutes at room temperature. KIT-225 cells (200,000 cells/well in 90 μl) were added to a 96-well assay plate (Citadel #775B-00) and stimulated with 10 μL of the cytokine mixture. The samples were mixed and incubated at 37° C. for 15 minutes. Following incubation, cells were fixed by addition of 300 μL 2% paraformaldehyde (Alfa Aesar #J61899) in phosphate buffered saline to each well and incubated at room temperature for 20 minutes. After fixation, cells were then centrifuged at 1,200 RPM for 5 minutes at room temperature, supernatants aspirated, and cell pellets washed with 800 μL of staining buffer, comprised of 0.5% heat-inactivated fetal bovine serum in PBS. After subsequent centrifugation and aspiration, cell pellets were resuspended in 350 μL of 90% methanol (RICCA Chemical Company #4823-32; pre-chilled at −20° C.) per well and incubated at 4° C. for 30 minutes. Following this incubation, cells were washed with 800 μL staining buffer, and then 150 μL of anti-pSTAT4 (BD #558137; dilution 1:150) or anti-pSTAT3 (BD #557815; dilution 1:150) antibody solution was added to each well, and plates were incubated at 4° C. overnight. Post-incubation, cells were washed and resuspended in staining buffer, and subsequently transferred and filtered into a 96-well plate (Falcon #353077). Flow cytometry analysis was performed on a LSR Fortessa Cell Analyzer (BD #647177) to determine mean fluorescence intensity for each sample. As expected, bispecific variants neutralized human recombinant cytokines IL-12 and IL-23, ˜50% less than the anti-p40 antibody in standard IgG format, p40-0003 and all bispecific variants exhibited similar neutralization of IL-12 or IL-23 (Table 21).

TABLE 21
Neutralization Activity of Bispecific Variants Against Recombinant Human TL1A in NF-κB Assay and
Recombinant Human IL-12 or IL-23 in KIT-225 Assay.

| | | | | KIT-225 STAT3 assay | | | |
|---|---|---|---|---|---|---|---|
| | | NF-κB assay | | IL-23 | | KIT-225 STAT4 assay | |
| | | | Bioactivity | | Bioactivity | IL-12 | |
| | | | relative to TL1A- | IC₅₀ | relative to p40- | IC₅₀ | Bioactivity relative |
| Protein | Modality | IC₅₀ [pM] | 0372 | [nM] | 0003 | [nM] | to p40-0003 |
| TL1A- | Monovalent | 101.5 | 1.0 | N/A | N/A | N/A | N/A |
| 0372 | bispecific | | | | | | |
| | antibody | | | | | | |
| p40-0003 | Bivalent | N/A | N/A | 1.6 | 1.0 | 3.3 | 1.0 |
| | antibody | | | | | | |
| p40TL1A- | Bispecific | 91.1 | 0.9 | 3.9 | 2.4 | 5.7 | 1.7 |
| 0001 | antibody | | | | | | |
| p40TL1A- | Bispecific | 104.9 | 1.0 | 3.4 | 2.1 | 6.6 | 2.0 |
| 0018 | antibody | | | | | | |
| p40TL1A- | Bispecific | 50.9 | 0.5 | 3.4 | 1.4 | 3.0 | 0.9 |
| 0021 | antibody | | | | | | |
| p40TL1A- | Bispecific | 105.9 | 1.0 | 3.6 | 2.2 | 4.3 | 1.3 |
| 0024 | antibody | | | | | | |
| p40TL1A- | Bispecific | 130.6 | 1.3 | 3.8 | 2.3 | 4.6 | 1.4 |
| 0032 | antibody | | | | | | |
| p40TL1A- | Bispecific | 131.2 | 1.3 | 2.3 | 1.4 | 4.8 | 1.4 |
| 0033 | antibody | | | | | | |

N/A = Not Applicable

### Example 15: Pharmacokinetics (PK) of p40/TL1A Bispecific Variant Antibodies in Tg32 Mice

The Tg32 homozygous mouse model was utilized to evaluate the PK properties of the bispecific variant antibodies. This assessment was conducted to gauge how similar the bispecific variants were relative to one another and to established PK properties of LS containing antibodies in the Tg32 model. In brief, 6-10 week-old mice (Jackson Labs, #014565) received a single 5 mg/kg IV dose, and plasma samples were collected over an eight week time interval (4 mice/group). The study was conducted at Pfizer, Inc., and was designed and executed within accordance of the Animal Use Protocol and adherence to the Pfizer institutional animal care and use committee regulations. Quantitative analysis of plasma samples was conducted using a ligand binding assay (total human IgG assay format) developed on the Gyrolab® platform. Sample concentrations are determined by interpolation from a standard curve that is fit using a 4-parameter logistic curve fit with 1/y2 response weighting. Non-compartmental analysis was performed using Watson 7.4 LIMS (Thermo, Inc., Philadelphia, PA). Results from the Tg32 transgenic model demonstrated that the serum half-life was notably shorter for bispecific variant p40TL1A-0001 as compared to the remaining bispecific variants. Further, variants p40TL1A-0018, 0021, 0024, 0032 and 0033 exhibited similar approximate half-lives when compared to an anti-TL1A antibody containing LS mutations. These combined findings would indicate shorter predicted human pharmacokinetic parameters for anti-TL1A antibodies engineered into a one-armed modality (Table 22).

TABLE 22
Terminal Half-lives Determined for LS Engineered
Anti-TL1A One-Armed Variants as Compared to Bivalent
Antibody TL1A-0002 and a Reference Anti-TL1A Antibody
Harboring LS Mutations in Tg32 Mice.

| | | | Terminal |
|---|---|---|---|
| | | | Half-life |
| Sample | Description | Modality | (Days) |
| TL1A Antibody | Bivalent | IgG, LS | 19.1^{A} |
| | antibody | | |
| p40TL1A-0001 | Bispecific | Dual Cell: | 4.9^{B} |
| | antibody | EEE/RRR, LS | |
| p40TL1A-0018 | Bispecific | Dual Cell: | 19.2^{B} |
| | antibody | KiH no Cys in | |
| | | CH3, LS | |
| p40TL1A-0021 | Bispecific | Dual Cell: | 13.8^{B} |
| | antibody | EE/RR, LS | |
| p40TL1A-0024 | Bispecific | Dual Cell: | 10.8^{B} |
| | antibody | E/R, LS | |
| p40TL1A-0032 | Bispecific | Single Cell: | 12.5^{B} |
| | antibody | KiH, S1-S1rev, | |
| | | LS | |
| p40TL1A-0033 | Bispecific | Single Cell: | 17.9^{B} |
| | antibody | KiH, modified | |
| | | Fd, LS | |

^{A}Terminal half-life based on 8-week analysis
^{B}Terminal half-life based on 2-week analysis

### Example 16: Pharmacokinetics (PK) of p40TL1A-0033 in Cynomolgus Monkeys

PK parameters for p40TL1A-0033 bispecific antibody was determined in cynomolgus monkeys. The test article was administered to 6 cynomolgus monkeys as a single IV bolus dose, with dose levels ranging from 0.01 mg/kg to 200 mg/kg. Blood samples were collected pre-dose and post-dose to 1344 hours (20 weeks). The study was conducted at UL Lafayette-New Iberia Research Center (New Iberia, LA) and executed within accordance of the Animal Use Protocol. Quantitative analysis of serum samples from two monkeys dosed at 50 and 200 mg/kg was conducted using two different ligand binding assays developed on the Gyrolab® platform. Biotinylated antibodies against the p40TL1A-0033 anti-TL1A binding domain or biotinylated antibodies against the p40TL1A-0033 anti-p40 binding domain were captured onto streptavidin-coated beads on the affinity capture column of the Gyrolab Bioaffy microstructure and used to bind p40TL1A-0033 in serum samples. Bound p40TL1A-0033 was detected with Alexa-647 labeled Mouse anti-human IgG Fc (Arigo Biolaboratories, catalog number ARG21813). A fluorescent signal on the column, representative of the amount of bound p40TL1A-0033, allowed for visualization of the antibody. Response Units are read by the Gyrolab instrument at 1% Photomultiplier tube (PMT) setting. Sample concentrations are determined by interpolation from a standard curve that is fit using a 5-parameter logistic curve fit with 1/y2 response weighting. Non-compartmental analysis was performed using Watson 7.4 LIMS (Thermo, Inc., Philadelphia, PA). Results from this cynomolgus monkey PK study show that the terminal half-life was 28 days for both ligand binding assays and is consistent with findings from the Tg32 study (Table 23 and Table 22, respectively).

TABLE 23
Terminal Half-life Determined for LS Engineered p40TL1A-
0033 Bispecific Antibody in Cynomolgus Monkey.

| | | | Terminal Half-life |
|---|---|---|---|
| Sample | Description | Modality | (Days) |
| p40TL1A- | Bispecific | Single Cell: | 28 |
| 0033 | Antibody | KiH, modified Fd, | |
| | | LS | |

### Example 17: Kinetic Evaluation of p40TL1A-0033 Bispecific Antibody Binding Human, Cynomolgus Monkey, Rabbit, Mouse and Rat TL1A, IL-12 and IL-23 Using Surface Plasmon Resonance

Cross-species studies using surface plasmon resonance (SPR) characterized the binding of p40TL1A-0033 to human, cynomolgus monkey, rabbit, mouse, and rat TL1A, IL-12 and IL-23 cytokines. In order to determine binding to TL1A, kinetic assays were conducted at 37° C. using a collection rate of 10 Hz on a Biacore™ T200 instrument (GE Healthcare). For this evaluation, biotinylated recombinant TL1A was captured on the surface of a Biotin CAPture Sensorchip (GE Healthcare, 28-9202-34) by injecting a concentration of 50 μg/mL for times ranging from 1-8 minutes depending on the species of TL1A at a flow rate of 10 μL/minute to a capture density of about 30-50 RU following the manufacturer's protocol. Next, various concentrations of the p40TL1A-0033, ranging from 3.7-900 nM, were injected over the surface. The binding affinities of p40TL1A-0033 to each species of TL1A (Table 24) are similar to those of the monovalent bispecific TL1A-0372, harboring the same anti-TL1A binding domain (Table 14). These results indicate that the affinity of the monovalent TL1A binding arm is largely unaffected by the format of the binding arm (modified Fd vs. conventional Fab) or the composition of the other arm in the bispecific (p40 Fab vs non-binding Fab). For the analysis of the p40 binding arm, kinetic assays were conducted at 37° C. using a collection rate of 10 Hz on a Biacore™ 8 K+ instrument (GE Healthcare). An anti-human IgG antibody (anti-human Fc, Jackson Immunoresearch, 109-001-008) was amine coupled to all four flow cells of a carboxymethylated dextran coated sensor chip (GE Healthcare,) using the manufacturer's recommended protocol. Next, p40TL1A-0033 was immobilized to ˜100 RU and then human and cynomolgus monkey IL-12 and IL-23, ranging in concentration from 0.556-15 nM, were injected over the surface. The binding kinetics of human IL-12 and IL-23 to p40TL1A-0033 are similar (Table 24). The binding kinetics of cynomolgus monkey IL-12 and IL-23 to p40TL1A-0033 are similar (Table 24). To determine if rabbit, mouse or rat IL-12 and IL-23 bind p40TL1A-0033 assays were conducted at 37° C. using a collection rate of 10 Hz on a Biacore™ 8 K+ instrument (GE Healthcare). An anti-human IgG antibody (anti-human Fc, Jackson Immunoresearch, 109-001-008) was amine coupled to all four flow cells of a carboxymethylated dextran coated sensor chip (GE Healthcare,) using the manufacturer's recommended protocol. Next, p40TL1A-0033 was immobilized to ˜100 RU and then 200 nM of rabbit, mouse or rat IL-12 (rabbit IL-12 SEQ ID NO: 400 and SEQ ID NO: 401, mouse IL-12 SEQ ID NO: 403 and SEQ ID NO: 404, and rat IL-12 SEQ ID NO: 406 and SEQ ID NO: 407) and IL-23 (rabbit IL-23 SEQ ID NO: 400 and SEQ ID NO: 402, mouse IL-23 SEQ ID NO: 403 and SEQ ID NO: 405, and rat IL-23 SEQ ID NO: 406 and SEQ ID NO: 408) was injected over the surface. No binding was observed; p40TL1A-0033 does not cross-react with rabbit, mouse or rat IL-12 or IL-23.

TABLE 24
Affinity Constants for p40TL1A-0033 Bispecific Antibody Binding
Human, Cynomolgus Monkey, Rabbit Mouse and Rat TL1A, IL-12
and IL-23 Measured using Surface Plasmon Resonance.

| Cytokine | ka (1/Ms) | kd (1/s) | K_{D} | |
|---|---|---|---|---|
| Human TL1A | 1.50E+05 | 5.05E−05 | 0.34 ± 0.04 | nM |
| Cynomolgus monkey | 1.17E+05 | 1.27E−04 | 1.09 ± 0.07 | nM |
| TL1A | | | | |
| Rabbit TL1A | 4.43E+04 | 9.60E−04 | 21.69 ± 0.23 | nM |
| Mouse TL1A | 2.67E+04 | 7.61E−03 | 288.28 ± 32.46 | nM |
| Rat TL1A | 5.24E+04 | 5.62E−02 | *1081.53 ± 94.82 | nM |
| Human IL-12 | 2.10E+06 | 2.58E−04 | 122.56 ± 5.94 | pM |
| Cynomolgus monkey | 2.38E+06 | 6.36E−04 | 267.12 ± 11.89 | pM |
| IL-12 | | | | |
| Rabbit IL-12 | N/A | N/A | NB | |
| Mouse IL-12 | N/A | N/A | NB | |
| Rat IL-12 | N/A | N/A | NB | |
| Human IL-23 | 2.82E+06 | 2.94E−04 | 104.42 ± 6.75 | pM |
| Cynomolgus monkey | 1.93E+06 | 5.42E−04 | 280.8 ± 16.9 | pM |
| IL-23 | | | | |

| | | | |
|---|---|---|---|
| Rabbit IL-23 | N/A | N/A | NB |
| Mouse IL-23 | N/A | N/A | NB |
| Rat IL-23 | N/A | N/A | NB |

*K_{D} value is approximate
N/A = Not Applicable;
NB = No binding

### Example 18: Neutralization Activity of p40/TL1A-0033 Bispecific Antibody Against Human and Cynomolgus Monkey TL1A

The NF-κB assay, as described above in EXAMPLE 2, was utilized to determine neutralization of human and cynomolgus monkey TL1A cytokine activity in cells for p40TL1A-0033 compared to that of monovalent bispecific TL1A-0372 and bivalent anti-TL1A TL1A-0002. Neutralization activity of the individual Fab binding domain within the bispecific modalities, p40TL1A-0033 and TL1A-0372, should be ˜50% less that the bivalent antibody TL1A-0002 since binding reflects monovalent versus bivalent neutralization ability. Results confirmed that p40TL1A-0033 and TL1A-neutralizes TL1A within 2-fold of the TL1A-0002 neutralization value and that neutralization across species is negligible, with absolute fold-changes of 1.7-fold (Table 25).

TABLE 25
Neutralization Activity of p40TL1A-
0033 Against Human and Cyno TL1A.

| | | | Cynomolgus |
|---|---|---|---|
| | | Human TL1A | monkey TL1A |
| | | IC₅₀ ± SD | IC₅₀ ± SD |
| Antibody | Modality | [pM] | [pM] |
| p40TL1A- | Bispecific antibody | 103.7 ± 19.1 | 171.6 ± 21.7 |
| 0033 | | | |
| TL1A-0372 | Monovalent | 106.2 ± 34.3 | 160.4 ± 2.1 |
| | bispecific antibody | | |
| TL1A-0002 | Bivalent antibody | 51.3 ± 20.1 | 85.4 ± 19.9 |

### Example 19: Neutralization Activity of p40/TL1A-0033 Bispecific Antibody Against Human and Cynomolgus Monkey IL-12 and IL-23

Bispecific p40TL1A-0033 was evaluated for its ability to prevent IL12-induced STAT4 phosphorylation or IL23-induced STAT3 phosphorylation in the KIT-225 T-cell line, as described above in EXAMPLE 14. Comparable potencies for human and cyno cytokine neutralization were observed in the respective bioactivity assays for IL-12, and IL-23 (Table 26). Difference in observed potencies for IL-12 and IL-23 neutralization across species were negligible, with absolute fold-changes of 1.2-fold (Table 26).

TABLE 26
Neutralization Activity of p40TL1A-0033
Against Human and Cyno IL-12 and IL-23.

| | Cynomolgus | | Cynomolgus |
|---|---|---|---|
| Human IL-12 | Monkey IL-12 | Human IL-23 | Monkey IL-23 |
| IC₅₀ [nM] | IC₅₀ [nM] | IC₅₀ [nM] | IC₅₀ [nM] |
| 1.110 ± 0.4 | 0.950 ± 0.5 | 3.725 ± 1.3 | 4.344 ± 2.1 |

### Example 20: Bioactivity of p40TL1A-0033 Bispecific Antibody Compared to p40-0003 Antibody in Neutralizing IL-12 Mediated STAT4 Phosphorylation and IL-23 Mediated STAT3 Phosphorylation in Whole Blood

To assess potency of the bispecific p40TL1A-0033 to inhibit proximal signaling of human IL-12 and IL-23 in a primary cell system, the bispecific antibody was evaluated in human whole blood and compared to the p40-0003 antibody. Whole blood provides a primary in vitro system with a complex network of immune cells, including T-cells which respond to IL-12 and IL-23. Whole blood from human donors was stimulated with fixed concentrations of IL-12 or IL-23 diluted in RPMI medium (Gibco #21870-076) in the presence of a titration of p40TL1A-0033 or p40-0003, 0.026-100 nM. For cytokine stimulations, 40 ng/mL (0.68 nM) of IL-12 was used to induce STAT4 activation and 150 ng/mL (2.73 nM) of IL-23 was used to induce STAT3 activation, which is consistent with an EC₈₅ level of activation for each stimulus. Cytokines were pre-incubated with p40TL1A-0033 or p40-0003 for 10 minutes at room temperature. Whole blood (90 μL/well) was added directly into a 96-well plate (Citadel #775B-00). The cytokine mixture (10 μL) was added, samples mixed, and the plate incubated at 37° C. for 20 minutes. Following incubation, 700 μL of 1×BD Lyse/Fix buffer (BD Biosciences, pre-warmed at 37° C.) was added to each well and samples were incubated for 30 minutes at room temperature. Plates were then centrifuged at 1,200 RPM for 5 minutes at room temperature, supernatants aspirated, and cell pellets washed with 800 μL of staining buffer, which is comprised of 0.5% heat-inactivated fetal bovine serum (Gibco #10082-147) diluted in PBS. After subsequent centrifugation and aspiration, cell pellets were resuspended in 350 μL/well of 90% methanol (RICCA Chemical Company #4823-32; pre-chilled at −20° C.) and incubated at 4° C. for 30 minutes. Following this incubation, cells were washed with staining buffer, and then 150 μL of anti-pSTAT4 (BD #558137; dilution 1:150) or anti-pSTAT3 (BD #557815; dilution 1:150) antibody solution was added to each well. Plates were incubated at 4° C. overnight. Post incubation, cells were washed and resuspended in staining buffer, and subsequently transferred and filtered into a 96-well plate (Falcon #353077). Flow cytometry analysis was performed on a LSR Fortessa Cell Analyzer (BD #647177), and mean fluorescence intensity was normalized to % STAT4 or STAT3 activation based on defined positive control (IL-12 or IL-23 treatment alone) and negative control wells (PBS alone).

p40TL1A-0033 inhibited IL-12-mediated STAT4 activation and IL-23-mediated STAT3 activation by ˜2.9-fold decreased potency (IC50) in both STAT activation assays compared to the bivalent anti-p40 mAb p40-0003, which is consistent with reduction in valency of the bispecific (Table 27). Importantly, the values for fold-change in potency (IC50) to p40-0003 observed in the whole blood assays for inhibition of STAT3 and STAT4 activation were similar to those obtained in the KIT-225 T-cell line (Table 21).

TABLE 27
p40TL1A-0033 and p40-0003 Neutralization of Human IL-12 and IL-23 in Whole Blood.

| | | Human IL-12 | | Human IL-23 | |
|---|---|---|---|---|---|
| Sample | Description | IC₅₀ ± SD [nM] | n (donors) | IC₅₀ ± SD [nM] | n (donors) |
| p40TL1A-0033 | Bispecific antibody | 0.720 + 0.600 | 4 | 0.262 + 0.200 | 4 |
| p40-0003 | Bivalent antibody | 12.8 + 4.70 | 4 | 4.51 + 1.80 | 4 |

### Example 21: Bioactivity of p40TL1A-0033 and p40TL1A-0021 Bispecific Antibody Variants in Inhibition of IFN-γ Secretion in Whole Blood

To test potency of bispecific variants in inhibiting pro-inflammatory IFN-γ secretion, a human whole blood assay was utilized in which p40TL1A-0033 and p40TL1A-0021 were titrated in presence of inflammatory factors including plate-bound immune-complex and LPS to induce endogenous release of human TL1A and IL-12, respectively. The assay design provides a complex cellular network marked by activation of blood monocytes by cross-linked antibodies and bacterial peptide resulting in release of endogenous TL1A and IL-12, which then serve to augment release of IFN-γ by T-cells and NK/NKT cells. To form immune complexes for induction of endogenous TL1A release from monocytes, 96 well polypropylene tissue culture microplates (Corning, Catalog number 353072) were coated with 50 μL of unconjugated human IgG (Jackson ImmunoResearch, Catalog number 009-000-003) diluted in PBS free of Ca²⁺ and Mg²⁺ (PBS-CMF) at a final concentration of 0.5 mg/mL and incubated at 37° C. for 1 hour, static. Plates were washed three times with 150 μL of PBS-CMF per well. Addition of 50 μL of anti-human IgG (Jackson ImmunoResearch, Catalog number 209-005-082) diluted in PBS-CMF at a final concentration of 0.02 mg/mL was performed to cross-link the secondary antibody to the human IgG coated wells. Formation of immune complexes was prepared by incubating plates at 37° C. for 1 hour, static. Remaining unbound anti-human IgG was washed away with 150 μL of PBS-CMF per well, repeated three times, then removed right before addition of human peripheral blood. For the IFN-γ release assay, human whole blood collected in sodium heparin spray-coated vacutainers (BD, Catalog number 367874) was distributed (185 μL per well) to the 96-well plates containing immune complex utilizing only the inner wells to avoid plate edge effects. T-cell receptor (TCR) activation was performed using anti-human CD3, clone UCHT1 (BD Biosciences #555329) in combination with anti-human CD28, clone 9.3 (BioXcell BE0248) prepared in 40 mM glucose in PBS-CMF buffer and added to whole blood (5 μL/well). Ultrapure LPS (from E. Coli 0111:B4, InvivoGen, Catalog number tlrl-3-pelps) diluted in endotoxin-free water was also prepared in 40 mM glucose in PBS-CMF buffer and added to the blood (5 μL/well). Final assay concentrations for anti-human CD3/28 and LPS were 100 ng/mL and 10 ng/mL, respectively. Human whole blood containing the activation stimuli was properly mixed using a 96 well standard pin replicator (Scinomix, Catalog number SCI-4010-OS). After addition of the activation stimuli, half-log serial dilutions of human IgG1 isotype control and the bispecific variant antibodies were prepared in PBS-CMF then added (5 μL/well) to the blood to obtain a final concentration response curve ranging from 0.019-60 nM. The blood was then mixed again with pin replicator and incubated with the corresponding stimuli conditions at 37° C. for 24 hours. Following the 24-hour incubation, the blood was centrifuged at 2,000×g for 10 minutes at 4° C. Plasma was harvested and assayed for human IFN-γ detection at a 1:500 dilution using the Meso Scale Discovery Human IFN-γ VPLEX Kit (MSD, Catalog number K151QOD-2). Samples were analyzed using the MSD SECTOR Imager 6000 to quantitate IFN-γ concentrations. Levels of IFN-γ were extrapolated from standard curve conversions to pg/mL units. Both bispecific variants p40TL1A-0033 and p40TL1A-0021 inhibited IFN-γ release from human whole blood with comparable potency (Table 28).

TABLE 28
p40TL1A-0033 and p40TL1A-0021 Inhibition of
IFN-γ Secretion in Human Whole Blood Assay.

| | IC₅₀ + SD | | |
|---|---|---|---|
| Sample | [nM] | Donors | Replicates |
| p40TL1A- | 1.39 + 0.840 | 4 | 6 |
| 0033 | | | |
| p40TL1A- | 0.619 + 0.283 | 3 | 5 |
| 0021 | | | |

### Example 22. Bioactivity of p40TL1A-0033 and p40TL1A-0021 Bispecific Antibody Variants in Inhibition of IFN-γ Secretion from Isolated Human CD4⁺ T-Cells

To evaluate the potency of the bispecific variants to inhibit IFN-γ release from human T-cells, a 5-fold titration of antibodies, 0.0005-40 nM, was tested in presence of recombinant human cytokines TL1A (R&D Systems, Catalog number 1319-TL-010/CF), IL-12 (R&D Systems, Catalog number 219-IL-005/CF), IL-15 (R&D Systems, Catalog number 247-ILB-025/CF), and IL-18 (R&D Systems, Catalog number 9124-IL-050/CF) in a CD4+ T-cell cytokine release assay. Isolation of CD4+ T-cells was performed from human peripheral blood leukopaks (STEMCELL Technologies, Catalog number 70500.1). Blood was spun down at 400×g for 10 minutes, and resultant cell pellet was re-suspended with 50 mL of PBS buffer and spun down. Red blood cells were lysed using ACK Lysing Buffer (Gibco, Catalog number A10492-01) for 5 minutes at room temperature and then rinsed twice with PBS. The CD4+ T cells were then isolated and purified using the EasySep Human CD4+ T cell isolation kit (STEMCELL Technologies, Catalog number 17952). Cells were plated in a 96 well plate (Corning, Catalog number 353072) at a density of 2×10⁵ cells per well, cultured in RPMI-1640 medium (Gibco, Catalog number, 11875-093) containing 10% human serum (Sigma, Catalog number H4522-100ML), 1× Pen/Strep antibiotic (Gibco, Catalog number 15070063), and 10 μg/mL of IL-2 (PeproTech, Catalog number 200-02-100UG). For activation and assessment of potency, cells were incubated with human recombinant TL1A (648 pM monomer; molar equivalent to 216 pM trimer), IL-12 (7.3 pM), IL-15 (23.5 pM), and IL-18 (128 pM) in the presence or absence of bispecific antibodies for 24 hours. After incubation of 24 hours, the cell supernatant was collected and assayed for human IFN-γ detection at a 1:100 dilution using the Meso Scale Discovery (MSD) assay system kits (MSD, Catalog number K151Q0D-4), quantitated for IFN-γ concentration using the MSD SECTOR Imager 6000. Levels of IFN-γ were extrapolated from a standard curve and converted to pg/mL units. Bispecific variants p40TL1A-0033 and p40TL1A-0021 demonstrated potent inhibition of IFN-γ secretion mediated by human TL1A and IL-12, in combination with IL-15 and IL-18 in primary CD4+ T-cells (Table 29).

TABLE 29
p40TL1A-0033 and -0021 Inhibition of IFN-γ Secretion
in Isolated Human CD4+ T-cells.

| Sample | IC₅₀ + SD [nM] | Donors | Replicates |
|---|---|---|---|
| p40TL1A- | 0.0810 ± 0.003 | 3 | 5 |
| 0033 | | | |
| p40TL1A- | 0.0650 ± 0.0130 | 5 | 5 |
| 0021 | | | |

### Example 23. Transient Expression, Purification, and Characterization of KiH mFd Anti-p40/TL1A Bispecific Format Variants

Three additional KiH mFd bispecific antibodies were constructed to examine the impact of both anti-TL1A and anti-p40 binding domains and orientations of the heterodimerization KiH mutations on the Fc also containing the previously described mutations to minimize effector function and extend serum half-life to offer increased dosing flexibility (Table 30).

TABLE 30
Description of the p40TL1A KiH mFd Bispecific Antibodies.

| | | Light | | |
|---|---|---|---|---|
| Name | Heavy Chain | Chain | VL-CL-Fc | VH-CH1 |
| p40TL1A-0033 | p40 with knob | p40 | TL1A with hole | TL1A |
| | mutations | | mutations | |
| p40TL1A-0040 | p40 with hole | p40 | TL1A with knob | TL1A |
| | mutations | | mutations | |
| p40TL1A-0041 | TL1A with | TL1A | p40 with hole | p40 |
| | knob | | mutations | |
| | mutations | | | |
| p40TL1A-0042 | TL1A with | TL1A | p40 with knob | p40 |
| | hole | | mutations | |
| | mutations | | | |

The p40TL1A bispecific molecules described above were transiently expressed using Expi293F™ host cells with the manufacturer's recommended protocol and 2 L of conditioned medium containing these p40TL1A bispecific proteins was produced. The general method for purifying single cell derived bispecific variants uses a three-column step: MabSelect™ SuRe™ LX (Cytiva Life Sciences), followed by Mono-S Cation Exchange (Cytiva Life Sciences), and Superdex 200 gel filtration (Cytiva Life Sciences) with the final formulation in PBS-CMF pH 7.2. The expression titer, final yield purified and percent of the expressed material that was purified are reported in Table 31. Results indicate that the expression titers were higher for the bispecific variants with anti-p40 mFd binding domains, p40TL1A-0041 and p40TL1A-0042, however, the amount of expressed protein that was purified was highest for p40TL1A-0033.

TABLE 31
Expression titer, final yield purified and percent
of the expressed material that was purified.

| | Expression | Final Yield | % expressed |
|---|---|---|---|
| Name | titer (mg/L) | (mg/L) | material purified |
| p40TL1A-0033 | 38.2 | 9.47 | 24.8% |
| p40TL1A-0040 | 41.4 | 3.28 | 7.92% |
| p40TL1A-0041 | 123.6 | 7 | 5.56% |
| p40TL1A-0042 | 187.5 | 20.28 | 10.8% |

Purified anti-p40/TL1A bispecific variants were subjected to extensive characterization to evaluate bioanalytical and biophysical properties of these complex molecules. In particular, the following methods were used to assess key molecular properties described in EXAMPLE 2: analytical size-exclusion chromatography (aSEC) to determine percent high molecular mass species (HMMS) or low molecular mass species (LMMS) as an indicator of aggregation or fragmentation respectively, thermal stability using Differential Scanning calorimetry (DSC), non-reduced cGE to assess percent peak of interest (POI), imaged capillary electrophoresis (iCE) for unstressed sample (T0) to evaluate charge heterogeneity, non-specificity evaluation and in silico immunogenicity assessment.

The summary of results for the biophysical and bioanalytical evaluation of the anti-p40/anti-TL1A variant bispecific antibody variants generated indicate that all generally have favorable molecular properties comparable to standard well-behaved antibodies (Table 32). The propensity to aggregate was deemed low for all four bispecific variants by analytical SEC analysis. All variants exhibit good thermal stability with Tm1 values >65° C. Integrity of intact bispecific is >97% for all variants as determined with non-reducing cGE assay. All bispecific variants present acceptable non-specificity scores.

TABLE 32
Summary of Key Biophysical and Bioanalytical Properties Evaluated for mFd KiH Bispecific Variants.

| | | | DSC | | | | | | | |
|---|---|---|---|---|---|---|---|---|---|---|
| | aSEC | | Tm1 | cGE % | iCE T0 | | | Non-specificity Scores | | |
| Bispecific Variant | % HMMS | % LMMS | (° C.) | Intact NR | % acidic | % main | % basic | AC-SINS | DNA | Insulin |
| p40TL1A-0033 | 0.44 | 0.01 | 72.48 | 97.8 | 29.4 | 63.6 | 7.0 | 1.1 | 5.9 | 8.12 |
| p40TL1A-0040 | 1.10 | BLOQ | 72.16 | 97.6 | 25.7 | 68.2 | 6.1 | 1.1 | 12.16 | 13.17 |
| p40TL1A-0041 | 0.21 | 0.02 | 72.02 | 97.1 | 27.8 | 66.7 | 5.5 | 1.1 | 4.9 | 6.12 |
| p40TL1A-0042 | 0.15 | 0.01 | 72.00 | 97.2 | 29.3 | 64.9 | 5.8 | 1.1 | 4.11 | 7.13 |

BLOQ = below limit of quantification

As described above in EXAMPLE 12, SPR was utilized to determine if the affinity constants of each arm of the bispecific variants binding human IL-23, containing the p40 subunit, and human TL1A were the same for each bispecific variant. The results show that all bispecific variants have similar affinity values for both cytokines (Table 33).

TABLE 33
Affinity Constants for Bispecific Variant Antibodies Binding Human IL-23 and TL1A Measured
using Surface Plasmon Resonance.

| Bispecific | Affinity constants binding IL-23 | | | Affinity constants binding TL1A | | |
|---|---|---|---|---|---|---|
| variant | ka (1/Ms) | kd (1/s) | K_{D} [pM] | ka (1/Ms) | kd (1/s) | K_{D} [pM] |
| p40TL1A-0033 | 2.28E+06 | 5.60E−04 | 194 | 1.32E+05 | 8.21E−05 | 622 |
| p40TL1A-0040 | 2.83E+06 | 5.50E−04 | 194 | 1.31E+05 | 8.22E−05 | 627 |
| p40TL1A-0041 | 2.29E+06 | 4.85E−04 | 213 | 1.31E+05 | 7.56E−05 | 577 |
| p40TL1A-0042 | 2.35E+06 | 4.88E−04 | 207 | 1.77E+05 | 7.42E−05 | 419 |

To determine whether if the format of different bispecific variants adversely impacted the potent TL1A neutralization ability of the binding domains, the NF-κB assay, as described above in EXAMPLE 2 (adapted to a 384-well format), was utilized to determine neutralization of human TL1A cytokine activity in cells for bispecific variants. The TF-1 reporter cells were added to a 384-well plate (25,000 cells per well) and stimulated with a fixed concentration of recombinant human TL1A (2 nM) in the presence of the bispecific variants titrated to a range of 2-20000 pM in a total volume of 40 μL and then incubated at 37° C. for 24 hours. Following this incubation, 40 μL of Beetle Luciferin (Promega E1601) was added to the wells for a final luciferin concentration of 150 μg/mL and incubated at 37° C. for 30 minutes. Luminescence was read on Envision (Perkin-Elmer) for NF-κB activity. The results show that all bispecific variants have similar bioactivity (Table 34).

TABLE 34
Neutralization Activity of Bispecific Variants
Against Recombinant Human TL1A in NF-κB Assay.

| Bispecific variant | NF-κB assay IC₅₀ [nM] |
|---|---|
| p40TL1A-0033 | 0.31 |
| p40TL1A-0040 | 0.27 |
| p40TL1A-0041 | 0.32 |
| p40TL1A-0042 | 0.32 |

### REFERENCES

1. Arch R, Zhang J U N, Madar M, Ishino T, Bard J, Finlay W, Cunningham O, Reilly C, Brams P, Devaux B, et al. Tumor necrosis factor-like ligand 1A specific antibodies and compositions and uses thereof. US: PFIZER and BRISTOL-MYERS SQUIBB COMPANY, 2017.
2. De Groot A S, Cousens L, Mingozzi F, Martin W. Tregitope Peptides: The Active Pharmaceutical Ingredient of IVIG? Clinical and Developmental Immunology 2013; 2013:493138.
3. Avery L B, Wade J, Wang M, Tam A, King A, Piche-Nicholas N, Kavosi M S, Penn S, Cirelli D, Kurz J C, et al. Establishing in vitro in vivo correlations to screen monoclonal antibodies for physicochemical properties related to favorable human pharmacokinetics. MAbs 2018; 10:244-55.
4. Liu Y, Caffry I, Wu J, Geng S B, Jain T, Sun T, Reid F, Cao Y, Estep P, Yu Y, et al. High-throughput screening for developability during early-stage antibody discovery using self-interaction nanoparticle spectroscopy. mAbs 2014; 6:483-92.
5. Junker F, Gordon J, Qureshi O. Fc Gamma Receptors and Their Role in Antigen Uptake, Presentation, and T Cell Activation. Frontiers in Immunology 2020; 11.
6. Ridgway J B, Presta L G, Carter P. ‘Knobs-into-holes’ engineering of antibody CH3 domains for heavy chain heterodimerization. Protein Eng 1996; 9:617-21.
7. Zalevsky J, Chamberlain A K, Horton H M, Karki S, Leung I W L, Sproule T J, Lazar G A, Roopenian D C, Desjarlais J R. Enhanced antibody half-life improves in vivo activity. Nature biotechnology 2010; 28:157-9.
8. Avery L B, Wang M, Kavosi M S, Joyce A, Kurz J C, Fan Y-Y, Dowty M E, Zhang M, Zhang Y, Cheng A, et al. Utility of a human FcRn transgenic mouse model in drug discovery for early assessment and prediction of human pharmacokinetics of monoclonal antibodies. mAbs 2016; 8:1064-78.
9. Ko S-Y, Pegu A, Rudicell R S, Yang Z-y, Joyce M G, Chen X, Wang K, Bao S, Kraemer T D, Rath T, et al. Enhanced neonatal Fc receptor function improves protection against primate SHIV infection. Nature 2014; 514:642-5.
10. Nnane I P, Han C, Jiao Q, Tam S H, Davis H M, Xu Z. Modification of the Fc Region of a Human Anti-oncostatin M Monoclonal Antibody for Higher Affinity to FcRn Receptor and Extension of Half-life in Cynomolgus Monkeys. Basic & Clinical Pharmacology & Toxicology 2017; 121:13-21.
11. Giles-Komar J, Knight David M, Peritt D, Scallon B, Shealy D. Anti-IL-12 antibodies and compositions thereof. US: CENTOCOR INC, 2005.
12. Danese S, Klopocka M, Scherl E J, Romatowski J, Allegretti J R, Peeva E, Vincent M S, Schoenbeck U, Ye Z, Hassan-Zahraee M, et al. Anti-TL1A Antibody P F-06480605 Safety and Efficacy for Ulcerative Colitis: A Phase 2a Single-Arm Study. Clin Gastroenterol Hepatol 2021; 19:2324-32.e6.
13. Hassan-Zahraee M, Ye Z, Xi L, Baniecki M L, Li X, Hyde C L, Zhang J, Raha N, Karlsson F, Quan J, et al. Antitumor Necrosis Factor-like Ligand 1A Therapy Targets Tissue Inflammation and Fibrosis Pathways and Reduces Gut Pathobionts in Ulcerative Colitis. Inflammatory Bowel Diseases 2021.
14. Ho W, Pons J, Rajpal A, Strop P. HETERODIMERIC PROTEINS AND METHODS FOR PRODUCING AND PURIFYING THEM. WO: RINAT NEUROSCIENCE CORP, 2011.
15. Strop P, Ho W H, Boustany L M, Abdiche Y N, Lindquist K C, Farias S E, Rickert M, Appah C T, Pascua E, Radcliffe T, et al. Generating bispecific human IgG1 and IgG2 antibodies from any antibody pair. J Mol Biol 2012; 420:204-19.
16. Bennett Eric M, Gimeno Ruth E, Higginson-Scott N, Marquette Kimberly A, Paulsen Janet E, Tchistiakova L. BISPECIFIC ANTIBODIES. WO: PFIZER, 2015.
17. Rubin et al, 2019 Am J Gastroenterol; 114:384-413.
18. Lichtenstein et al 2018 Am J Gastroenterol; 113:481-517.
19. Wallace et al, 2014, World J Gastroenterol; 20:6-21.
20. Migone et al, 2002, Immunity 16(3):479-492.
21. Prehn et al, 2004, Clin. Immunol. 112(1):66-77.
22. Shih et al, 2009, Eur J Immunol 39(11):3239-3250.
23. Papadakis et al, 2004, J Immunol 172(11):7002-7007
24. Papadakis et al, 2005, J Immunol 174(8):4985-4990.
25. Pappu et al, 2008, J Exp Med 205(5):1049-1062.
26. Takedatsu et al, 2008, Gastroenterology 135(2):552-567.
27. Meylan et al., 2008, Immunity 29(1):79-89.
28. Hsu and Viney, 2011, Mucosal Immun. 4(4):368-370.
29. Yamazaki et al, 2005. Hum Mol Genet 14(22):3499-3506.
30. Barrett et al, 2008, Nat Genet 40(8):955-962.
31. Kakuta et al, 2009, Hum Mol Genet 18(6):1089-1098.
32. Jostins et al, 2012, Nature 491(7422):119-124.
33. Yamazaki et al, 2013, Gastroenterology 144(4):781-788.
34. Bamias et al, 2003, J Immunol 171(9):4868-4874.
35. Bamias et al, 2006, Proc Natl Acad Sci USA 103(22):8441-8446.
36. Kamada et al, 2010, Inflamm Bowel Dis 16(4):568-575.
37. Meylan et al, 2011, Immunol Rev 244(1):188-196.
38. Taraban et al, 2011, Mucosal Immunol 4(2):186-196.
39. Bamias et al, 2012, Dig Liver Dis. 44(1):30-36.

TABLE 30
TL1A and p40 Antibody Variants Sequence Listing

| | Heavy Chain (HC) | | | | | | | | | | Light Chain (LC) | | | | | | |
|---|---|---|---|---|---|---|---|---|---|---|---|---|---|---|---|---|---|
| | HC | HC | HC | | | | | | | | LC | LC | LC | | | | |
| Antibody | CDR1 | CDR2 | CDR3 | JH | V_{H} | CH1 | HINGE | CH2 | CH3 | HC | CDR1 | CDR2 | CDR3 | JK | V_{L} | CL | LC |
| TL1A-0002 | 1 | 2 | 3 | 4 | 5 | 6 | 7 | 8 | 9 | 10 | 11 | 12 | 13 | 14 | 15 | 16 | 17 |
| TL1A-0042 | 1 | 2 | 3 | 4 | 5 | 6 | 7 | 8 | 9 | 10 | 11 | 12 | 13 | 14 | 18 | 16 | 19 |
| TL1A-0043 | 1 | 2 | 3 | 4 | 5 | 6 | 7 | 8 | 9 | 10 | 11 | 12 | 13 | 14 | 20 | 16 | 21 |
| TL1A-0045 | 1 | 2 | 3 | 4 | 22 | 6 | 7 | 8 | 9 | 23 | 11 | 12 | 13 | 14 | 15 | 16 | 17 |
| TL1A-0047 | 1 | 2 | 3 | 4 | 24 | 6 | 7 | 8 | 9 | 25 | 11 | 12 | 13 | 14 | 15 | 16 | 17 |
| TL1A-0048 | 1 | 2 | 3 | 4 | 26 | 6 | 7 | 8 | 9 | 27 | 11 | 12 | 13 | 14 | 15 | 16 | 17 |
| TLIA-0049 | 1 | 2 | 3 | 4 | 22 | 6 | 7 | 8 | 9 | 23 | 11 | 12 | 13 | 14 | 18 | 16 | 19 |
| TL1A-0051 | 1 | 2 | 3 | 4 | 24 | 6 | 7 | 8 | 9 | 25 | 11 | 12 | 13 | 14 | 18 | 16 | 19 |
| TL1A-0052 | 1 | 2 | 3 | 4 | 26 | 6 | 7 | 8 | 9 | 27 | 11 | 12 | 13 | 14 | 18. | 16 | 19 |
| TL1A-0053 | 1 | 2 | 3 | 4 | 22 | 6 | 7 | g | 9 | 23 | 11 | 12 | 13 | 14 | 20 | 16 | 21 |
| TL1A-0055 | 1 | 2 | 3 | 4 | 24 | 6 | 7 | 8 | 9 | 25 | 11 | 12 | 13 | 14 | 20 | 16 | 21 |
| TL1A-0056 | 1 | 2 | 3 | 4 | 26 | 6 | 7 | 8 | 9 | 27 | 11 | 12 | 13 | 14 | 20 | 16 | 21 |
| TL1A-0167 | 1 | 2 | 3 | 4 | 28 | 6 | 7 | 8 | 9 | 29 | 11 | 12 | 13 | 14 | 15 | 16 | 17 |
| TL1A-0256 | 30 | 31 | 32 | 4 | 33 | 6 | 7 | 8 | 9 | 34 | 11 | 12 | 13 | 14 | 18 | 16 | 19 |
| TL1A-0257 | 30 | 31 | 35 | 4 | 36 | 6 | 7 | 8 | 9 | 37 | 11 | 12 | 13 | 14 | 18 | 16 | 19 |
| TL1A-0258 | 30 | 38 | 32 | 4 | 39 | 6 | 7 | 8 | 9 | 40 | 11 | 12 | 13 | 14 | 18 | 16 | 19 |
| TL1A-0260 | 30 | 41 | 32 | 4 | 42 | 6 | 7 | 8 | 9 | 43 | 11 | 12 | 13 | 14 | 18 | 16 | 19 |
| TL1A-0261 | 30 | 41 | 35 | 4 | 44 | 6 | 7 | 8 | 9 | 45 | 11 | 12 | 13 | 14 | 18 | 16 | 19 |
| TL1A-0262 | 30 | 46 | 32 | 4 | 47 | 6 | 7 | 8 | 9 | 48 | 11 | 12 | 13 | 14 | 18 | 16 | 19 |
| TL1A-0263 | 30 | 46 | 35 | 4 | 49 | 6 | 7 | 8 | 9 | 50 | 11 | 12 | 13 | 14 | 18 | 16 | 19 |
| TL1A-0264 | 30 | 51 | 32 | 4 | 52 | 6 | 7 | 8 | 9 | 53 | 11 | 12 | 13 | 14 | 18 | 16 | 19 |
| TL1A-0265 | 30 | 51 | 35 | 4 | 54 | 6 | 7 | 8 | 9 | 55 | 11 | 12 | 13 | 14 | 18 | 16 | 19 |
| TL1A-0266 | 30 | 56 | 32 | 4 | 57 | 6 | 7 | 8 | 9 | 58 | 11 | 12 | 13 | 14 | 18 | 16 | 19 |
| TL1A-0267 | 30 | 56 | 35 | 4 | 59 | 6 | 7 | 8 | 9 | 60 | 11 | 12 | 13 | 14 | 18 | 16 | 19 |
| TL1A-0270 | 61 | 31 | 32 | 4 | 62 | 6 | 7 | 8 | 9 | 63 | 11 | 12 | 13 | 14 | 18 | 16 | 19 |
| TL1A-0271 | 61 | 31 | 35 | 4 | 64 | 6 | 7 | 8 | 9 | 85 | 11 | 12 | 13 | 14 | 18 | 16 | 19 |
| TL1A-0272 | 61 | 38 | 32 | 4 | 66 | 6 | 7 | 8 | 9 | 67 | 11 | 12 | 13 | 14 | 18 | 16 | 19 |
| TL1A-0273 | 61 | 38 | 35 | 4 | 68 | 6 | 7 | 8 | 9 | 69 | 11 | 12 | 13 | 14 | 18 | 16 | 19 |
| TL1A-0274 | 61 | 41 | 32 | 4 | 70 | 6 | 7 | 8 | 9 | 71 | 11 | 12 | 13 | 14 | 18 | 16 | 19 |
| TL1A-0275 | 61 | 41 | 35 | 4 | 72 | 6 | 7 | 8 | 9 | 73 | 11 | 12 | 13 | 14 | 18 | 16 | 19 |
| TLIA-0276 | 61 | 46 | 32 | 4 | 74 | 6 | 7 | 8 | 9 | 75 | 11 | 12 | 13 | 14 | 18 | 16 | 19 |
| TL1A-0277 | 61 | 46 | 35 | 4 | 76 | 6 | 7 | 8 | 9 | 77 | 11 | 12 | 13 | 14 | 18 | 16 | 19 |
| TL1A-0278 | 61 | 51 | 32 | 4 | 78 | 6 | 7 | 8 | 9 | 79 | 11 | 12 | 13 | 14 | 18 | 16 | 19 |
| TL1A-0279 | 61 | 51 | 35 | 4 | 80 | 6 | 7 | 8 | 9 | 81 | 11 | 12 | 13 | 14 | 18 | 16 | 19 |
| TL1A-0280 | 61 | 56 | 32 | 4 | 82 | 6 | 7 | 8 | 9 | 83 | 11 | 12 | 13 | 14 | 18 | 16 | 19 |
| TL1A-0281 | 61 | 56 | 35 | 4 | 84 | 6 | 7 | 8 | 9 | 85 | 11 | 12 | 13 | 14 | 18 | 16 | 19 |
| TL1A-0284 | 86 | 31 | 32 | 4 | 87 | 6 | 7 | 8 | 9 | 88 | 11 | 12 | 13 | 14 | 18 | 16 | 19 |
| TL1A-0285 | 86 | 31 | 35 | 4 | 89 | 6 | 7 | 8 | 9 | 90 | 11 | 12 | 13 | 14 | 18 | 16 | 19 |
| TL1A-0286 | 86 | 38 | 32 | 4 | 91 | 6 | 7 | 8 | 9 | 92 | 11 | 12 | 13 | 14 | 18 | 16 | 19 |
| TL1A-0287 | 86 | 38 | 35 | 4 | 93 | 6 | 7 | 8 | 9 | 94 | 11 | 12 | 13 | 14 | 18 | 16 | 19 |
| TL1A-0288 | 86 | 41 | 32 | 4 | 95 | 6 | 7 | 8 | 9 | 98 | 11 | 12 | 13 | 14 | 18 | 16 | 19 |
| TL1A-0289 | 86 | 41 | 35 | 4 | 97 | 6 | 7 | 8 | 9 | 98 | 11 | 12 | 13 | 14 | 18 | 16 | 19 |
| TL1A-0291 | 86 | 46 | 35 | 4 | 99 | 6 | 7 | 8 | 9 | 100 | 11 | 12 | 13 | 14 | 18 | 16 | 19 |
| TL1A-0292 | 86 | 51 | 32 | 4 | 101 | 6 | 7 | 8 | 9 | 102 | 11 | 12 | 13 | 14 | 18 | 16 | 19 |
| TL1A-0293 | 86 | 51 | 35 | 4 | 103 | 6 | 7 | 8 | 9 | 104 | 11 | 12 | 13 | 14 | 18 | 16 | 19 |
| TL1A-0294 | 86 | 56 | 32 | 4 | 105 | 6 | 7 | 8 | 9 | 106 | 11 | 12 | 13 | 14 | 18 | 16 | 19 |
| TLIA-0295 | 86 | 56 | 35 | 4 | 107 | 6 | 7 | 8 | 9 | 108 | 11 | 12 | 13 | 14 | 18 | 16 | 19 |
| TL1A-0303 | 109 | 41 | 35 | 4 | 110 | 6 | 7 | 8 | 9 | 111 | 11 | 12 | 13 | 14 | 18 | 16 | 19 |
| TL1A-0304 | 109 | 46 | 32 | 4 | 112 | 6 | 7 | 8 | 9 | 113 | 11 | 12 | 13 | 14 | 18 | 16 | 19 |
| TL1A-0305 | 109 | 46 | 35 | 4 | 114 | 6 | 7 | 8 | 9 | 115 | 11 | 12 | 13 | 14 | 18 | 16 | 19 |
| TL1A-0306 | 109 | 51 | 32 | 4 | 116 | 6 | 7 | 8 | 9 | 117 | 11 | 12 | 13 | 14 | 18 | 16 | 19 |
| TL1A-0307 | 109 | 51 | 35 | 4 | 118 | 6 | 7 | 8 | 9 | 119 | 11 | 12 | 13 | 14 | 18 | 16 | 19 |
| TL1A-0308 | 109 | 56 | 32 | 4 | 124 | 6 | 7 | 8 | 9 | 121 | 11 | 12 | 13 | 14 | 18 | 16 | 19 |
| TL1A-0309 | 109 | 56 | 35 | 4 | 122 | 6 | 7 | 8 | 9 | 123 | 11 | 12 | 13 | 14 | 18 | 16 | 19 |
| TL1A-0324 | 1 | 124 | 125 | 4 | 126 | 6 | 7 | 8 | 9 | 127 | 11 | 12 | 13 | 14 | 18 | 16 | 19 |
| [xTL1A]-0336 | 128 | 129 | 130 | 4 | 131 | 6 | 7 | 8 | 9 | 132 | 11 | 12 | 13 | 14 | 18 | 16 | 19 |
| [xTL1A]-0338 | 128 | 129 | 133 | 4 | 134 | 6 | 7 | 8 | 9 | 135 | 11 | 12 | 13 | 14 | 18 | 16 | 19 |
| [xTL1A]-0341 | 128 | 129 | 136 | 1 | 137 | 6 | 7 | 8 | 9 | 138 | 11 | 12 | 13 | 14 | 18 | 36 | 19 |
| [xTL1A]-0347 | 139 | 140 | 130 | 1 | 141 | 6 | 7 | 8 | 9 | 142 | 11 | 12 | 13 | 14 | 18 | 16 | 19 |
| [xTL1A]-0349 | 139 | 140 | 133 | 4 | 143 | 6 | 7 | 8 | 9 | 144 | 11 | 12 | 13 | 14 | 18 | 16 | 19 |
| [xTL1A]-0352 | 139 | 140 | 136 | 4 | 145 | 6 | 7 | 8 | 9 | 146 | 11 | 12 | 13 | 14 | 18 | 16 | 19 |
| [xTL1A]-0358 | 147 | 148 | 130 | 4 | 149 | 6 | 7 | 8 | 9 | 150 | 11 | 12 | 13 | 14 | 18 | 16 | 19 |
| [xTL1A]-0360 | 147 | 148 | 133 | 4 | 151 | 6 | 7 | 8 | 9 | 152 | 11 | 12 | 13 | 14 | 18 | 16 | 19 |
| [xTL1A]-0363 | 147 | 148 | 136 | 4 | 153 | 6 | 7 | 8 | 9 | 154 | 11 | 12 | 13 | 14 | 18 | 16 | 19 |
| p40-0003 | 167 | 168 | 169 | 170 | 171 | 6 | 7 | 8 | 9 | 172 | 173 | 174 | 175 | 176 | 177 | 16 | 178 |

TABLE 31
p40TL1A Bispecific Variants.

| | p40TL1A Bispecific Variant | | | | | |
|---|---|---|---|---|---|---|
| Domain | p40TL1A-0001 | p40TL1A-0018 | p40TL1A-0021 | p40TL1A-0024 | p40TL1A-0032 | p40TL1A-0033 |
| p40 HC CDR1 | 167 | 167 | 167 | 167 | 167 | 167 |
| p40 HC CDR2 | 168 | 168 | 168 | 168 | 168 | 168 |
| p40 HC CDR3 | 169 | 169 | 169 | 169 | 169 | 169 |
| p40 JH | 170 | 170 | 170 | 170 | 170 | 170 |
| p40 V_{H} | 171 | 171 | 171 | 171 | 171 | 171 |
| p40 CH1 | 6 | 6 | 6 | 6 | 195 | 6 |
| p40 HINGE | 179 | 7 | 189 | 7 | 7 | 7 |
| p40 CH2 | 8 | 8 | 8 | 8 | 8 | 8 |
| p40 CH3 | 180 | 185 | 180 | 180 | 160 | 160 |
| p40 HC | 181 | 186 | 190 | 193 | 196 | 203 |
| p40 LC CDR1 | 173 | 173 | 173 | 173 | 173 | 173 |
| p40 LC CDR2 | 174 | 174 | 174 | 174 | 174 | 174 |
| p40 LC CDR3 | 175 | 175 | 175 | 175 | 175 | 175 |
| p40 JK | 176 | 176 | 176 | 176 | 176 | 176 |
| p40 V_{L} | 177 | 177 | 177 | 177 | 177 | 177 |
| p40 CL | 16 | 16 | 16 | 16 | 197 | 16 |
| p40 LC | 178 | 178 | 178 | 178 | 198 | 178 |
| TL1A HC CDR1 | 30 | 30 | 30 | 30 | 30 | N/A |
| TL1A HC CDR2 | 41 | 41 | 41 | 41 | 41 | N/A |
| TL1A HC CDR3 | 32 | 32 | 32 | 32 | 32 | N/A |
| TL1A JH | 4 | 4 | 4 | 4 | 4 | N/A |
| TL1A V_{H} | 42 | 42 | 42 | 42 | 42 | N/A |
| TL1A CH1 | 6 | 6 | 6 | 6 | 199 | N/A |
| TL1A HINGE | 182 | 7 | 191 | 7 | 7 | N/A |
| TL1A CH2 | 8 | 8 | 8 | 8 | 8 | N/A |
| TL1A CH3 | 183 | 187 | 183 | 183 | 162 | N/A |
| TL1A HC | 184 | 188 | 192 | 194 | 200 | N/A |
| TL1A LC CDR1 | 11 | 11 | 11 | 11 | 11 | N/A |
| TL1A LC CDR2 | 12 | 12 | 12 | 12 | 12 | N/A |
| TL1A LC CDR3 | 13 | 13 | 13 | 13 | 13 | N/A |
| TL1A JK | 14 | 14 | 14 | 14 | 14 | N/A |
| TL1A V_{L} | 18 | 18 | 18 | 18 | 18 | N/A |
| TL1A CL | 16 | 16 | 16 | 16 | 201 | N/A |
| TL1A LC | 19 | 19 | 19 | 19 | 202 | N/A |
| TL1A LC CDR1 | N/A | N/A | N/A | N/A | N/A | 11 |
| TL1A LC CDR2 | N/A | N/A | N/A | N/A | N/A | 12 |
| TL1A LC CDR3 | N/A | N/A | N/A | N/A | N/A | 13 |
| TL1A JK | N/A | N/A | N/A | N/A | N/A | 14 |
| TL1A V_{L} | N/A | N/A | N/A | N/A | N/A | 18 |
| TL1A CL | N/A | N/A | N/A | N/A | N/A | 16 |
| TL1A Lower | N/A | N/A | N/A | N/A | N/A | 204 |
| HINGE | | | | | | |
| TL1A CH2 | N/A | N/A | N/A | N/A | N/A | 8 |
| TL1A CH3 | N/A | N/A | N/A | N/A | N/A | 162 |
| TL1A LC-Fc | N/A | N/A | N/A | N/A | N/A | 205 |
| TL1A HC CDR1 | N/A | N/A | N/A | N/A | N/A | 30 |
| TL1A HC CDR2 | N/A | N/A | N/A | N/A | N/A | 41 |
| TL1A HC CDR3 | N/A | N/A | N/A | N/A | N/A | 32 |
| TL1A JH | N/A | N/A | N/A | N/A | N/A | 4 |
| TL1A V_{H} | N/A | N/A | N/A | N/A | N/A | 42 |
| TL1A CH1 | N/A | N/A | N/A | N/A | N/A | 6 |
| TL1A Upper | N/A | N/A | N/A | N/A | N/A | 206 |
| HINGE | | | | | | |
| TL1A Modified | N/A | N/A | N/A | N/A | N/A | 207 |
| Fd | | | | | | |

N/A = non-applicable

TABLE 32
One armed and monovalent bispecific antibodies

| | Anti-TL1A One-armed or Monovalent Bispecific Variants | | | | |
|---|---|---|---|---|---|
| Variant | TL1A-0057 | TL1A-0326 | TL1A-0327 | TL1A-0328 | TL1A-0372 |
| HC (Functional) CDR1 | 1 | 30 | 30 | 109 | 30 |
| HC (Functional) CDR2 | 2 | 41 | 56 | 56 | 41 |
| HC (Functional) CDR3 | 3 | 32 | 32 | 32 | 32 |
| JH (Functional) | 4 | 4 | 4 | 4 | 4 |
| VH (Functional) | 5 | 42 | 57 | 120 | 42 |
| CH1 (Functional) | 6 | 6 | 6 | 6 | 6 |
| HINGE (Functional) | 7 | 7 | 7 | 7 | 7 |
| CH2 (Functional) | 8 | 8 | 8 | 8 | 8 |
| CH3 (Functional) | 155 | 160 | 160 | 160 | 160 |
| TL1A HC (Functional) | 156 | 161 | 164 | 165 | 161 |
| HINGE (Truncated) | 157 | 157 | 157 | 157 | N/A |
| CH2 (Truncated) | 8 | 8 | 8 | 8 | N/A |
| CH3 (Truncated) | 158 | 162 | 162 | 162 | N/A |
| HC (Truncated) | 159 | 163 | 163 | 163 | N/A |
| HC [xTL1A] CDR1 | N/A | N/A | N/A | N/A | 139 |
| HC [xTL1A] CDR2 | N/A | N/A | N/A | N/A | 140 |
| HC [xTL1A] CDR3 | N/A | N/A | N/A | N/A | 133 |
| JH [xTL1A] | N/A | N/A | N/A | N/A | 4 |
| VH [xTL1A] | N/A | N/A | N/A | N/A | 143 |
| CH1 [xTL1A] | N/A | N/A | N/A | N/A | 6 |
| HINGE [xTL1A] | N/A | N/A | N/A | N/A | 7 |
| CH2 [xTL1A] | N/A | N/A | N/A | N/A | 8 |
| CH3 [xTL1A] | N/A | N/A | N/A | N/A | 162 |
| [xTL1A] HC (Disabled) | N/A | N/A | N/A | N/A | 166 |
| LC(TL1A) CDR1 | 11 | 11 | 11 | 11 | 11 |
| LC(TL1A) CDR2 | 12 | 12 | 12 | 12 | 12 |
| LC(TL1A) CDR3 | 13 | 13 | 13 | 13 | 13 |
| JK(TL1A) | 14 | 14 | 14 | 14 | 14 |
| V_{L}(TL1A) | 15 | 18 | 18 | 18 | 18 |
| CL(TL1A) | 16 | 16 | 16 | 16 | 16 |
| TL1A LC | 17 | 19 | 19 | 19 | 19 |

N/A = not applicable

TABLE 33
SEQUENCE LISTING.

| SEQ | DESCRIPTION | SEQUENCE |
|---|---|---|
| 1 | TL1A-0002 HC | GYDFTYYGIS |
| | CDR1 | |
| 2 | TL1A-0002 HC | WISTYNGNTHYARMLQG |
| | CDR2 | |
| 3 | TL1A-0002 HC | ENYYGSGAYRGGMDV |
| | CDR3 | |
| 4 | TL1A-0002 JH | WGQGTTVTVSS |
| 5 | TL1A-0002 VH | QVQLVQSGAEVKKPGASVKVSCKASGYDFTYYGISWVRQAPGQGLEWMGWISTYNGNTHYAR |
| | | MLQGRVTMTTDTSTRTAYMELRSLRSDDTAVYYCARENYYGSGAYRGGMDVWGQGTTVTVSS |
| 6 | CH1 | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKV |
| 7 | HINGE | EPKSCDKTHTCPPCP |
| 8 | CH2 | APEAAGAPSVFLFPPKPKDTLMISRTPEVTCVVVDVSHEDPEVKENWYVDGVEVHNAKTKPR |
| | | EEQYNSTYRVVSVLTVLHQDWLNGKEYKCKVSNKALPAPIEKTISKAK |
| 9 | CH3 | GQPREPQVYTLPPSREEMTKNQVSLTCLVKGFYPSDIAVEWESNGQPENNYKTTPPVLDSDG |
| | | SFFLYSKLTVDKSRWQQGNVFSCSVMHEALHNHYTQKSLSLSPG |
| 10 | TL1A-0002 HC | QVQLVQSGAEVKKPGASVKVSCKASGYDFTYYGISWVRQAPGQGLEWMGWISTYNGNTHYAR |
| | | MLQGRVTMTTDTSTRTAYMELRSLRSDDTAVYYCARENYYGSGAYRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 11 | TL1A-0002 LC | RASQSVSSYLA |
| | CDR1 | |
| 12 | TL1A-0002 LC | DASNRAT |
| | CDR2 | |
| 13 | TL1A-0002 LC | QQRSNWPWT |
| | CDR3 | |
| 14 | TL1A-0002 JK | FGQGTKVEIK |
| 15 | TL1A-0002 VL | EIVLTQSPATLSLSPGERATLSCRASQSVSSYLAWYQQKPGQAPRLLIYDASNRATGIPARF |
| | | SGSGSGTDFTLTISSLEPEDFAVYYCQQRSNWPWTFGQGTKVEIK |
| 16 | CL (Kappa) | (R)TVAAPSVFIFPPSDEQLKSGTASVVCLLNNFYPREAKVQWKVDNALQSGNSQESVTEQD |
| | | SKDSTYSLSSTLTLSKADYEKHKVYACEVTHQGLSSPVTKSENRGEC |
| 17 | TL1A-0002 LC | EIVLTQSPATLSLSPGERATLSCRASQSVSSYLAWYQQKPGQAPRLLIYDASNRATGIPARF |
| | | SGSGSGTDFTLTISSLEPEDFAVYYCQQRSNWPWTFGQGTKVEIKRTVAAPSVFIFPPSDEQ |
| | | LKSGTASVVCLLNNFYPREAKVQWKVDNALQSGNSQESVTEQDSKDSTYSLSSTLTLSKADY |
| | | EKHKVYACEVTHQGLSSPVTKSENRGEC |
| 18 | TL1A-0042 VL | DIQMTQSPSSLSASVGDRVTITCRASQSVSSYLAWYQQKPGKAPKLLIYDASNRATGVPSRF |
| | | SGSGSGTDFTLTISSLQPEDFATYYCQQRSNWPWTFGQGTKVEIK |
| 19 | TL1A-0042 LC | DIQMTQSPSSLSASVGDRVTITCRASQSVSSYLAWYQQKPGKAPKLLIYDASNRATGVPSRF |
| | | SGSGSGTDFTLTISSLQPEDFATYYCQQRSNWPWTFGQGTKVEIKRTVAAPSVFIFPPSDEQ |
| | | LKSGTASVVCLLNNFYPREAKVQWKVDNALQSGNSQESVTEQDSKDSTYSLSSTLTLSKADY |
| | | EKHKVYACEVTHQGLSSPVTKSENRGEC |
| 20 | TL1A-0043 VL | EIVMTQSPATLSLSPGERATLSCRASQSVSSYLAWYQQKPGQAPRLLIYDASNRATGIPARF |
| | | SGSGSGTDFTLTISSLQPEDFAVYYCQQRSNWPWTFGQGTKVEIK |
| 21 | TL1A-0043 LC | EIVMTQSPATLSLSPGERATLSCRASQSVSSYLAWYQQKPGQAPRLLIYDASNRATGIPARF |
| | | SGSGSGTDFTLTISSLQPEDFAVYYCQQRSNWPWTFGQGTKVEIKRTVAAPSVFIFPPSDEQ |
| | | LKSGTASVVCLLNNFYPREAKVQWKVDNALQSGNSQESVTEQDSKDSTYSLSSTLTLSKADY |
| | | EKHKVYACEVTHQGLSSPVTKSENRGEC |
| 22 | TL1A-0045 VH | QVQLVQSGAEVKKPGASVKVSCKASGYDFTYYGISWVRQAPGQGLEWMGWISTYNGNTHYAR |
| | | MLQGRVTMTRDTSTSTVYMELSSLRSEDTAVYYCARENYYGSGAYRGGMDVWGQGTTVTVSS |
| 23 | TL1A-0045 HC | QVQLVQSGAEVKKPGASVKVSCKASGYDFTYYGISWVRQAPGQGLEWMGWISTYNGNTHYAR |
| | | MLQGRVTMTRDTSTSTVYMELSSLRSEDTAVYYCARENYYGSGAYRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 24 | TL1A-0047 VH | EVQLVESGGGLVQPGGSLRLSCAASGYDFTYYGISWVRQAPGKGLEWVAWISTYNGNTHYAR |
| | | MLQGRFTISRDNAKNSLYLQMNSLRAEDTAVYYCARENYYGSGAYRGGMDVWGQGTTVTVSS |
| 25 | TL1A-0047 HC | EVQLVESGGGLVQPGGSLRLSCAASGYDFTYYGISWVRQAPGKGLEWVAWISTYNGNTHYAR |
| | | MLQGRFTISRDNAKNSLYLQMNSLRAEDTAVYYCARENYYGSGAYRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 26 | TL1A-0048 VH | QVQLVQSGAEVKKPGASVKVSCKASGYDFTYYGISWVRQAPGQGLEWMGWISTYNGNTHYAR |
| | | MLQGRVTMTRDTSISTAYMELSRLRSDDTAVYYCARENYYGSGAYRGGMDVWGQGTTVTVSS |
| 27 | TL1A-0048 HC | QVQLVQSGAEVKKPGASVKVSCKASGYDFTYYGISWVRQAPGQGLEWMGWISTYNGNTHYAR |
| | | MLQGRVTMTRDTSISTAYMELSRLRSDDTAVYYCARENYYGSGAYRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 28 | TL1A-0167 VH | QVQLVQSGAEVKKPGASVKVSCKASGYDFTYYGISWVRQAPGQGLEWMGWISTYNGNTHYAR |
| | | MLQGRVTMTTDTSTSTAYMELRSLRSDDTAVYYCARENYYGSGAYRGGMDVWGQGTTVTVSS |
| 29 | TL1A-0167 HC | QVQLVQSGAEVKKPGASVKVSCKASGYDFTYYGISWVRQAPGQGLEWMGWISTYNGNTHYAR |
| | | MLQGRVTMTTDTSTSTAYMELRSLRSDDTAVYYCARENYYGSGAYRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 30 | TL1A-0256 | GYDYTYYGIS |
| | CDRH1 | |
| 31 | TL1A-0256 | WISTYNDNTHYAPSLQG |
| | CDRH2 | |
| 32 | TL1A-0256 | ESYYGSGAFRGGMDV |
| | CDRH3 | |
| 33 | TL1A-0256 VH | QVQLVQSGAEVKKPGASVKVSCKASGYDYTYYGISWVRQAPGQGLEWMGWISTYNDNTHYAP |
| | | SLQGRVTMTTDTSTSTAYMELRSLRSDDTAVYYCARESYYGSGAFRGGMDVWGQGTTVTVSS |
| 34 | TL1A-0256 HC | QVQLVQSGAEVKKPGASVKVSCKASGYDYTYYGISWVRQAPGQGLEWMGWISTYNDNTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 35 | TL1A-0257 | ESYYGSGAHRGGMDV |
| | CDRH3 | |
| 36 | TL1A-0257 VH | QVQLVQSGAEVKKPGASVKVSCKASGYDYTYYGISWVRQAPGQGLEWMGWISTYNDNTHYAP |
| | | SLQGRVTMTTDTSTSTAYMELRSLRSDDTAVYYCARESYYGSGAHRGGMDVWGQGTTVTVSS |
| 37 | TL1A-0257 HC | QVQLVQSGAEVKKPGASVKVSCKASGYDYTYYGISWVRQAPGQGLEWMGWISTYNDNTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 38 | TL1A-0258 | WISTYNDNTHYAQKLQG |
| | CDRH2 | |
| 39 | TL1A-0258 VH | QVQLVQSGAEVKKPGASVKVSCKASGYDYTYYGISWVRQAPGQGLEWMGWISTYNDNTHYAQ |
| | | KLQGRVTMTTDTSTSTAYMELRSLRSDDTAVYYCARESYYGSGAFRGGMDVWGQGTTVTVSS |
| 40 | TL1A-0258 HC | QVQLVQSGAEVKKPGASVKVSCKASGYDYTYYGISWVRQAPGQGLEWMGWISTYNDNTHYAQ |
| | | KLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKENWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 41 | TL1A-0260 | WISTYNTGTHYAPSLQG |
| | CDRH2 | |
| 42 | TL1A-0260 VH | QVQLVQSGAEVKKPGASVKVSCKASGYDYTYYGISWVRQAPGQGLEWMGWISTYNTGTHYAP |
| | | SLQGRVTMTTDTSTSTAYMELRSLRSDDTAVYYCARESYYGSGAFRGGMDVWGQGTTVTVSS |
| 43 | TL1A-0260 HC | QVQLVQSGAEVKKPGASVKVSCKASGYDYTYYGISWVRQAPGQGLEWMGWISTYNTGTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 44 | TL1A-0261 VH | QVQLVQSGAEVKKPGASVKVSCKASGYDYTYYGISWVRQAPGQGLEWMGWISTYNTGTHYAP |
| | | SLQGRVTMTTDTSTSTAYMELRSLRSDDTAVYYCARESYYGSGAHRGGMDVWGQGTTVTVSS |
| 45 | TL1A-0261 HC | QVQLVQSGAEVKKPGASVKVSCKASGYDYTYYGISWVRQAPGQGLEWMGWISTYNTGTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 46 | TL1A-0262 | WISTYNTGTHYAQKLQG |
| | CDRH2 | |
| 47 | TL1A-0262 VH | QVQLVQSGAEVKKPGASVKVSCKASGYDYTYYGISWVRQAPGQGLEWMGWISTYNTGTHYAQ |
| | | KLQGRVTMTTDTSTSTAYMELRSLRSDDTAVYYCARESYYGSGAFRGGMDVWGQGTTVTVSS |
| 48 | TL1A-0262 HC | QVQLVQSGAEVKKPGASVKVSCKASGYDYTYYGISWVRQAPGQGLEWMGWISTYNTGTHYAQ |
| | | KLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 49 | TL1A-0263 VH | QVQLVQSGAEVKKPGASVKVSCKASGYDYTYYGISWVRQAPGQGLEWMGWISTYNTGTHYAQ |
| | | KLQGRVTMTTDTSTSTAYMELRSLRSDDTAVYYCARESYYGSGAHRGGMDVWGQGTTVTVSS |
| 50 | TL1A-0263 HC | QVQLVQSGAEVKKPGASVKVSCKASGYDYTYYGISWVRQAPGQGLEWMGWISTYNTGTHYAQ |
| | | KLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 51 | TL1A-0264 | WISTYNTVTHYAPSLQG |
| | CDRH2 | |
| 52 | TL1A-0264 VH | QVQLVQSGAEVKKPGASVKVSCKASGYDYTYYGISWVRQAPGQGLEWMGWISTYNTVTHYAP |
| | | SLQGRVTMTTDTSTSTAYMELRSLRSDDTAVYYCARESYYGSGAFRGGMDVWGQGTTVTVSS |
| 53 | TL1A-0264 HC | QVQLVQSGAEVKKPGASVKVSCKASGYDYTYYGISWVRQAPGQGLEWMGWISTYNTVTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 54 | TL1A-0265 VH | QVQLVQSGAEVKKPGASVKVSCKASGYDYTYYGISWVRQAPGQGLEWMGWISTYNTVTHYAP |
| | | SLQGRVTMTTDTSTSTAYMELRSLRSDDTAVYYCARESYYGSGAHRGGMDVWGQGTTVTVSS |
| 55 | TL1A-0265 HC | QVQLVQSGAEVKKPGASVKVSCKASGYDYTYYGISWVRQAPGQGLEWMGWISTYNTVTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 56 | TL1A-0266 | WISTYNTNTHYAPSLQG |
| | CDRH2 | |
| 57 | TL1A-0266 VH | QVQLVQSGAEVKKPGASVKVSCKASGYDYTYYGISWVRQAPGQGLEWMGWISTYNTNTHYAP |
| | | SLQGRVTMTTDTSTSTAYMELRSLRSDDTAVYYCARESYYGSGAFRGGMDVWGQGTTVTVSS |
| 58 | TL1A-0266 HC | QVQLVQSGAEVKKPGASVKVSCKASGYDYTYYGISWVRQAPGQGLEWMGWISTYNTNTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 59 | TL1A-0267 VH | QVQLVQSGAEVKKPGASVKVSCKASGYDYTYYGISWVRQAPGQGLEWMGWISTYNTNTHYAP |
| | | SLQGRVTMTTDTSTSTAYMELRSLRSDDTAVYYCARESYYGSGAHRGGMDVWGQGTTVTVSS |
| 60 | TL1A-0267 HC | QVQLVQSGAEVKKPGASVKVSCKASGYDYTYYGISWVRQAPGQGLEWMGWISTYNTNTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 61 | TL1A-0270 HC | GYDFSYYGIS |
| | CDR H1 | |
| 62 | TL1A-0270 VH | QVQLVQSGAEVKKPGASVKVSCKASGYDFSYYGISWVRQAPGQGLEWMGWISTYNDNTHYAP |
| | | SLQGRVTMTTDTSTSTAYMELRSLRSDDTAVYYCARESYYGSGAFRGGMDVWGQGTTVTVSS |
| 63 | TL1A-0270 HC | QVQLVQSGAEVKKPGASVKVSCKASGYDFSYYGISWVRQAPGQGLEWMGWISTYNDNTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 64 | TL1A-0271 VH | QVQLVQSGAEVKKPGASVKVSCKASGYDFSYYGISWVRQAPGQGLEWMGWISTYNDNTHYAP |
| | | SLQGRVTMTTDTSTSTAYMELRSLRSDDTAVYYCARESYYGSGAHRGGMDVWGQGTTVTVSS |
| 65 | TL1A-0271 HC | QVQLVQSGAEVKKPGASVKVSCKASGYDFSYYGISWVRQAPGQGLEWMGWISTYNDNTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 66 | TL1A-0272 VH | QVQLVQSGAEVKKPGASVKVSCKASGYDFSYYGISWVRQAPGQGLEWMGWISTYNDNTHYAQ |
| | | KLQGRVTMTTDTSTSTAYMELRSLRSDDTAVYYCARESYYGSGAFRGGMDVWGQGTTVTVSS |
| 67 | TL1A-0272 HC | QVQLVQSGAEVKKPGASVKVSCKASGYDFSYYGISWVRQAPGQGLEWMGWISTYNDNTHYAQ |
| | | KLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 68 | TL1A-0273 VH | QVQLVQSGAEVKKPGASVKVSCKASGYDFSYYGISWVRQAPGQGLEWMGWISTYNDNTHYAQ |
| | | KLQGRVTMTTDTSTSTAYMELRSLRSDDTAVYYCARESYYGSGAHRGGMDVWGQGTTVTVSS |
| 69 | TL1A-0273 HC | QVQLVQSGAEVKKPGASVKVSCKASGYDFSYYGISWVRQAPGQGLEWMGWISTYNDNTHYAQ |
| | | KLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 70 | TL1A-0274 VH | QVQLVQSGAEVKKPGASVKVSCKASGYDFSYYGISWVRQAPGQGLEWMGWISTYNTGTHYAP |
| | | SLQGRVTMTTDTSTSTAYMELRSLRSDDTAVYYCARESYYGSGAFRGGMDVWGQGTTVTVSS |
| 71 | TL1A-0274 HC | QVQLVQSGAEVKKPGASVKVSCKASGYDFSYYGISWVRQAPGQGLEWMGWISTYNTGTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKENWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 72 | TL1A-0275 VH | QVQLVQSGAEVKKPGASVKVSCKASGYDFSYYGISWVRQAPGQGLEWMGWISTYNTGTHYAP |
| | | SLQGRVTMTTDTSTSTAYMELRSLRSDDTAVYYCARESYYGSGAHRGGMDVWGQGTTVTVSS |
| 73 | TL1A-0275 HC | QVQLVQSGAEVKKPGASVKVSCKASGYDFSYYGISWVRQAPGQGLEWMGWISTYNTGTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 74 | TL1A-0276 VH | QVQLVQSGAEVKKPGASVKVSCKASGYDFSYYGISWVRQAPGQGLEWMGWISTYNTGTHYAQ |
| | | KLQGRVTMTTDTSTSTAYMELRSLRSDDTAVYYCARESYYGSGAFRGGMDVWGQGTTVTVSS |
| 75 | TL1A-0276 HC | QVQLVQSGAEVKKPGASVKVSCKASGYDFSYYGISWVRQAPGQGLEWMGWISTYNTGTHYAQ |
| | | KLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 76 | TL1A-0277 VH | QVQLVQSGAEVKKPGASVKVSCKASGYDFSYYGISWVRQAPGQGLEWMGWISTYNTGTHYAQ |
| | | KLQGRVTMTTDTSTSTAYMELRSLRSDDTAVYYCARESYYGSGAHRGGMDVWGQGTTVTVSS |
| 77 | TL1A-0277 HC | QVQLVQSGAEVKKPGASVKVSCKASGYDFSYYGISWVRQAPGQGLEWMGWISTYNTGTHYAQ |
| | | KLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 78 | TL1A-0278 VH | QVQLVQSGAEVKKPGASVKVSCKASGYDFSYYGISWVRQAPGQGLEWMGWISTYNTVTHYAP |
| | | SLQGRVTMTTDTSTSTAYMELRSLRSDDTAVYYCARESYYGSGAFRGGMDVWGQGTTVTVSS |
| 79 | TL1A-0278 HC | QVQLVQSGAEVKKPGASVKVSCKASGYDFSYYGISWVRQAPGQGLEWMGWISTYNTVTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKENWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 80 | TL1A-0279 VH | QVQLVQSGAEVKKPGASVKVSCKASGYDFSYYGISWVRQAPGQGLEWMGWISTYNTVTHYAP |
| | | SLQGRVTMTTDTSTSTAYMELRSLRSDDTAVYYCARESYYGSGAHRGGMDVWGQGTTVTVSS |
| 81 | TL1A-0279 HC | QVQLVQSGAEVKKPGASVKVSCKASGYDFSYYGISWVRQAPGQGLEWMGWISTYNTVTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 82 | TL1A-0280 VH | QVQLVQSGAEVKKPGASVKVSCKASGYDFSYYGISWVRQAPGQGLEWMGWISTYNTNTHYAP |
| | | SLQGRVTMTTDTSTSTAYMELRSLRSDDTAVYYCARESYYGSGAFRGGMDVWGQGTTVTVSS |
| 83 | TL1A-0280 HC | QVQLVQSGAEVKKPGASVKVSCKASGYDFSYYGISWVRQAPGQGLEWMGWISTYNTNTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 84 | TL1A-0281 VH | QVQLVQSGAEVKKPGASVKVSCKASGYDFSYYGISWVRQAPGQGLEWMGWISTYNTNTHYAP |
| | | SLQGRVTMTTDTSTSTAYMELRSLRSDDTAVYYCARESYYGSGAHRGGMDVWGQGTTVTVSS |
| 85 | TL1A-0281 HC | QVQLVQSGAEVKKPGASVKVSCKASGYDFSYYGISWVRQAPGQGLEWMGWISTYNTNTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 86 | TL1A-0284 HC | GYDFTYYGVS |
| | CDRH1 | |
| 87 | TL1A-0284 VH | QVQLVQSGAEVKKPGASVKVSCKASGYDFTYYGVSWVRQAPGQGLEWMGWISTYNDNTHYAP |
| | | SLQGRVTMTTDTSTSTAYMELRSLRSDDTAVYYCARESYYGSGAFRGGMDVWGQGTTVTVSS |
| 88 | TL1A-0284 HC | QVQLVQSGAEVKKPGASVKVSCKASGYDFTYYGVSWVRQAPGQGLEWMGWISTYNDNTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 89 | TL1A-0285 VH | QVQLVQSGAEVKKPGASVKVSCKASGYDFTYYGVSWVRQAPGQGLEWMGWISTYNDNTHYAP |
| | | SLQGRVTMTTDTSTSTAYMELRSLRSDDTAVYYCARESYYGSGAHRGGMDVWGQGTTVTVSS |
| 90 | TL1A-0285 HC | QVQLVQSGAEVKKPGASVKVSCKASGYDFTYYGVSWVRQAPGQGLEWMGWISTYNDNTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 91 | TL1A-0286 VH | QVQLVQSGAEVKKPGASVKVSCKASGYDFTYYGVSWVRQAPGQGLEWMGWISTYNDNTHYAQ |
| | | KLQGRVTMTTDTSTSTAYMELRSLRSDDTAVYYCARESYYGSGAFRGGMDVWGQGTTVTVSS |
| 92 | TL1A-0286 HC | QVQLVQSGAEVKKPGASVKVSCKASGYDFTYYGVSWVRQAPGQGLEWMGWISTYNDNTHYAQ |
| | | KLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 93 | TL1A-0287 VH | QVQLVQSGAEVKKPGASVKVSCKASGYDFTYYGVSWVRQAPGQGLEWMGWISTYNDNTHYAQ |
| | | KLQGRVTMTTDTSTSTAYMELRSLRSDDTAVYYCARESYYGSGAHRGGMDVWGQGTTVTVSS |
| 94 | TL1A-0287 HC | QVQLVQSGAEVKKPGASVKVSCKASGYDFTYYGVSWVRQAPGQGLEWMGWISTYNDNTHYAQ |
| | | KLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKENWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 95 | TL1A-0288 VH | QVQLVQSGAEVKKPGASVKVSCKASGYDFTYYGVSWVRQAPGQGLEWMGWISTYNTGTHYAP |
| | | SLQGRVTMTTDTSTSTAYMELRSLRSDDTAVYYCARESYYGSGAFRGGMDVWGQGTTVTVSS |
| 96 | TL1A-0288 HC | QVQLVQSGAEVKKPGASVKVSCKASGYDFTYYGVSWVRQAPGQGLEWMGWISTYNTGTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 97 | TL1A-0289 VH | QVQLVQSGAEVKKPGASVKVSCKASGYDFTYYGVSWVRQAPGQGLEWMGWISTYNTGTHYAP |
| | | SLQGRVTMTTDTSTSTAYMELRSLRSDDTAVYYCARESYYGSGAHRGGMDVWGQGTTVTVSS |
| 98 | TL1A-0289 HC | QVQLVQSGAEVKKPGASVKVSCKASGYDFTYYGVSWVRQAPGQGLEWMGWISTYNTGTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKENWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 99 | TL1A-0291 VH | QVQLVQSGAEVKKPGASVKVSCKASGYDFTYYGVSWVRQAPGQGLEWMGWISTYNTGTHYAQ |
| | | KLQGRVTMTTDTSTSTAYMELRSLRSDDTAVYYCARESYYGSGAHRGGMDVWGQGTTVTVSS |
| 100 | TL1A-0291 HC | QVQLVQSGAEVKKPGASVKVSCKASGYDFTYYGVSWVRQAPGQGLEWMGWISTYNTGTHYAQ |
| | | KLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPG |
| 101 | TL1A-0292 VH | QVQLVQSGAEVKKPGASVKVSCKASGYDFTYYGVSWVRQAPGQGLEWMGWISTYNTVTHYAP |
| | | SLQGRVTMTTDTSTSTAYMELRSLRSDDTAVYYCARESYYGSGAFRGGMDVWGQGTTVTVSS |
| 102 | TL1A-0292 HC | QVQLVQSGAEVKKPGASVKVSCKASGYDFTYYGVSWVRQAPGQGLEWMGWISTYNTVTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 103 | TL1A-0293 VH | QVQLVQSGAEVKKPGASVKVSCKASGYDFTYYGVSWVRQAPGQGLEWMGWISTYNTVTHYAP |
| | | SLQGRVTMTTDTSTSTAYMELRSLRSDDTAVYYCARESYYGSGAHRGGMDVWGQGTTVTVSS |
| 104 | TL1A-0293 HC | QVQLVQSGAEVKKPGASVKVSCKASGYDFTYYGVSWVRQAPGQGLEWMGWISTYNTVTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 105 | TL1A-0294 VH | QVQLVQSGAEVKKPGASVKVSCKASGYDFTYYGVSWVRQAPGQGLEWMGWISTYNTNTHYAP |
| | | SLQGRVTMTTDTSTSTAYMELRSLRSDDTAVYYCARESYYGSGAFRGGMDVWGQGTTVTVSS |
| 106 | TL1A-0294 HC | QVQLVQSGAEVKKPGASVKVSCKASGYDFTYYGVSWVRQAPGQGLEWMGWISTYNTNTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 107 | TL1A-0295 VH | QVQLVQSGAEVKKPGASVKVSCKASGYDFTYYGVSWVRQAPGQGLEWMGWISTYNTNTHYAP |
| | | SLQGRVTMTTDTSTSTAYMELRSLRSDDTAVYYCARESYYGSGAHRGGMDVWGQGTTVTVSS |
| 108 | TL1A-0295 HC | QVQLVQSGAEVKKPGASVKVSCKASGYDFTYYGVSWVRQAPGQGLEWMGWISTYNTNTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 109 | TL1A-0303 HC | GYDFTYYGIG |
| | CDRH1 | |
| 110 | TL1A-0303 VH | QVQLVQSGAEVKKPGASVKVSCKASGYDFTYYGIGWVRQAPGQGLEWMGWISTYNTGTHYAP |
| | | SLQGRVTMTTDTSTSTAYMELRSLRSDDTAVYYCARESYYGSGAHRGGMDVWGQGTTVTVSS |
| 111 | TL1A-0303 HC | QVQLVQSGAEVKKPGASVKVSCKASGYDFTYYGIGWVRQAPGQGLEWMGWISTYNTGTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 112 | TL1A-0304 VH | QVQLVQSGAEVKKPGASVKVSCKASGYDFTYYGIGWVRQAPGQGLEWMGWISTYNTGTHYAQ |
| | | KLQGRVTMTTDTSTSTAYMELRSLRSDDTAVYYCARESYYGSGAFRGGMDVWGQGTTVTVSS |
| 113 | TL1A-0304 HC | QVQLVQSGAEVKKPGASVKVSCKASGYDFTYYGIGWVRQAPGQGLEWMGWISTYNTGTHYAQ |
| | | KLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 114 | TL1A-0305 VH | QVQLVQSGAEVKKPGASVKVSCKASGYDFTYYGIGWVRQAPGQGLEWMGWISTYNTGTHYAQ |
| | | KLQGRVTMTTDTSTSTAYMELRSLRSDDTAVYYCARESYYGSGAHRGGMDVWGQGTTVTVSS |
| 115 | TL1A-0305 HC | QVQLVQSGAEVKKPGASVKVSCKASGYDFTYYGIGWVRQAPGQGLEWMGWISTYNTGTHYAQ |
| | | KLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 116 | TL1A-0306 VH | QVQLVQSGAEVKKPGASVKVSCKASGYDFTYYGIGWVRQAPGQGLEWMGWISTYNTVTHYAP |
| | | SLQGRVTMTTDTSTSTAYMELRSLRSDDTAVYYCARESYYGSGAFRGGMDVWGQGTTVTVSS |
| 117 | TL1A-0306 HC | QVQLVQSGAEVKKPGASVKVSCKASGYDFTYYGIGWVRQAPGQGLEWMGWISTYNTVTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 118 | TL1A-0307 VH | QVQLVQSGAEVKKPGASVKVSCKASGYDFTYYGIGWVRQAPGQGLEWMGWISTYNTVTHYAP |
| | | SLQGRVTMTTDTSTSTAYMELRSLRSDDTAVYYCARESYYGSGAHRGGMDVWGQGTTVTVSS |
| 119 | TL1A-0307 HC | QVQLVQSGAEVKKPGASVKVSCKASGYDFTYYGIGWVRQAPGQGLEWMGWISTYNTVTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 120 | TL1A-0308 VH | QVQLVQSGAEVKKPGASVKVSCKASGYDFTYYGIGWVRQAPGQGLEWMGWISTYNTNTHYAP |
| | | SLQGRVTMTTDTSTSTAYMELRSLRSDDTAVYYCARESYYGSGAFRGGMDVWGQGTTVTVSS |
| 121 | TL1A-0308 HC | QVQLVQSGAEVKKPGASVKVSCKASGYDFTYYGIGWVRQAPGQGLEWMGWISTYNTNTHYAP |
| | | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 122 | TL1A-0309 VH | QVQLVQSGAEVKKPGASVKVSCKASGYDFTYYGIGWVRQAPGQGLEWMGWISTYNTNTHYAP |
| | | SLQGRVTMTTDTSTSTAYMELRSLRSDDTAVYYCARESYYGSGAHRGGMDVWGQGTTVTVSS |
| 123 | TL1A-0309 HC | QVQLVQSGAEVKKPGASVKVSCKASGYDFTYYGIGWVRQAPGQGLEWMGWISTYNTNTHYAP |
| | | SLQGRVTMTTDTSTSTAYMELRSLRSDDTAVYYCARESYYGSGAHRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 124 | TL1A-0324 | WISTYNTNTHYARMLQG |
| | CDRH2 | |
| 125 | TL1A-0324 | ESYYGSGAYRGGMDV |
| | CDRH3 | |
| 126 | TL1A-0324 VH | QVQLVQSGAEVKKPGASVKVSCKASGYDFTYYGISWVRQAPGQGLEWMGWISTYNTNTHYAR |
| | | MLQGRVTMTTDTSTSTAYMELRSLRSDDTAVYYCARESYYGSGAYRGGMDVWGQGTTVTVSS |
| 127 | TL1A-0324 HC | QVQLVQSGAEVKKPGASVKVSCKASGYDFTYYGISWVRQAPGQGLEWMGWISTYNTNTHYAR |
| | | MLQGRVTMTTDTSTSTAYMELRSLRSDDTAVYYCARESYYGSGAYRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 128 | [xTL1A]-0336 | GYTFTSYGIS |
| | CDRH1 | |
| 129 | [xTL1A]-0336 | WISAYNGNTNYAQKLQG |
| | CDRH2 | |
| 130 | [xTL1A]-0336 | ESYYGSEAFRAGMDV |
| | CDRH3 | |
| 131 | [xTL1A]-0336 | QVQLVQSGAEVKKPGASVKVSCKASGYTFTSYGISWVRQAPGQGLEWMGWISAYNGNTNYAQ |
| | VH | KLQGRVTMTTDTSTSTAYMELRSLRSDDTAVYYCARESYYGSEAFRAGMDVWGQGTTVTVSS |
| 132 | [xTL1A]-0336 | QVQLVQSGAEVKKPGASVKVSCKASGYTFTSYGISWVRQAPGQGLEWMGWISAYNGNTNYAQ |
| | HC | KLQGRVTMTTDTSTSTAYMELRSLRSDDTAVYYCARESYYGSEAFRAGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKENWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 133 | [xTL1A]-0338 | ESSYGSEAFRGGMDV |
| | CDRH3 | |
| 134 | [xTL1A]-0338 | QVQLVQSGAEVKKPGASVKVSCKASGYTFTSYGISWVRQAPGQGLEWMGWISAYNGNTNYAQ |
| | VH | KLQGRVTMTTDTSTSTAYMELRSLRSDDTAVYYCARESSYGSEAFRGGMDVWGQGTTVTVSS |
| 135 | [xTL1A]-0338 | QVQLVQSGAEVKKPGASVKVSCKASGYTFTSYGISWVRQAPGQGLEWMGWISAYNGNTNYAQ |
| | HC | KLQGRVTMTTDTSTSTAYMELRSLRSDDTAVYYCARESSYGSEAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 136 | [xTL1A]-0341 | ESSYGSEAFRAGMDV |
| | CDRH3 | |
| 137 | [xTL1A]-0341 | QVQLVQSGAEVKKPGASVKVSCKASGYTFTSYGISWVRQAPGQGLEWMGWISAYNGNTNYAQ |
| | VH | KLQGRVTMTTDTSTSTAYMELRSLRSDDTAVYYCARESSYGSEAFRAGMDVWGQGTTVTVSS |
| 138 | [xTL1A]-0341 | QVQLVQSGAEVKKPGASVKVSCKASGYTFTSYGISWVRQAPGQGLEWMGWISAYNGNTNYAQ |
| | HC | KLQGRVTMTTDTSTSTAYMELRSLRSDDTAVYYCARESSYGSEAFRAGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 139 | [xTL1A]-0347 | GFTFSSYAMS |
| | CDRH1 | |
| 140 | [xTL1A]-0347 | AISGSGGSTYYADSVKG |
| | CDRH2 | |
| 141 | [xTL1A]-0347 | EVQLLESGGGLVQPGGSLRLSCAASGFTFSSYAMSWVRQAPGKGLEWVSAISGSGGSTYYAD |
| | VH | SVKGRFTISRDNSKNTLYLQMNSLRAEDTAVYYCARESYYGSEAFRAGMDVWGQGTTVTVSS |
| 142 | [xTL1A]-0347 | EVQLLESGGGLVQPGGSLRLSCAASGFTFSSYAMSWVRQAPGKGLEWVSAISGSGGSTYYAD |
| | HC | SVKGRFTISRDNSKNTLYLQMNSLRAEDTAVYYCARESYYGSEAFRAGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 143 | [xTL1A]-0349 | EVQLLESGGGLVQPGGSLRLSCAASGFTFSSYAMSWVRQAPGKGLEWVSAISGSGGSTYYAD |
| | VH | SVKGRFTISRDNSKNTLYLQMNSLRAEDTAVYYCARESSYGSEAFRGGMDVWGQGTTVTVSS |
| 144 | [xTL1A]-0349 | EVQLLESGGGLVQPGGSLRLSCAASGFTFSSYAMSWVRQAPGKGLEWVSAISGSGGSTYYAD |
| | HC | SVKGRFTISRDNSKNTLYLQMNSLRAEDTAVYYCARESSYGSEAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 145 | [xTL1A]-0352 | EVQLLESGGGLVQPGGSLRLSCAASGFTFSSYAMSWVRQAPGKGLEWVSAISGSGGSTYYAD |
| | VH | SVKGRFTISRDNSKNTLYLQMNSLRAEDTAVYYCARESSYGSEAFRAGMDVWGQGTTVTVSS |
| 146 | [xTL1A]-0352 | EVQLLESGGGLVQPGGSLRLSCAASGFTFSSYAMSWVRQAPGKGLEWVSAISGSGGSTYYAD |
| | HC | SVKGRFTISRDNSKNTLYLQMNSLRAEDTAVYYCARESSYGSEAFRAGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 147 | [xTL1A]-0358 | GYTFTGYYMH |
| | CDRH1 | |
| 148 | [xTL1A]-0358 | WINPNSGGTNYAQKFQG |
| | CDRH2 | |
| 149 | [xTL1A]-0358 | QVQLVQSGAEVKKPGASVKVSCKASGYTFTGYYMHWVRQAPGQGLEWMGWINPNSGGTNYAQ |
| | VH | KFQGRVTMTRDTSISTAYMELSRLRSDDTAVYYCARESYYGSEAFRAGMDVWGQGTTVTVSS |
| 150 | [xTL1A]-0358 | QVQLVQSGAEVKKPGASVKVSCKASGYTFTGYYMHWVRQAPGQGLEWMGWINPNSGGTNYAQ |
| | HC | KFQGRVTMTRDTSISTAYMELSRLRSDDTAVYYCARESYYGSEAFRAGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 151 | [xTL1A]-0360 | QVQLVQSGAEVKKPGASVKVSCKASGYTFTGYYMHWVRQAPGQGLEWMGWINPNSGGTNYAQ |
| | VH | KFQGRVTMTRDTSISTAYMELSRLRSDDTAVYYCARESSYGSEAFRGGMDVWGQGTTVTVSS |
| 152 | [xTL1A]-0360 | QVQLVQSGAEVKKPGASVKVSCKASGYTFTGYYMHWVRQAPGQGLEWMGWINPNSGGTNYAQ |
| | HC | KFQGRVTMTRDTSISTAYMELSRLRSDDTAVYYCARESSYGSEAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 153 | [xTL1A]-0363 | QVQLVQSGAEVKKPGASVKVSCKASGYTFTGYYMHWVRQAPGQGLEWMGWINPNSGGTNYAQ |
| | VH | KFQGRVTMTRDTSISTAYMELSRLRSDDTAVYYCARESSYGSEAFRAGMDVWGQGTTVTVSS |
| 154 | [xTL1A]-0363 | QVQLVQSGAEVKKPGASVKVSCKASGYTFTGYYMHWVRQAPGQGLEWMGWINPNSGGTNYAQ |
| | HC | KFQGRVTMTRDTSISTAYMELSRLRSDDTAVYYCARESSYGSEAFRAGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQGNVFSCSV |
| | | MHEALHNHYTQKSLSLSPGK |
| 155 | TL1A-0057 CH3 | GQPREPQVCTLPPSREEMTKNQVSLWCLVKGFYPSDIAVEWESNGQPENNYKTTPPVLDSDG |
| | (Functional) | SFFLYSKLTVDKSRWQQGNVFSCSVMHEALHNHYTQKSLSLSPGK |
| 156 | TL1A-0057 | QVQLVQSGAEVKKPGASVKVSCKASGYDFTYYGISWVRQAPGQGLEWMGWISTYNGNTHYAR |
| | Functional HC | MLQGRVTMTTDTSTRTAYMELRSLRSDDTAVYYCARENYYGSGAYRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVCTLPPSREEMTKNQVSL |
| | | WCLVKGFYPSDIAVEWESNGQPENNYKTTPPVLDSDGSFFLYSKLTVDKSRWQQGNVFSCSV |
| | | MHEALHNHYTQKSLSLSPGK |
| 157 | TL1A-0057 | EPKSSDKTHTCPPCP |
| | HINGE | |
| | (Truncated) | |
| 158 | TL1A-0057 CH3 | GQPREPQVYTLPPCREEMTKNQVSLSCAVKGFYPSDIAVEWESNGQPENNYKTTPPVLDSDG |
| | (Truncated) | SFFLVSKLTVDKSRWQQGNVFSCSVMHEALHNHYTQKSLSLSPGK |
| 159 | TL1A-0057 HC | EPKSSDKTHTCPPCPAPEAAGAPSVFLFPPKPKDTLMISRTPEVTCVVVDVSHEDPEVKFNW |
| | (Truncated) | YVDGVEVHNAKTKPREEQYNSTYRVVSVLTVLHQDWLNGKEYKCKVSNKALPAPIEKTISKA |
| | | KGQPREPQVYTLPPCREEMTKNQVSLSCAVKGFYPSDIAVEWESNGQPENNYKTTPPVLDSD |
| | | GSFFLVSKLTVDKSRWQQGNVFSCSVMHEALHNHYTQKSLSLSPGK |
| 160 | TL1A-0326 CH3 | GQPREPQVCTLPPSREEMTKNQVSLWCLVKGFYPSDIAVEWESNGQPENNYKTTPPVLDSDG |
| | (Functional) | SFFLYSKLTVDKSRWQQGNVFSCSVLHEALHSHYTQKSLSLSPGK |
| 161 | TL1A-0326 HC | QVQLVQSGAEVKKPGASVKVSCKASGYDYTYYGISWVRQAPGQGLEWMGWISTYNTGTHYAP |
| | (Functional) | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVCTLPPSREEMTKNQVSL |
| | | WCLVKGFYPSDIAVEWESNGQPENNYKTTPPVLDSDGSFFLYSKLTVDKSRWQQGNVFSCSV |
| | | LHEALHSHYTQKSLSLSPGK |
| 162 | TL1A-0326 CH3 | GQPREPQVYTLPPCREEMTKNQVSLSCAVKGFYPSDIAVEWESNGQPENNYKTTPPVLDSDG |
| | (Truncated) | SFFLVSKLTVDKSRWQQGNVFSCSVLHEALHSHYTQKSLSLSPGK |
| 163 | TL1A-0326 HC | EPKSSDKTHTCPPCPAPEAAGAPSVFLFPPKPKDTLMISRTPEVTCVVVDVSHEDPEVKENW |
| | (Truncated) | YVDGVEVHNAKTKPREEQYNSTYRVVSVLTVLHQDWLNGKEYKCKVSNKALPAPIEKTISKA |
| | | KGQPREPQVYTLPPCREEMTKNQVSLSCAVKGFYPSDIAVEWESNGQPENNYKTTPPVLDSD |
| | | GSFFLVSKLTVDKSRWQQGNVFSCSVLHEALHSHYTQKSLSLSPGK |
| 164 | TL1A-0327 HC | QVQLVQSGAEVKKPGASVKVSCKASGYDYTYYGISWVRQAPGQGLEWMGWISTYNTNTHYAP |
| | (Functional) | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVCTLPPSREEMTKNQVSL |
| | | WCLVKGFYPSDIAVEWESNGQPENNYKTTPPVLDSDGSFFLYSKLTVDKSRWQQGNVFSCSV |
| | | LHEALHSHYTQKSLSLSPGK |
| 165 | TL1A-0328 HC | QVQLVQSGAEVKKPGASVKVSCKASGYDFTYYGIGWVRQAPGQGLEWMGWISTYNTNTHYAP |
| | (Functional) | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVCTLPPSREEMTKNQVSL |
| | | WCLVKGFYPSDIAVEWESNGQPENNYKTTPPVLDSDGSFFLYSKLTVDKSRWQQGNVFSCSV |
| | | LHEALHSHYTQKSLSLSPGK |
| 166 | TL1A- | EVQLLESGGGLVQPGGSLRLSCAASGFTFSSYAMSWVRQAPGKGLEWVSAISGSGGSTYYAD |
| | 0372 [xTL1A] | SVKGRFTISRDNSKNTLYLQMNSLRAEDTAVYYCARESSYGSEAFRGGMDVWGQGTTVTVSS |
| | HC (Disabled) | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPCREEMTKNQVSL |
| | | SCAVKGFYPSDIAVEWESNGQPENNYKTTPPVLDSDGSFFLVSKLTVDKSRWQQGNVFSCSV |
| | | LHEALHSHYTQKSLSLSPGK |
| 167 | p40-0003 CDRH1 | GYSFTTYWLG |
| 168 | p40-0003 CDRH2 | IMSPVDSDIRYSPSFQG |
| 169 | p40-0003 CDRH3 | RRPGQGYFDF |
| 170 | p40-0003 JH | WGQGTLVTVSS |
| 171 | p40-0003 VH | EVQLVQSGAEVKKPGESLKISCKGSGYSFTTYWLGWVRQMPGKGLDWIGIMSPVDSDIRYSP |
| | | SFQGQVTMSVDKSITTAYLQWNSLKASDTAMYYCARRRPGQGYFDFWGQGTLVTVSS |
| 172 | p40-0003 HC | EVQLVQSGAEVKKPGESLKISCKGSGYSFTTYWLGWVRQMPGKGLDWIGIMSPVDSDIRYSP |
| | | SFQGQVTMSVDKSITTAYLQWNSLKASDTAMYYCARRRPGQGYFDFWGQGTLVTVSSASTKG |
| | | PSVFPLAPSSKSTSGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPEAAGAPSVFLFPPK |
| | | PKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRWQQGNVFSCSVMHEAL |
| | | HNHYTQKSLSLSPGK |
| 173 | p40-0003 CDRL1 | RASQGISSWLA |
| 174 | p40-0003 CDRL2 | AASSLQS |
| 175 | p40-0003 CDRL3 | QQYNIYPYT |
| 176 | p40-0003 JK | FGQGTKLEIK |
| 177 | p40-0003 VL | DIQMTQSPSSLSASVGDRVTITCRASQGISSWLAWYQQKPEKAPKSLIYAASSLQSGVPSRF |
| | | SGSGSGTDFTLTISSLQPEDFATYYCQQYNIYPYTFGQGTKLEIK |
| 178 | p40-0003 LC | DIQMTQSPSSLSASVGDRVTITCRASQGISSWLAWYQQKPEKAPKSLIYAASSLQSGVPSRF |
| | | SGSGSGTDFTLTISSLQPEDFATYYCQQYNIYPYTFGQGTKLEIKRTVAAPSVFIFPPSDEQ |
| | | LKSGTASVVCLLNNFYPREAKVQWKVDNALQSGNSQESVTEQDSKDSTYSLSSTLTLSKADY |
| | | EKHKVYACEVTHQGLSSPVTKSFNRGEC |
| 179 | p40TL1A-0001 | EPKSCRKTHTCPRCP |
| | p40 HINGE | |
| 180 | p40TL1A-0001 | GQPREPQVYTLPPSREEMTKNQVSLTCLVKGFYPSDIAVEWESNGQPENNYKTTPPVLDSDG |
| | p40 CH3 | SFFLYSRLTVDKSRWQQGNVFSCSVLHEALHSHYTQKSLSLSPGK |
| 181 | p40TL1A-0001 | EVQLVQSGAEVKKPGESLKISCKGSGYSFTTYWLGWVRQMPGKGLDWIGIMSPVDSDIRYSP |
| | p40 HC | SFQGQVTMSVDKSITTAYLQWNSLKASDTAMYYCARRRPGQGYFDFWGQGTLVTVSSASTKG |
| | | PSVFPLAPSSKSTSGGTAALGCLVKDYFPEPVTVSWNSGALTSGVHTFPAVLQSSGLYSLSS |
| | | VVTVPSSSLGTQTYICNVNHKPSNTKVDKKVEPKSCRKTHTCPRCPAPEAAGAPSVFLFPPK |
| | | PKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMTKNQVSLTCLVK |
| | | GFYPSDIAVEWESNGQPENNYKTTPPVLDSDGSFFLYSRLTVDKSRWQQGNVFSCSVLHEAL |
| | | HSHYTQKSLSLSPGK |
| 182 | p40TL1A-0001 | EPKSCEKTHTCPECP |
| | TL1A HINGE | |
| 183 | p40TL1A-0001 | GQPREPQVYTLPPSREEMTKNQVSLTCEVKGFYPSDIAVEWESNGQPENNYKTTPPVLDSDG |
| | TL1A CH3 | SFFLYSKLTVDKSRWQQGNVFSCSVLHEALHSHYTQKSLSLSPGK |
| 184 | p40TL1A-0001 | QVQLVQSGAEVKKPGASVKVSCKASGYDYTYYGISWVRQAPGQGLEWMGWISTYNTGTHYAP |
| | TL1A HC | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCEKTHTCPECPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCEVKGFYPSDIAVEWESNGQPENNYKTTPPVLDSDGSFFLYSKLTVDKSRWQQGNVFSCSV |
| | | LHEALHSHYTQKSLSLSPGK |
| 185 | p40TL1A-0018 | GQPREPQVYTLPPSREEMTKNQVSLWCLVKGFYPSDIAVEWESNGQPENNYKTTPPVLDSDG |
| | p40 CH3 | SFFLYSKLTVDKSRWQQGNVFSCSVLHEALHSHYTQKSLSLSPGK |
| 186 | p40TL1A-0018 | EVQLVQSGAEVKKPGESLKISCKGSGYSFTTYWLGWVRQMPGKGLDWIGIMSPVDSDIRYSP |
| | p40 HC | SFQGQVTMSVDKSITTAYLQWNSLKASDTAMYYCARRRPGQGYFDFWGQGTLVTVSSASTKG |
| | | PSVFPLAPSSKSTSGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPEAAGAPSVFLFPPK |
| | | PKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMTKNQVSLWCLVK |
| | | GFYPSDIAVEWESNGQPENNYKTTPPVLDSDGSFFLYSKLTVDKSRWQQGNVFSCSVLHEAL |
| | | HSHYTQKSLSLSPGK |
| 187 | p40TL1A-0018 | GQPREPQVYTLPPSREEMTKNQVSLSCAVKGFYPSDIAVEWESNGQPENNYKTTPPVLDSDG |
| | TL1A CH3 | SFFLVSKLTVDKSRWQQGNVFSCSVLHEALHSHYTQKSLSLSPGK |
| 188 | p40TL1A-0018 | QVQLVQSGAEVKKPGASVKVSCKASGYDYTYYGISWVRQAPGQGLEWMGWISTYNTGTHYAP |
| | TLIA HC | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | SCAVKGFYPSDIAVEWESNGQPENNYKTTPPVLDSDGSFFLVSKLTVDKSRWQQGNVFSCSV |
| | | LHEALHSHYTQKSLSLSPGK |
| 189 | p40TL1A-0021 | EPKSCRKTHTCPPCP |
| | p40 HINGE | |
| 190 | p40TL1A-0021 | EVQLVQSGAEVKKPGESLKISCKGSGYSFTTYWLGWVRQMPGKGLDWIGIMSPVDSDIRYSP |
| | p40 HC | SFQGQVTMSVDKSITTAYLQWNSLKASDTAMYYCARRRPGQGYFDFWGQGTLVTVSSASTKG |
| | | PSVFPLAPSSKSTSGGTAALGCLVKDYFPEPVTVSWNSGALTSGVHTFPAVLQSSGLYSLSS |
| | | VVTVPSSSLGTQTYICNVNHKPSNTKVDKKVEPKSCRKTHTCPPCPAPEAAGAPSVFLFPPK |
| | | PKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMTKNQVSLTCLVK |
| | | GFYPSDIAVEWESNGQPENNYKTTPPVLDSDGSFFLYSRLTVDKSRWQQGNVFSCSVLHEAL |
| | | HSHYTQKSLSLSPGK |
| 191 | p40TL1A-0021 | EPKSCEKTHTCPPCP |
| | TL1A HINGE | |
| 192 | p40TL1A-0021 | QVQLVQSGAEVKKPGASVKVSCKASGYDYTYYGISWVRQAPGQGLEWMGWISTYNTGTHYAP |
| | TL1A HC | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCEKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCEVKGFYPSDIAVEWESNGQPENNYKTTPPVLDSDGSFFLYSKLTVDKSRWQQGNVFSCSV |
| | | LHEALHSHYTQKSLSLSPGK |
| 193 | p40TL1A-0024 | EVQLVQSGAEVKKPGESLKISCKGSGYSFTTYWLGWVRQMPGKGLDWIGIMSPVDSDIRYSP |
| | p40 HC | SFQGQVTMSVDKSITTAYLQWNSLKASDTAMYYCARRRPGQGYFDFWGQGTLVTVSSASTKG |
| | | PSVFPLAPSSKSTSGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPEAAGAPSVELFPPK |
| | | PKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMTKNQVSLTCLVK |
| | | GFYPSDIAVEWESNGQPENNYKTTPPVLDSDGSFFLYSRLTVDKSRWQQGNVFSCSVLHEAL |
| | | HSHYTQKSLSLSPGK |
| 194 | p40TL1A-0024 | QVQLVQSGAEVKKPGASVKVSCKASGYDYTYYGISWVRQAPGQGLEWMGWISTYNTGTHYAP |
| | TL1A HC | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPEAAGAPSVE |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPSREEMTKNQVSL |
| | | TCEVKGFYPSDIAVEWESNGQPENNYKTTPPVLDSDGSFFLYSKLTVDKSRWQQGNVFSCSV |
| | | LHEALHSHYTQKSLSLSPGK |
| 195 | p40TL1A-0032 | ASTKGPSVFPKAPSSKSTSGGTAALGCLVKDYFPEPVTVSWNSGALTSGVHTFPAVLQSSGL |
| | p40 CH1 | YSLSSSVTVPSSSLGTQTYICNVNHKPSNTKVDKKV |
| 196 | p40TL1A-0032 | EVQLVQSGAEVKKPGESLKISCKGSGYSFTTYWLGWVRQMPGKGLDWIGIMSPVDSDIRYSP |
| | p40 HC | SFQGQVTMSVDKSITTAYLQWNSLKASDTAMYYCARRRPGQGYFDFWGQGTLVTVSSASTKG |
| | | PSVFPKAPSSKSTSGGTAALGCLVKDYFPEPVTVSWNSGALTSGVHTFPAVLQSSGLYSLSS |
| | | SVTVPSSSLGTQTYICNVNHKPSNTKVDKKVEPKSCDKTHTCPPCPAPEAAGAPSVFLFPPK |
| | | PKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVCTLPPSREEMTKNQVSLWCLVK |
| | | GFYPSDIAVEWESNGQPENNYKTTPPVLDSDGSFFLYSKLTVDKSRWQQGNVFSCSVLHEAL |
| | | HSHYTQKSLSLSPGK |
| 197 | p40TL1A-0032 | (R)TVAAPSVFIFPPSDEQLKSGTASVSCLLNNFYPREAKVQWKVDNALQSGNSQESVTEQD |
| | p40 CL | SKDSTYSLDSTLTLSKADYEKHKVYACEVTHQGLSSPVTKSENRGEC |
| 198 | p40TL1A-0032 | DIQMTQSPSSLSASVGDRVTITCRASQGISSWLAWYQQKPEKAPKSLIYAASSLQSGVPSRF |
| | p40 LC | SGSGSGTDFTLTISSLQPEDFATYYCQQYNIYPYTFGQGTKLEIKRTVAAPSVFIFPPSDEQ |
| | | LKSGTASVSCLLNNFYPREAKVQWKVDNALQSGNSQESVTEQDSKDSTYSLDSTLTLSKADY |
| | | EKHKVYACEVTHQGLSSPVTKSENRGEC |
| 199 | p40TL1A-0032 | ASTKGPSVFPEAPSSKSTSGGTAALGCLVKDYFPEPVTVSWNSGALTSGVHTFPAVLQSSGL |
| | TL1A CH1 | YSLGSVVTVPSSSLGTQTYICNVNHKPSNTKVDKKV |
| 200 | p40TL1A-0032 | QVQLVQSGAEVKKPGASVKVSCKASGYDYTYYGISWVRQAPGQGLEWMGWISTYNTGTHYAP |
| | TL1A HC | SLQGRVTMTTDTSTSTAYMELRSLRSDDTAVYYCARESYYGSGAFRGGMDVWGQGTTVTVSS |
| | | ASTKGPSVFPEAPSSKSTSGGTAALGCLVKDYFPEPVTVSWNSGALTSGVHTFPAVLQSSGL |
| | | YSLGSVVTVPSSSLGTQTYICNVNHKPSNTKVDKKVEPKSCDKTHTCPPCPAPEAAGAPSVF |
| | | LFPPKPKDTLMISRTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPCREEMTKNQVSL |
| | | SCAVKGFYPSDIAVEWESNGQPENNYKTTPPVLDSDGSFFLVSKLTVDKSRWQQGNVFSCSV |
| | | LHEALHSHYTQKSLSLSPGK |
| 201 | p40TL1A-0032 | (R)TVAAPSVFIFPPSDEQLKSGTASVSCLLNNFYPREAKVQWKVDNALQSGNSQESVTEQD |
| | TL1A CL | SKDSTYSLKSTLTLSKADYEKHKVYACEVTHQGLSSPVTKSENRGEC |
| 202 | p40TL1A-0032 | DIQMTQSPSSLSASVGDRVTITCRASQSVSSYLAWYQQKPGKAPKLLIYDASNRATGVPSRF |
| | TL1A LC | SGSGSGTDFTLTISSLQPEDFATYYCQQRSNWPWTFGQGTKVEIKRTVAAPSVFIFPPSDEQ |
| | | LKSGTASVSCLLNNFYPREAKVQWKVDNALQSGNSQESVTEQDSKDSTYSLKSTLTLSKADY |
| | | EKHKVYACEVTHQGLSSPVTKSENRGEC |
| 203 | p40TL1A-0033 | EVQLVQSGAEVKKPGESLKISCKGSGYSFTTYWLGWVRQMPGKGLDWIGIMSPVDSDIRYSP |
| | p40 HC | SFQGQVTMSVDKSITTAYLQWNSLKASDTAMYYCARRRPGQGYFDFWGQGTLVTVSSASTKG |
| | | PSVFPLAPSSKSTSGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPEAAGAPSVFLFPPK |
| | | PKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVCTLPPSREEMTKNQVSLWCLVK |
| | | GFYPSDIAVEWESNGQPENNYKTTPPVLDSDGSFFLYSKLTVDKSRWQQGNVFSCSVLHEAL |
| | | HSHYTQKSLSLSPGK |
| 204 | p40TL1A-0033 | DKTHTCPPCP |
| | TL1A Lower | |
| | HINGE Region | |
| 205 | p40TL1A-0033 | DIQMTQSPSSLSASVGDRVTITCRASQSVSSYLAWYQQKPGKAPKLLIYDASNRATGVPSRF |
| | TL1A LC-Fc | SGSGSGTDFTLTISSLQPEDFATYYCQQRSNWPWTFGQGTKVEIKRTVAAPSVFIFPPSDEQ |
| | | LKSGTASVVCLLNNFYPREAKVQWKVDNALQSGNSQESVTEQDSKDSTYSLSSTLTLSKADY |
| | | EKHKVYACEVTHQGLSSPVTKSFNRGECDKTHTCPPCPAPEAAGAPSVFLFPPKPKDTLMIS |
| | | RTPEVTCVVVDVSHEDPEVKFNWYVDGVEVHNAKTKPREEQYNSTYRVVSVLTVLHQDWING |
| | | KEYKCKVSNKALPAPIEKTISKAKGQPREPQVYTLPPCREEMTKNQVSLSCAVKGFYPSDIA |
| | | VEWESNGQPENNYKTTPPVLDSDGSFFLVSKLTVDKSRWQQGNVFSCSVLHEALHSHYTQKS |
| | | LSLSPGK |
| 206 | p40TL1A-0033 | EPKSC |
| | TL1A Upper | |
| | HINGE Region | |
| 207 | p40TL1A-0033 | QVQLVQSGAEVKKPGASVKVSCKASGYDYTYYGISWVRQAPGQGLEWMGWISTYNTGTHYAP |
| | TL1A mFd | SLQGRVTMTTDTSTSTAYMELRSLRSDDTAVYYCARESYYGSGAFRGGMDVWGQGTTVTVSS |
| | | ASTKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSC |
| 208 | Human TL1A | MGALKGQEFAPSHQQVYAPLRADGDKPRAHLTVVRQTPTQHFKNQFPALHWEHELGLAFTKN |
| | AAI04463.1 | RMNYTNKFLLIPESGDYFIYSQVTFRGMTSESSEIRQAGRPNKPDSITVVITKVTDSYPEPT |
| | | QLLMGTKSVSEVGSNWFQPIYLGAMFSLQEGDKLMVNVSDISLVDYTKEDKTFFGAFLL |
| 209 | Cynomolgus | TGLKGQEFAPSHQQVYAPLRADGDKPRAHLTVVRQTPTQHLKNQFPALHWEHELGLAFTKNR |
| | Monkey TL1A | MNYTNKFLLIPESGDYFVYSQVTFRGMTSECSEIRQAGRPNKPDSITVVITKVTDSYPEPTQ |
| | XP_005581018.1 | LLMGTKSVCEVGSNWFQPIYLGAMFSLQEGDKLMVNVSDISLVDYTKEDKTFFGAFLL |
| 210 | Rabbit TL1A | TGLKGREFGPSQQRAYMPLRADGNKPRAHLTAVKQTPTQPLRNHFPALHWEHELGLAFTKNR |
| | XP_002720561.2 | MNYTNKFLVIPESGDYFVYSQVTFRGTTSECGVINQRRRQTKPDSIVVVITKVTDNYPEPAQ |
| | | LLTGTKSVCEMGNWFQPIYLGAMESLEEGDKLMVNVSDVSLVDYTKEDKTFFGAFLL |
| 211 | Mouse TL1A | GLEGAITEERSEPSPQQVYSPPRGKPRAHLTIKKQTPAPHLKNQLSALHWEHDLGMAFTKNG |
| | Q5UBV8 | MKYINKSLVIPESGDYFIYSQITFRGTTSVCGDISRGRRPNKPDSITVVITKVADSYPEPAR |
| | | LLTGSKSVCEISNNWFQSLYLGAMFSLEEGDRLMVNVSDISLVDYTKEDKTFFGAFLL |
| 212 | Rat TL1A | TGVTEERSAPSAQPVYTPSRDKPKAHLTIMRQTPVPHLKNELAALHWENNLGMAFTKNRMNY |
| | Q8K3Y7 | TNKFLVIPESGDYFIYSQITFRGTTSECGDISRVRRPKKPDSITVVITKVADSYPEPAHLLT |
| | | GTKSVCEISSNWFQPIYLGAMFSLEEGDRLMVNVSDISLVDYTKEDKTFFGAFLI |
| 213 | Human IL-12p40 | MCHQQLVISWFSLVFLASPLVAIWELKKDVYVVELDWYPDAPGEMVVLTCDTPEEDGITWTL |
| | (IL12B) | DQSSEVLGSGKTLTIQVKEFGDAGQYTCHKGGEVLSHSLLLLHKKEDGIWSTDILKDQKEPK |
| | NP_002178 | NKTFLRCEAKNYSGRFTCWWLTTISTDLTFSVKSSRGSSDPQGVTCGAATLSAERVRGDNKE |
| | | YEYSVECQEDSACPAAEESLPIEVMVDAVHKLKYENYTSSFFIRDIIKPDPPKNLQLKPLKN |
| | | SRQVEVSWEYPDTWSTPHSYFSLTFCVQVQGKSKREKKDRVFTDKTSATVICRKNASISVRA |
| | | QDRYYSSSWSEWASVPCS |
| 214 | Cynomolgus | MCHQQLVISWFSLVFLASPLMAIWELKKDVYVVELDWYPDAPGEMVVLTCDTPEEDGITWTL |
| | monkey IL- | DQSGEVLGSGKTLTIQVKEFGDAGQYTCHKGGEALSHSLLLLHKKEDGIWSTDVLKDQKEPK |
| | 12p40 | NKTFLRCEAKNYSGRFTCWWLTTISTDLTFSVKSSRGSSNPQGVTCGAVTLSAERVRGDNKE |
| | (IL12B) | YEYSVECQEDSACPAAEERLPIEVMVDAIHKLKYENYTSSFFIRDIIKPDPPKNLQLKPLKN |
| | NP_001274204 | SRQVEVSWEYPDTWSTPHSYFSLTFCIQVQGKSKREKKDRIFTDKTSATVICRKNASFSVQA |
| | | QDRYYSSSWSEWASVPCS |
| 215 | Human IL-12p35 | MWPPGSASQPPPSPAAATGLHPAARPVSLQCRLSMCPARSLLLVATLVLLDHLSLARNLPVA |
| | (IL12A) | TPDPGMFPCLHHSQNLLRAVSNMLQKARQTLEFYPCTSEEIDHEDITKDKTSTVEACLPLEL |
| | NP_000873 | TKNESCLNSRETSFITNGSCLASRKTSFMMALCLSSIYEDLKMYQVEFKTMNAKLLMDPKRQ |
| | | IFLDQNMLAVIDELMQALNFNSETVPQKSSLEEPDFYKTKIKLCILLHAFRIRAVTIDRVMS |
| | | YLNAS |
| 216 | Cynomolgus | MWPPGSASQPPPSPATATGLHPAARPEPLQCRLSMCPARSLLLVATLVLLDYLSLARNLSVA |
| | monkey IL- | TPGPEMFPCLHHSQNLLKAASNTLQKARQILEFYPCTSEEIDHEDITKDKTSTVDACLPLEL |
| | 12p35 | IKNESCLNSRETSFITNGSCLASRKTSFMMALCLRSIYEDLKMYQVEFKTMNAKLLRDPKRQ |
| | (IL12A) | IFLDQNILGVIDELMQALNFNSETVPQKSSLEEPDFYKTKIKLCILLHAFRIRAVTIDRVMS |
| | XP_005546300 | YLNAS |
| 217 | Human IL-23p19 | MLGSRAVMLLLLLPWTAQGRAVPGGSSPAWTQCQQLSQKLCTLAWSAHPLVGHMDLREEGDE |
| | (IL23A) | ETTNDVPHIQCGDGCDPQGLRDNSQFCLQRIHQGLIFYEKLLGSDIFTGEPSLLPDSPVGQL |
| | NP_057668 | HASLLGLSQLLQPEGHHWETQQIPSLSPSQPWQRLLLRFKILRSLQAFVAVAARVFAHGAAT |
| | | LSP |
| 218 | Cynomolgus IL- | MLGSRAVMLLLLLSWTAQGRAVPGGSSPAWAQCQQLSQKLCTLAWSAHPLVGHMDLREEGDE |
| | 23p19 (IL23A) | ETTNDVPHIQCGDGCDPQGLRDNSQFCLQRIRQGLIFYEKLLGSDIFTGEPSLLPDSPVGQL |
| | NP_001274588 | HASLLGLSQLLQPEGHHWETQQIPSPSPSQPWQRLLLRFKILRSLQAFVAVAARVFAHGAAT |
| | | LSP |
| 219 | Rabbit IL- | IWELEKDVYVVELDWYPDAPGETVVLTCDTSEEDGITWTSEQSNKVLGSGKTLTILVKEFGD |
| | 12p40 (IL12B) | AGQYTCHKGDKVLGHSKVLLHKKEDGIWSTDILKDQKEPKSKTFLKCEAKNYSGHFTCWWLT |
| | KT279675.1 | AISRDVKFSVKSNRGSSDPQGVTCGVPTLSAERVSVDHTEYKFSVECQEDNACPAAEESLHL |
| | | EVMLDAIHKLKYENYTSSFFIRDIVKPDPPQNLQMKPLKNSRHVEVSWEYPDTWSTPHSYFS |
| | | LTFCVQVQNKNKKEKKDRLCVDKTSATVMCHKDAKIRVQARDRYYSSSWSEWAFVSCNGGGE |
| | | NLYFQ |
| 220 | Rabbit IL- | RNLPVATPDLGLFQCLNHSQNLLRAVSDTLHKARQTLEFYSCTSEEIDHEDITKDKTSTVKA |
| | 12p35 (IL12A) | CLPLELTKNESCLASRETSLIINGSCLSSGKTSSMVTLCLSSIYEDLKMYQVEFMAMNAKLL |
| | XM_008266409.2 | MDSKRQIFLDQDMLGAIEELMQALNSNSGAVPQNPSLEELDFYKTKIKLCILLHAFRIRVVT |
| | | IDRMMSYLSSSGGGENLYFQ |
| 221 | Rabbit IL- | RAVPGDSSPAWTQCQQLSQKLCTLAWSAHPQMGPMELLREEGDEETTSDDVPHIQCEDGCDP |
| | 23p19 (IL23A) | QGLRDNTQFCLQRIRQGLFFYEKLLGSDIFTGEPALLPDGPVGQLHASLLGLSQLLQPEGHH |
| | XM_002711079.3 | RETPQTPSLHPSQPWQRRLLRFKILRSLQAFLAVAARVFAHGAATLSPGGGENLYFQ |
| 222 | Mouse IL-12p40 | MWELEKDVYVVEVDWTPDAPGETVNLTCDTPEEDDITWTSDQRHGVIGSGKTLTITVKEFLD |
| | (IL12B) | AGQYTCHKGGETLSHSHLLLHKKENGIWSTEILKNFKNKTFLKCEAPNYSGRFTCSWLVQRN |
| | P43432 | MDLKFNIKSSSSSPDSRAVTCGMASLSAEKVTLDQRDYEKYSVSCQEDVTCPTAEETLPIEL |
| | | ALEARQQNKYENYSTSFFIRDIIKPDPPKNLQMKPLKNSQVEVSWEYPDSWSTPHSYFSLKF |
| | | FVRIQRKKEKMKETEEGCNQKGAFLVEKTSTEVQCKGGNVCVQAQDRYYNSSCSKWACVPCR |
| | | VRS |
| 223 | Mouse IL-12p35 | RVIPVSGPARCLSQSRNLLKTTDDMVKTAREKLKHYSCTAEDIDHEDITRDQTSTLKTCLPL |
| | (IL12A) | ELHKNESCLATRETSSTTRGSCLPPQKTSLMMTLCLGSIYEDLKMYQTEFQAINAALQNHNH |
| | NP_032377 | QQIILDKGMLVAIDELMQSLNHNGETLRQKPPVGEADPYRVKMKLCILLHAFSTRVVTINRV |
| | | MGYLSSA |
| 224 | Mouse IL-23p19 | LAVPRSSSPDWAQCQQLSRNLCMLAWNAHAPAGHMNLLREEEDEETKNNVPRIQCEDGCDPQ |
| | (IL23A) | GLKDNSQFCLQRIRQGLAFYKHLLDSDIFKGEPALLPDSPMEQLHTSLLGLSQLLQPEDHPR |
| | | ETQQMPSLSSSQQWQRPLLRSKILRSLQAFLAIAARVFAHGAATLTEPLVPTA |
| 225 | Rat IL-12p40 | MWELEKDVYVVEVDWRPDAPGETVTLTCDSPEEDDITWTSDQRRGVIGSGKTLTITVREFLD |
| | (IL12B) | AGQYTCHRGGETLSHSHLLLHKKENGIWSTEILKNFKNKTFLKCEAPNYSGRFTCSWLVHRN |
| | NP_072133 | TDLKFNIKSSSSSPESRAVTCGRASLSAEKVTLNQRDYEKYSVACQEDVTCPTAEETLPIEL |
| | | VVEAQQQNKYENYSTSFFIRDIIKPDPPKNLQVKPLKNSQVEVSWEYPDSWSTPHSYFSLKF |
| | | FVRIQRKKEKTKETEEECNQKGAFLVEKTSAEVQCKGANICVQAQDRYYNSSCSKWTCVPCR |
| | | GRS |
| 226 | Rat IL-12p35 | RVIPVSGPAKCLNQSQNLLKTTDDMVRTAREKLKHYSCTAGDIDHEDITRDKTSTLEACLPL |
| | (IL12A) | ELHKNESCLATKETSSIIRGSCLPPQKTSLMMTLCLGSIYEDLKMYQSEFQAINAALQSHNH |
| | Q9R103 | QQITLDRNMLMAIDELMRSLNHSGETLHQKAPMGEADPYRVKMKLCILLHAFSTRVMTINRV |
| | | MNYLSSS |
| 227 | Rat IL-23p19 | LAVPRSSSPDWAQCQQLSRNLCTLAWSAHTPVGQMDLLREEGEEETKSDVPRIQCGDGCDPQ |
| | (IL23A) | GLKDNSQFCLQRIRQGLVFYKHLLDSDIFTGEPSLLPDSPVDQLHTSLLGLSQLLQPEDHHW |
| | NP_569094 | ETQQMPRLSPSQQWQRSLLRSKILRSLQAFLAIAARVFAHGAATLTEPLVPTA |
| 228 | TL1A-0260 VH | CAGGTTCAGCTGGTGCAGTCTGGAGCTGAGGTGAAGAAGCCTGGGGCCTCAGTGAAGGTCTC |
| | | CTGCAAGGCCTCTGGTTACGACTATACCTACTATGGTATCAGCTGGGTGCGACAGGCCCCTG |
| | | GACAAGGGCTTGAGTGGATGGGATGGATCAGCACTTACAATACTGGCACACACTATGCACCC |
| | | AGCCTCCAGGGCAGAGTCACCATGACCACAGACACATCCACCAGCACAGCCTACATGGAGCT |
| | | GAGGAGCCTGAGATCTGACGACACCGCCGTGTATTACTGTGCAAGAGAGAGCTACTATGGTT |
| | | CCGGGGCTTTCAGAGGGGGTATGGACGTCTGGGGCCAAGGGACCACAGTCACCGTCTCCTCA |
| 229 | TL1A-0260 VL | GACATCCAGATGACCCAGTCTCCATCCTCCCTGTCTGCATCTGTAGGAGACAGAGTCACCAT |
| | | CACTTGCAGGGCCAGTCAGAGTGTTAGCAGCTACCTGGCCTGGTATCAGCAGAAACCAGGGA |
| | | AAGCCCCTAAGCTCCTGATCTATGATGCATCCAACAGGGCCACTGGGGTCCCATCAAGGTTC |
| | | AGTGGCAGTGGATCTGGGACAGATTTCACTCTCACCATCAGCAGTCTGCAACCTGAAGATTT |
| | | TGCAACTTACTACTGTCAGCAGCGTAGCAACTGGCCCTGGACCTTCGGCCAAGGGACCAAGG |
| | | TGGAAATCAAA |
| 230 | [xTL1A]-0349 | GAGGTGCAGCTGTTGGAGTCTGGGGGAGGCTTGGTGCAGCCTGGGGGGTCCCTGAGACTCTC |
| | VL | CTGTGCAGCCTCTGGATTCACCTTTAGCAGCTATGCCATGAGCTGGGTCCGCCAGGCTCCAG |
| | | GGAAGGGGCTGGAGTGGGTCTCAGCTATTAGTGGTAGTGGTGGTAGCACATACTACGCAGAC |
| | | TCCGTGAAGGGCCGGTTCACCATCTCCAGAGACAATTCCAAGAACACACTGTATCTGCAAAT |
| | | GAACAGCCTGAGAGCCGAGGACACCGCCGTGTATTACTGTGCAAGAGAGAGCTCCTATGGTT |
| | | CCGAGGCTTTCAGAGGGGGTATGGACGTCTGGGGCCAAGGGACCACAGTCACCGTCTCCTCA |
| 231 | TL1A-0326-HC | CAGGTTCAGCTGGTGCAGTCTGGAGCTGAGGTGAAGAAGCCTGGGGCCTCAGTGAAGGTCTC |
| | | CTGCAAGGCCTCTGGTTACGACTATACCTACTATGGTATCAGCTGGGTGCGACAGGCCCCTG |
| | | GACAAGGGCTTGAGTGGATGGGATGGATCAGCACTTACAATACTGGCACACACTATGCACCC |
| | | AGCCTCCAGGGCAGAGTCACCATGACCACAGACACATCCACCAGCACAGCCTACATGGAGCT |
| | | GAGGAGCCTGAGATCTGACGACACCGCCGTGTATTACTGTGCAAGAGAGAGCTACTATGGTT |
| | | CCGGGGCTTTCAGAGGGGGTATGGACGTCTGGGGCCAAGGGACCACAGTCACCGTCTCCTCA |
| | | GCGTCGACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGTGGA |
| | | ACTCAGGCGCCCTGACCAGCGGCGTGCACACCTTCCCGGCTGTCCTACAGTCCTCAGGACTC |
| | | TACTCCCTCAGCAGCGTGGTGACCGTGCCCTCCAGCAGCTTGGGCACCCAGACCTACATCTG |
| | | CAACGTGAATCACAAGCCCAGCAACACCAAGGTGGACAAGAAAGTTGAGCCCAAATCTTGTG |
| | | ACAAAACTCACACATGCCCACCGTGCCCAGCACCTGAAGCCGCTGGGGCACCGTCAGTCTTC |
| | | CTCTTCCCCCCAAAACCCAAGGACACCCTCATGATCTCCCGGACCCCTGAGGTCACATGCGT |
| | | GGTGGTGGACGTGAGCCACGAAGACCCTGAGGTCAAGTTCAACTGGTACGTGGACGGCGTGG |
| | | AGGTGCATAATGCCAAGACAAAGCCGCGGGAGGAGCAGTACAACAGCACGTACCGTGTGGTC |
| | | AGCGTCCTCACCGTCCTGCACCAGGACTGGCTGAATGGCAAGGAGTACAAGTGCAAGGTCTC |
| | | CAACAAAGCCCTCCCAGCCCCCATCGAGAAAACCATCTCCAAAGCCAAAGGGCAGCCCCGAG |
| | | AACCACAGGTGTGCACCCTGCCCCCATCCCGGGAGGAGATGACCAAGAACCAGGTCAGCCTG |
| | | TGGTGCCTGGTCAAAGGCTTCTATCCCAGCGACATCGCCGTGGAGTGGGAGAGCAATGGGCA |
| | | GCCGGAGAACAACTACAAGACCACGCCTCCCGTGCTGGACTCCGACGGCTCCTTCTTCCTCT |
| | | ATAGCAAGCTCACCGTGGACAAGAGCAGGTGGCAGCAGGGGAACGTCTTCTCATGCTCCGTG |
| | | CTGCATGAGGCTCTGCACAGCCACTACACGCAGAAGAGCCTCTCCCTGTCCCCCGGAAAA |
| 232 | [xTL1A]-0349- | GAGGTGCAGCTGTTGGAGTCTGGGGGAGGCTTGGTGCAGCCTGGGGGGTCCCTGAGACTCTC |
| | HC | CTGTGCAGCCTCTGGATTCACCTTTAGCAGCTATGCCATGAGCTGGGTCCGCCAGGCTCCAG |
| | | GGAAGGGGCTGGAGTGGGTCTCAGCTATTAGTGGTAGTGGTGGTAGCACATACTACGCAGAC |
| | | TCCGTGAAGGGCCGGTTCACCATCTCCAGAGACAATTCCAAGAACACACTGTATCTGCAAAT |
| | | GAACAGCCTGAGAGCCGAGGACACCGCCGTGTATTACTGTGCAAGAGAGAGCTCCTATGGTT |
| | | CCGAGGCTTTCAGAGGGGGTATGGACGTCTGGGGCCAAGGGACCACAGTCACCGTCTCCTCA |
| | | GCGTCGACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGTGGA |
| | | ACTCAGGCGCCCTGACCAGCGGCGTGCACACCTTCCCGGCTGTCCTACAGTCCTCAGGACTC |
| | | TACTCCCTCAGCAGCGTGGTGACCGTGCCCTCCAGCAGCTTGGGCACCCAGACCTACATCTG |
| | | CAACGTGAATCACAAGCCCAGCAACACCAAGGTGGACAAGAAAGTTGAGCCCAAATCTTGTG |
| | | ACAAAACTCACACATGCCCACCGTGCCCAGCACCTGAAGCCGCTGGGGCACCGTCAGTCTTC |
| | | CTCTTCCCCCCAAAACCCAAGGACACCCTCATGATCTCCCGGACCCCTGAGGTCACATGCGT |
| | | GGTGGTGGACGTGAGCCACGAAGACCCTGAGGTCAAGTTCAACTGGTACGTGGACGGCGTGG |
| | | AGGTGCATAATGCCAAGACAAAGCCGCGGGAGGAGCAGTACAACAGCACGTACCGTGTGGTC |
| | | AGCGTCCTCACCGTCCTGCACCAGGACTGGCTGAATGGCAAGGAGTACAAGTGCAAGGTCTC |
| | | CAACAAAGCCCTCCCAGCCCCCATCGAGAAAACCATCTCCAAAGCCAAAGGGCAGCCCCGAG |
| | | AACCACAGGTGTACACCCTGCCCCCATGCCGGGAGGAGATGACCAAGAACCAGGTCAGCCTG |
| | | TCCTGCGCCGTCAAAGGCTTCTATCCCAGCGACATCGCCGTGGAGTGGGAGAGCAATGGGCA |
| | | GCCGGAGAACAACTACAAGACCACGCCTCCCGTGCTGGACTCCGACGGCTCCTTCTTCCTCG |
| | | TTAGCAAGCTCACCGTGGACAAGAGCAGGTGGCAGCAGGGGAACGTCTTCTCATGCTCCGTG |
| | | CTGCATGAGGCTCTGCACAGCCACTACACGCAGAAGAGCCTCTCCCTGTCCCCCGGAAAA |
| 233 | TL1A-LC | GACATCCAGATGACCCAGTCTCCATCCTCCCTGTCTGCATCTGTAGGAGACAGAGTCACCAT |
| | | CACTTGCAGGGCCAGTCAGAGTGTTAGCAGCTACCTGGCCTGGTATCAGCAGAAACCAGGGA |
| | | AAGCCCCTAAGCTCCTGATCTATGATGCATCCAACAGGGCCACTGGGGTCCCATCAAGGTTC |
| | | AGTGGCAGTGGATCTGGGACAGATTTCACTCTCACCATCAGCAGTCTGCAACCTGAAGATTT |
| | | TGCAACTTACTACTGTCAGCAGCGTAGCAACTGGCCCTGGACCTTCGGCCAAGGGACCAAGG |
| | | TGGAAATCAAACGAACTGTGGCTGCACCATCTGTCTTCATCTTCCCGCCATCTGATGAGCAG |
| | | TTGAAATCTGGAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAGTGTCACAGAGC |
| | | AGGACAGCAAGGACAGCACCTACAGCCTCAGCAGCACCCTGACGCTGAGCAAAGCAGACTAC |
| | | GAGAAACACAAAGTCTACGCCTGCGAAGTCACCCATCAGGGCCTGAGCTCGCCCGTCACAAA |
| | | GAGCTTCAACAGGGGAGAGTGT |
| 234 | TL1AmFd | CAGGTTCAGCTGGTGCAGTCTGGAGCTGAGGTGAAGAAGCCTGGGGCCTCAGTGAAGGTCTC |
| | | CTGCAAGGCCTCTGGTTACGACTATACCTACTATGGTATCAGCTGGGTGCGACAGGCCCCTG |
| | | GACAAGGGCTTGAGTGGATGGGATGGATCAGCACTTACAATACTGGCACACACTATGCACCC |
| | | AGCCTCCAGGGCAGAGTCACCATGACCACAGACACATCCACCAGCACAGCCTACATGGAGCT |
| | | GAGGAGCCTGAGATCTGACGACACCGCCGTGTATTACTGTGCAAGAGAGAGCTACTATGGTT |
| | | CCGGGGCTTTCAGAGGGGGTATGGACGTCTGGGGCCAAGGGACCACAGTCACCGTCTCCTCA |
| | | GCGTCGACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGTGGA |
| | | ACTCAGGCGCCCTGACCAGCGGCGTGCACACCTTCCCGGCTGTCCTACAGTCCTCAGGACTC |
| | | TACTCCCTCAGCAGCGTGGTGACCGTGCCCTCCAGCAGCTTGGGCACCCAGACCTACATCTG |
| | | CAACGTGAATCACAAGCCCAGCAACACCAAGGTGGACAAGAAAGTTGAGCCCAAATCTTGT |
| 235 | TL1A-LC-Fc | GACATCCAGATGACCCAGTCTCCATCCTCCCTGTCTGCATCTGTAGGAGACAGAGTCACCAT |
| | | CACTTGCAGGGCCAGTCAGAGTGTTAGCAGCTACCTGGCCTGGTATCAGCAGAAACCAGGGA |
| | | AAGCCCCTAAGCTCCTGATCTATGATGCATCCAACAGGGCCACTGGGGTCCCATCAAGGTTC |
| | | AGTGGCAGTGGATCTGGGACAGATTTCACTCTCACCATCAGCAGTCTGCAACCTGAAGATTT |
| | | TGCAACTTACTACTGTCAGCAGCGTAGCAACTGGCCCTGGACCTTCGGCCAAGGGACCAAGG |
| | | TGGAAATCAAACGTACGGTGGCTGCACCATCTGTCTTCATCTTCCCGCCATCTGATGAGCAG |
| | | TTGAAATCTGGAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAGTGTCACAGAGC |
| | | AGGACAGCAAGGACAGCACCTACAGCCTCAGCAGCACCCTGACGCTGAGCAAAGCAGACTAC |
| | | GAGAAACACAAAGTCTACGCCTGCGAAGTCACCCATCAGGGCCTGAGCTCGCCCGTCACAAA |
| | | GAGCTTCAACAGGGGAGAGTGTGACAAAACTCACACATGCCCACCGTGCCCAGCACCTGAAG |
| | | CCGCTGGGGCACCGTCAGTCTTCCTCTTCCCCCCAAAACCCAAGGACACCCTCATGATCTCC |
| | | CGGACCCCTGAGGTCACATGCGTGGTGGTGGACGTGAGCCACGAAGACCCTGAGGTCAAGTT |
| | | CAACTGGTACGTGGACGGCGTGGAGGTGCATAATGCCAAGACAAAGCCGCGGGAGGAGCAGT |
| | | ACAACAGCACGTACCGTGTGGTCAGCGTCCTCACCGTCCTGCACCAGGACTGGCTGAATGGC |
| | | AAGGAGTACAAGTGCAAGGTCTCCAACAAAGCCCTCCCAGCCCCCATCGAGAAAACCATCTC |
| | | CAAAGCCAAAGGGCAGCCCCGAGAACCACAGGTGTACACCCTGCCCCCATGCCGGGAGGAGA |
| | | TGACCAAGAACCAGGTCAGCCTGTCCTGCGCGGTCAAAGGCTTCTATCCCAGCGACATCGCC |
| | | GTGGAGTGGGAGAGCAATGGGCAGCCGGAGAACAACTACAAGACCACGCCTCCCGTGCTGGA |
| | | CTCCGACGGCTCCTTCTTCCTCGTTAGCAAGCTCACCGTGGACAAGAGCAGGTGGCAGCAGG |
| | | GGAACGTCTTCTCATGCTCCGTGCTGCATGAGGCTCTGCACAGCCACTACACGCAGAAGAGC |
| | | CTCTCCCTGTCCCCGGGAAAA |
| 236 | p40-HC | GAGGTGCAGCTGGTGCAGTCTGGAGCAGAGGTGAAAAAGCCCGGGGAGTCTCTGAAGATCTC |
| | | CTGTAAGGGTTCTGGATACAGCTTTACCACCTACTGGCTGGGCTGGGTGCGCCAGATGCCCG |
| | | GGAAAGGCCTGGATTGGATAGGGATCATGAGCCCTGTGGACTCTGATATTAGATACAGCCCC |
| | | TCCTTCCAAGGCCAGGTCACCATGTCAGTGGACAAGTCCATCACCACCGCCTACCTGCAGTG |
| | | GAATAGCCTGAAGGCCTCCGACACCGCCATGTATTACTGTGCCAGACGACGCCCTGGTCAAG |
| | | GTTATTTCGATTTTTGGGGGCAAGGAACATTGGTGACCGTCAGTTCAGCGTCGACCAAGGGC |
| | | CCATCGGTCTTCCCCCTGGCACCCTCCTCCAAGAGCACCTCTGGGGGCACAGCGGCCCTGGG |
| | | CTGCCTGGTCAAGGACTACTTCCCCGAACCGGTGACGGTGTCGTGGAACTCAGGCGCCCTGA |
| | | CCAGCGGCGTGCACACCTTCCCGGCTGTCCTACAGTCCTCAGGACTCTACTCCCTCAGCAGC |
| | | GTGGTGACCGTGCCCTCCAGCAGCTTGGGCACCCAGACCTACATCTGCAACGTGAATCACAA |
| | | GCCCAGCAACACCAAGGTGGACAAGAAAGTTGAGCCCAAATCTTGTGACAAAACTCACACAT |
| | | GCCCACCGTGCCCAGCACCTGAAGCCGCTGGGGCACCGTCAGTCTTCCTCTTCCCCCCAAAA |
| | | CCCAAGGACACCCTCATGATCTCCCGGACCCCTGAGGTCACATGCGTGGTGGTGGACGTGAG |
| | | CCACGAAGACCCTGAGGTCAAGTTCAACTGGTACGTGGACGGCGTGGAGGTGCATAATGCCA |
| | | AGACAAAGCCGCGGGAGGAGCAGTACAACAGCACGTACCGTGTGGTCAGCGTCCTCACCGTC |
| | | CTGCACCAGGACTGGCTGAATGGCAAGGAGTACAAGTGCAAGGTCTCCAACAAAGCCCTCCC |
| | | AGCCCCCATCGAGAAAACCATCTCCAAAGCCAAAGGGCAGCCCCGAGAACCACAGGTGTGCA |
| | | CCCTGCCCCCATCCCGGGAGGAGATGACCAAGAACCAGGTCAGCCTGTGGTGCCTGGTCAAA |
| | | GGCTTCTATCCCAGCGACATCGCCGTGGAGTGGGAGAGCAATGGGCAGCCGGAGAACAACTA |
| | | CAAGACCACGCCTCCCGTGCTGGACTCCGACGGCTCCTTCTTCCTCTATAGCAAGCTCACCG |
| | | TGGACAAGAGCAGGTGGCAGCAGGGGAACGTCTTCTCATGCTCCGTGCTGCATGAGGCTCTG |
| | | CACAGCCACTACACGCAGAAGAGCCTCTCCCTGTCCCCGGGAAAA |
| 237 | p40-LC | GACATCCAGATGACCCAGTCTCCATCCTCACTGTCTGCATCTGTGGGAGACAGAGTCACCAT |
| | | CACTTGTCGGGCCAGTCAGGGTATTAGCAGCTGGCTGGCCTGGTATCAGCAGAAACCAGAGA |
| | | AAGCCCCTAAGTCCCTGATCTATGCTGCATCCAGTTTGCAAAGTGGGGTCCCATCAAGGTTC |
| | | AGCGGCAGTGGATCTGGGACAGATTTCACTCTCACCATCAGCAGCCTGCAGCCTGAAGATTT |
| | | TGCAACTTATTACTGCCAACAGTATAATATTTATCCCTACACCTTCGGACAAGGTACAAAAC |
| | | TGGAGATCAAACGAACTGTGGCTGCACCATCTGTCTTCATCTTCCCGCCATCTGATGAGCAG |
| | | TTGAAATCTGGAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAGTGTCACAGAGC |
| | | AGGACAGCAAGGACAGCACCTACAGCCTCAGCAGCACCCTGACGCTGAGCAAAGCAGACTAC |
| | | GAGAAACACAAAGTCTACGCCTGCGAAGTCACCCATCAGGGCCTGAGCTCGCCCGTCACAAA |
| | | GAGCTTCAACAGGGGAGAGTGT |
| 238 | p40 VH | GAGGTGCAGCTGGTGCAGTCTGGAGCAGAGGTGAAAAAGCCCGGGGAGTCTCTGAAGATCTC |
| | | CTGTAAGGGTTCTGGATACAGCTTTACCACCTACTGGCTGGGCTGGGTGCGCCAGATGCCCG |
| | | GGAAAGGCCTGGATTGGATAGGGATCATGAGCCCTGTGGACTCTGATATTAGATACAGCCCC |
| | | TCCTTCCAAGGCCAGGTCACCATGTCAGTGGACAAGTCCATCACCACCGCCTACCTGCAGTG |
| | | GAATAGCCTGAAGGCCTCCGACACCGCCATGTATTACTGTGCCAGACGACGCCCTGGTCAAG |
| | | GTTATTTCGATTTTTGGGGGCAAGGAACATTGGTGACCGTCAGTTCA |
| 239 | p40 VL | GACATCCAGATGACCCAGTCTCCATCCTCACTGTCTGCATCTGTGGGAGACAGAGTCACCAT |
| | | CACTTGTCGGGCCAGTCAGGGTATTAGCAGCTGGCTGGCCTGGTATCAGCAGAAACCAGAGA |
| | | AAGCCCCTAAGTCCCTGATCTATGCTGCATCCAGTTTGCAAAGTGGGGTCCCATCAAGGTTC |
| | | AGCGGCAGTGGATCTGGGACAGATTTCACTCTCACCATCAGCAGCCTGCAGCCTGAAGATTT |
| | | TGCAACTTATTACTGCCAACAGTATAATATTTATCCCTACACCTTCGGACAAGGTACAAAAC |
| | | TGGAGATCAAA |

1. An isolated antibody comprising a first antigen binding site that binds to TL1A and a second antigen binding site that binds to p40, wherein the first antigen binding site comprises a TL1A-binding heavy chain variable region (TL1A-VH) and a TL1A-binding light chain variable region (TL1A-VL), wherein the second antigen binding site comprises a p40-binding heavy chain variable region (p40-VH) and a p40-binding light chain variable region (p40-VL), and wherein the p40 binding domain comprises a CDR-L1 sequence according to SEQ ID NO: 173; a CDR-L2 sequence according to SEQ ID NO: 174, and a CDR-L3 sequence according to SEQ ID NO: 175, a CDR-H1 sequence according to SEQ ID NO: 167, a CDR-H2 sequence according to SEQ ID NO: 168, and a CDR-H3 sequence according to SEQ ID NO: 169, and wherein the TL1A binding domain comprises a CDR-L1 sequence according to SEQ ID NO: 11; a CDR-L2 sequence according to SEQ ID NO: 12, and a CDR-L3 sequence according to SEQ ID NO: 13, and further comprises a set of heavy chain CDRs selected from the list:
(i) a CDR-H1 sequence according to SEQ ID NO: 30; a CDR-H2 sequence according to SEQ ID NO: 41; a CDR-H3 sequence according to SEQ ID NO: 32;
(ii) a CDR-H1 sequence according to SEQ ID NO: 30; a CDR-H2 sequence according to SEQ ID NO: 41; a CDR-H3 sequence according to SEQ ID NO: 35;
(iii) a CDR-H1 sequence according to SEQ ID NO: 30; a CDR-H2 sequence according to SEQ ID NO: 46; a CDR-H3 sequence according to SEQ ID NO: 32;
(iv) a CDR-H1 sequence according to SEQ ID NO: 30; a CDR-H2 sequence according to SEQ ID NO: 46; a CDR-H3 sequence according to SEQ ID NO: 35;
(v) a CDR-H1 sequence according to SEQ ID NO: 30; a CDR-H2 sequence according to SEQ ID NO: 51; a CDR-H3 sequence according to SEQ ID NO: 32;
(vi) a CDR-H1 sequence according to SEQ ID NO: 30; a CDR-H2 sequence according to SEQ ID NO: 51; a CDR-H3 sequence according to SEQ ID NO: 35;
(vii) a CDR-H1 sequence according to SEQ ID NO: 30; a CDR-H2 sequence according to SEQ ID NO: 56; a CDR-H3 sequence according to SEQ ID NO: 32;
(viii) a CDR-H1 sequence according to SEQ ID NO: 30; a CDR-H2 sequence according to SEQ ID NO: 56; a CDR-H3 sequence according to SEQ ID NO: 35;
(ix) a CDR-H1 sequence according to SEQ ID NO: 61; a CDR-H2 sequence according to SEQ ID NO: 41; a CDR-H3 sequence according to SEQ ID NO: 32;
(x) a CDR-H1 sequence according to SEQ ID NO: 61; a CDR-H2 sequence according to SEQ ID NO: 41; a CDR-H3 sequence according to SEQ ID NO: 35;
(xi) a CDR-H1 sequence according to SEQ ID NO: 61; a CDR-H2 sequence according to SEQ ID NO: 51; a CDR-H3 sequence according to SEQ ID NO: 32;
(xii) a CDR-H1 sequence according to SEQ ID NO: 61; a CDR-H2 sequence according to SEQ ID NO: 56; a CDR-H3 sequence according to SEQ ID NO: 32;
(xiii) a CDR-H1 sequence according to SEQ ID NO: 86; a CDR-H2 sequence according to SEQ ID NO: 31; a CDR-H3 sequence according to SEQ ID NO: 35;
(xiv) a CDR-H1 sequence according to SEQ ID NO: 86; a CDR-H2 sequence according to SEQ ID NO: 38; a CDR-H3 sequence according to SEQ ID NO: 35;
(xv) a CDR-H1 sequence according to SEQ ID NO: 86; a CDR-H2 sequence according to SEQ ID NO: 41; a CDR-H3 sequence according to SEQ ID NO: 32;
(xvi) a CDR-H1 sequence according to SEQ ID NO: 86; a CDR-H2 sequence according to SEQ ID NO: 41; a CDR-H3 sequence according to SEQ ID NO: 35;
(xvii) a CDR-H1 sequence according to SEQ ID NO: 86; a CDR-H2 sequence according to SEQ ID NO: 46; a CDR-H3 sequence according to SEQ ID NO: 35;
(xviii) a CDR-H1 sequence according to SEQ ID NO: 86; a CDR-H2 sequence according to SEQ ID NO: 51; a CDR-H3 sequence according to SEQ ID NO: 32;
(xix) a CDR-H1 sequence according to SEQ ID NO: 86; a CDR-H2 sequence according to SEQ ID NO: 51; a CDR-H3 sequence according to SEQ ID NO: 35;
(xx) a CDR-H1 sequence according to SEQ ID NO: 86; a CDR-H2 sequence according to SEQ ID NO: 56; a CDR-H3 sequence according to SEQ ID NO: 32;
(xxi) a CDR-H1 sequence according to SEQ ID NO: 109; a CDR-H2 sequence according to SEQ ID NO: 41; a CDR-H3 sequence according to SEQ ID NO: 35; and
(xxii) a CDR-H1 sequence according to SEQ ID NO: 109; a CDR-H2 sequence according to SEQ ID NO: 56; a CDR-H3 sequence according to SEQ ID NO: 32.

2. An isolated antibody comprising a first antigen binding site that binds to TL1A and a second antigen binding site that binds to p40, wherein the first antigen binding site comprises a TL1A-binding heavy chain variable region (TL1A-VH) and a TL1A-binding light chain variable region (TL1A-VL), wherein the second antigen binding site comprises a p40-binding heavy chain variable region (p40-VH) and a p40-binding light chain variable region (p40-VL), and wherein
(i) the p40 binding domain comprises a CDR-L1 sequence according to SEQ ID NO: 173; a CDR-L2 sequence according to SEQ ID NO: 174, and a CDR-L3 sequence according to SEQ ID NO: 175, a CDR-H1 sequence according to SEQ ID NO: 167, a CDR-H2 sequence according to SEQ ID NO: 168, and a CDR-H3 sequence according to SEQ ID NO: 169, and
(ii) the TL1A binding domain comprises a CDR-L1 sequence according to SEQ ID NO: 11; a CDR-L2 sequence according to SEQ ID NO: 12, and a CDR-L3 sequence according to SEQ ID NO: 13, a CDR-H1 sequence according to SEQ ID NO: 30; a CDR-H2 sequence according to SEQ ID NO: 41; and a CDR-H3 sequence according to SEQ ID NO: 32.

3. The antibody of any claim 1, comprising a TL1A-VH framework sequence derived from a human germline VH sequence selected from the group consisting of IGHV1-18*01, IGHV1-18*01, IGHV1-18*01, IGHV1-46*01, IGHV3-7*01, IGHV1-2*02, IGHV1-46*01, IGHV3-7*01, IGHV1-2*02, IGHV1-46*01, IGHV3-7*01, IGHV1-2*02, and a TL1A-VL framework sequence derived from a human germline VL sequence selected from the group consisting of IGKV1-39*01, IGKV3-11*01, IGKV3D-7*01, IGKV3-11*01, IGKV3-11*01, IGKV3-11*01, IGKV1-39*01, IGKV1-39*01, IGKV1-39*01, IGKV3D-7*01, IGKV3D-7*01, IGKV3D-7*01.

4. The antibody of claim 1, wherein the TL1A-VL comprises an amino acid sequence according to SEQ ID NO: 18, and wherein the TL1A-VH comprises an amino acid sequence according to a sequence selected from the group consisting of SEQ ID NO: 42, SEQ ID NO: 44, SEQ ID NO: 47, SEQ ID NO:49, SEQ ID NO:52, SEQ ID NO: 54, SEQ ID NO: 57, SEQ ID NO: 70, SEQ ID NO: 72, SEQ ID NO: 78, SEQ ID NO: 80, SEQ ID NO: 89, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 110, and SEQ ID NO:120.

5. The antibody of claim 1, comprising the TL1A-VH sequence of SEQ ID NO: 42, and the L1A-VL of SEQ ID NO: 18.

6. The antibody of claim 1, comprising a TL1A-VH sequence encoded by a nucleic acid sequence of SEQ ID NO: 228, and comprising a TL1A-VL sequence encoded by a nucleic acid sequence of SEQ ID NO: 229.

7. The antibody of claim 1, comprising a p40-VH framework sequence derived from a human germline VH sequence selected from the group consisting of DP3, DP7, DP73, DP75, and DP88, and comprising a p40-VL framework sequence derived from a human germline VL sequence selected from the group consisting of DPK4, DPK5, DPK7, DPK8, and DPK9.

8. The antibody of claim 1, wherein the p40-VL comprises an amino acid sequence according to SEQ ID NO: 177, and wherein the p40-VH comprises an amino acid sequence according to SEQ ID NO: 171.

9. The antibody of claim 1, comprising a p40-VH sequence encoded by a nucleic acid sequence of SEQ ID NO: 238 and comprising a p40-VL sequence encoded by a nucleic acid sequence of SEQ ID NO: 239.

10. The antibody of claim 1, wherein the antibody comprises a first, second, third, and fourth polypeptide chain, such that the first and third polypeptide chains together form the TL1A-Fab, and the second and fourth polypeptide chains together form the p40-Fab.

11. The antibody of claim 10, wherein the first, second, third, and fourth polypeptide chains have a combination of sequences in accordance with one of the groups selected from the following list:
(i) the first polypeptide sequence is in accordance with SEQ ID NO: 205, the second polypeptide is in accordance with SEQ ID NO: 203, the third polypeptide is in accordance with SEQ ID NO: 207, and the fourth polypeptide is in accordance with SEQ ID NO: 178;
(ii) the first polypeptide sequence is in accordance with SEQ ID NO: 188, the second polypeptide is in accordance with SEQ ID NO: 186, the third polypeptide is in accordance with SEQ ID NO: 19, and the fourth polypeptide is in accordance with SEQ ID NO: 178;
(iii) the first polypeptide sequence is in accordance with SEQ ID NO: 192, the second polypeptide is in accordance with SEQ ID NO: 190, the third polypeptide is in accordance with SEQ ID NO: 19, and the fourth polypeptide is in accordance with SEQ ID NO: 178;
(iv) the first polypeptide sequence is in accordance with SEQ ID NO: 194, the second polypeptide is in accordance with SEQ ID NO: 193, the third polypeptide is in accordance with SEQ ID NO: 19, and the fourth polypeptide is in accordance with SEQ ID NO: 178; and
(v) the first polypeptide sequence is in accordance with SEQ ID NO: 200, the second polypeptide is in accordance with SEQ ID NO: 196, the third polypeptide is in accordance with SEQ ID NO: 202, and the fourth polypeptide is in accordance with SEQ ID NO: 198.

12. The antibody of claim 11, wherein the first polypeptide sequence is in accordance with SEQ ID NO: 205, the second polypeptide is in accordance with SEQ ID NO: 203, the third polypeptide is in accordance with SEQ ID NO: 207, and the fourth polypeptide is in accordance with SEQ ID NO: 178.

13. An isolated antibody that specifically binds to TL1A and p40, wherein the antibody comprises a first, second, third, and fourth polypeptide chain, such that the first and third polypeptide chains together form a TL1A binding region, and the second and fourth polypeptide chains together form a p40 binding region, and wherein the first polypeptide sequence is in accordance with SEQ ID NO: 205, the second polypeptide is in accordance with SEQ ID NO: 203, the third polypeptide is in accordance with SEQ ID NO: 207, and the fourth polypeptide is in accordance with SEQ ID NO: 178.

14. An isolated antibody that specifically binds to TL1A and p40, wherein the antibody comprises a first, second, third, and fourth polypeptide chain, such that the first and third polypeptide chains together form a TL1A binding region, and the second and fourth polypeptide chains together form a p40 binding region, and wherein the first polypeptide sequence comprises a sequence encoded by the plasmid deposited at the ATCC and having ATCC Accession No. PTA-127349, the second polypeptide comprises a sequence encoded by the plasmid deposited at the ATCC and having ATCC Accession No. PTA-127346, the third polypeptide comprises a sequence encoded by the plasmid deposited at the ATCC and having ATCC Accession No. PTA-127350, and the fourth polypeptide comprises a sequence encoded by the plasmid deposited at the ATCC and having ATCC Accession No. PTA-127203.

15. The antibody of claim 1, characterized by a score of less than 2 in an affinity-capture self-interaction nanoparticle spectroscopy (AC SINS) assay.

16. The antibody of claim 1, characterized in that the charge heterogeneity of the antibody is at least 50% non-acid and non-basic as measured in an antibody imaged capillary electrophoresis (iCE) for an unstressed sample (T0) to evaluate.

17. The antibody of claim 1, wherein the antibody binds to immobilized human TL1A with a binding affinity of less than 1 nM, as measured by SPR, and or wherein the immobilized antibody binds to human p40 with a binding affinity of less than 1 nM, as measured by SPR.

18. The antibody of claim 1, wherein Ab binds p40(IL-23) and TL1A simultaneously in an SPR assay wherein the antibody is injected over immobilized TL1A and the resulting complex subsequently treated with IL-23.

19. The antibody of claim 1, wherein the antibody has a terminal half-life of at least 10 days in TG32 mice, based on a two-week analysis and/or wherein the antibody has a terminal half-life of at least 21 days in cynomolgus monkeys.

20. The antibody of 1, for use as a medicament, wherein the use is for the treatment of one or more selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis.

21. A pharmaceutical composition comprising a therapeutically effective amount of the antibody of claim 1, and a pharmaceutically acceptable carrier.

22. An isolated polynucleotide, comprising one or more nucleotide sequences encoding the antibody of claim 1, or encoding one or more of the first, second, third, and fourth polypeptides of an antibody that binds to both TL1A and p40, comprising
(i) an TL1A-VH sequence encoded by a nucleic acid comprising the nucleic acid sequence of SEQ ID NO: 228, and an TL1A-VL encoded by a nucleic acid comprising the nucleic acid sequence of SEQ ID NO: 229; and
(ii) an p40-VH sequence encoded by a nucleic acid comprising the nucleic acid sequence of SEQ ID NO: 238, and an p40-VL sequence encoded by a nucleic acid comprising the nucleic acid sequence of SEQ ID NO: 239.

23. An isolated polynucleotide encoding one or more of the first, second, third, and fourth polypeptides of an antibody that binds to both TL1A and p40, comprising
(i) an TL1A-VH bearing polypeptide sequence encoded by a nucleic acid comprising the nucleic acid sequence of SEQ ID NO: 234, and an TL1A-VL bearing polypeptide encoded by a nucleic acid comprising the nucleic acid sequence of SEQ ID NO: 235; and
(ii) an p40-HC sequence encoded by a nucleic acid comprising the nucleic acid sequence of SEQ ID NO: 236, and an p40-LC sequence encoded by a nucleic acid comprising the nucleic acid sequence of SEQ ID NO: 237.

24. A vector comprising the polynucleotide of claim 22.

25. An isolated host cell comprising the vector of claim 24.

26. A method of producing an isolated antibody, comprising culturing the host cell of claim under conditions that result in production of the antibody, and recovering the antibody.


SEQUENCE LISTING (239 sequences)

SEQ ID NO: 1 - protein, 10 aa, synthetic construct
  REGION 1..10: note = Synthetic Construct
GYDFTYYGIS

SEQ ID NO: 2 - protein, 17 aa, synthetic construct
  REGION 1..17: note = Synthetic Construct
WISTYNGNTHYARMLQG

SEQ ID NO: 3 - protein, 15 aa, synthetic construct
  REGION 1..15: note = Synthetic Construct
ENYYGSGAYRGGMDV

SEQ ID NO: 4 - protein, 11 aa, synthetic construct
  REGION 1..11: note = Synthetic Construct
WGQGTTVTVSS

SEQ ID NO: 5 - protein, 124 aa, synthetic construct
  REGION 1..124: note = Synthetic Construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 6 - protein, 98 aa, synthetic construct
  REGION 1..98: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 7 - protein, 15 aa, synthetic construct
  REGION 1..15: note = Synthetic Construct
EPKSCDKTHTCPPCP

SEQ ID NO: 8 - protein, 110 aa, synthetic construct
  REGION 1..110: note = Synthetic Construct
APEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 9 - protein, 106 aa, synthetic construct
  REGION 1..106: note = Synthetic Construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 454 aa, synthetic construct
  REGION 1..454: note = Synthetic Construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 11 aa, synthetic construct
  REGION 1..11: note = Synthetic Construct
RASQSVSSYLA

SEQ ID NO: 12 - protein, 7 aa, synthetic construct
  REGION 1..7: note = Synthetic Construct
DASNRAT

SEQ ID NO: 13 - protein, 9 aa, synthetic construct
  REGION 1..9: note = Synthetic Construct
QQRSNWPWT

SEQ ID NO: 14 - protein, 10 aa, synthetic construct
  REGION 1..10: note = Synthetic Construct
FGQGTKVEIK

SEQ ID NO: 15 - protein, 107 aa, synthetic construct
  REGION 1..107: note = Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 16 - protein, 107 aa, synthetic construct [sequence omitted]

SEQ ID NO: 17 - protein, 214 aa, synthetic construct
  REGION 1..214: note = Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 107 aa, synthetic construct
  REGION 1..107: note = Synthetic Construct
DIQMTQSPSSLSASVGDRVTITCRASQSVSSYLAWYQQKPGKAPKLLIYDASNRATGVPSRFSGSGSGTDFTLTISSLQPEDFATYYCQQRSNWPWTFGQGTKVEIK

SEQ ID NO: 19 - protein, 214 aa, synthetic construct
  REGION 1..214: note = Synthetic Construct
DIQMTQSPSSLSASVGDRVTITCRASQSVSSYLAWYQQKPGKAPKLLIYDASNRATGVPSRFSGSGSGTDFTLTISSLQPEDFATYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 107 aa, synthetic construct
  REGION 1..107: note = Synthetic Construct
EIVMTQSPATLSLSPGERATLSCRASQSVSSYLAWYQQKPGQAPRLLIYDASNRATGIPARFSGSGSGTDFTLTISSLQPEDFAVYYCQQRSNWPWTFGQGTKVEIK

SEQ ID NO: 21 - protein, 214 aa, synthetic construct
  REGION 1..214: note = Synthetic Construct
EIVMTQSPATLSLSPGERATLSCRASQSVSSYLAWYQQKPGQAPRLLIYDASNRATGIPARFSGSGSGTDFTLTISSLQPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 124 aa, synthetic construct
  REGION 1..124: note = Synthetic Construct
QVQLVQSGAEVKKPGASVKVSCKASGYDFTYYGISWVRQAPGQGLEWMGWISTYNGNTHYARMLQGRVTMTRDTSTSTVYMELSSLRSEDTAVYYCARENYYGSGAYRGGMDVWGQGTTVTVSS

SEQ ID NO: 23 - protein, 454 aa, synthetic construct
  REGION 1..454: note = Synthetic Construct
QVQLVQSGAEVKKPGASVKVSCKASGYDFTYYGISWVRQAPGQGLEWMGWISTYNGNTHYARMLQGRVTMTRDTSTSTVYMELSSLRSEDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 124 aa, synthetic construct
  REGION 1..124: note = Synthetic Construct
EVQLVESGGGLVQPGGSLRLSCAASGYDFTYYGISWVRQAPGKGLEWVAWISTYNGNTHYARMLQGRFTISRDNAKNSLYLQMNSLRAEDTAVYYCARENYYGSGAYRGGMDVWGQGTTVTVSS

SEQ ID NO: 25 - protein, 454 aa, synthetic construct
  REGION 1..454: note = Synthetic Construct
EVQLVESGGGLVQPGGSLRLSCAASGYDFTYYGISWVRQAPGKGLEWVAWISTYNGNTHYARMLQGRFTISRDNAKNSLYLQMNSLRAEDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 124 aa, synthetic construct
  REGION 1..124: note = Synthetic Construct
QVQLVQSGAEVKKPGASVKVSCKASGYDFTYYGISWVRQAPGQGLEWMGWISTYNGNTHYARMLQGRVTMTRDTSISTAYMELSRLRSDDTAVYYCARENYYGSGAYRGGMDVWGQGTTVTVSS

SEQ ID NO: 27 - protein, 454 aa, synthetic construct
  REGION 1..454: note = Synthetic Construct
QVQLVQSGAEVKKPGASVKVSCKASGYDFTYYGISWVRQAPGQGLEWMGWISTYNGNTHYARMLQGRVTMTRDTSISTAYMELSRLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 124 aa, synthetic construct
  REGION 1..124: note = Synthetic Construct
QVQLVQSGAEVKKPGASVKVSCKASGYDFTYYGISWVRQAPGQGLEWMGWISTYNGNTHYARMLQGRVTMTTDTSTSTAYMELRSLRSDDTAVYYCARENYYGSGAYRGGMDVWGQGTTVTVSS

SEQ ID NO: 29 - protein, 454 aa, synthetic construct
  REGION 1..454: note = Synthetic Construct
QVQLVQSGAEVKKPGASVKVSCKASGYDFTYYGISWVRQAPGQGLEWMGWISTYNGNTHYARMLQGRVTMTTDTSTSTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 10 aa, synthetic construct
  REGION 1..10: note = Synthetic Construct
GYDYTYYGIS

SEQ ID NO: 31 - protein, 17 aa, synthetic construct
  REGION 1..17: note = Synthetic Construct
WISTYNDNTHYAPSLQG

SEQ ID NO: 32 - protein, 15 aa, synthetic construct
  REGION 1..15: note = Synthetic Construct
ESYYGSGAFRGGMDV

SEQ ID NO: 33 - protein, 124 aa, synthetic construct
  REGION 1..124: note = Synthetic Construct
QVQLVQSGAEVKKPGASVKVSCKASGYDYTYYGISWVRQAPGQGLEWMGWISTYNDNTHYAPSLQGRVTMTTDTSTSTAYMELRSLRSDDTAVYYCARESYYGSGAFRGGMDVWGQGTTVTVSS

SEQ ID NO: 34 - protein, 454 aa, synthetic construct
  REGION 1..454: note = Synthetic Construct
QVQLVQSGAEVKKPGASVKVSCKASGYDYTYYGISWVRQAPGQGLEWMGWISTYNDNTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 15 aa, synthetic construct
  REGION 1..15: note = Synthetic Construct
ESYYGSGAHRGGMDV

SEQ ID NO: 36 - protein, 124 aa, synthetic construct
  REGION 1..124: note = Synthetic Construct
QVQLVQSGAEVKKPGASVKVSCKASGYDYTYYGISWVRQAPGQGLEWMGWISTYNDNTHYAPSLQGRVTMTTDTSTSTAYMELRSLRSDDTAVYYCARESYYGSGAHRGGMDVWGQGTTVTVSS

SEQ ID NO: 37 - protein, 454 aa, synthetic construct
  REGION 1..454: note = Synthetic Construct
QVQLVQSGAEVKKPGASVKVSCKASGYDYTYYGISWVRQAPGQGLEWMGWISTYNDNTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 17 aa, synthetic construct
  REGION 1..17: note = Synthetic Construct
WISTYNDNTHYAQKLQG

SEQ ID NO: 39 - protein, 124 aa, synthetic construct
  REGION 1..124: note = Synthetic Construct
QVQLVQSGAEVKKPGASVKVSCKASGYDYTYYGISWVRQAPGQGLEWMGWISTYNDNTHYAQKLQGRVTMTTDTSTSTAYMELRSLRSDDTAVYYCARESYYGSGAFRGGMDVWGQGTTVTVSS

SEQ ID NO: 40 - protein, 454 aa, synthetic construct
  REGION 1..454: note = Synthetic Construct
QVQLVQSGAEVKKPGASVKVSCKASGYDYTYYGISWVRQAPGQGLEWMGWISTYNDNTHYAQKLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 17 aa, synthetic construct
  REGION 1..17: note = Synthetic Construct
WISTYNTGTHYAPSLQG

SEQ ID NO: 42 - protein, 124 aa, synthetic construct
  REGION 1..124: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAFRGGMDVWGQGTTVTVSS

SEQ ID NO: 43 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 124 aa, synthetic construct
  REGION 1..124: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAHRGGMDVWGQGTTVTVSS

SEQ ID NO: 45 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 17 aa, synthetic construct
  REGION 1..17: note = Synthetic Construct
WISTYNTGTHYAQKLQG

SEQ ID NO: 47 - protein, 124 aa, synthetic construct
  REGION 1..124: note = Synthetic Construct
QVQLVQSGAEVKKPGASVKVSCKASGYDYTYYGISWVRQAPGQGLEWMGWISTYNTGTHYAQKLQGRVTMTTDTSTSTAYMELRSLRSDDTAVYYCARESYYGSGAFRGGMDVWGQGTTVTVSS

SEQ ID NO: 48 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAQKLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 124 aa, synthetic construct
  REGION 1..124: note = Synthetic Construct
QVQLVQSGAEVKKPGASVKVSCKASGYDYTYYGISWVRQAPGQGLEWMGWISTYNTGTHYAQKLQGRVTMTTDTSTSTAYMELRSLRSDDTAVYYCARESYYGSGAHRGGMDVWGQGTTVTVSS

SEQ ID NO: 50 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAQKLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 17 aa, synthetic construct
  REGION 1..17: note = Synthetic Construct
WISTYNTVTHYAPSLQG

SEQ ID NO: 52 - protein, 124 aa, synthetic construct
  REGION 1..124: note = Synthetic Construct
QVQLVQSGAEVKKPGASVKVSCKASGYDYTYYGISWVRQAPGQGLEWMGWISTYNTVTHYAPSLQGRVTMTTDTSTSTAYMELRSLRSDDTAVYYCARESYYGSGAFRGGMDVWGQGTTVTVSS

SEQ ID NO: 53 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTVTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 124 aa, synthetic construct
  REGION 1..124: note = Synthetic Construct
QVQLVQSGAEVKKPGASVKVSCKASGYDYTYYGISWVRQAPGQGLEWMGWISTYNTVTHYAPSLQGRVTMTTDTSTSTAYMELRSLRSDDTAVYYCARESYYGSGAHRGGMDVWGQGTTVTVSS

SEQ ID NO: 55 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTVTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 17 aa, synthetic construct
  REGION 1..17: note = Synthetic Construct
WISTYNTNTHYAPSLQG

SEQ ID NO: 57 - protein, 124 aa, synthetic construct
  REGION 1..124: note = Synthetic Construct
QVQLVQSGAEVKKPGASVKVSCKASGYDYTYYGISWVRQAPGQGLEWMGWISTYNTNTHYAPSLQGRVTMTTDTSTSTAYMELRSLRSDDTAVYYCARESYYGSGAFRGGMDVWGQGTTVTVSS

SEQ ID NO: 58 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTNTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 124 aa, synthetic construct
  REGION 1..124: note = Synthetic Construct
QVQLVQSGAEVKKPGASVKVSCKASGYDYTYYGISWVRQAPGQGLEWMGWISTYNTNTHYAPSLQGRVTMTTDTSTSTAYMELRSLRSDDTAVYYCARESYYGSGAHRGGMDVWGQGTTVTVSS

SEQ ID NO: 60 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTNTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 10 aa, synthetic construct
  REGION 1..10: note = Synthetic Construct
GYDFSYYGIS

SEQ ID NO: 62 - protein, 124 aa, synthetic construct
  REGION 1..124: note = Synthetic Construct
QVQLVQSGAEVKKPGASVKVSCKASGYDFSYYGISWVRQAPGQGLEWMGWISTYNDNTHYAPSLQGRVTMTTDTSTSTAYMELRSLRSDDTAVYYCARESYYGSGAFRGGMDVWGQGTTVTVSS

SEQ ID NO: 63 - protein, 454 aa, synthetic construct
  REGION 1..454: note = Synthetic Construct
QVQLVQSGAEVKKPGASVKVSCKASGYDFSYYGISWVRQAPGQGLEWMGWISTYNDNTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 124 aa, synthetic construct
  REGION 1..124: note = Synthetic Construct
QVQLVQSGAEVKKPGASVKVSCKASGYDFSYYGISWVRQAPGQGLEWMGWISTYNDNTHYAPSLQGRVTMTTDTSTSTAYMELRSLRSDDTAVYYCARESYYGSGAHRGGMDVWGQGTTVTVSS

SEQ ID NO: 65 - protein, 454 aa, synthetic construct
  REGION 1..454: note = Synthetic Construct
QVQLVQSGAEVKKPGASVKVSCKASGYDFSYYGISWVRQAPGQGLEWMGWISTYNDNTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 124 aa, synthetic construct
  REGION 1..124: note = Synthetic Construct
QVQLVQSGAEVKKPGASVKVSCKASGYDFSYYGISWVRQAPGQGLEWMGWISTYNDNTHYAQKLQGRVTMTTDTSTSTAYMELRSLRSDDTAVYYCARESYYGSGAFRGGMDVWGQGTTVTVSS

SEQ ID NO: 67 - protein, 454 aa, synthetic construct
  REGION 1..454: note = Synthetic Construct
QVQLVQSGAEVKKPGASVKVSCKASGYDFSYYGISWVRQAPGQGLEWMGWISTYNDNTHYAQKLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 124 aa, synthetic construct
  REGION 1..124: note = Synthetic Construct
QVQLVQSGAEVKKPGASVKVSCKASGYDFSYYGISWVRQAPGQGLEWMGWISTYNDNTHYAQKLQGRVTMTTDTSTSTAYMELRSLRSDDTAVYYCARESYYGSGAHRGGMDVWGQGTTVTVSS

SEQ ID NO: 69 - protein, 454 aa, synthetic construct
  REGION 1..454: note = Synthetic Construct
QVQLVQSGAEVKKPGASVKVSCKASGYDFSYYGISWVRQAPGQGLEWMGWISTYNDNTHYAQKLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 124 aa, synthetic construct
  REGION 1..124: note = Synthetic Construct
QVQLVQSGAEVKKPGASVKVSCKASGYDFSYYGISWVRQAPGQGLEWMGWISTYNTGTHYAPSLQGRVTMTTDTSTSTAYMELRSLRSDDTAVYYCARESYYGSGAFRGGMDVWGQGTTVTVSS

SEQ ID NO: 71 - protein, 454 aa, synthetic construct
  REGION 1..454: note = Synthetic Construct
QVQLVQSGAEVKKPGASVKVSCKASGYDFSYYGISWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 124 aa, synthetic construct
  REGION 1..124: note = Synthetic Construct
QVQLVQSGAEVKKPGASVKVSCKASGYDFSYYGISWVRQAPGQGLEWMGWISTYNTGTHYAPSLQGRVTMTTDTSTSTAYMELRSLRSDDTAVYYCARESYYGSGAHRGGMDVWGQGTTVTVSS

SEQ ID NO: 73 - protein, 454 aa, synthetic construct
  REGION 1..454: note = Synthetic Construct
QVQLVQSGAEVKKPGASVKVSCKASGYDFSYYGISWVRQAPGQGLEWMGWISTYNTGTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 124 aa, synthetic construct
  REGION 1..124: note = Synthetic Construct
QVQLVQSGAEVKKPGASVKVSCKASGYDFSYYGISWVRQAPGQGLEWMGWISTYNTGTHYAQKLQGRVTMTTDTSTSTAYMELRSLRSDDTAVYYCARESYYGSGAFRGGMDVWGQGTTVTVSS

SEQ ID NO: 75 - protein, 454 aa, synthetic construct
  REGION 1..454: note = Synthetic Construct
QVQLVQSGAEVKKPGASVKVSCKASGYDFSYYGISWVRQAPGQGLEWMGWISTYNTGTHYAQKLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 124 aa, synthetic construct
  REGION 1..124: note = Synthetic Construct
QVQLVQSGAEVKKPGASVKVSCKASGYDFSYYGISWVRQAPGQGLEWMGWISTYNTGTHYAQKLQGRVTMTTDTSTSTAYMELRSLRSDDTAVYYCARESYYGSGAHRGGMDVWGQGTTVTVSS

SEQ ID NO: 77 - protein, 454 aa, synthetic construct
  REGION 1..454: note = Synthetic Construct
QVQLVQSGAEVKKPGASVKVSCKASGYDFSYYGISWVRQAPGQGLEWMGWISTYNTGTHYAQKLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 124 aa, synthetic construct
  REGION 1..124: note = Synthetic Construct
QVQLVQSGAEVKKPGASVKVSCKASGYDFSYYGISWVRQAPGQGLEWMGWISTYNTVTHYAPSLQGRVTMTTDTSTSTAYMELRSLRSDDTAVYYCARESYYGSGAFRGGMDVWGQGTTVTVSS

SEQ ID NO: 79 - protein, 454 aa, synthetic construct
  REGION 1..454: note = Synthetic Construct
QVQLVQSGAEVKKPGASVKVSCKASGYDFSYYGISWVRQAPGQGLEWMGWISTYNTVTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 124 aa, synthetic construct
  REGION 1..124: note = Synthetic Construct
QVQLVQSGAEVKKPGASVKVSCKASGYDFSYYGISWVRQAPGQGLEWMGWISTYNTVTHYAPSLQGRVTMTTDTSTSTAYMELRSLRSDDTAVYYCARESYYGSGAHRGGMDVWGQGTTVTVSS

SEQ ID NO: 81 - protein, 454 aa, synthetic construct
  REGION 1..454: note = Synthetic Construct
QVQLVQSGAEVKKPGASVKVSCKASGYDFSYYGISWVRQAPGQGLEWMGWISTYNTVTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 124 aa, synthetic construct
  REGION 1..124: note = Synthetic Construct
QVQLVQSGAEVKKPGASVKVSCKASGYDFSYYGISWVRQAPGQGLEWMGWISTYNTNTHYAPSLQGRVTMTTDTSTSTAYMELRSLRSDDTAVYYCARESYYGSGAFRGGMDVWGQGTTVTVSS

SEQ ID NO: 83 - protein, 454 aa, synthetic construct
  REGION 1..454: note = Synthetic Construct
QVQLVQSGAEVKKPGASVKVSCKASGYDFSYYGISWVRQAPGQGLEWMGWISTYNTNTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 124 aa, synthetic construct
  REGION 1..124: note = Synthetic Construct
QVQLVQSGAEVKKPGASVKVSCKASGYDFSYYGISWVRQAPGQGLEWMGWISTYNTNTHYAPSLQGRVTMTTDTSTSTAYMELRSLRSDDTAVYYCARESYYGSGAHRGGMDVWGQGTTVTVSS

SEQ ID NO: 85 - protein, 454 aa, synthetic construct
  REGION 1..454: note = Synthetic Construct
QVQLVQSGAEVKKPGASVKVSCKASGYDFSYYGISWVRQAPGQGLEWMGWISTYNTNTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 10 aa, synthetic construct
  REGION 1..10: note = Synthetic Construct
GYDFTYYGVS

SEQ ID NO: 87 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNDNTHYAPSLQGRVTMTTDTSTSTAYMELRSLRSDDTAVYYCARESYYGSGAFRGGMDVWGQGTTVTVSS

SEQ ID NO: 88 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNDNTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNDNTHYAPSLQGRVTMTTDTSTSTAYMELRSLRSDDTAVYYCARESYYGSGAHRGGMDVWGQGTTVTVSS

SEQ ID NO: 90 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNDNTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNDNTHYAQKLQGRVTMTTDTSTSTAYMELRSLRSDDTAVYYCARESYYGSGAFRGGMDVWGQGTTVTVSS

SEQ ID NO: 92 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNDNTHYAQKLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNDNTHYAQKLQGRVTMTTDTSTSTAYMELRSLRSDDTAVYYCARESYYGSGAHRGGMDVWGQGTTVTVSS

SEQ ID NO: 94 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNDNTHYAQKLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNTGTHYAPSLQGRVTMTTDTSTSTAYMELRSLRSDDTAVYYCARESYYGSGAFRGGMDVWGQGTTVTVSS

SEQ ID NO: 96 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNTGTHYAPSLQGRVTMTTDTSTSTAYMELRSLRSDDTAVYYCARESYYGSGAHRGGMDVWGQGTTVTVSS

SEQ ID NO: 98 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNTGTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNTGTHYAQKLQGRVTMTTDTSTSTAYMELRSLRSDDTAVYYCARESYYGSGAHRGGMDVWGQGTTVTVSS

SEQ ID NO: 100 - protein, 453 aa, synthetic construct
  REGION 1..453: note = Synthetic Construct
QVQLVQSGAEVKKPGASVKVSCKASGYDFTYYGVSWVRQAPGQGLEWMGWISTYNTGTHYAQKLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNTVTHYAPSLQGRVTMTTDTSTSTAYMELRSLRSDDTAVYYCARESYYGSGAFRGGMDVWGQGTTVTVSS

SEQ ID NO: 102 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNTVTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNTVTHYAPSLQGRVTMTTDTSTSTAYMELRSLRSDDTAVYYCARESYYGSGAHRGGMDVWGQGTTVTVSS

SEQ ID NO: 104 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNTVTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNTNTHYAPSLQGRVTMTTDTSTSTAYMELRSLRSDDTAVYYCARESYYGSGAFRGGMDVWGQGTTVTVSS

SEQ ID NO: 106 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNTNTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 124 aa, synthetic construct
  REGION 1..124: note = Synthetic Construct
QVQLVQSGAEVKKPGASVKVSCKASGYDFTYYGVSWVRQAPGQGLEWMGWISTYNTNTHYAPSLQGRVTMTTDTSTSTAYMELRSLRSDDTAVYYCARESYYGSGAHRGGMDVWGQGTTVTVSS

SEQ ID NO: 108 - protein, 454 aa, synthetic construct
  REGION 1..454: note = Synthetic Construct
QVQLVQSGAEVKKPGASVKVSCKASGYDFTYYGVSWVRQAPGQGLEWMGWISTYNTNTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 10 aa, synthetic construct
  REGION 1..10: note = Synthetic Construct
GYDFTYYGIG

SEQ ID NO: 110 - protein, 124 aa, synthetic construct
  REGION 1..124: note = Synthetic Construct
QVQLVQSGAEVKKPGASVKVSCKASGYDFTYYGIGWVRQAPGQGLEWMGWISTYNTGTHYAPSLQGRVTMTTDTSTSTAYMELRSLRSDDTAVYYCARESYYGSGAHRGGMDVWGQGTTVTVSS

SEQ ID NO: 111 - protein, 454 aa, synthetic construct
  REGION 1..454: note = Synthetic Construct
QVQLVQSGAEVKKPGASVKVSCKASGYDFTYYGIGWVRQAPGQGLEWMGWISTYNTGTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 124 aa, synthetic construct
  REGION 1..124: note = Synthetic Construct
QVQLVQSGAEVKKPGASVKVSCKASGYDFTYYGIGWVRQAPGQGLEWMGWISTYNTGTHYAQKLQGRVTMTTDTSTSTAYMELRSLRSDDTAVYYCARESYYGSGAFRGGMDVWGQGTTVTVSS

SEQ ID NO: 113 - protein, 454 aa, synthetic construct
  REGION 1..454: note = Synthetic Construct
QVQLVQSGAEVKKPGASVKVSCKASGYDFTYYGIGWVRQAPGQGLEWMGWISTYNTGTHYAQKLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 124 aa, synthetic construct
  REGION 1..124: note = Synthetic Construct
QVQLVQSGAEVKKPGASVKVSCKASGYDFTYYGIGWVRQAPGQGLEWMGWISTYNTGTHYAQKLQGRVTMTTDTSTSTAYMELRSLRSDDTAVYYCARESYYGSGAHRGGMDVWGQGTTVTVSS

SEQ ID NO: 115 - protein, 454 aa, synthetic construct
  REGION 1..454: note = Synthetic Construct
QVQLVQSGAEVKKPGASVKVSCKASGYDFTYYGIGWVRQAPGQGLEWMGWISTYNTGTHYAQKLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 124 aa, synthetic construct
  REGION 1..124: note = Synthetic Construct
QVQLVQSGAEVKKPGASVKVSCKASGYDFTYYGIGWVRQAPGQGLEWMGWISTYNTVTHYAPSLQGRVTMTTDTSTSTAYMELRSLRSDDTAVYYCARESYYGSGAFRGGMDVWGQGTTVTVSS

SEQ ID NO: 117 - protein, 454 aa, synthetic construct
  REGION 1..454: note = Synthetic Construct
QVQLVQSGAEVKKPGASVKVSCKASGYDFTYYGIGWVRQAPGQGLEWMGWISTYNTVTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 124 aa, synthetic construct
  REGION 1..124: note = Synthetic Construct
QVQLVQSGAEVKKPGASVKVSCKASGYDFTYYGIGWVRQAPGQGLEWMGWISTYNTVTHYAPSLQGRVTMTTDTSTSTAYMELRSLRSDDTAVYYCARESYYGSGAHRGGMDVWGQGTTVTVSS

SEQ ID NO: 119 - protein, 454 aa, synthetic construct
  REGION 1..454: note = Synthetic Construct
QVQLVQSGAEVKKPGASVKVSCKASGYDFTYYGIGWVRQAPGQGLEWMGWISTYNTVTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 124 aa, synthetic construct
  REGION 1..124: note = Synthetic Construct
QVQLVQSGAEVKKPGASVKVSCKASGYDFTYYGIGWVRQAPGQGLEWMGWISTYNTNTHYAPSLQGRVTMTTDTSTSTAYMELRSLRSDDTAVYYCARESYYGSGAFRGGMDVWGQGTTVTVSS

SEQ ID NO: 121 - protein, 454 aa, synthetic construct
  REGION 1..454: note = Synthetic Construct
QVQLVQSGAEVKKPGASVKVSCKASGYDFTYYGIGWVRQAPGQGLEWMGWISTYNTNTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 124 aa, synthetic construct
  REGION 1..124: note = Synthetic Construct
QVQLVQSGAEVKKPGASVKVSCKASGYDFTYYGIGWVRQAPGQGLEWMGWISTYNTNTHYAPSLQGRVTMTTDTSTSTAYMELRSLRSDDTAVYYCARESYYGSGAHRGGMDVWGQGTTVTVSS

SEQ ID NO: 123 - protein, 454 aa, synthetic construct
  REGION 1..454: note = Synthetic Construct
QVQLVQSGAEVKKPGASVKVSCKASGYDFTYYGIGWVRQAPGQGLEWMGWISTYNTNTHYAPSLQGRVTMTTDTSTSTAYMELRSLRSDDTAVYYCARESYYGSGAHRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 17 aa, synthetic construct
  REGION 1..17: note = Synthetic Construct
WISTYNTNTHYARMLQG

SEQ ID NO: 125 - protein, 15 aa, synthetic construct
  REGION 1..15: note = Synthetic Construct
ESYYGSGAYRGGMDV

SEQ ID NO: 126 - protein, 124 aa, synthetic construct
  REGION 1..124: note = Synthetic Construct
QVQLVQSGAEVKKPGASVKVSCKASGYDFTYYGISWVRQAPGQGLEWMGWISTYNTNTHYARMLQGRVTMTTDTSTSTAYMELRSLRSDDTAVYYCARESYYGSGAYRGGMDVWGQGTTVTVSS

SEQ ID NO: 127 - protein, 454 aa, synthetic construct
  REGION 1..454: note = Synthetic Construct
QVQLVQSGAEVKKPGASVKVSCKASGYDFTYYGISWVRQAPGQGLEWMGWISTYNTNTHYARMLQGRVTMTTDTSTSTAYMELRSLRSDDTAVYYCARESYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 10 aa, synthetic construct
  REGION 1..10: note = Synthetic Construct
GYTFTSYGIS

SEQ ID NO: 129 - protein, 17 aa, synthetic construct
  REGION 1..17: note = Synthetic Construct
WISAYNGNTNYAQKLQG

SEQ ID NO: 130 - protein, 15 aa, synthetic construct
  REGION 1..15: note = Synthetic Construct
ESYYGSEAFRAGMDV

SEQ ID NO: 131 - protein, 124 aa, synthetic construct
  REGION 1..124: note = Synthetic Construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESYYGSEAFRAGMDVWGQGTTVTVSS

SEQ ID NO: 132 - protein, 454 aa, synthetic construct
  REGION 1..454: note = Synthetic Construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESYYGSEAFRAGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 15 aa, synthetic construct
  REGION 1..15: note = Synthetic Construct
ESSYGSEAFRGGMDV

SEQ ID NO: 134 - protein, 124 aa, synthetic construct
  REGION 1..124: note = Synthetic Construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESSYGSEAFRGGMDVWGQGTTVTVSS

SEQ ID NO: 135 - protein, 454 aa, synthetic construct
  REGION 1..454: note = Synthetic Construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESSYGSEAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 15 aa, synthetic construct
  REGION 1..15: note = Synthetic Construct
ESSYGSEAFRAGMDV

SEQ ID NO: 137 - protein, 124 aa, synthetic construct
  REGION 1..124: note = Synthetic Construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESSYGSEAFRAGMDVWGQGTTVTVSS

SEQ ID NO: 138 - protein, 454 aa, synthetic construct
  REGION 1..454: note = Synthetic Construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESSYGSEAFRAGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 10 aa, synthetic construct
  REGION 1..10: note = Synthetic Construct
GFTFSSYAMS

SEQ ID NO: 140 - protein, 17 aa, synthetic construct
  REGION 1..17: note = Synthetic Construct
AISGSGGSTYYADSVKG

SEQ ID NO: 141 - protein, 124 aa, synthetic construct
  REGION 1..124: note = Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARESYYGSEAFRAGMDVWGQGTTVTVSS

SEQ ID NO: 142 - protein, 454 aa, synthetic construct
  REGION 1..454: note = Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARESYYGSEAFRAGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 124 aa, synthetic construct
  REGION 1..124: note = Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARESSYGSEAFRGGMDVWGQGTTVTVSS

SEQ ID NO: 144 - protein, 454 aa, synthetic construct
  REGION 1..454: note = Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARESSYGSEAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 124 aa, synthetic construct
  REGION 1..124: note = Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARESSYGSEAFRAGMDVWGQGTTVTVSS

SEQ ID NO: 146 - protein, 454 aa, synthetic construct
  REGION 1..454: note = Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARESSYGSEAFRAGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 10 aa, synthetic construct
  REGION 1..10: note = Synthetic Construct
GYTFTGYYMH

SEQ ID NO: 148 - protein, 17 aa, synthetic construct
  REGION 1..17: note = Synthetic Construct
WINPNSGGTNYAQKFQG

SEQ ID NO: 149 - protein, 124 aa, synthetic construct
  REGION 1..124: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARESYYGSEAFRAGMDVWGQGTTVTVSS

SEQ ID NO: 150 - protein, 454 aa, synthetic construct
  REGION 1..454: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARESYYGSEAFRAGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 124 aa, synthetic construct
  REGION 1..124: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARESSYGSEAFRGGMDVWGQGTTVTVSS

SEQ ID NO: 152 - protein, 454 aa, synthetic construct
  REGION 1..454: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARESSYGSEAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 124 aa, synthetic construct
  REGION 1..124: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARESSYGSEAFRAGMDVWGQGTTVTVSS

SEQ ID NO: 154 - protein, 454 aa, synthetic construct
  REGION 1..454: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARESSYGSEAFRAGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 107 aa, synthetic construct
  REGION 1..107: note = Synthetic Construct
GQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 454 aa, synthetic construct
  REGION 1..454: note = Synthetic Construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 15 aa, synthetic construct
  REGION 1..15: note = Synthetic Construct
EPKSSDKTHTCPPCP

SEQ ID NO: 158 - protein, 107 aa, synthetic construct
  REGION 1..107: note = Synthetic Construct
GQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 159 - protein, 232 aa, synthetic construct
  REGION 1..232: note = Synthetic Construct
EPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 160 - protein, 107 aa, synthetic construct
  REGION 1..107: note = Synthetic Construct
GQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 161 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 162 - protein, 107 aa, synthetic construct
  REGION 1..107: note = Synthetic Construct
GQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 163 - protein, 232 aa, synthetic construct
  REGION 1..232: note = Synthetic Construct
EPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 164 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTNTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 165 - protein, 454 aa, synthetic construct
  REGION 1..454: note = Synthetic Construct
QVQLVQSGAEVKKPGASVKVSCKASGYDFTYYGIGWVRQAPGQGLEWMGWISTYNTNTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 166 - protein, 454 aa, synthetic construct
  REGION 1..454: note = Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARESSYGSEAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 167 - protein, 10 aa, synthetic construct
  REGION 1..10: note = Synthetic Construct
GYSFTTYWLG

SEQ ID NO: 168 - protein, 17 aa, synthetic construct
  REGION 1..17: note = Synthetic Construct
IMSPVDSDIRYSPSFQG

SEQ ID NO: 169 - protein, 10 aa, synthetic construct
  REGION 1..10: note = Synthetic Construct
RRPGQGYFDF

SEQ ID NO: 170 - protein, 11 aa, synthetic construct
  REGION 1..11: note = Synthetic Construct
WGQGTLVTVSS

SEQ ID NO: 171 - protein, 119 aa, synthetic construct
  REGION 1..119: note = Synthetic Construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 172 - protein, 449 aa, synthetic construct
  REGION 1..449: note = Synthetic Construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 11 aa, synthetic construct
  REGION 1..11: note = Synthetic Construct
RASQGISSWLA

SEQ ID NO: 174 - protein, 7 aa, synthetic construct
  REGION 1..7: note = Synthetic Construct
AASSLQS

SEQ ID NO: 175 - protein, 9 aa, synthetic construct
  REGION 1..9: note = Synthetic Construct
QQYNIYPYT

SEQ ID NO: 176 - protein, 10 aa, synthetic construct
  REGION 1..10: note = Synthetic Construct
FGQGTKLEIK

SEQ ID NO: 177 - protein, 107 aa, synthetic construct
  REGION 1..107: note = Synthetic Construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 178 - protein, 214 aa, synthetic construct
  REGION 1..214: note = Synthetic Construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 15 aa, synthetic construct
  REGION 1..15: note = Synthetic Construct
EPKSCRKTHTCPRCP

SEQ ID NO: 180 - protein, 107 aa, synthetic construct
  REGION 1..107: note = Synthetic Construct
GQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 181 - protein, 449 aa, synthetic construct
  REGION 1..449: note = Synthetic Construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPRCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 182 - protein, 15 aa, synthetic construct
  REGION 1..15: note = Synthetic Construct
EPKSCEKTHTCPECP

SEQ ID NO: 183 - protein, 107 aa, synthetic construct
  REGION 1..107: note = Synthetic Construct
GQPREPQVYTLPPSREEMTKNQVSLTCEVKGFYPSDIAVEWESNGQPENNYKTTPPVLDSDGSFFLYSKLTVDKSRWQQGNVFSCSVLHEALHSHYTQKSLSLSPGK

SEQ ID NO: 184 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPECPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVLHEALHSHYTQKSLSLSPGK

SEQ ID NO: 185 - protein, 107 aa, synthetic construct
  REGION 1..107: note = Synthetic Construct
GQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 186 - protein, 449 aa, synthetic construct
  REGION 1..449: note = Synthetic Construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 187 - protein, 107 aa, synthetic construct
  REGION 1..107: note = Synthetic Construct
GQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 188 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 189 - protein, 15 aa, synthetic construct
  REGION 1..15: note = Synthetic Construct
EPKSCRKTHTCPPCP

SEQ ID NO: 190 - protein, 449 aa, synthetic construct
  REGION 1..449: note = Synthetic Construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 191 - protein, 15 aa, synthetic construct
  REGION 1..15: note = Synthetic Construct
EPKSCEKTHTCPPCP

SEQ ID NO: 192 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVLHEALHSHYTQKSLSLSPGK

SEQ ID NO: 193 - protein, 449 aa, synthetic construct
  REGION 1..449: note = Synthetic Construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 194 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVLHEALHSHYTQKSLSLSPGK

SEQ ID NO: 195 - protein, 98 aa, synthetic construct
  REGION 1..98: note = Synthetic Construct
ASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKV

SEQ ID NO: 196 - protein, 449 aa, synthetic construct
  REGION 1..449: note = Synthetic Construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 197 - protein, 107 aa, synthetic construct
  REGION 1..107: note = Synthetic Construct
RTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 198 - protein, 214 aa, synthetic construct
  REGION 1..214: note = Synthetic Construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 199 - protein, 98 aa, synthetic construct
  REGION 1..98: note = Synthetic Construct
ASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKV

SEQ ID NO: 200 - protein, 454 aa, synthetic construct
  REGION 1..454: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 201 - protein, 107 aa, synthetic construct
  REGION 1..107: note = Synthetic Construct
RTVAAPSVFIFPPSDEQLKSGTASVSCLLNNFYPREAKVQWKVDNALQSGNSQESVTEQDSKDSTYSLKSTLTLSKADYEKHKVYACEVTHQGLSSPVTKSFNRGEC

SEQ ID NO: 202 - protein, 214 aa, synthetic construct
  REGION 1..214: note = Synthetic Construct
DIQMTQSPSSLSASVGDRVTITCRASQSVSSYLAWYQQKPGKAPKLLIYDASNRATGVPSRFSGSGSGTDFTLTISSLQPEDFATYYCQQRSNWPWTFGQGTKVEIKRTVAAPSVFIFPPSDEQLKSGTASVSCLLNNFYPREAKVQWKVDNALQSGNSQESVTEQDSKDSTYSLKSTLTLSKADYEKHKVYACEVTHQGLSSPVTKSFNRGEC

SEQ ID NO: 203 - protein, 449 aa, synthetic construct
  REGION 1..449: note = Synthetic Construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 204 - protein, 10 aa, synthetic construct
  REGION 1..10: note = Synthetic Construct
DKTHTCPPCP

SEQ ID NO: 205 - protein, 441 aa, synthetic construct
  REGION 1..441: note = Synthetic Construct
DIQMTQSPSSLSASVGDRVTITCRASQSVSSYLAWYQQKPGKAPKLLIYDASNRATGVPSRFSGSGSGTDFTLTISSLQPEDFATYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 206 - protein, 5 aa, synthetic construct
  REGION 1..5: note = Synthetic Construct
EPKSC

SEQ ID NO: 207 - protein, 227 aa, synthetic construct
  REGION 1..227: note = Synthetic Construct
QVQLVQSGAEVKKPGASVKVSCKASGYDYTYYGISWVRQAPGQGLEWMGWISTYNTGTHYAPSLQGRVTMTTDTSTSTAYMELRSLRSDDTAVYYCARESYYGSGAFRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 208 - protein, 183 aa, synthetic construct
  REGION 1..183: note = Synthetic Construct
MGALKGQEFAPSHQQVYAPLRADGDKPRAHLTVVRQTPTQHFKNQFPALHWEHELGLAFTKNRMNYTNKFLLIPESGDYFIYSQVTFRGMTSESSEIRQAGRPNKPDSITVVITKVTDSYPEPTQLLMGTKSVSEVGSNWFQPIYLGAMFSLQEGDKLMVNVSDISLVDYTKEDKTFFGAFLL

SEQ ID NO: 209 - protein, 182 aa, synthetic construct
  REGION 1..182: note = Synthetic Construct
TGLKGQEFAPSHQQVYAPLRADGDKPRAHLTVVRQTPTQHLKNQFPALHWEHELGLAFTKNRMNYTNKFLLIPESGDYFVYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 210 - protein, 181 aa, synthetic construct
  REGION 1..181: note = Synthetic Construct
TGLKGREFGPSQQRAYMPLRADGNKPRAHLTAVKQTPTQPLRNHFPALHWEHELGLAFTKNRMNYTNKFLVIPESGDYFVYSQVTFRGTTSECGVINQRRRQTKPDSIVVVITKVTDNYPEPAQLLTGTKSVCEMGNWFQPIYLGAMFSLEEGDKLMVNVSDVSLVDYTKEDKTFFGAFLL

SEQ ID NO: 211 - protein, 182 aa, synthetic construct
  REGION 1..182: note = Synthetic Construct
GLEGAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITVVITKVADSYPEPARLLTGSKSVCEISNNWFQSLYLGAMFSLEEGDRLMVNVSDISLVDYTKEDKTFFGAFLL

SEQ ID NO: 212 - protein, 179 aa, synthetic construct
  REGION 1..179: note = Synthetic Construct
TGVTEERSAPSAQPVYTPSRDKPKAHLTIMRQTPVPHLKNELAALHWENNLGMAFTKNRMNYTNKFLVIPESGDYFIYSQITFRGTTSECGDISRVRRPKKPDSITVVITKVADSYPEPAHLLTGTKSVCEISSNWFQPIYLGAMFSLEEGDRLMVNVSDISLVDYTKEDKTFFGAFLI

SEQ ID NO: 213 - protein, 328 aa, synthetic construct
  REGION 1..328: note = Synthetic Construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 214 - protein, 328 aa, synthetic construct
  REGION 1..328: note = Synthetic Construct
MCHQQLVISWFSLVFLASPLMAIWELKKDVYVVELDWYPDAPGEMVVLTCDTPEEDGITWTLDQSGEVLGSGKTLTIQVKEFGDAGQYTCHKGGEALSHSLLLLHKKEDGIWSTDVLKDQKEPKNKTFLRCEAKNYSGRFTCWWLTTISTDLTFSVKSSRGSSNPQGVTCGAVTLSAERVRGDNKEYEYSVECQEDSACPAAEERLPIEVMVDAIHKLKYENYTSSFFIRDIIKPDPPKNLQLKPLKNSRQVEVSWEYPDTWSTPHSYFSLTFCIQVQGKSKREKKDRIFTDKTSATVICRKNASFSVQAQDRYYSSSWSEWASVPCS

SEQ ID NO: 215 - protein, 253 aa, synthetic construct
  REGION 1..253: note = Synthetic Construct
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 216 - protein, 253 aa, synthetic construct
  REGION 1..253: note = Synthetic Construct
MWPPGSASQPPPSPATATGLHPAARPEPLQCRLSMCPARSLLLVATLVLLDYLSLARNLSVATPGPEMFPCLHHSQNLLKAASNTLQKARQILEFYPCTSEEIDHEDITKDKTSTVDACLPLELIKNESCLNSRETSFITNGSCLASRKTSFMMALCLRSIYEDLKMYQVEFKTMNAKLLRDPKRQIFLDQNILGVIDELMQALNFNSETVPQKSSLEEPDFYKTKIKLCILLHAFRIRAVTIDRVMSYLNAS

SEQ ID NO: 217 - protein, 189 aa, synthetic construct
  REGION 1..189: note = Synthetic Construct
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 218 - protein, 189 aa, synthetic construct
  REGION 1..189: note = Synthetic Construct
MLGSRAVMLLLLLSWTAQGRAVPGGSSPAWAQCQQLSQKLCTLAWSAHPLVGHMDLREEGDEETTNDVPHIQCGDGCDPQGLRDNSQFCLQRIRQGLIFYEKLLGSDIFTGEPSLLPDSPVGQLHASLLGLSQLLQPEGHHWETQQIPSPSPSQPWQRLLLRFKILRSLQAFVAVAARVFAHGAATLSP

SEQ ID NO: 219 - protein, 315 aa, synthetic construct
  REGION 1..315: note = Synthetic Construct
IWELEKDVYVVELDWYPDAPGETVVLTCDTSEEDGITWTSEQSNKVLGSGKTLTILVKEFGDAGQYTCHKGDKVLGHSKVLLHKKEDGIWSTDILKDQKEPKSKTFLKCEAKNYSGHFTCWWLTAISRDVKFSVKSNRGSSDPQGVTCGVPTLSAERVSVDHTEYKFSVECQEDNACPAAEESLHLEVMLDAIHKLKYENYTSSFFIRDIVKPDPPQNLQMKPLKNSRHVEVSWEYPDTWSTPHSYFSLTFCVQVQNKNKKEKKDRLCVDKTSATVMCHKDAKIRVQARDRYYSSSWSEWAFVSCNGGGENLYFQ

SEQ ID NO: 220 - protein, 206 aa, synthetic construct
  REGION 1..206: note = Synthetic Construct
RNLPVATPDLGLFQCLNHSQNLLRAVSDTLHKARQTLEFYSCTSEEIDHEDITKDKTSTVKACLPLELTKNESCLASRETSLIINGSCLSSGKTSSMVTLCLSSIYEDLKMYQVEFMAMNAKLLMDSKRQIFLDQDMLGAIEELMQALNSNSGAVPQNPSLEELDFYKTKIKLCILLHAFRIRVVTIDRMMSYLSSSGGGENLYFQ

SEQ ID NO: 221 - protein, 181 aa, synthetic construct
  REGION 1..181: note = Synthetic Construct
RAVPGDSSPAWTQCQQLSQKLCTLAWSAHPQMGPMELLREEGDEETTSDDVPHIQCEDGCDPQGLRDNTQFCLQRIRQGLFFYEKLLGSDIFTGEPALLPDGPVGQLHASLLGLSQLLQPEGHHRETPQTPSLHPSQPWQRRLLRFKILRSLQAFLAVAARVFAHGAATLSPGGGENLYFQ

SEQ ID NO: 222 - protein, 313 aa, synthetic construct
  REGION 1..313: note = Synthetic Construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 223 - protein, 193 aa, synthetic construct
  REGION 1..193: note = Synthetic Construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 224 - protein, 177 aa, synthetic construct
  REGION 1..177: note = Synthetic Construct
LAVPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTA

SEQ ID NO: 225 - protein, 313 aa, synthetic construct
  REGION 1..313: note = Synthetic Construct
MWELEKDVYVVEVDWRPDAPGETVTLTCDSPEEDDITWTSDQRRGVIGSGKTLTITVREFLDAGQYTCHRGGETLSHSHLLLHKKENGIWSTEILKNFKNKTFLKCEAPNYSGRFTCSWLVHRNTDLKFNIKSSSSSPESRAVTCGRASLSAEKVTLNQRDYEKYSVACQEDVTCPTAEETLPIELVVEAQQQNKYENYSTSFFIRDIIKPDPPKNLQVKPLKNSQVEVSWEYPDSWSTPHSYFSLKFFVRIQRKKEKTKETEEECNQKGAFLVEKTSAEVQCKGANICVQAQDRYYNSSCSKWTCVPCRGRS

SEQ ID NO: 226 - protein, 193 aa, synthetic construct
  REGION 1..193: note = Synthetic Construct
RVIPVSGPAKCLNQSQNLLKTTDDMVRTAREKLKHYSCTAGDIDHEDITRDKTSTLEACLPLELHKNESCLATKETSSIIRGSCLPPQKTSLMMTLCLGSIYEDLKMYQSEFQAINAALQSHNHQQITLDRNMLMAIDELMRSLNHSGETLHQKAPMGEADPYRVKMKLCILLHAFSTRVMTINRVMNYLSSS

SEQ ID NO: 227 - protein, 177 aa, synthetic construct
  REGION 1..177: note = Synthetic Construct
LAVPRSSSPDWAQCQQLSRNLCTLAWSAHTPVGQMDLLREEGEEETKSDVPRIQCGDGCDPQGLKDNSQFCLQRIRQGLVFYKHLLDSDIFTGEPSLLPDSPVDQLHTSLLGLSQLLQPEDHHWETQQMPRLSPSQQWQRSLLRSKILRSLQAFLAIAARVFAHGAATLTEPLVPTA

SEQ ID NO: 228 - DNA, 372 bp, unidentified
  source 1..372: mol_type = other DNA
caggttcagctggtgcagtctggagctgaggtgaagaagcctggggcctcagtgaaggtctcctgcaaggcctctggttacgactatacctactatggtatcagctgggtgcgacaggcccctggacaagggcttgagtggatgggatggatcagcacttacaatactggcacacactatgcacccagcctccagggcagagtcaccatgaccacagacacatccaccagcacagcctacatggagctgaggagcctgagatctgacgacaccgccgtgtattactgtgcaagagagagctactatggttccggggctttcagagggggtatggacgtctggggccaagggaccacagtcaccgtctcctca

SEQ ID NO: 229 - DNA, 321 bp, synthetic construct
  source 1..321: mol_type = other DNA
gacatccagatgacccagtctccatcctccctgtctgcatctgtaggagacagagtcaccatcacttgcagggccagtcagagtgttagcagctacctggcctggtatcagcagaaaccagggaaagcccctaagctcctgatctatgatgcatccaacagggccactggggtcccatcaaggttcagtggcagtggatctgggacagatttcactctcaccatcagcagtctgcaacctgaagattttgcaacttactactgtcagcagcgtagcaactggccctggaccttcggccaagggaccaaggtggaaatcaaa

SEQ ID NO: 230 - DNA, 372 bp, synthetic construct
  source 1..372: mol_type = other DNA
gaggtgcagctgttggagtctgggggaggcttggtgcagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacactgtatctgcaaatgaacagcctgagagccgaggacaccgccgtgtattactgtgcaagagagagctcctatggttccgaggctttcagagggggtatggacgtctggggccaagggaccacagtcaccgtctcctca

SEQ ID NO: 231 - DNA, 1362 bp, synthetic construct
  source 1..1362: mol_type = other DNA
caggttcagctggtgcagtctggagctgaggtgaagaagcctggggcctcagtgaaggtctcctgcaaggcctctggttacgactatacctactatggtatcagctgggtgcgacaggcccctggacaagggcttgagtggatgggatggatcagcacttacaatactggcacacactatgcacccagcctccagggcagagtcaccatgaccacagacacatccaccagcacagcctacatggagctgaggagcctgagatctgacgacaccgccgtgtattactgtgcaagagagagctactatggttccggggctttcagagggggtatggacgtctggggccaagggaccacagtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtcccccggaaaa

SEQ ID NO: 232 - DNA, 1362 bp, synthetic construct
  source 1..1362: mol_type = other DNA
gaggtgcagctgttggagtctgggggaggcttggtgcagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacactgtatctgcaaatgaacagcctgagagccgaggacaccgccgtgtattactgtgcaagagagagctcctatggttccgaggctttcagagggggtatggacgtctggggccaagggaccacagtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgctgcatgaggctctgcacagccactacacgcagaagagcctctccctgtcccccggaaaa

SEQ ID NO: 233 - DNA, 642 bp, synthetic construct
  source 1..642: mol_type = other DNA
gacatccagatgacccagtctccatcctccctgtctgcatctgtaggagacagagtcaccatcacttgcagggccagtcagagtgttagcagctacctggcctggtatcagcagaaaccagggaaagcccctaagctcctgatctatgatgcatccaacagggccactggggtcccatcaaggttcagtggcagtggatctgggacagatttcactctcaccatcagcagtctgcaacctgaagattttgcaacttactactgtcagcagcgtagcaactggccctggaccttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 234 - DNA, 681 bp, synthetic construct
  source 1..681: mol_type = other DNA
caggttcagctggtgcagtctggagctgaggtgaagaagcctggggcctcagtgaaggtctcctgcaaggcctctggttacgactatacctactatggtatcagctgggtgcgacaggcccctggacaagggcttgagtggatgggatggatcagcacttacaatactggcacacactatgcacccagcctccagggcagagtcaccatgaccacagacacatccaccagcacagcctacatggagctgaggagcctgagatctgacgacaccgccgtgtattactgtgcaagagagagctactatggttccggggctttcagagggggtatggacgtctggggccaagggaccacagtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 235 - DNA, 1323 bp, synthetic construct
  source 1..1323: mol_type = other DNA
gacatccagatgacccagtctccatcctccctgtctgcatctgtaggagacagagtcaccatcacttgcagggccagtcagagtgttagcagctacctggcctggtatcagcagaaaccagggaaagcccctaagctcctgatctatgatgcatccaacagggccactggggtcccatcaaggttcagtggcagtggatctgggacagatttcactctcaccatcagcagtctgcaacctgaagattttgcaacttactactgtcagcagcgtagcaactggccctggaccttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggaggagatgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgctgcatgaggctctgcacagccactacacgcagaagagcctctccctgtccccgggaaaa

SEQ ID NO: 236 - DNA, 1347 bp, synthetic construct
  source 1..1347: mol_type = other DNA
gaggtgcagctggtgcagtctggagcagaggtgaaaaagcccggggagtctctgaagatctcctgtaagggttctggatacagctttaccacctactggctgggctgggtgcgccagatgcccgggaaaggcctggattggatagggatcatgagccctgtggactctgatattagatacagcccctccttccaaggccaggtcaccatgtcagtggacaagtccatcaccaccgcctacctgcagtggaatagcctgaaggcctccgacaccgccatgtattactgtgccagacgacgccctggtcaaggttatttcgatttttgggggcaaggaacattggtgaccgtcagttcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggaaaa

SEQ ID NO: 237 - DNA, 642 bp, synthetic construct
  source 1..642: mol_type = other DNA
gacatccagatgacccagtctccatcctcactgtctgcatctgtgggagacagagtcaccatcacttgtcgggccagtcagggtattagcagctggctggcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatatttatccctacaccttcggacaaggtacaaaactggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 238 - DNA, 357 bp, synthetic construct
  source 1..357: mol_type = other DNA
gaggtgcagctggtgcagtctggagcagaggtgaaaaagcccggggagtctctgaagatctcctgtaagggttctggatacagctttaccacctactggctgggctgggtgcgccagatgcccgggaaaggcctggattggatagggatcatgagccctgtggactctgatattagatacagcccctccttccaaggccaggtcaccatgtcagtggacaagtccatcaccaccgcctacctgcagtggaatagcctgaaggcctccgacaccgccatgtattactgtgccagacgacgccctggtcaaggttatttcgatttttgggggcaaggaacattggtgaccgtcagttca

SEQ ID NO: 239 - DNA, 321 bp, synthetic construct
  source 1..321: mol_type = other DNA
gacatccagatgacccagtctccatcctcactgtctgcatctgtgggagacagagtcaccatcacttgtcgggccagtcagggtattagcagctggctggcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatatttatccctacaccttcggacaaggtacaaaactggagatcaaa